(19) United States
(12) Patent Application Publication
(10) Pub. No.: US 2017/0273922 A1
(43) Pub. Date: Sep. 28, 2017

(54) UREA AND BIS-UREA BASED COMPOUNDS AND ANALOGUES THEREOF USEFUL IN THE TREATMENT OF ANDROGEN RECEPTOR MEDIATED DISEASES OR DISORDERS

(71) Applicant: THE ROYAL INSTITUTION FOR THE ADVACEMENT OF LEARNING/MCGILL UNIVERSITY, MONTREAL (CA)

(72) Inventors: JIAN HUI WU, SAINT-LAURENT (CA); XIAOHONG TIAN, URUMQI XINJIANG (CN); WEIGUO LIU, CHENGDU SICHUAN (CN)

(21) Appl. No.: 15/514,037

(22) PCT Filed: Oct. 2, 2015
(86) PCT No.: PCT/CA2015/050994

§ 371 Date: Mar. 24, 2017

Related U.S. Application Data
(60) Provisional application No. 62/059,597, filed on Oct. 3, 2014.

(51) Int. Cl. A61K 31/17; A61K 31/36; A61K 31/381; A61K 31/404; A61K 31/4164; A61K 31/44; A61K 31/421; A61K 31/495; A61K 31/4439; A61K 31/50; A61K 31/505; A61K 31/513; A61K 31/5375; C07C 275/24; C07C 275/40; C07D 401/12; A61K 31/341

(52) U.S. Cl. CPC A61K 31/17; A61K 31/341; A61K 31/36; A61K 31/381; A61K 31/404; A61K 31/4164; A61K 31/44; A61K 31/421; A61K 31/495; A61K 31/4439; A61K 31/50; A61K 31/505; A61K 31/513; A61K 31/5375; C07C 275/24; C07C 275/40; C07D 401/12

(57) ABSTRACT

Urea-based and bis-urea based compounds and analogues thereof are disclosed. These compounds are useful in the treatment of androgen-dependent diseases or disorders and androgen receptor-mediated diseases or disorders. Specifically, the compounds are useful in the treatment of diseases or disorders that are AR negative.

### FIELD OF THE INVENTION

The invention relates generally to compounds, their preparation and their use in the treatment of medical conditions that may or may not involve hormones. In certain embodiments, the compounds are useful in the treatment of androgen-dependent diseases or disorders and androgen receptor (AR)-mediated diseases or disorders. In other embodiments, the compounds are useful in the treatment of diseases or disorders that are AR negative.

### BACKGROUND OF THE INVENTION

The growth and survival of androgen-dependent cells such as prostate cancer cells critically depend on the signaling of the AR. The AR comprises three functional domains: the N-terminal domain (NTD), the DNA-binding domain (DBD) and the ligand-binding domain (LBD). Androgens activate the AR by binding at the AR-LBD. Current therapeutic strategy for advanced prostate cancer is to reduce serum level of androgens (via castration) and by disrupting binding of androgens to the AR-LBD by antiandrogens. Thus, treatment focuses on blocking the AR signaling and the battle field is at the AR-LBD (FIG. 1). While this treatment is initially effective, lethal ‘castration-resistant’ prostate cancer (CRPC) arises as a result of oncogenic re-activation of the AR.

Various laboratory and clinical studies have revealed that the AR-LBD is not a good ‘battle field’ for inhibiting the AR activation. Firstly, mutations in the AR-LBD could render the LBD-directed antiandrogen useless. In particular, enzalutamide is the second-generation of antiandrogen that was approved by FDA in 2012 to treat CRPC, but many patients have already developed resistance to this drug as the treatment selects for the AR mutant with F876L mutation at the LBD, which is paradoxically activated by enzalutamide. Secondly, an even more alarming problem is the emergence of AR variants lacking the LBD (such as AR-V7) in CRPC patients and such AR variants are constitutively active even in the absence of androgens, resulting in resistance to LBD-directed antiandrogens such as enzalutamide and androgen-depleting agents such as abiraterone. Unfortunately, all of the FDA-approved antiandrogens are directed towards the AR-LBD and are therefore inactive against AR-v7 (FIG. 2).

Prostate cancer cells are very versatile in circumventing therapeutic block of activation of the AR. The rationale to develop chemical inhibitors that target the AR-NTD has at least two folds. Firstly, the AR-NTD is the “Achilles' heel” of AR activity.¹ All of the known mechanisms that could account for AR reactivation in CRPC cells critically depend on the AR-NTD to reactivate AR. Secondly, among the NTD, DBD and LBD domains, the NTD is the most different domain between the AR and other members of steroid receptors (FIG. 3). The AR-NTD is intrinsically disordered under physiological conditions and is considered difficult to being by chemical compounds.

As outlined herein above, current mainstay treatment for advanced (metastatic) prostate cancer is to suppress the AR signaling by androgen deprivation therapy (ADT) via castration and use of antiandrogens. Currently available antiandrogens, such as enzalutamide, bicalutamide and nilutamide, are chemical compounds that inhibit the AR transcriptional activation by binding with the hormone-binding pocket of the AR-LBD (FIG. 1a). In men with metastatic prostate cancer treated with ADT, progression to the lethal disease state (CRPC) almost always occurs following a period of various clinical responses.² Docetaxel-based chemotherapy provides only a modest improvement in overall CRPC patient survival (few months).^{3,4} To date, the median survival time for CRPC patients is <2 years.^{3,4}

Recent emerging biological observations in prostate cancer have provided the explanation for the failure of the ADT in CRPC and the rationale for developing novel AR inhibitors for the CRPC. The most important pieces of these observations are as follows:⁵ i) Most CRPC cells are still dependent on the AR signaling for proliferation and survival and the AR therefore remains as the drug target for the CRPC; ii) In CRPC cells, the AR is activated by multiple mechanisms that can no longer be suppressed by castration and currently available antiandrogens; and iii) Accumulated evidence indicates the existence of multiple different malignant clones that could have developed different mechanisms of resistance to castration and antiandrogens in the same CRPC patients.⁶

The proposed mechanisms that may account for the sustained AR activation in the CRPC cells are as follows: 1) Elevated level of AR, resulting in AR activation at low level of androgen due to mass action; 2) Mutations in AR, rendering the AR promiscuous so that it can be activated by a broad range of non-androgen ligands, even antiandrogens; 3) Conversion of adrenal androgens to testosterone and intratumoral synthesis of androgens in CRPC cells; and 4) Androgen-independent activation of the AR via cross-talk with other factors/pathways.⁷⁻⁹ Recently, a series of AR splice variants lacking the LBD (referred to as AR-Vs) have been discovered from cell lines and patients. Several AR-Vs, such as AR-v7 and AR^{v567es}, have been shown to be constitutively active even in the absence of the androgens, and lack the ability to bind the androgens due to truncation of the AR-LBD.¹⁰⁻¹² Thus, expression of constitutively active AR-Vs could be an important mechanism underlying the sustained AR signaling in CRPC and development of resistance to AR-LBD-directed therapies.

In patients, Hu et al. found that AR-v7 showed an average 20-fold higher expression in CRPC when compared with hormone-naïve prostate cancer specimens, and among the hormone-naïve prostate cancer, higher expression of AR-v7 predicted biochemical recurrence following surgical treatment.¹⁰ Guo et al. found that AR-v7 (referred to as AR3 in Guo's work) is significantly up-regulated during prostate cancer progression, and AR-v7 expression level is correlated with the risk of tumor recurrence after radical prostatectomy.¹¹ Sun et al. have demonstrated that castration resistance in human prostate cancer is conferred by frequently occurring AR splice variants. Importantly, of 46 metastases derived from 13 patients with CRPC, 20 out of 46 (43%) expressed AR^{v567es}, 11 out of 46 (24%) expressed AR-v7.¹² Several specimens contained more than one AR variant, and nearly all of the specimen that contained one or more of the variants also contained full-length AR.¹² By a novel immunohistochemical approach, Zhang et al. have investigated the prevalence of AR-Vs in multiple metastatic sites of 42 CRPC patients. The study found that 23 out of 42 patients (55%) had at least one metastatic site with decreased C-terminal AR immunoreactivity and they concluded that C-terminal truncated AR splice variants occur frequently in CRPC metastases.¹³

Another recent study found that expression of AR-v7 and AR^{v567es} are detected in ⅓ (33%) of all prostate cancer bone metastases in patients and levels of these AR-Vs are increased in CRPC. More importantly, detectable AR^{v567es} and/or AR-v7 mRNA was associated with short patient survival.¹⁴ The pioneer work of Dr. Sadar and his team have demonstrated that it is feasible to target the AR-NTD and inhibit AR variant lacking the LBD by a small organic molecule called EPI-001.15 EPI-001 is a derivative of bisphenol A diglycidic ether, which was reported in the work of Biles et al. (1999).¹⁶

To date, EPI-001 is the best characterized compound targeting the AR-NTD.^{15,17} The IC50 of EPI-001 in PSA-luc reporter assay in LNCaP cells was 12.63±4.33 μM.¹⁷ On other hand, the F876L mutation at full-length AR is sufficient to confer enzalutamide resistance in cell lines and xenograft model.¹⁸ Importantly, the AR F876L mutant is detected in CRPC patients treated with an enzalutamide analogue (ARN-509), suggesting selective outgrowth of AR F876L is a clinically relevant mechanism of enzalutamide resistance.¹⁹ A series of mutations in AR-LBD, such as T877A, H874Y, W741C, L701H and V715M were identified from tissue specimens of CRPC patients, and found to produce mutated ARs which can be activated by a series of non-androgen ligands even the antiandrogens.^{7,20-24}

There is a need for compounds that act as antiandrogens. More specifically, there is a need for compounds that target the AR, its mutants and its variants; in particular the N-terminal domain of the AR (AR-NTD).

As indicated above, the compound EPI-001 known in the art targets the AR-NTD.¹⁵ The compounds of the invention are structurally different from EPI-001. In embodiments of the invention, the chemical structure of the compounds comprises at least one urea moiety. A few compounds of similar structures are disclosed in U.S. Pat. No. 6,093,742, however, for completely different uses.

In addition to the prostate cancer, recent studies indicated that the AR is an important mediator of other tumors, such as for example the breast cancer, hepatocellular carcinoma and ovarian cancer.

### SUMMARY OF THE INVENTION

The inventors have designed and prepared novel chemical compounds. The compounds may be used in the treatment of medical conditions that may or may not involve hormones. In certain embodiments, the compounds may be used in the treatment of androgen-dependent diseases or disorders and androgen receptor (AR)-mediated diseases or disorders. In other embodiments, the compounds may be used in the treatment of diseases or disorders that are AR negative.

The disease or disorder may be selected from: prostate cancer including AR positive prostate cancers and AR negative prostate cancers, castration-resistant prostate cancers, breast cancer including AR positive breast cancers and AR negative breast cancers as well as ovarian cancer, hepatocellular carcinoma, endometrial cancer, benign prostatic hyperplasia, endometriosis, male pattern baldness, spinal and bulbar muscular atrophy.

The compounds according to the invention may target the AR and/or its mutants and/or its variants (AR-Vs). In particular, the compounds according to the invention may target the N-terminal domain of the androgen receptor (AR-NTD). More specifically, the compounds may antagonize a series of the clinically-relevant mutants of the full-length ARs, such as for example the F876L mutated AR. Also, the compounds may inhibit the constitutive activity of AR-Vs, such as for example AR-v7, which lacks the LBD. Moreover, the compounds may antagonize the aberrant AR signaling in CRPC cells that express AR-Vs, such as for example AR-v7. The compounds according to the invention may modulate other targets different from the AR.

The invention thus provides for the following according to aspects thereof:
(1) A compound of general formula A or B below, or a pharmaceutically acceptable salt thereof, or a solvate or hydrate thereof,

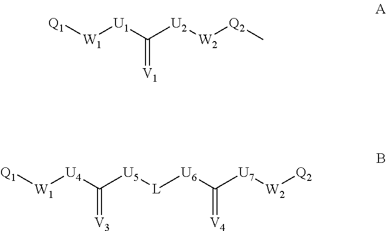

wherein:
U₁, U₂, U₄, U₅, U₆ and U₇ are each independently selected from a heteroatom and NR₁R₂ wherein R₁ and R₂ are each independently selected from H, alkyl, cycloalkyl, alkene, alkyne, aryl and alkylaryl, a 5 to 8-member ring comprising one or more heteroatom which are the same or different, or R₁ and R₂ together form a 5 to 8-member ring comprising one or more heteroatom; optionally, the ring is substituted with a substituent selected from alkyl, cycloalkyl alkoxy, alkoxy, thioalkoxy, OH, SH, NH₂, a halogen atom, a halogeno alkyl, a halogeno alkoxy, a halogeno thioalkoxy, CN, NO₂, SO₂ and COOH;
V₁, V₃ and V₄ are each independently selected from a heteroatom and carbon atom;
W₁ and W₂ are each independently present of absent, and are each independently selected from alkylene, alkenyl, alkynyl, a 5 to 20-member ring or bicycle ring comprising one or more heteroatom which are the same or different; optionally, the ring or bicycle ring is substituted with a group selected from alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, CN, NO₂, SO₂, COOH and NR₃R₄ wherein R₃ and R₄ are each independently selected from H, alkyl, cycloalkyl, alkene, alkyne, aryl and alkylaryl, or R₃ and R₄ together form a 5 to 8-member ring optionally comprising one or more heteroatom which are the same or different;
Q₁ is selected from alkyl, cycloalkyl, alkene, alkyne, aryl and alkylaryl, a 5 to 20-member ring or bicycle ring optionally comprising one or more heteroatom which are the same or different; optionally, the ring or bicycle ring is substituted with a substituent selected from alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, CN, NO₂, SO₂, COOH, acyloxycarbonyl, NR₃R₄ and C(═O)NR₃R₄ wherein R₃ and R₄ are each independently selected from H, alkyl, cycloalkyl, alkene, alkyne, aryl and alkylaryl, or R₃ and R₄ together form a 5 to 8-member ring optionally comprising one or more heteroatom which are the same or different; optionally, the 5 to 8-member ring is attached to an alkyl, a cycloalkyl, an alkene, an alkynyl, an aryl, aralkylryl or an acyloxycarbonyl; optionally, two consecutive substituents on the 5 to 20-member ring or bicycle ring together form a 5 to 8-member ring optionally comprising one or more heteroatom which are the same or different;
Q₂ is as defined above for Q₁, or is -Q′₂-U₃—C(═V₂)Q₃, wherein: U₃ is as defined above for U₁, U₂, U₄, U₅, U₆ and U₇; V₂ is as defined above for V₁, V₃ and V₄; and Q′₂ and Q₃ are each independently as defined above for Q₁;
L is selected from alkylene, alkenyl, alkynyl, a 5 to 20-member ring or bicycle ring comprising one or more heteroatom which are the same or different; optionally, the ring or bicycle ring is substituted with a group selected from alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, CN, NO₂, SO₂, COOH and NR₃R₄ wherein R₃ and R₄ are each independently selected from H, alkyl, cycloalkyl, alkene, alkyne, aryl and alkylaryl, or R₃ and R₄ together form a 5 to 8-member ring optionally comprising one or more heteroatom which are the same or different;
optionally L together with either U₅ or U₆ or both U₅ and U₆ form a 5 to 20-member ring or bicycle ring optionally comprising one or more heteroatom which are the same or different; optionally, the ring or bicycle ring is substituted with a substituent selected from alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, CN, NO₂, SO₂, COOH acyloxycarbonyl, NR₃R₄ and C(═O)NR₃R₄ wherein R₃ and R₄ are each independently selected from H, alkyl, cycloalkyl, alkene, alkyne, aryl and alkylaryl, or R₃ and R₄ together form a 5 to 8-member ring optionally comprising one or more heteroatom which are the same or different; optionally, the 5 to 8-member ring is attached to an alkyl, a cycloalkyl, an alkene, an alkynyl, an aryl, analkylryl or an acyloxycarbonyl; optionally, two consecutive substituents on the 5 to 20-member ring or bicycle ring together form a 5 to 8-member ring optionally comprising one or more heteroatom which are the same or different;
the heteroatom is selected from O, N and S.
(2) A compound according to (1) above having the general formula A1

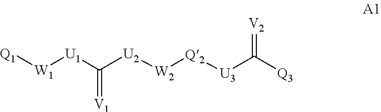

(3) A compound according to (2) above having the general formula A2

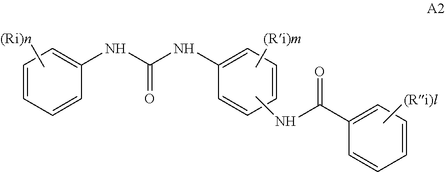

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different;
m is an integer selected from 0 to 4, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different; and
l is an integer selected from 0 to 5, and each R″i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R″i together form a 5 to 8-member ring which optionally comprises one or more heteroatom which are the same or different.
(4) A compound according to (3) above, which is selected from the group of compounds depicted below

| ID | Structure |
|---|---|
| 746 | 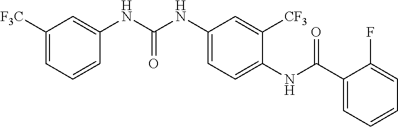 [SMILES: O=C(Nc1cccc(C(F)(F)F)c1)Nc1ccc(NC(=O)c2ccccc2F)c(C(F)(F)F)c1] |
| 747 | 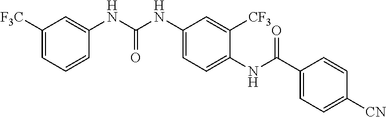 [SMILES: N#Cc1ccc(C(=O)Nc2ccc(NC(=O)Nc3cccc(C(F)(F)F)c3)cc2C(F)(F)F)cc1] |
| 808 | 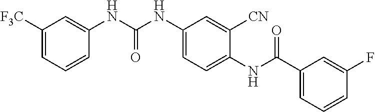 [SMILES: N#Cc1cc(NC(=O)Nc2cccc(C(F)(F)F)c2)ccc1NC(=O)c1cccc(F)c1] |
| 743 | 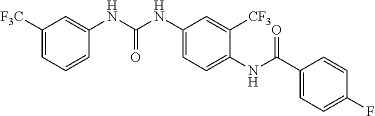 [SMILES: O=C(Nc1cccc(C(F)(F)F)c1)Nc1ccc(NC(=O)c2ccc(F)cc2)c(C(F)(F)F)c1] |
| 806 | 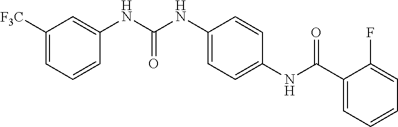 [SMILES: O=C(Nc1ccc(NC(=O)c2ccccc2F)cc1)Nc1cccc(C(F)(F)F)c1] |
| 814 | 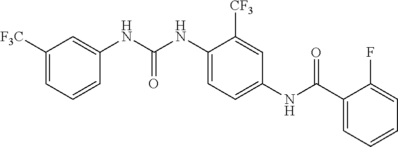 [SMILES: O=C(Nc1cccc(C(F)(F)F)c1)Nc1ccc(NC(=O)c2ccccc2F)cc1C(F)(F)F] |
| 815 | 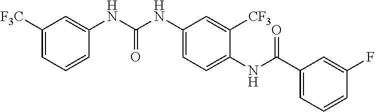 [SMILES: O=C(Nc1cccc(C(F)(F)F)c1)Nc1ccc(NC(=O)c2cccc(F)c2)c(C(F)(F)F)c1] |
| 813 | 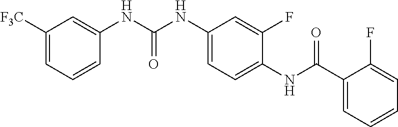 [SMILES: O=C(Nc1cccc(C(F)(F)F)c1)Nc1ccc(NC(=O)c2ccccc2F)c(F)c1] |
| 863 | 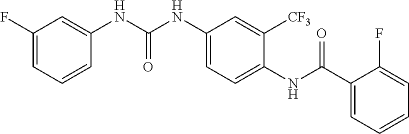 [SMILES: O=C(Nc1cccc(F)c1)Nc1ccc(NC(=O)c2ccccc2F)c(C(F)(F)F)c1] |
| 886 | 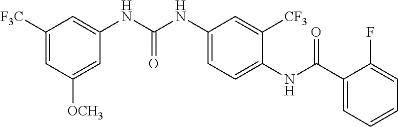 [SMILES: COc1cc(NC(=O)Nc2ccc(NC(=O)c3ccccc3F)c(C(F)(F)F)c2)cc(C(F)(F)F)c1] |
| 896 | 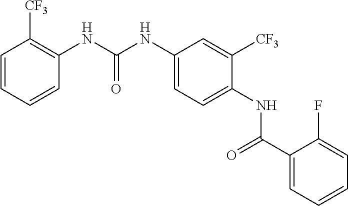 [SMILES: O=C(Nc1ccc(NC(=O)c2ccccc2F)c(C(F)(F)F)c1)Nc1ccccc1C(F)(F)F] |
| 849 | 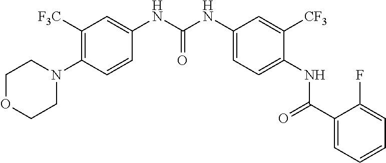 [SMILES: O=C(Nc1ccc(NC(=O)c2ccccc2F)c(C(F)(F)F)c1)Nc1ccc(N2CCOCC2)c(C(F)(F)F)c1] |
| 816 | 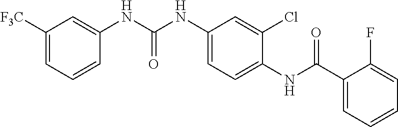 [SMILES: O=C(Nc1cccc(C(F)(F)F)c1)Nc1ccc(NC(=O)c2ccccc2F)c(Cl)c1] |
| 820 | 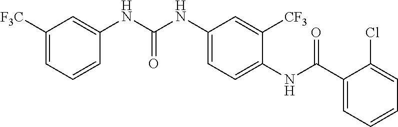 [SMILES: O=C(Nc1cccc(C(F)(F)F)c1)Nc1ccc(NC(=O)c2ccccc2Cl)c(C(F)(F)F)c1] |
| 825 | 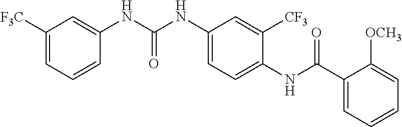 [SMILES: COc1ccccc1C(=O)Nc1ccc(NC(=O)Nc2cccc(C(F)(F)F)c2)cc1C(F)(F)F] |
| 864 | 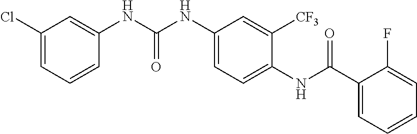 [SMILES: O=C(Nc1cccc(Cl)c1)Nc1ccc(NC(=O)c2ccccc2F)c(C(F)(F)F)c1] |
| 897 | 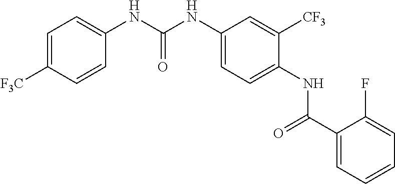 [SMILES: O=C(Nc1ccc(C(F)(F)F)cc1)Nc1ccc(NC(=O)c2ccccc2F)c(C(F)(F)F)c1] |
| 879 | 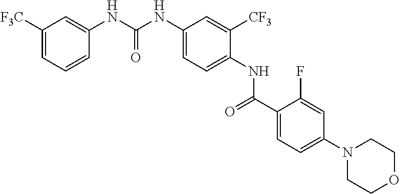 [SMILES: O=C(Nc1cccc(C(F)(F)F)c1)Nc1ccc(NC(=O)c2ccc(N3CCOCC3)cc2F)c(C(F)(F)F)c1] |
| 878 | 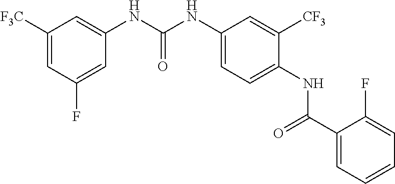 [SMILES: O=C(Nc1cc(F)cc(C(F)(F)F)c1)Nc1ccc(NC(=O)c2ccccc2F)c(C(F)(F)F)c1] |
| 862 | 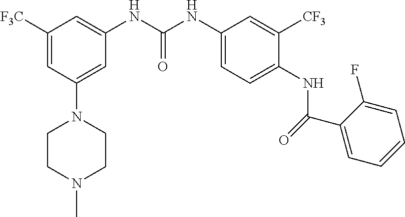 [SMILES: CN1CCN(c2cc(NC(=O)Nc3ccc(NC(=O)c4ccccc4F)c(C(F)(F)F)c3)cc(C(F)(F)F)c2)CC1] |
| 900 | 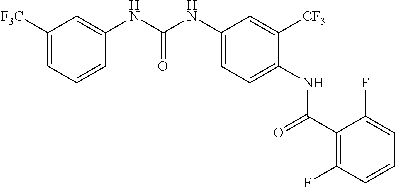 [SMILES: O=C(Nc1cccc(C(F)(F)F)c1)Nc1ccc(NC(=O)c2c(F)cccc2F)c(C(F)(F)F)c1] |
| 894 | 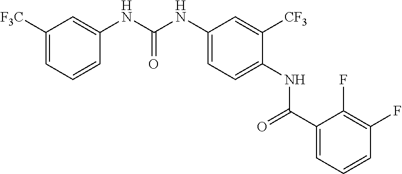 [SMILES: O=C(Nc1cccc(C(F)(F)F)c1)Nc1ccc(NC(=O)c2cccc(F)c2F)c(C(F)(F)F)c1] |
| 902 | 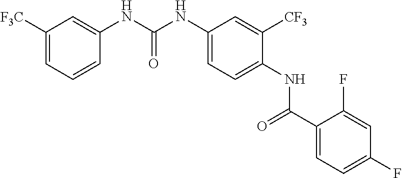 [SMILES: O=C(Nc1cccc(C(F)(F)F)c1)Nc1ccc(NC(=O)c2ccc(F)cc2F)c(C(F)(F)F)c1] |
| 907 | 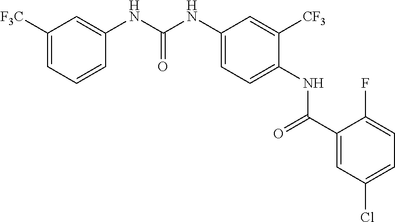 [SMILES: O=C(Nc1cccc(C(F)(F)F)c1)Nc1ccc(NC(=O)c2cc(Cl)ccc2F)c(C(F)(F)F)c1] |
| 861 | 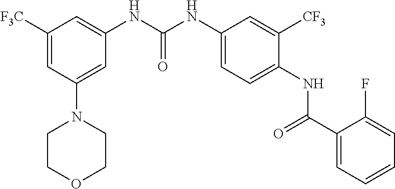 [SMILES: O=C(Nc1cc(N2CCOCC2)cc(C(F)(F)F)c1)Nc1ccc(NC(=O)c2ccccc2F)c(C(F)(F)F)c1] |
| 890 | 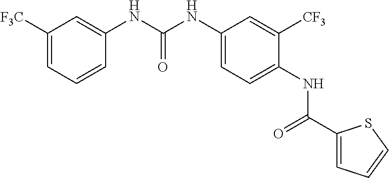 [SMILES: O=C(Nc1cccc(C(F)(F)F)c1)Nc1ccc(NC(=O)c2cccs2)c(C(F)(F)F)c1] |
| 906 | 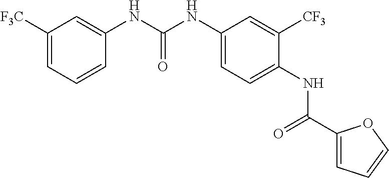 [SMILES: O=C(Nc1cccc(C(F)(F)F)c1)Nc1ccc(NC(=O)c2ccco2)c(C(F)(F)F)c1] |
| 901 | 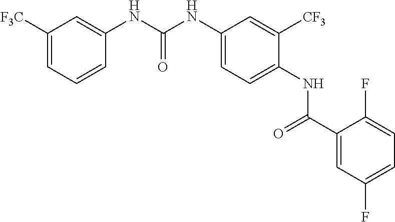 [SMILES: O=C(Nc1cccc(C(F)(F)F)c1)Nc1ccc(NC(=O)c2cc(F)ccc2F)c(C(F)(F)F)c1] |
| 903 | 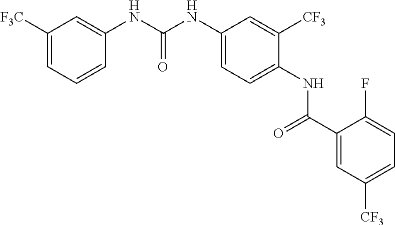 [SMILES: O=C(Nc1cccc(C(F)(F)F)c1)Nc1ccc(NC(=O)c2cc(C(F)(F)F)ccc2F)c(C(F)(F)F)c1] |
| 911 | 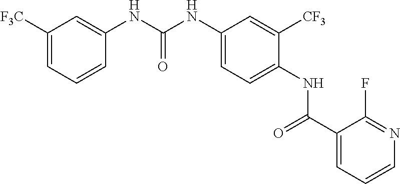 [SMILES: O=C(Nc1cccc(C(F)(F)F)c1)Nc1ccc(NC(=O)c2cccnc2F)c(C(F)(F)F)c1] |
| 952 | 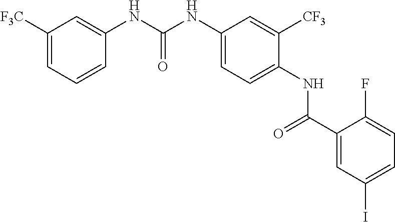 [SMILES: O=C(Nc1cccc(C(F)(F)F)c1)Nc1ccc(NC(=O)c2cc(I)ccc2F)c(C(F)(F)F)c1] |
| 971 | 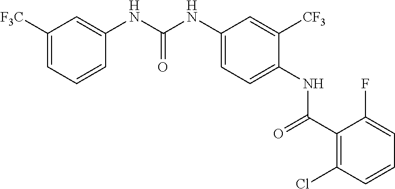 [SMILES: O=C(Nc1cccc(C(F)(F)F)c1)Nc1ccc(NC(=O)c2c(F)cccc2Cl)c(C(F)(F)F)c1] |
| 912 | 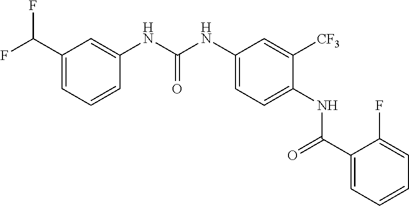 [SMILES: O=C(Nc1cccc(C(F)F)c1)Nc1ccc(NC(=O)c2ccccc2F)c(C(F)(F)F)c1] |
| 930 | 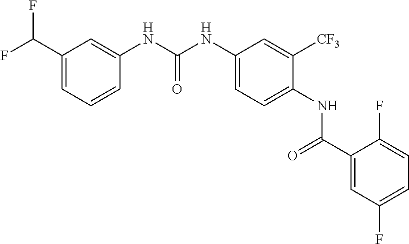 [SMILES: O=C(Nc1cccc(C(F)F)c1)Nc1ccc(NC(=O)c2cc(F)ccc2F)c(C(F)(F)F)c1] |
| 945 | 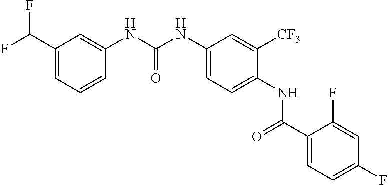 [SMILES: O=C(Nc1cccc(C(F)F)c1)Nc1ccc(NC(=O)c2ccc(F)cc2F)c(C(F)(F)F)c1] |
| 921 | 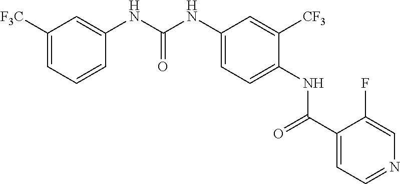 [SMILES: O=C(Nc1cccc(C(F)(F)F)c1)Nc1ccc(NC(=O)c2ccncc2F)c(C(F)(F)F)c1] |
| 923 | 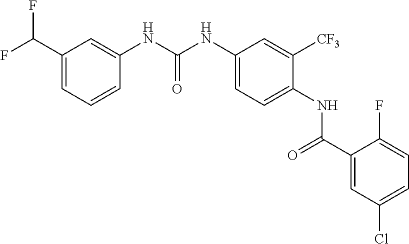 [SMILES: O=C(Nc1cccc(C(F)F)c1)Nc1ccc(NC(=O)c2cc(Cl)ccc2F)c(C(F)(F)F)c1] |
| 941 | 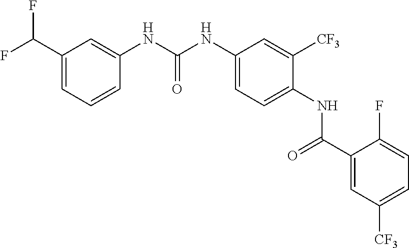 [SMILES: O=C(Nc1cccc(C(F)F)c1)Nc1ccc(NC(=O)c2cc(C(F)(F)F)ccc2F)c(C(F)(F)F)c1] |
| 983 | 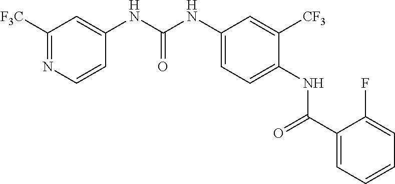 [SMILES: O=C(Nc1ccnc(C(F)(F)F)c1)Nc1ccc(NC(=O)c2ccccc2F)c(C(F)(F)F)c1] |
| 908 | 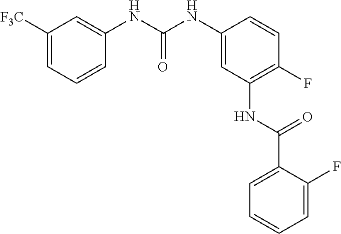 [SMILES: O=C(Nc1cccc(C(F)(F)F)c1)Nc1ccc(F)c(NC(=O)c2ccccc2F)c1] |
| 910 | 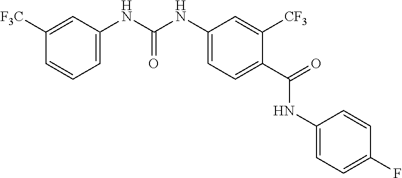 [SMILES: O=C(Nc1cccc(C(F)(F)F)c1)Nc1ccc(C(=O)Nc2ccc(F)cc2)c(C(F)(F)F)c1] |
| 914 | 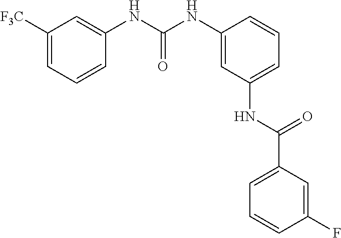 [SMILES: O=C(Nc1cccc(NC(=O)c2cccc(F)c2)c1)Nc1cccc(C(F)(F)F)c1] |
| 928 | 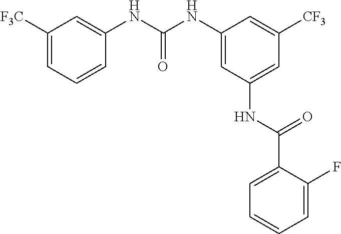 [SMILES: O=C(Nc1cccc(C(F)(F)F)c1)Nc1cc(NC(=O)c2ccccc2F)cc(C(F)(F)F)c1] |
| 942 | 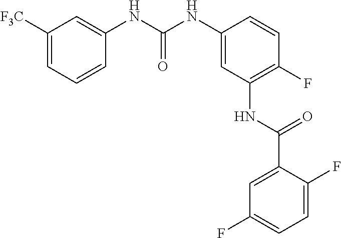 [SMILES: O=C(Nc1cccc(C(F)(F)F)c1)Nc1ccc(F)c(NC(=O)c2cc(F)ccc2F)c1] |
| 909 | 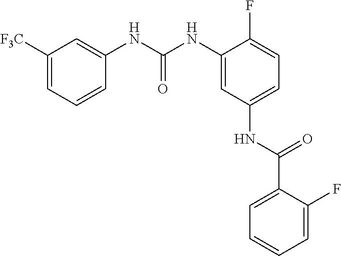 [SMILES: O=C(Nc1cccc(C(F)(F)F)c1)Nc1cc(NC(=O)c2ccccc2F)ccc1F] |
| 913 | 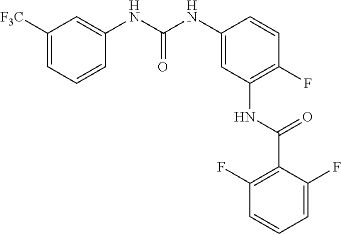 [SMILES: O=C(Nc1cccc(C(F)(F)F)c1)Nc1ccc(F)c(NC(=O)c2c(F)cccc2F)c1] |
| 915 | 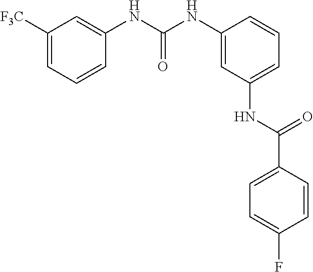 [SMILES: O=C(Nc1cccc(NC(=O)c2ccc(F)cc2)c1)Nc1cccc(C(F)(F)F)c1] |
| 929 | 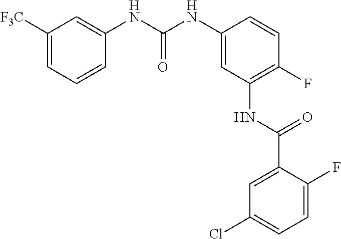 [SMILES: O=C(Nc1cccc(C(F)(F)F)c1)Nc1ccc(F)c(NC(=O)c2cc(Cl)ccc2F)c1] |
| 943 | 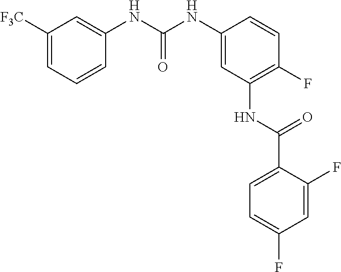 [SMILES: O=C(Nc1cccc(C(F)(F)F)c1)Nc1ccc(F)c(NC(=O)c2ccc(F)cc2F)c1] |
| 944 | 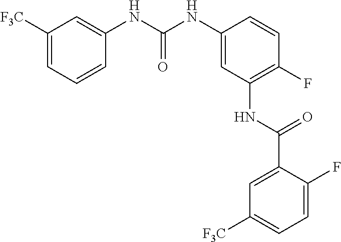 [SMILES: O=C(Nc1cccc(C(F)(F)F)c1)Nc1ccc(F)c(NC(=O)c2cc(C(F)(F)F)ccc2F)c1] |
| 947 | 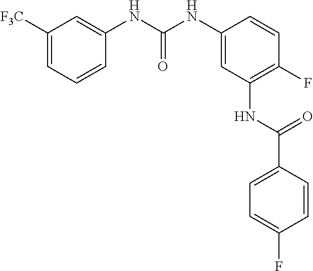 [SMILES: O=C(Nc1cccc(C(F)(F)F)c1)Nc1ccc(F)c(NC(=O)c2ccc(F)cc2)c1] |
| 954 | 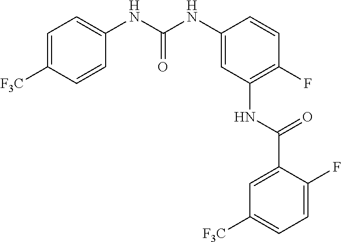 [SMILES: O=C(Nc1ccc(C(F)(F)F)cc1)Nc1ccc(F)c(NC(=O)c2cc(C(F)(F)F)ccc2F)c1] |
| 957 | 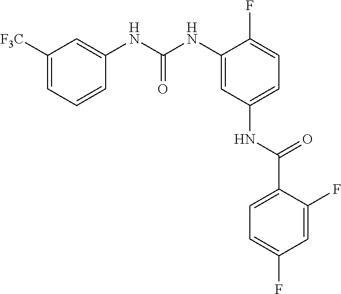 [SMILES: O=C(Nc1cccc(C(F)(F)F)c1)Nc1cc(NC(=O)c2ccc(F)cc2F)ccc1F] |
| 946 | 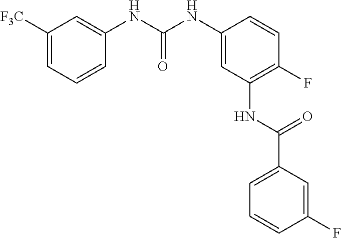 [SMILES: O=C(Nc1cccc(C(F)(F)F)c1)Nc1ccc(F)c(NC(=O)c2cccc(F)c2)c1] |
| 948 | 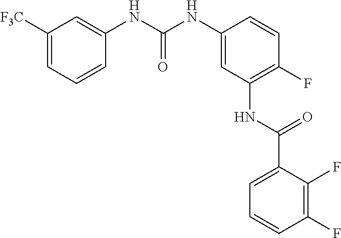 [SMILES: O=C(Nc1cccc(C(F)(F)F)c1)Nc1ccc(F)c(NC(=O)c2cccc(F)c2F)c1] |
| 951 | 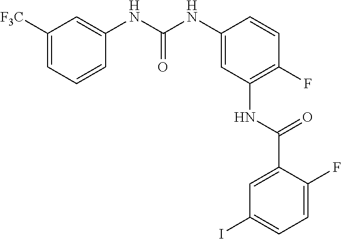 [SMILES: O=C(Nc1cccc(C(F)(F)F)c1)Nc1ccc(F)c(NC(=O)c2cc(I)ccc2F)c1] |
| 956 | 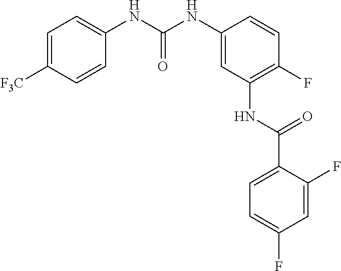 [SMILES: O=C(Nc1ccc(C(F)(F)F)cc1)Nc1ccc(F)c(NC(=O)c2ccc(F)cc2F)c1] |
| 958 | 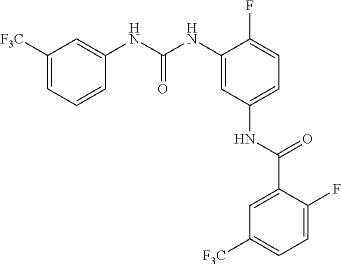 [SMILES: O=C(Nc1cccc(C(F)(F)F)c1)Nc1cc(NC(=O)c2cc(C(F)(F)F)ccc2F)ccc1F] |
| 959 | 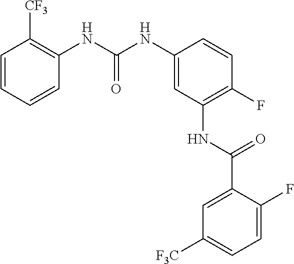 [SMILES: O=C(Nc1ccc(F)c(NC(=O)c2cc(C(F)(F)F)ccc2F)c1)Nc1ccccc1C(F)(F)F] |
| 963 | 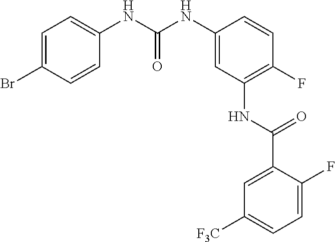 [SMILES: O=C(Nc1ccc(Br)cc1)Nc1ccc(F)c(NC(=O)c2cc(C(F)(F)F)ccc2F)c1] |
| 961 | 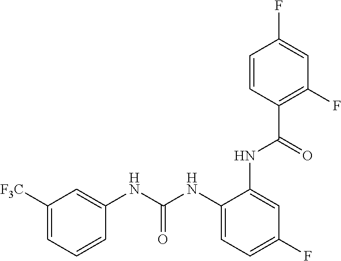 [SMILES: O=C(Nc1cccc(C(F)(F)F)c1)Nc1ccc(F)cc1NC(=O)c1ccc(F)cc1F] |
| 965 | 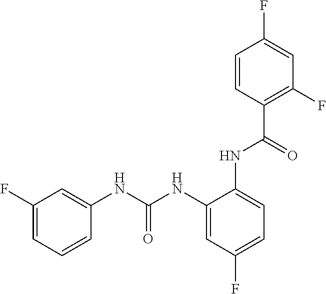 [SMILES: O=C(Nc1cccc(F)c1)Nc1cc(F)ccc1NC(=O)c1ccc(F)cc1F] |
| 969 | 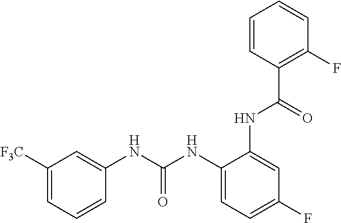 [SMILES: O=C(Nc1cccc(C(F)(F)F)c1)Nc1ccc(F)cc1NC(=O)c1ccccc1F] |
| 960 | 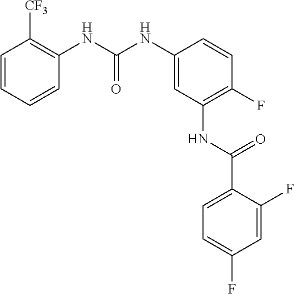 [SMILES: O=C(Nc1ccc(F)c(NC(=O)c2ccc(F)cc2F)c1)Nc1ccccc1C(F)(F)F] |
| 970 | 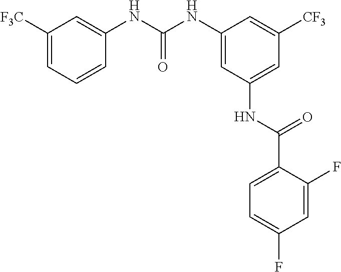 [SMILES: O=C(Nc1cccc(C(F)(F)F)c1)Nc1cc(NC(=O)c2ccc(F)cc2F)cc(C(F)(F)F)c1] |
| 962 | 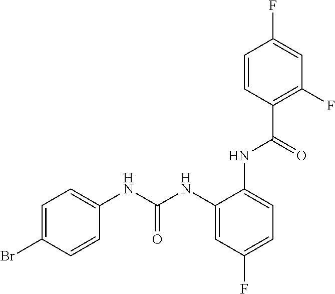 [SMILES: O=C(Nc1ccc(Br)cc1)Nc1cc(F)ccc1NC(=O)c1ccc(F)cc1F] |
| 968 | 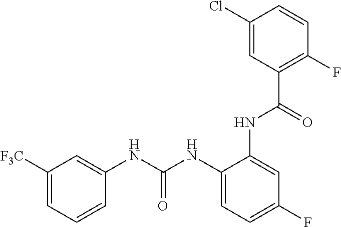 [SMILES: O=C(Nc1cccc(C(F)(F)F)c1)Nc1ccc(F)cc1NC(=O)c1cc(Cl)ccc1F] |
| 974 | 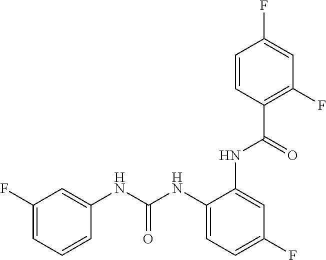 [SMILES: O=C(Nc1cccc(F)c1)Nc1ccc(F)cc1NC(=O)c1ccc(F)cc1F] |
| 975 | 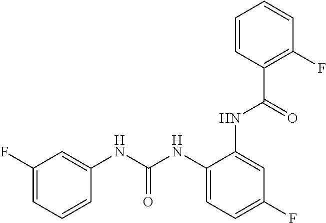 [SMILES: O=C(Nc1cccc(F)c1)Nc1ccc(F)cc1NC(=O)c1ccccc1F] |
| 976 | 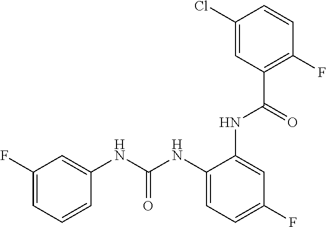 [SMILES: O=C(Nc1cccc(F)c1)Nc1ccc(F)cc1NC(=O)c1cc(Cl)ccc1F] |

(5) A compound according to (3) above, which is

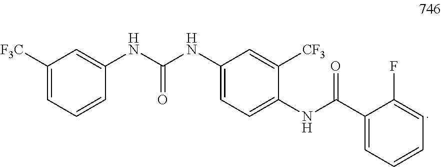 [SMILES: O=C(Nc1cccc(C(F)(F)F)c1)Nc1ccc(NC(=O)c2ccccc2F)c(C(F)(F)F)c1]

(6) A compound according to (1) above having the general formula A2′

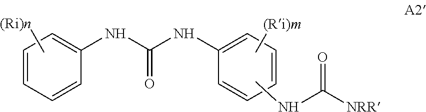

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different;
m is an integer selected from 0 to 4, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different; and
at least one of R and R′ is as Q₁, or R and R′ are as R₃ and R₄.
(7) A compound according to (6) above, which is selected from the group of compounds depicted below

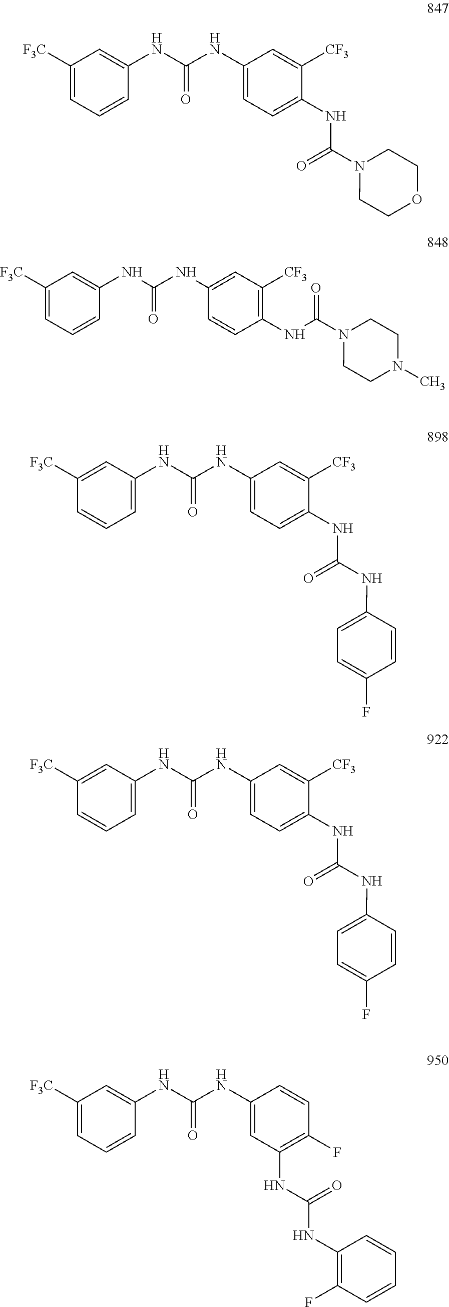 [SMILES: O=C(Nc1cccc(C(F)(F)F)c1)Nc1ccc(NC(=O)N2CCOCC2)c(C(F)(F)F)c1 ; CN1CCN(C(=O)Nc2ccc(NC(=O)Nc3cccc(C(F)(F)F)c3)cc2C(F)(F)F)CC1 ; O=C(Nc1cccc(C(F)(F)F)c1)Nc1ccc(NC(=O)Nc2ccc(F)cc2)c(C(F)(F)F)c1 ; O=C(Nc1cccc(C(F)(F)F)c1)Nc1ccc(NC(=O)Nc2ccc(F)cc2)c(C(F)(F)F)c1 ; O=C(Nc1cccc(C(F)(F)F)c1)Nc1ccc(F)c(NC(=O)Nc2ccccc2F)c1]

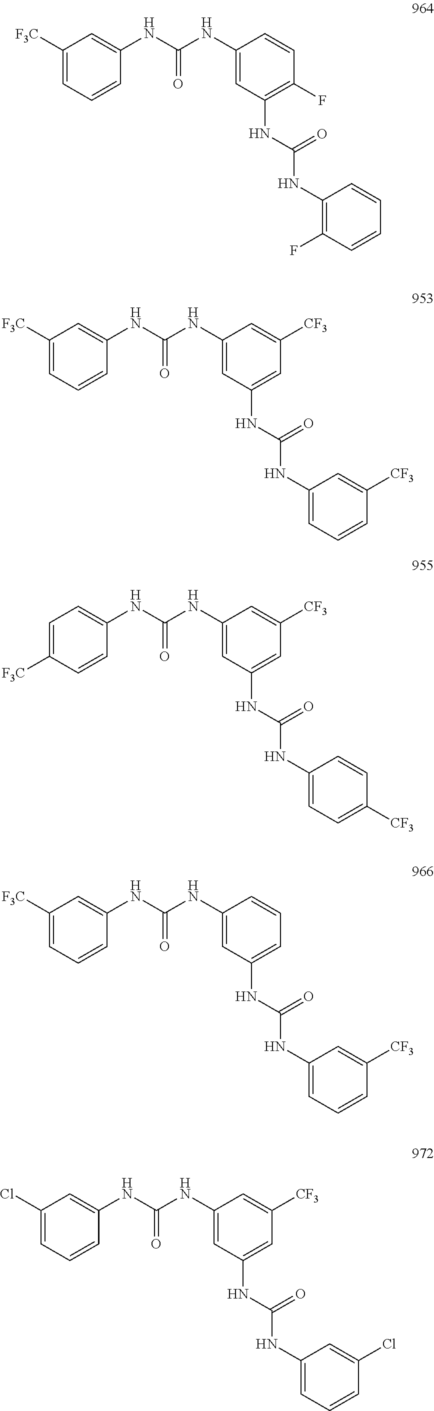 [SMILES: O=C(Nc1cccc(C(F)(F)F)c1)Nc1ccc(F)c(NC(=O)Nc2ccccc2F)c1 ; O=C(Nc1cccc(C(F)(F)F)c1)Nc1cc(NC(=O)Nc2cccc(C(F)(F)F)c2)cc(C(F)(F)F)c1 ; O=C(Nc1ccc(C(F)(F)F)cc1)Nc1cc(NC(=O)Nc2ccc(C(F)(F)F)cc2)cc(C(F)(F)F)c1 ; O=C(Nc1cccc(NC(=O)Nc2cccc(C(F)(F)F)c2)c1)Nc1cccc(C(F)(F)F)c1 ; O=C(Nc1cccc(Cl)c1)Nc1cc(NC(=O)Nc2cccc(Cl)c2)cc(C(F)(F)F)c1]

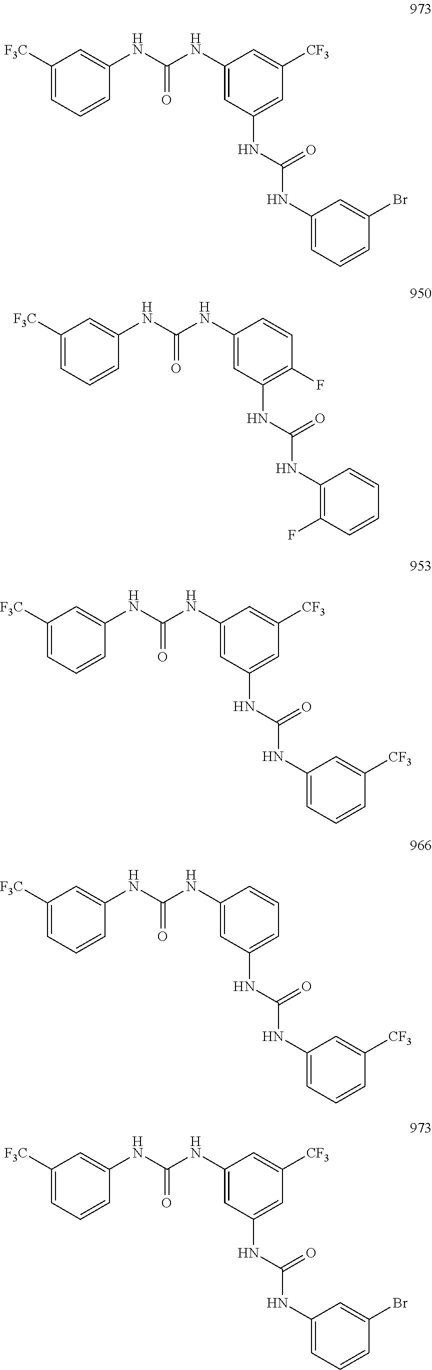 [SMILES: O=C(Nc1cccc(Br)c1)Nc1cc(NC(=O)Nc2cccc(C(F)(F)F)c2)cc(C(F)(F)F)c1 ; O=C(Nc1cccc(C(F)(F)F)c1)Nc1ccc(F)c(NC(=O)Nc2ccccc2F)c1 ; O=C(Nc1cccc(C(F)(F)F)c1)Nc1cc(NC(=O)Nc2cccc(C(F)(F)F)c2)cc(C(F)(F)F)c1 ; O=C(Nc1cccc(NC(=O)Nc2cccc(C(F)(F)F)c2)c1)Nc1cccc(C(F)(F)F)c1 ; O=C(Nc1cccc(Br)c1)Nc1cc(NC(=O)Nc2cccc(C(F)(F)F)c2)cc(C(F)(F)F)c1]

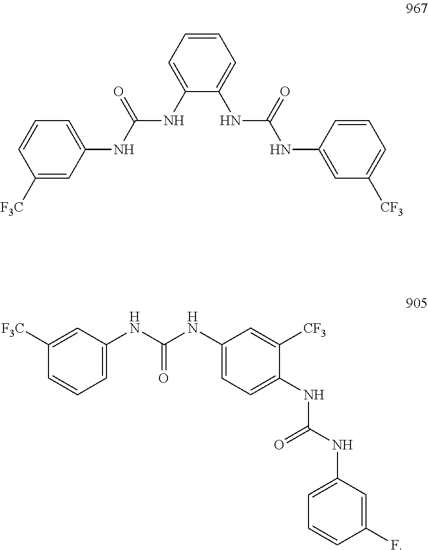 [SMILES: O=C(Nc1cccc(C(F)(F)F)c1)Nc1ccccc1NC(=O)Nc1cccc(C(F)(F)F)c1 ; O=C(Nc1cccc(C(F)(F)F)c1)Nc1ccc(NC(=O)Nc2cccc(F)c2)c(C(F)(F)F)c1]

(8) A compound according to (1) above having the general formula A3

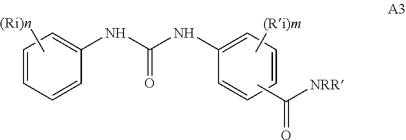

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different;
m is an integer selected from 0 to 4, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different; and
at least one of R and R′ is as Q₁, or R and R′ are as R₃ and R₄.
(9) A compound according to (8) above, which is selected from the group of compounds depicted below

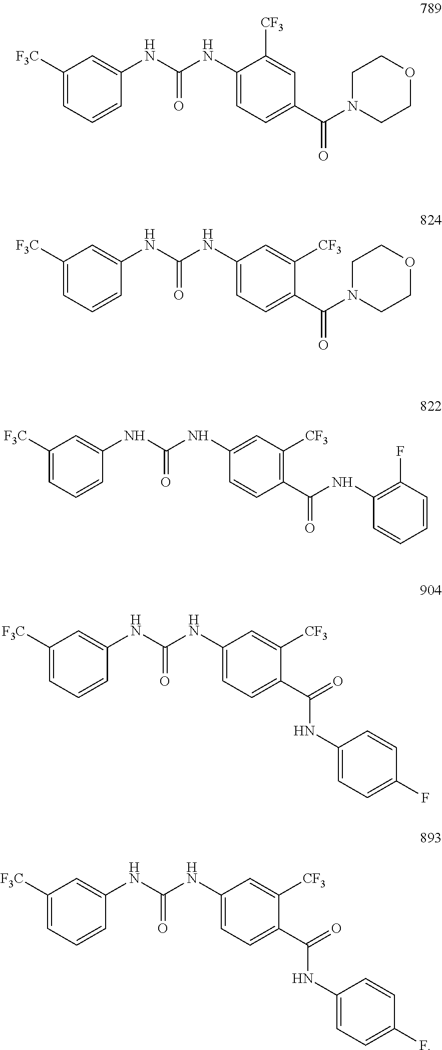 [SMILES: O=C(Nc1cccc(C(F)(F)F)c1)Nc1ccc(C(=O)N2CCOCC2)cc1C(F)(F)F ; O=C(Nc1cccc(C(F)(F)F)c1)Nc1ccc(C(=O)N2CCOCC2)c(C(F)(F)F)c1 ; O=C(Nc1cccc(C(F)(F)F)c1)Nc1ccc(C(=O)Nc2ccccc2F)c(C(F)(F)F)c1 ; O=C(Nc1cccc(C(F)(F)F)c1)Nc1ccc(C(=O)Nc2ccc(F)cc2)c(C(F)(F)F)c1 ; O=C(Nc1cccc(C(F)(F)F)c1)Nc1ccc(C(=O)Nc2ccc(F)cc2)c(C(F)(F)F)c1]

(10) A compound according to (8) above, which is

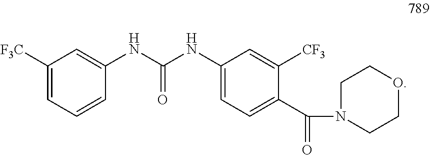 [SMILES: O=C(Nc1cccc(C(F)(F)F)c1)Nc1ccc(C(=O)N2CCOCC2)c(C(F)(F)F)c1]

(11) A compound according to (1) above having the general formula A3′

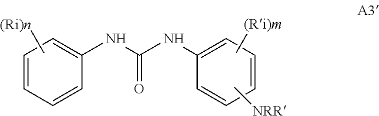

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different;
m is an integer selected from 0 to 4, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different; and
at least one of R and R′ is as Q₁, or R and R′ are as R₃ and R₄.
(12) A compound according to (11) above, which is selected from the group of compounds depicted below

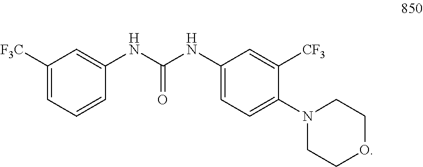 [SMILES: O=C(Nc1cccc(C(F)(F)F)c1)Nc1ccc(N2CCOCC2)c(C(F)(F)F)c1]

(13) A compound according to (1) above having the general formula A3″

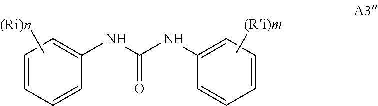

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different; and
m is an integer selected from 0 to 4, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different.
(14) A compound according to (13) above, which is selected from the group of compounds depicted below

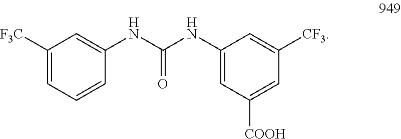 [SMILES: O=C(Nc1cccc(C(F)(F)F)c1)Nc1cc(C(=O)O)cc(C(F)(F)F)c1]

(15) A compound according to (1) above having the general formula A4

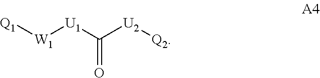

(16) A compound according to (15) above having the general formula A5

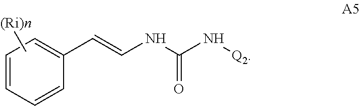

(17) A compound according to (16) above, which is selected from the group of compounds defined as outlined below

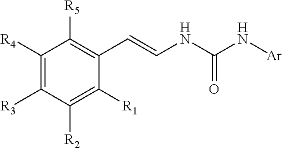

| | Substituents | | | | | | | | |
|---|---|---|---|---|---|---|---|---|---|
| | | | | Ar substituents | | | | | |
| ID | R₁ | R₂ | R₄ | | R₆ | R₇ | R₈ | R₉ | R₁₀ |
| 480 | CF₃ | | CF₃ | B | | | CN | | |
| 481 | CF₃ | | CF₃ | A | | | CN | | |
| 482 | | CF₃ | CF₃ | B | | | CN | | |
| 483 | | CF₃ | CF₃ | A | | | CN | | |
| 487 | CF₃ | | CF₃ | A | | | NO₂ | | |
| 489 | | CF₃ | CF₃ | B | | | NO₂ | | |
| 503 | | CF₃ | CF₃ | B | | | | | |
| 504 | CF₃ | | CF₃ | B | | | | | |
| 510 | | | CF₃ | B | | | CN | | |
| 511 | | | CF₃ | A | | | CN | | |
| 512 | | | | B | | | CN | | |
| 527 | | CF₃ | CF₃ | A | | | | | Br |
| 528 | | CF₃ | CF₃ | D | | | | | |
| 531 | | CF₃ | CF₃ | E | | | | | |
| 533 | | CF₃ | CF₃ | B | Cl | | | | |
| 535 | | CF₃ | CF₃ | B | | F | | | |
| 536 | | CF₃ | CF₃ | B | | | CH₃ | | |
| 537 | | CF₃ | CF₃ | B | | | | | CH₃ |
| 538 | | CF₃ | CF₃ | E | | CF₃ | | | |
| 539 | | CF₃ | CF₃ | B | | Br | | | |
| 540 | | CF₃ | CF₃ | B | | | Br | | |
| 541 | | CF₃ | CF₃ | B | CH₃ | | | | |
| 543 | | CF₃ | CF₃ | E | | Ph | | | |
| 546 | | CF₃ | CF₃ | B | | CH₃ | | | |
| 548 | | CF₃ | CF₃ | C | | | CF₃ | | |
| 549 | | CF₃ | CF₃ | C | | CF₃ | | | |
| 550 | | | CF₃ | B | | F | | | |
| 551 | | | CF₃ | B | Cl | | | | |
| 552 | | | CF₃ | B | | Br | | | |
| 553 | | | CF₃ | B | | | Br | | |
| 554 | | | CF₃ | B | | | CH₃ | | |
| 555 | | | CF₃ | B | CH₃ | | | | |
| 556 | | | CF₃ | B | | CH₃ | | | |
| 557 | | | CF₃ | B | | | | | CH₃ |
| 558 | | | CF₃ | D | | | | | |
| 559 | | | CF₃ | E | | | | | |
| 560 | | | CF₃ | E | | CF₃ | | | |
| 561 | | | CF₃ | E | | Ph | | | |
| 564 | | | CF₃ | C | | | CF₃ | | |
| 583 | CF₃ | | CF₃ | D | | | | | |

| | |
|---|---|
| 542 | 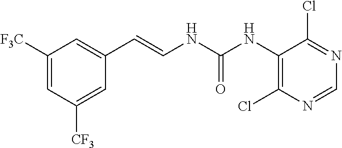 [SMILES: O=C(N/C=C/c1cc(C(F)(F)F)cc(C(F)(F)F)c1)Nc1c(Cl)ncnc1Cl] |
| 544 | 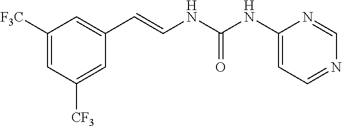 [SMILES: O=C(N/C=C/c1cc(C(F)(F)F)cc(C(F)(F)F)c1)Nc1ccncn1] |
| 545 | 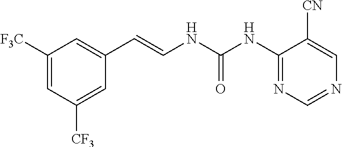 [SMILES: N#Cc1cncnc1NC(=O)N/C=C/c1cc(C(F)(F)F)cc(C(F)(F)F)c1] |
| 562 | 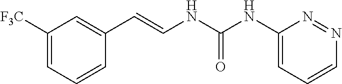 [SMILES: O=C(N/C=C/c1cccc(C(F)(F)F)c1)Nc1cccnn1] |
| 766 | 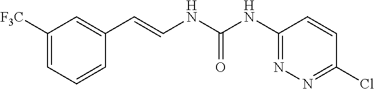 [SMILES: O=C(N/C=C/c1cccc(C(F)(F)F)c1)Nc1ccc(Cl)nn1] |
| 875 | 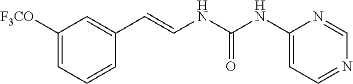 [SMILES: O=C(N/C=C/c1cccc(OC(F)(F)F)c1)Nc1ccncn1] |

R₃ = R₅ = H

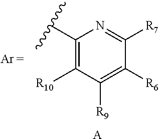

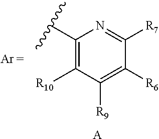

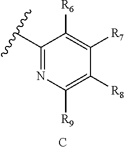

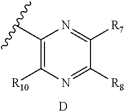

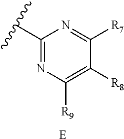

(18) A compound according to (16) above, which is selected from the group of compounds consisting of 480, 481, 482, 483, 528, 531, 533, 535, 538, 544, 549, 766 and 562.
19. A compound according to (16) above, which is

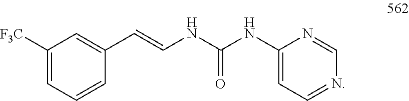 [SMILES: O=C(N/C=C/c1cccc(C(F)(F)F)c1)Nc1ccncn1]

(20) A compound according to (1) above having the general formula A6

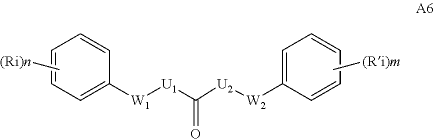

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different; and
m is an integer selected from 0 to 5, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different.
(21) A compound according to (20) above having the general formula A7

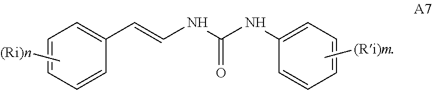

(22) A compound according to (21) above, which is selected from the group of compounds defined as outlined below

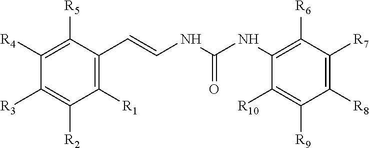

| | Substituents | | | | | | | | | |
|---|---|---|---|---|---|---|---|---|---|---|
| ID | R₁ | R₂ | R₃ | R₄ | R₅ | R₆ | R₇ | R₈ | R₉ | R₁₀ |
| 403 | | | | CF₃ | | | CF₃ | | | |
| 404 | | | | | CF₃ | | CF₃ | NO₂ | | |
| 405 | | | | | CF₃ | | | CN | | |
| 406 | | | | | CF₃ | | CF₃ | | | |
| 407 | | | CF₃ | | | | CF₃ | NO₂ | | |
| 408 | | | CF₃ | | | | CF₃ | | | |
| 409 | | | | CH₃O | | | CF₃ | NO₂ | | |
| 410 | | | | CF₃ | | | CF₃ | CN | | |
| 411 | | | | CH₃O | | | CF₃ | | | |
| 412 | | | | F | | | CF₃ | NO₂ | | |
| 413 | | | | CF₃ | | | | | | |
| 414 | | | | F | | | CF₃ | | | |
| 415 | | | | | | | CF₃ | | | |
| 416 | | | | CF₃ | | | | CN | | |
| 417 | | | | CF₃ | | | CH₃O | | | |
| 421 | | | CF₃ | | | | CF₃ | CN | | |
| 429 | CF₃ | | | | | | CH₃O | | | |
| 430 | | | | CH₃O | | | CH₃O | | | |
| 433 | | | | F | | | CF₃ | CN | | |
| 435 | | | | CF₃ | | | | N(CH₃)₂ | | |
| 436 | CF₃ | | | CF₃ | | | CF₃ | NO₂ | | |
| 437 | | | | CF₃O | | | CF₃ | NO₂ | | |
| 438 | | CF₃ | | CF₃ | | | CF₃ | NO₂ | | |
| 441 | | | | CF₃ | | CH₃O | | NO₂ | | |
| 445 | | | | CF₃ | | CH₃ | | | | |
| 446 | | | | CF₃ | | | | 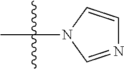 [SMILES: *n1ccnc1] | | |
| 449 | | | | CF₃ | | | | NO₂ | | |
| 456 | | | | NO₂ | | | CF₃ | NO₂ | | |
| 462 | | | CF₃ | | | | | NH₂ | | |
| 463 | | CF₃ | | CF₃ | | | CF₃ | CN | | |
| 464 | CF₃ | | | CF₃ | | | CF₃ | CN | | |
| 468 | CF₃ | | | CF₃ | | | | CN | | |
| 469 | | CF₃ | | CF₃ | | | | CN | | |
| 472 | CF₃ | | | CF₃ | | CH₃O | | NO₂ | | |
| 473 | CF₃ | | | CF₃ | | | | NO₂ | | |
| 474 | CF₃ | | | CF₃ | | CH₃ | | NO₂ | | |
| 488 | CF₃ | | | CF₃ | | | Cl | CN | | |
| 490 | | CF₃ | | CF₃ | | | Cl | CN | | |
| 723 | | CF₃ | | CF₃ | | | | N(CH₃)₂ | | |

(23) A compound according to (21) above, which is selected from the group of compounds consisting of 410, 414, 416, 433, 436, 438, 449, 463, 464, 468, 469, 488, 490 and 723.
(24) A compound according to (21) above, which is compound 410 or 469.
(25) A compound according to (1) above having the general formula A8

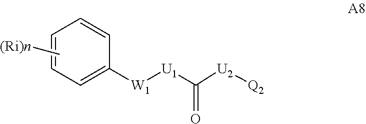

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different.
(26) A compound according to (25) above having the general formula A9

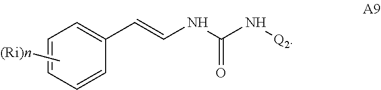

(27) A compound according to (26) above, which is selected from the group of compounds depicted below

| ID | Structure |
|---|---|
| 418 | 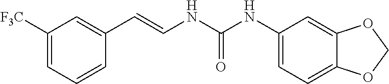 [SMILES: O=C(N/C=C/c1cccc(C(F)(F)F)c1)Nc1ccc2c(c1)OCO2] |
| 427 | 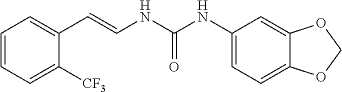 [SMILES: O=C(N/C=C/c1ccccc1C(F)(F)F)Nc1ccc2c(c1)OCO2] |
| 431 | 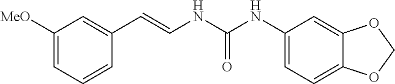 [SMILES: COc1cccc(/C=C/NC(=O)Nc2ccc3c(c2)OCO3)c1] |
| 432 | 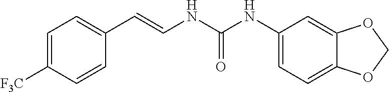 [SMILES: O=C(N/C=C/c1ccc(C(F)(F)F)cc1)Nc1ccc2c(c1)OCO2] |
| 515 | 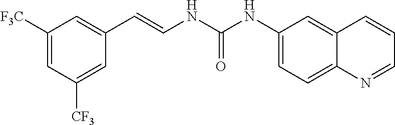 [SMILES: O=C(N/C=C/c1cc(C(F)(F)F)cc(C(F)(F)F)c1)Nc1ccc2ncccc2c1] |
| 516 | 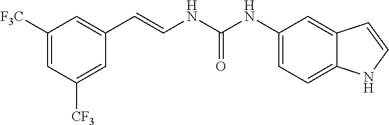 [SMILES: O=C(N/C=C/c1cc(C(F)(F)F)cc(C(F)(F)F)c1)Nc1ccc2[nH]ccc2c1] |
| 517 | 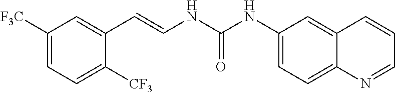 [SMILES: O=C(N/C=C/c1cc(C(F)(F)F)ccc1C(F)(F)F)Nc1ccc2ncccc2c1] |
| 518 | 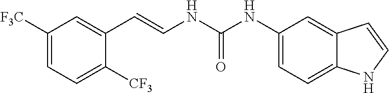 [SMILES: O=C(N/C=C/c1cc(C(F)(F)F)ccc1C(F)(F)F)Nc1ccc2[nH]ccc2c1] |
| 519 | 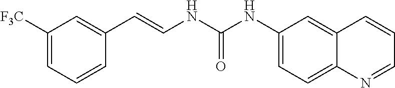 [SMILES: O=C(N/C=C/c1cccc(C(F)(F)F)c1)Nc1ccc2ncccc2c1] |
| 520 | 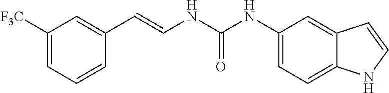 [SMILES: O=C(N/C=C/c1cccc(C(F)(F)F)c1)Nc1ccc2[nH]ccc2c1] |
| 523 | 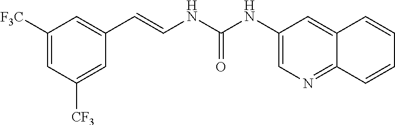 [SMILES: O=C(N/C=C/c1cc(C(F)(F)F)cc(C(F)(F)F)c1)Nc1cnc2ccccc2c1] |
| 524 | 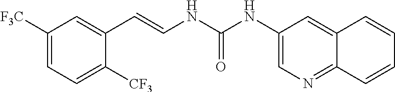 [SMILES: O=C(N/C=C/c1cc(C(F)(F)F)ccc1C(F)(F)F)Nc1cnc2ccccc2c1] |
| 525 | 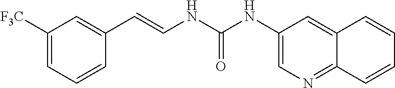 [SMILES: O=C(N/C=C/c1cccc(C(F)(F)F)c1)Nc1cnc2ccccc2c1] |

(28) A compound according to (26) above, which is selected from the group of compounds consisting of 517, 520, 523 and 524.
(29) A compound according to (1) above having the general formula A10

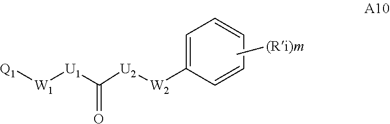

wherein:
m is an integer selected from 0 to 5, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different.
(30) A compound according to (29) above having the general formula A11

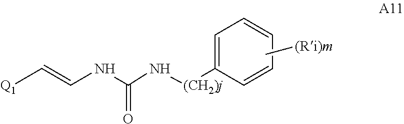

wherein:
j is an integer selected from 0 to 6.
(31) A compound according to (29) above, which is selected from the group of compounds depicted below

| ID | Structure |
|---|---|
| 419 | 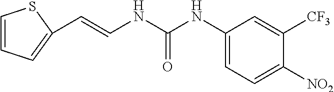 [SMILES: O=C(N/C=C/c1cccs1)Nc1ccc([N+](=O)[O-])c(C(F)(F)F)c1] |
| 420 | 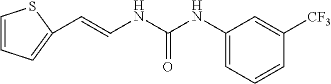 [SMILES: O=C(N/C=C/c1cccs1)Nc1cccc(C(F)(F)F)c1] |
| 424 | 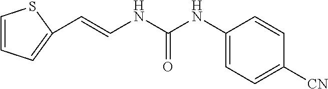 [SMILES: N#Cc1ccc(NC(=O)N/C=C/c2cccs2)cc1] |
| 425 | 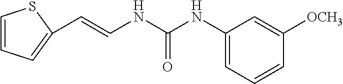 [SMILES: COc1cccc(NC(=O)N/C=C/c2cccs2)c1] |
| 426 | 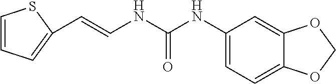 [SMILES: O=C(N/C=C/c1cccs1)Nc1ccc2c(c1)OCO2] |
| 428 | 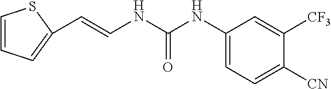 [SMILES: N#Cc1ccc(NC(=O)N/C=C/c2cccs2)cc1C(F)(F)F] |
| 434 | 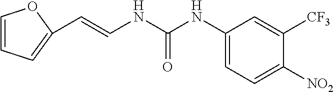 [SMILES: O=C(N/C=C/c1ccco1)Nc1ccc([N+](=O)[O-])c(C(F)(F)F)c1] |
| 443 | 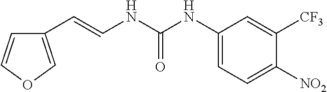 [SMILES: O=C(N/C=C/c1ccoc1)Nc1ccc([N+](=O)[O-])c(C(F)(F)F)c1] |
| 444 | 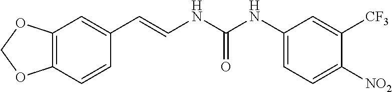 [SMILES: O=C(N/C=C/c1ccc2c(c1)OCO2)Nc1ccc([N+](=O)[O-])c(C(F)(F)F)c1] |
| 447 | 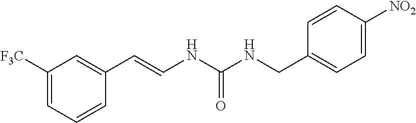 [SMILES: O=C(N/C=C/c1cccc(C(F)(F)F)c1)NCc1ccc([N+](=O)[O-])cc1] |
| 450 | 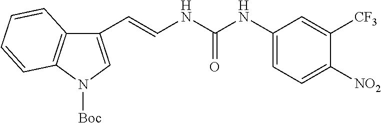 [SMILES: CC(C)(C)OC(=O)n1cc(/C=C/NC(=O)Nc2ccc([N+](=O)[O-])c(C(F)(F)F)c2)c2ccccc21] |
| 453 | 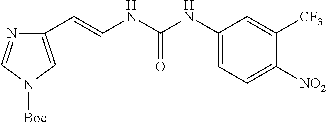 [SMILES: CC(C)(C)OC(=O)n1cnc(/C=C/NC(=O)Nc2ccc([N+](=O)[O-])c(C(F)(F)F)c2)c1] |
| 454 | 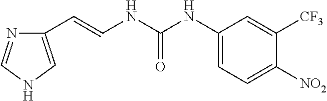 [SMILES: O=C(N/C=C/c1c[nH]cn1)Nc1ccc([N+](=O)[O-])c(C(F)(F)F)c1] |
| 459 | 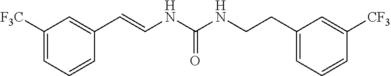 [SMILES: O=C(N/C=C/c1cccc(C(F)(F)F)c1)NCCc1cccc(C(F)(F)F)c1] |
| 460 | 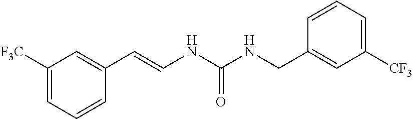 [SMILES: O=C(N/C=C/c1cccc(C(F)(F)F)c1)NCc1cccc(C(F)(F)F)c1] |
| 461 | 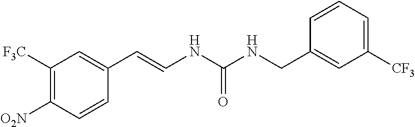 [SMILES: O=C(N/C=C/c1ccc([N+](=O)[O-])c(C(F)(F)F)c1)NCc1cccc(C(F)(F)F)c1] |
| 633 | 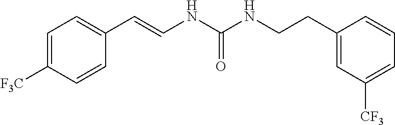 [SMILES: O=C(N/C=C/c1ccc(C(F)(F)F)cc1)NCCc1cccc(C(F)(F)F)c1] |
| 634 | 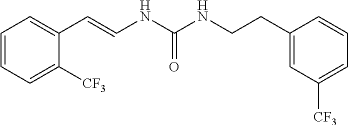 [SMILES: O=C(N/C=C/c1ccccc1C(F)(F)F)NCCc1cccc(C(F)(F)F)c1] |
| 635 | 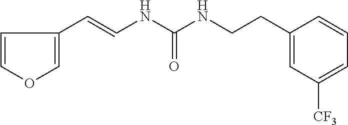 [SMILES: O=C(N/C=C/c1ccoc1)NCCc1cccc(C(F)(F)F)c1] |
| 642 | 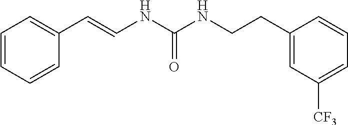 [SMILES: O=C(N/C=C/c1ccccc1)NCCc1cccc(C(F)(F)F)c1] |

(32) A compound according to (29) above, which is selected from the group of compounds consisting of compounds 419, 420, 428, 434, 447, 448, 450, 461 and 635.
(33) A compound according to (29) above having the general formula A12

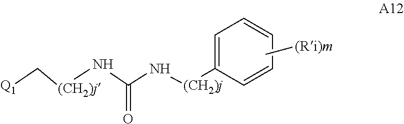

wherein:
j′ is an integer from 0 to 6, independently of j.
(34) A compound according to (33) above, which is

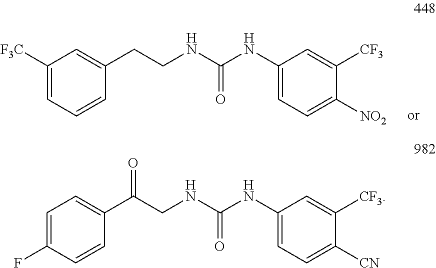 [SMILES: O=C(NCCc1cccc(C(F)(F)F)c1)Nc1ccc([N+](=O)[O-])c(C(F)(F)F)c1 ; N#Cc1ccc(NC(=O)NCC(=O)c2ccc(F)cc2)cc1C(F)(F)F]

(35) A compound according to (1) above having the general formula A13

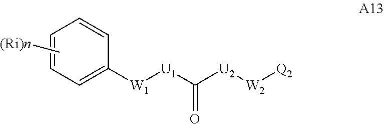

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different.
(36) A compound according to (33) above having the general formula A14

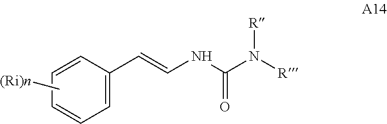

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different; and
at least one of R″ and R′″ is as Q₁, or R″ and R′″ are as R₁ and R₂.
(37) A compound according to (36) above, which is selected from the group of compounds depicted below

| ID | Structure |
|---|---|
| 534 | 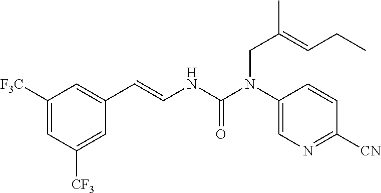 [SMILES: CC/C=C(\C)CN(C(=O)N/C=C/c1cc(C(F)(F)F)cc(C(F)(F)F)c1)c1ccc(C#N)nc1] |
| 547 | 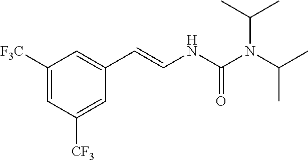 [SMILES: CC(C)N(C(=O)N/C=C/c1cc(C(F)(F)F)cc(C(F)(F)F)c1)C(C)C] |
| 563 | 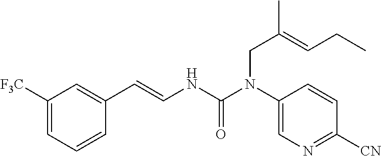 [SMILES: CC/C=C(\C)CN(C(=O)N/C=C/c1cccc(C(F)(F)F)c1)c1ccc(C#N)nc1] |
| 591 | 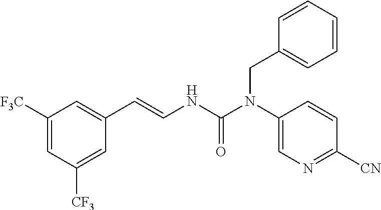 [SMILES: N#Cc1ccc(N(Cc2ccccc2)C(=O)N/C=C/c2cc(C(F)(F)F)cc(C(F)(F)F)c2)cn1] |
| 620 | 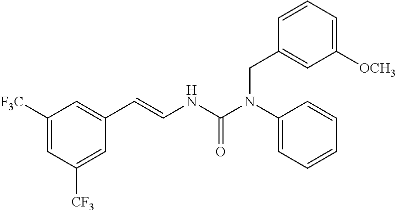 [SMILES: COc1cccc(CN(C(=O)N/C=C/c2cc(C(F)(F)F)cc(C(F)(F)F)c2)c2ccccc2)c1] |
| 621 | 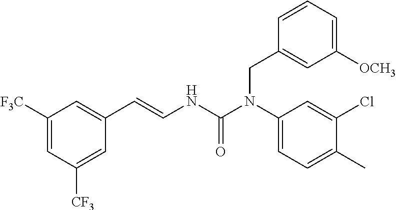 [SMILES: COc1cccc(CN(C(=O)N/C=C/c2cc(C(F)(F)F)cc(C(F)(F)F)c2)c2ccc(C)c(Cl)c2)c1] |
| 622 | 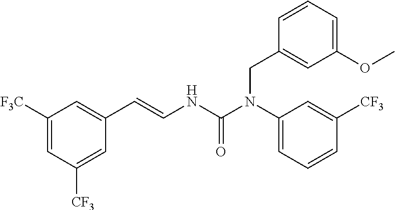 [SMILES: COc1cccc(CN(C(=O)N/C=C/c2cc(C(F)(F)F)cc(C(F)(F)F)c2)c2cccc(C(F)(F)F)c2)c1] |
| 623 | 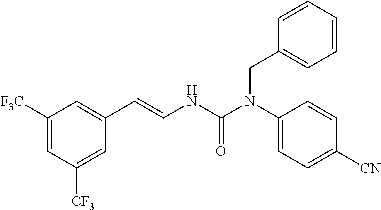 [SMILES: N#Cc1ccc(N(Cc2ccccc2)C(=O)N/C=C/c2cc(C(F)(F)F)cc(C(F)(F)F)c2)cc1] |

(38) A compound according to (36) above, which is selected from the group of compounds consisting of compounds 534, 591 and 622.
(39) A compound according to (1) above having the general formula A15

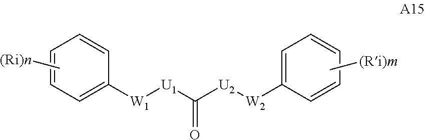

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different; and
m is an integer selected from 0 to 5, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different.
(40) A compound according to (39) above having the general formula A16

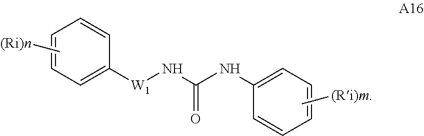

(41) A compound according to (40) above, which is selected from the group of compounds depicted in the table below

| ID. | Structure |
|---|---|
| 804 | 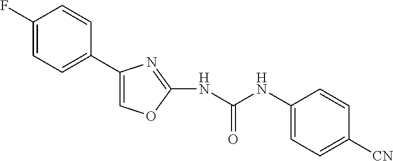 [SMILES: N#Cc1ccc(NC(=O)Nc2nc(-c3ccc(F)cc3)co2)cc1] |
| 790 | 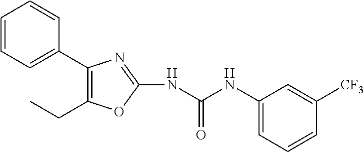 [SMILES: CCc1oc(NC(=O)Nc2cccc(C(F)(F)F)c2)nc1-c1ccccc1] |
| 791 | 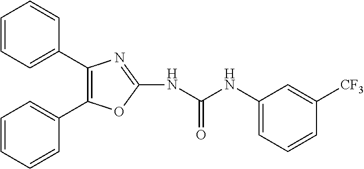 [SMILES: O=C(Nc1cccc(C(F)(F)F)c1)Nc1nc(-c2ccccc2)c(-c2ccccc2)o1] |
| 797 | 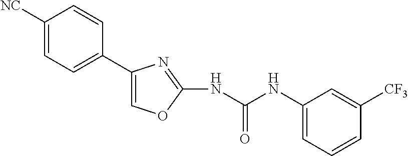 [SMILES: N#Cc1ccc(-c2coc(NC(=O)Nc3cccc(C(F)(F)F)c3)n2)cc1] |
| 798 | 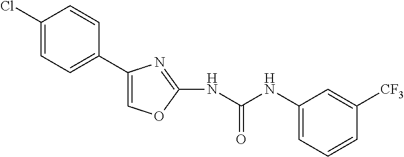 [SMILES: O=C(Nc1cccc(C(F)(F)F)c1)Nc1nc(-c2ccc(Cl)cc2)co1] |
| 799 | 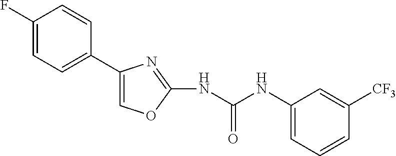 [SMILES: O=C(Nc1cccc(C(F)(F)F)c1)Nc1nc(-c2ccc(F)cc2)co1] |
| 803 | 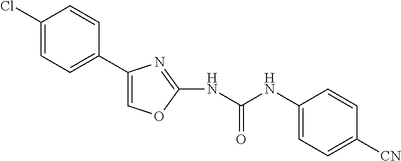 [SMILES: N#Cc1ccc(NC(=O)Nc2nc(-c3ccc(Cl)cc3)co2)cc1] |
| 805 | 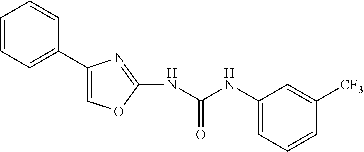 [SMILES: O=C(Nc1cccc(C(F)(F)F)c1)Nc1nc(-c2ccccc2)co1] |
| 802 | 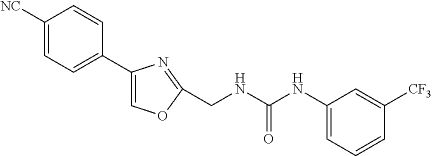 [SMILES: N#Cc1ccc(-c2coc(CNC(=O)Nc3cccc(C(F)(F)F)c3)n2)cc1] |
| 783 | 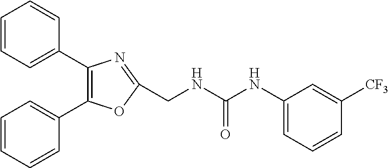 [SMILES: O=C(NCc1nc(-c2ccccc2)c(-c2ccccc2)o1)Nc1cccc(C(F)(F)F)c1] |
| 788 | 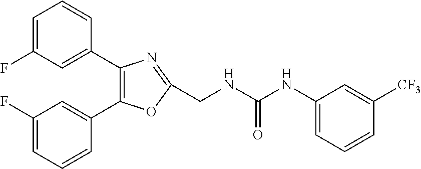 [SMILES: O=C(NCc1nc(-c2cccc(F)c2)c(-c2cccc(F)c2)o1)Nc1cccc(C(F)(F)F)c1] |
| 885 | 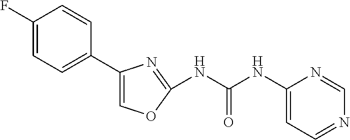 [SMILES: O=C(Nc1ccncn1)Nc1nc(-c2ccc(F)cc2)co1] |

(42) A compound according to (40) above, which is selected from the group of compounds consisting of compounds 804, 788 and 790.
(43) A compound according to (1) above having the general formula A17

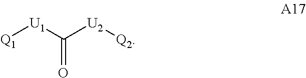

(44) A compound according to (43) above having the general formula A18

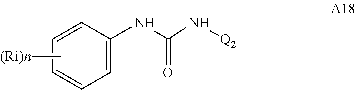

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different.
(45) A compound according to (44) above, which is selected from the group of compounds defined as outlined below

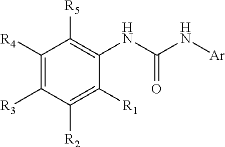

566 Analogues of Formula (I)

| | Substituents | | | | | | | | |
|---|---|---|---|---|---|---|---|---|---|
| | | | | Ar | | | | | |
| ID | R₂ | R₃ | R₄ | | R₆ | R₇ | R₈ | R₉ | R₁₀ |
| 484 | | | CF₃ | A | | | CN | | |
| 486 | | | CF₃ | B | | | CN | | |
| 491 | | | CF₃ | A | | | NO₂ | | |
| 495 | CF₃ | | CF₃ | A | | | CN | | |
| 496 | CF₃ | | CF₃ | A | | | NO₂ | | |
| 498 | | CF₃ | | B | | | CN | | |
| 499 | | CF₃ | | A | | | CN | | |
| 501 | | CF₃ | | A | | | NO₂ | | |
| 506 | CF₃ | | CF₃ | B | | | | | |
| 507 | | CF₃ | | B | | | | | |
| 565 | CF₃ | | CF₃ | B | Cl | | | | |
| 566 | CF₃ | | CF₃ | B | | | Br | | |
| 567 | | | CF₃ | B | | F | | | |
| 568 | | | CF₃ | B | Cl | | | | |
| 569 | | | CF₃ | B | | | Br | | |
| 570 | | | CF₃ | B | CH₃ | | | | |
| 571 | | | CF₃ | D | | | | | |
| 572 | | | CF₃ | E | | Ph | | | |
| 573 | CF₃ | | CF₃ | B | | F | | | |
| 575 | | | CF₃ | C | | | CF₃ | | |
| 576 | CF₃ | | CF₃ | D | | | | | |
| 579 | CF₃ | | CF₃ | B | | | CH₃ | | |
| 580 | | | CF₃ | E | | CF₃ | | | |
| 584 | CF₃ | | CF₃ | E | | CF₃ | | | |
| 739 | CF₃ | | | B | | | F | | |
| 740 | CF₃ | | | B | | | Cl | | |
| 741 | CF₃ | | | B | | Cl | Cl | | |
| 754 | CF₃ | | CF₃ | B | | | F | | |
| 755 | CF₃ | | CF₃ | B | | | Cl | | |
| 758 | CF₃ | | CF₃ | B | | Cl | Cl | | |
| 763 | CF₃ | | CF₃ | B | CH₃ | | | | Cl |
| 764 | CF₃ | | CF₃ | B | CH₃ | | F | | |
| 773 | CN | | | | | | Br | | |

| | |
|---|---|
| 522 | 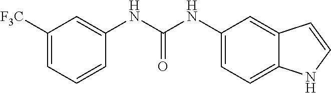 [SMILES: O=C(Nc1cccc(C(F)(F)F)c1)Nc1ccc2[nH]ccc2c1] |
| 530 | 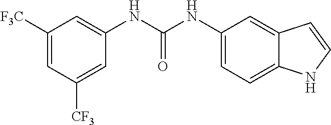 [SMILES: O=C(Nc1cc(C(F)(F)F)cc(C(F)(F)F)c1)Nc1ccc2[nH]ccc2c1] |
| 574 | 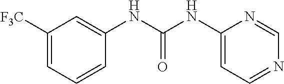 [SMILES: O=C(Nc1cccc(C(F)(F)F)c1)Nc1ccncn1] |
| 578 | 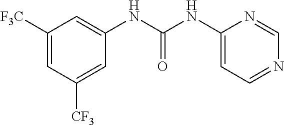 [SMILES: O=C(Nc1cc(C(F)(F)F)cc(C(F)(F)F)c1)Nc1ccncn1] |
| 737 | 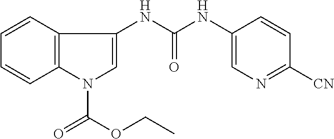 [SMILES: CCOC(=O)n1cc(NC(=O)Nc2ccc(C#N)nc2)c2ccccc21] |
| 738 | 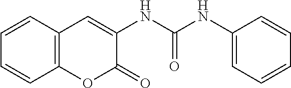 [SMILES: O=C(Nc1ccccc1)Nc1cc2ccccc2oc1=O] |
| 744 | 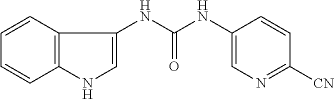 [SMILES: N#Cc1ccc(NC(=O)Nc2c[nH]c3ccccc23)cn1] |
| 753 | 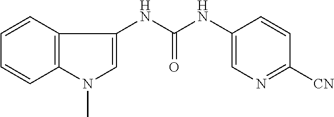 [SMILES: Cn1cc(NC(=O)Nc2ccc(C#N)nc2)c2ccccc21] |

R₁ = R₅ = H

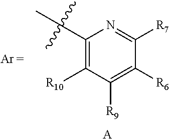

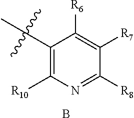

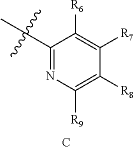

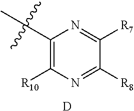

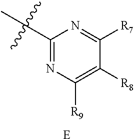

(46) A compound according to (44) above, which is selected from the group of compounds consisting of 484, 486, 495, 496, 498, 566, 569, 572, 576, 579, 580, 578, 739, 530 and 744.
(47) A compound according to (44) above, which is

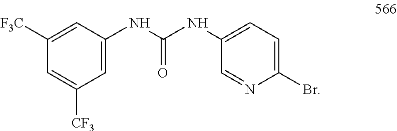 [SMILES: O=C(Nc1ccc(Br)nc1)Nc1cc(C(F)(F)F)cc(C(F)(F)F)c1]

(48) A compound according to (1) above having the general formula A19

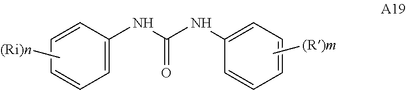

wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different; and
m is an integer selected from 0 to 5, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive R′i together form a 5 to 8-member ring which optionally comprises one or more heteroatom which are the same or different.
(49) A compound according to (48) above, which is selected from the group of compounds defined as outlined below

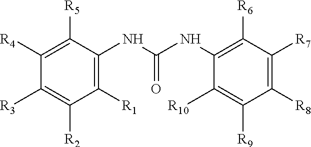

### 566 Analogues of Formula (II)

| | Substituents | | | | | | | | | |
|---|---|---|---|---|---|---|---|---|---|---|
| ID | R₁ | R₂ | R₃ | R₄ | R₅ | R₆ | R₇ | R₈ | R₉ | R₁₀ |
| 442 | | | | CF₃ | | | CF₃ | NO₂ | | |
| 465 | | | NO₂ | CF₃ | | | CF₃ | CN | | |
| 467 | | | | CF₃ | | | CF₃ | CN | | |
| 492 | | | | CF₃ | | | Cl | CN | | |
| 494 | | CF₃ | | CF₃ | | | CF₃ | CN | | |
| 500 | | CF₃ | | CF₃ | | | Cl | CN | | |
| 502 | | | CF₃ | | | | Cl | CN | | |
| 509 | | | CF₃ | | | | CF₃ | CN | | |
| 646 | | OCH₃ | | | | | Cl | CN | | |
| 647 | | Cl | | | | | Cl | CN | | |
| 680 | | | | | | | Cl | CN | | |
| 701 | | OCH₃ | | | | | CF₃ | CN | | |
| 702 | | | | | | | CF₃ | CN | | |
| 703 | | CH₃ | | | | | CF₃ | CN | | |
| 704 | | F | | | | | CF₃ | CN | | |
| 705 | | Cl | | | | | CF₃ | CN | | |
| 706 | CF₃ | | | CF₃ | | | CF₃ | CN | | |
| 736 | | CF₃ | | | | | CF₃ | NH₂ | | |
| 745 | | CF₃ | | | | | | NH₂ | | |
| 772 | | CN | | | | | Cl | CN | | |
| 774 | | CN | | | | | CN | CN | | |
| 792 | | CF₃ | | CF₃ | | | F | CN | | |
| 829 | | CF₃ | F | | | | CF₃ | NO₂ | | |
| 887 | | CF3 | | OCH₃ | | | CF3 | | | |

(50) A compound according to (48) above, which is selected from the group of compounds consisting of 442, 467, 492, 494, 500, 502, 509 and 792.
(51) A compound according to (1) above having the general formula B1

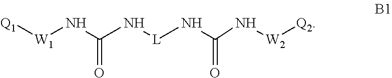

(52) A compound according to (51) above having the general formula B2

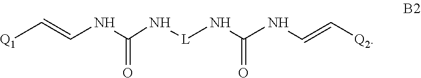

(53) A compound according to (52) above, which is selected from the group of compounds depicted below

| ID | Structure |
|---|---|
| 439 | 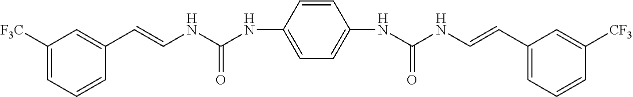 [SMILES: O=C(N/C=C/c1cccc(C(F)(F)F)c1)Nc1ccc(NC(=O)N/C=C/c2cccc(C(F)(F)F)c2)cc1] |
| 440 | 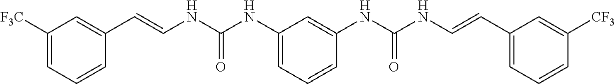 [SMILES: O=C(N/C=C/c1cccc(C(F)(F)F)c1)Nc1cccc(NC(=O)N/C=C/c2cccc(C(F)(F)F)c2)c1] |
| 451 | 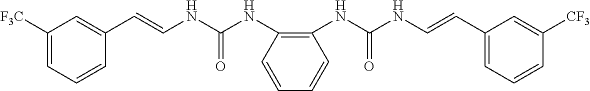 [SMILES: O=C(N/C=C/c1cccc(C(F)(F)F)c1)Nc1ccccc1NC(=O)N/C=C/c1cccc(C(F)(F)F)c1] |
| 452 | 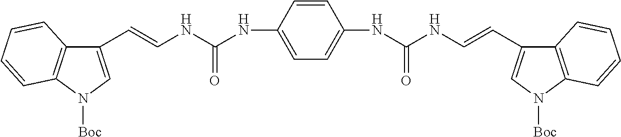 [SMILES: CC(C)(C)OC(=O)n1cc(/C=C/NC(=O)Nc2ccc(NC(=O)N/C=C/c3cn(C(=O)OC(C)(C)C)c4ccccc34)cc2)c2ccccc21] |
| 455 | 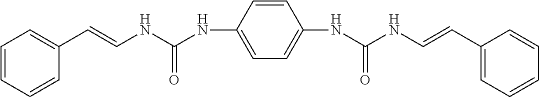 [SMILES: O=C(N/C=C/c1ccccc1)Nc1ccc(NC(=O)N/C=C/c2ccccc2)cc1] |
| 457 | 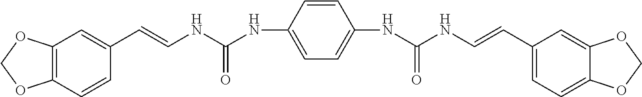 [SMILES: O=C(N/C=C/c1ccc2c(c1)OCO2)Nc1ccc(NC(=O)N/C=C/c2ccc3c(c2)OCO3)cc1] |
| 458 | 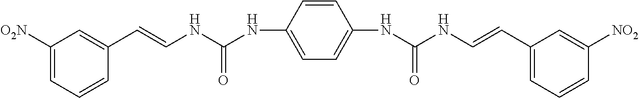 [SMILES: O=C(N/C=C/c1cccc([N+](=O)[O-])c1)Nc1ccc(NC(=O)N/C=C/c2cccc([N+](=O)[O-])c2)cc1] |
| 466 | 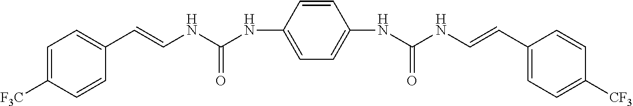 [SMILES: O=C(N/C=C/c1ccc(C(F)(F)F)cc1)Nc1ccc(NC(=O)N/C=C/c2ccc(C(F)(F)F)cc2)cc1] |
| 532 | 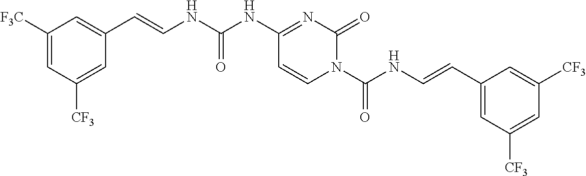 [SMILES: O=C(N/C=C/c1cc(C(F)(F)F)cc(C(F)(F)F)c1)Nc1ccn(C(=O)N/C=C/c2cc(C(F)(F)F)cc(C(F)(F)F)c2)c(=O)n1] |

(54) A compound according to (52) above, which is selected from the group of compounds consisting of 439, 440, 451, 466 and 532.
(55) A compound according to (17), (22), (27), (31), (37) or (45) above, which is selected from the group of compounds consisting of 410, 481, 528, 531, 538, 421, 436, 438, 464, 468, 517, 428, 461, 534, 566, 495, 496 and 578.
(56) A compound according to any one of (1) to (55) above, which targets the N-terminal domain of the androgen receptor (AR-NTD).
(57) A compound according to any one of (1) to (55) above, which targets mutants of the androgen receptor, preferably the F876L mutated androgen receptor.
(58) A compound according to any one of (1) to (55) above, which targets androgen receptor variants, preferably the androgen receptor variant lacking the ligand-binding domain (LBD) such as for example AR-v7 and AR^{v567es}.
(59) A compound according to (55) above, which targets cancer cells lacking any androgen receptor (AR negative cells), preferably DU145 or PC3 cells.
(60) A pharmaceutical composition comprising a compound as defined in any one of (1) to (55) above, and a pharmaceutically acceptable carrier.
(61) A method of treating a medical condition that may or may not involve hormones, comprising administering to a subject a therapeutically effective amount of a compound as defined in any one of (1) to (55) above or a therapeutically effective amount of a pharmaceutical composition as defined in (60) above.
(62) A method according to (61) above, wherein the medical condition is selected from: androgen-dependent diseases or disorders and androgen receptor-mediated diseases or disorders.
(63) A method according to (61) above, wherein the medical condition is selected from: prostate cancer including AR positive prostate cancers, castration-resistant prostate cancers, breast cancer including AR positive breast cancers, ovarian cancer, hepatocellular carcinoma, endometrial cancer, benign prostatic hyperplasia, endometriosis, male pattern baldness, spinal and bulbar muscular atrophy.
(64) A method according to (61) above, wherein the medical condition is selected from: prostate cancer including AR negative prostate cancers, breast cancer including AR negative breast cancers, ovarian cancer, hepatocellular carcinoma, endometrial cancer, benign prostatic hyperplasia, endometriosis, male pattern baldness, spinal and bulbar muscular atrophy.
(65) A method according to (61) above, wherein the medical condition is prostate cancer, including castration-resistant prostate cancers and advanced prostate cancers.
(66) Use of a compound as defined in any one of (1) to (55) above or a pharmaceutical composition as defined in (60) above, for treating in a subject a medical condition that may or may not involve hormones.
(67) A use according to (66) above, wherein the medical condition is selected from: androgen-dependent diseases or disorders and androgen receptor-mediated diseases or disorders.
(68) A use according to (66) above, wherein the medical condition is selected from: prostate cancer including AR positive prostate cancers, castration-resistant prostate cancers, breast cancer including AR positive breast cancers, ovarian cancer, hepatocellular carcinoma, endometrial cancer, benign prostatic hyperplasia, endometriosis, male pattern baldness, spinal and bulbar muscular atrophy.
(69) A use according to (66) above, wherein the medical condition is selected from: prostate cancer including AR negative prostate cancers, breast cancer including AR negative breast cancers, ovarian cancer, hepatocellular carcinoma, endometrial cancer, benign prostatic hyperplasia, endometriosis, male pattern baldness, spinal and bulbar muscular atrophy.
(70) A use according to (66) above, wherein the medical condition is prostate cancer, including castration-resistant prostate cancers and advanced prostate cancers.
(71) Use of a compound as defined in any one of (1) to (55) above, in the manufacture of a medicament for treating a medical condition that may or may not involve hormones.
(72) A use according to (71) above, wherein the medical condition is selected from: androgen-dependent diseases or disorders and androgen receptor-mediated diseases or disorders.
(73) A use according to (71) above, wherein the medical condition is selected from: prostate cancer including AR positive prostate cancers, castration-resistant prostate cancers, breast cancer including AR positive breast cancers, ovarian cancer, hepatocellular carcinoma, endometrial cancer, benign prostatic hyperplasia, endometriosis, male pattern baldness, spinal and bulbar muscular atrophy.
(74) A use according to (71) above, wherein the medical condition is selected from: prostate cancer including AR negative prostate cancers, breast cancer including AR negative breast cancers, ovarian cancer, hepatocellular carcinoma, endometrial cancer, benign prostatic hyperplasia, endometriosis, male pattern baldness, spinal and bulbar muscular atrophy.
(75) A use according to (71) above, wherein the medical condition is prostate cancer, including castration-resistant prostate cancers and advanced prostate cancers.
(76) A compound as defined in any one of (1) to (55) above, for use in the treatment of a medical condition that may or may not involve hormones.
(77) A compound according to (76) above, wherein the medical condition is selected from: androgen-dependent diseases or disorders and androgen receptor-mediated diseases or disorders.
(78) A compound according to (76) above, wherein the medical condition is selected from: prostate cancer including AR positive prostate cancers, castration-resistant prostate cancers, breast cancer including AR positive breast cancers, ovarian cancer, hepatocellular carcinoma, endometrial cancer, benign prostatic hyperplasia, endometriosis, male pattern baldness, spinal and bulbar muscular atrophy.
(79) A compound according to (76) above, wherein the medical condition is selected from: prostate cancer including AR negative prostate cancers, breast cancer including AR negative breast cancers, ovarian cancer, hepatocellular carcinoma, endometrial cancer, benign prostatic hyperplasia, endometriosis, male pattern baldness, spinal and bulbar muscular atrophy.
(80) A compound according to (76) above, wherein the medical condition is prostate cancer, including castration-resistant prostate cancers and advanced prostate cancers.
(81) A method according to (61) above or use according to (66) above, further comprising treating the subject with a second cancer therapy.
(82) A method according to (61) above or use according to (66) above, wherein the compound is administered intravenously, intra-arterially, subcutaneously, topically or intramuscularly.
(83) A method according to (61) above or use according to (66) above, wherein the cancer is multi-drug resistant, metastatic and/or recurrent.
(84) A method according to any one of (61) and (81) to (83) above or use according to (66) above, wherein the method or use comprises inhibiting cancer growth, killing cancer cells, reducing tumor burden, reducing tumor size, improving the subject's quality of life and/or prolonging the subject's length of life.
(85) A method according to any one of (61) and (81) to (83) above or use according to (66) above, wherein the subject is a human.
(86) A method according to any one of (61) and (81) to (83) above or use according to (66) above, wherein the subject is a non-human animal.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1: The current therapeutic modalities for advanced prostate cancer: A) Androgen depleting agents; B) Antiandrogens; and the chemical structures of all of the FDA-approved antiandrogen enzalutamide, bicalutamide, flutamide and nilutamide. The structure of abiraterone is also shown.

FIG. 2: Emergence of AR-v7 confers resistance to all of the FDA-approved antiandrogens and the androgen-depleting agent abiraterone.

FIG. 3: The AR-NTD is an attractive drug target: A) All of the known mechanisms that could account for AR reactivation in CRPC cells are critically depending on the AR-NTD to reactivate AR; B) Among the NTD, DBD and LBD domains, the NTD is the most different domain between the AR and other members of steroid receptors.

FIG. 4: A and B Compounds according to embodiments of the invention.

FIG. 5: Our workflow to identify novel inhibitors that target the AR-NTD.

FIG. 6: We have used two methods for verifying whether the compound targets the AR-NTD. A) Method 1 utilizes the fusion protein VP16-AR(507-919). The fusion protein VP16-AR(509-919) lacks the AR-LBD but retains the AR-DBD and AR-LBD. Thus, DHT-induced activation of VP16-AR(509-919) could be inhibited by the AR-LBD targeting agents, but cannot be inhibited by the AR-NTD-directed inhibitors. In contrast, AR-NTD inhibitors are active against the AR-v7 and full-length AR. B) Method 2 utilizes the DBD of IRF3 (referred to as IRF3DBD) and fusion protein of IRF3DBD fused with AR-NTD (referred to as IRF3DBD-AR-NTD). The IRF3-DBD alone is transcriptionally inactive as it needs a transactivation domain at the C-terminus. We found that when the AR-NTD is fused with the IRF3-DBD domain, the resulted fusion protein has a good transcriptional activity, which could therefore be inhibited by the AR-NTD inhibitors. The AR-NTD consists of two transactivation units referred to as TAU1 containing the core sequence of AR residues 178-182 and TAU5 containing AR residues 360-529 with the core sequence of residues 435-439.

FIG. 7: These results indicate that compounds 562 and 746 are targeting the AR-NTD. Compounds 562 and 746 dose-dependently inhibit AR-v7 and WT full-length AR, but are inactive against the VP16-AR(507-919) (A-C). As AR(507-919) is the NTD-deleted AR, VP16-AR(507-919) fusion protein is also referred to as VP16-AR(delNTD). In contrast, LBD-targeting enzalutamide (ENZ) and bicalutamide (BIC) are inactive against AR-v7, but remain active against the full-length AR and VP16-AR(delNTD) (A-C). Experimental details: A) For the NT, HEK293 cells were co-transfected with pIRES vector, PSA-luc reporter and pRL-TK plasmids. For all of the other wells, pIRES-AR-v7, PSA-luc and pRL-TK plasmids were transiently transfected into HEK293 cells. Transfected cells were exposed to DMSO vehicle or compounds in phenol red-free medium and 10% charcoal-stripped FBS (CS-FBS) for 24 h. B) and C) Plasmids expressing WT full-length AR or VP16-AR(507-919), PSA-luc and pRL-TK were co-transfected into PC3 or HEK293 cells. Cells were exposed to DMSO vehicle, 10 nM DHT alone or compounds in the presence of 10 nM DHT for 24 h, in triplicate. ENZ, enzalutamide; Bic, Bicalutamide.

FIG. 8: The results indicated that compounds 562, 566 and 746 are targeting the AR-NTD. NT, HEK293 cells were co-transfected with IRF3DBD, ISRE-luc reporter and pRL-TK. For all of the others, cells were co-transfected with full-length IRF3 or IRF3DBD-AR(1-547) expressing plasmids and with the ISRE-luc reporter and pRL-TK plasmids. Cells were exposed to DMSO vehicle or compounds for 24 h, in triplicate. As IRF3DBD-AR(1-547) contains the DBD of IRF3, ISRE-luc reporter instead of the PSA-luc reporter was used in our assays.

FIG. 9: Selectivity: compounds 562, 566 and 746 do not interfere with the transcriptional activation of the PR and GR. A) PC3 cells express endogenous GR. MMTV-Luc and pRL-TK were transiently co-transfected into PC3 cells. Cells were exposed to DMSO, 10 nM DEX (a GR agonist) or compounds in the presence and absence of 10 nM DEX for 24 h; B) MMTV-Luc and PR expressing plasmids and pRL-TK were co-transfected into PC3 cells and cells were exposed to DMSO, 10 nM R5020 (a PR agonist) or compounds in the presence and absence of R5020 for 24 h, in triplicate.

FIG. 10: Compounds 562 and 746 inhibit DHT-induced transactivation of the F876L, W741C, T877A and H874Y mutants of full-length ARs. In contrast, enzalutamide cannot inhibit the F876L and bicalutamide cannot inhibit the W741C. Plasmids expressing the WT or mutants of full-length AR, PSA-luc and pRL-TK were co-transfected into PC3 cells. Cells were exposed to DMSO vehicle, 10 nM DHT alone or compounds in the presence of 10 nM DHT for 24 h, in triplicate.

FIG. 11: A) Compounds 562 and 746 suppressed DHT-induced AR activation in LNCaP cells which endogenously express the T877A mutant; B) Western blot analysis indicated compound 746 at 2.5 μM suppressed DHT-induced expression of the PSA in LNCaP cells. Cells were exposed to DMSO vehicle, 10 nM DHT alone or compounds in the presence of 10 nM DHT in phenol-red free medium plus 10% charcoal-stripped FBS (CS-FBS) for 24 h; C) Compound 746 suppressed DHT-induced AR activation in 22Rv1 cells which endogenously express the H874Y mutant of full-length AR. Cells were transiently transfected with PSA-luc and pRL-TK, and then exposed to DMSO vehicle, 10 nM DHT alone or compounds in the presence of 10 nM DHT for 24 h, in triplicate. ENZ, enzalutamide; BIC, bicalutamide; EPI, EPI-001. Compound EPI-001 was purchased from Sigma-Aldrich (catalog number: 92427),

FIG. 12: A) Forced expression of AR-v7 confers resistance to enzalutamide and bicalutamide when LNCaP cells were transiently transfected with pIRES-AR-v7 plasmid. In contrast, compounds 562 and 746 remain active in such cells. NT, LNCaP cells were transiently transfected with pIRES vector, PSA-luc and pRL-TK. For all others, LNCaP cells were transfected with pIRES-AR-v7, PSA-luc and pRL-TK plasmids and exposed to DMSO vehicle or compounds for 24 h; B) Western blot analysis by AR-v7 antibody confirmed expression of AR-v7 protein when and only when LNCaP cells are transfected with pIRES-AR-v7 plasmid; C) The 22Rv1 cells endogenously express both full-length AR and the LBD-truncated AR variants (AR-Vs), including the AR-v7. The ARs were detected by the NTD directed AR antibody (Santa Cruz, N20); D) Endogenous expression of the LBD-truncated AR variants in 22Rv1 cells confer resistance to enzalutamide and bicaluamide, but compounds 562 and 746 remain active in such system. More specifically, to evaluate effect of compounds on the constitutive activation of the endogenous LBD-truncated AR variants in 22Rv1 cells, the cells were cultured in androgen-deleted medium (phenol red-free RPMI 1640 plus 10% CS-FBS) for 3 days to make sure the full-length AR is silenced. Cells were then transiently transfected with PSA-Luc and pRL-TK and exposed to DMSO or compounds for 24 h, in triplicate. NT, not transfected with PSA-luc.

FIG. 13: Compounds according to embodiments of the invention.

FIG. 14: The assay indicated that compounds 442, 467 and 492 inhibit the constitutive activation of AR-v7 (A) and are targeting the AR-NTD (B). A) For the NT, HEK293 cells were co-transfected with pIRES vector, PSA-luc and pRL-TK plasmids. For all of the others, pIRES-AR-v7, PSA-luc reporter and pRL-TK plasmids were transiently transfected into HEK293 cells. Transfected cells were exposed to DMSO vehicle or compounds in phenol red-free medium and 10% charcoal-stripped FBS (CS-FBS) for 24 h. *p<0.05, **p<0.001 and ***p<0.0001 when compared with the DMSO vehicle; B) For the VP16-AR(507-919) assay, plasmids expressing VP16-AR(507-919), PSA-luc reporter and pRL-TK (internal control) were co-transfected into HEK293 cells. Cells were exposed to DMSO vehicle, 1 nM DHT alone or compounds in the presence of 1 nM DHT for 24 h. Bic, Bicalutamide.

FIG. 15: The assay further confirmed that compounds 442 and 467 are targeting the AR-NTD. HEK293 cells were co-transfected with IRF3DBD(1-133) or IRF3DBD-AR(1-547) or IRF3DBD-AR(181-547) or full-length IRF3 expressing plasmids and with the ISRE-luc reporter and the Renilla luciferase pRL-TK plasmids. Cells were exposed to DMSO vehicle or compounds for 24 h. **p<0.001 when compared with DMSO vehicle control.

FIG. 16: Compounds 442, 467 and 492 are inactive against transcriptional function of the GR. AR and GR are close homology proteins and both of them belong to steroid receptor family. The assay indicated that compounds 442 and 467 do not suppress GR transcriptional activation induced by its agonist dexamethasone (DEX) and are non-agonist of the GR when compounds were evaluated in the absence of DEX. MMTV-luc reporter and pRL-TK plasmids were transiently co-transfected into PC3 cells, which endogenously express GR. Transfected cells were exposed to DMSO vehicle, 10 nM DEX alone or compounds in the presence of 10 nM DEX (A) or in the absence of DEX (B). DEX is an agonist of the GR.

FIG. 17: Compounds 442, 467 and 492 dose-dependently suppressed DHT-induced activation of the AR wild type and the F876L, W741C, T877A and H874Y mutants in AR-dependent reporter assays in PC3 cells (A-E). For the NT, pCMV vector, PSA-luc (reporter) and pRL-TK (internal control) plasmids were co-transfected into PC3 cells and the cells were exposed to 10 nM DHT. For all of the others, plasmid expressing full-length AR wild type or mutants, PSA-luc and pRL-TK plasmids were co-transfected into PC3 cells. Cells were exposed to DMSO vehicle, 10 nM DHT alone or compounds at designated doses in the presence of 10 nM DHT for 24 h. Experiments were in duplicates and repeated at least three times. Bic (bicalutamide) and ENZ (enzalutamide) at 5 μM were included as positive controls.

FIG. 18: Compounds 442, 467 and 492 are non-agonist of the full-length AR WT, F876L, W741C and T877A mutants in AR-dependent reporter assays in PC3 cells (A-D). For the NT, pCMV vector, PSA-luc (reporter) and pRL-TK (internal control) plasmids were co-transfected into PC3 cells and the cells were exposed to 10 nM DHT. For all of the others, plasmid expressing full-length AR mutants, PSA-luc and pRL-TK plasmids were co-transfected into PC3 cells. Cells were exposed to DMSO vehicle, 10 nM DHT alone or compounds at designated concentration (μM) in the absence of DHT for 24 h. Experiments were in duplicates and repeated at least twice. ENZ, enzalutamide; Bic, bicalutamide; OHF, hydroxyflutamide.

FIG. 19: Compounds 442 and 467 potently inhibit DHT-inducted activation of the endogenous AR in LNCaP cells (A). Importantly, by increasing DHT from 1 nM to 10 nM, the inhibitory activities of compounds 442 and 467 were not affected, which is expected for the AR-NTD targeting agents, but the activity of LBD-targeting agent Bic was substantially attenuated (B). PLSA-luc and pRL-TK plasmids were transiently co-transfected into LNCaP cells, which express endogenous AR T877A mutant, and cells were exposed to DMSO vehicle control, DHT alone or with the indicated compounds for 24 h. For the NT, cells were transfected with empty vector and pRL-TK plasmid and exposed to 1 nM or 10 nM DHT. Experiments were in duplicate and repeated three times. RLU, relative luciferase unit; C) Western blot analysis revealed that compounds 442 and 467 dose-dependently suppressed PSA expression and induced apoptosis in LNCaP cells. LNCaP cells in whole medium were exposed to DMSO vehicle control or compounds for 24 h.

FIG. 20: Compounds 442 and 467, but not the LBD-targeting Bic and ENZ, significantly suppressed constitutive activation of the endogenous AR-Vs in the 22Rv1 cells, and induced apoptosis (A-C). A) The 22Rv1 cells express substantial level of AR-Vs, which include AR-V7 and other AR variants lacking the LBD. The ARs were probed by N-terminal directed AR antibody (N20, Santa Cruz); B) The 22Rv1 cells were androgen-starved (in phenol red-free medium+10% CS-FBS) for 3 days to ensure the full-length AR expressed in 22Rv1 are not activated. The 22Rv1 cells were subsequently co-transfected with PSA-luc and pRL-TK plasmids. Cells were exposed to DMSO vehicle or compounds in phenol red-free medium+10% CS-FBS for 24 h. NT, only pRL-TK and empty vector were transfected into the cells. *p<0.05, **p<0.001, ***p<0.0001 when compared with DMSO control. n.s., non-significant; C) compounds 442 and 467 induced apoptosis in 22Rv1 cells. Cells in phenol red-free medium+10% CS-FBS were exposed to DMSO or compounds for 24 h and harvested for Western blot analysis.

FIG. 21: A) In PSA-Luc/AR-v7 reporter assay in HEK293 cells, compounds 562, 566 and 746 at 2.5 μM and EPI-001 at 25 μM inhibit the constitutive activation of wild-type AR-v7; B) Compound 566, EPI-001, compound 562 and compound 746 are active against the endogenous AR-Vs in 22Rv1 cells. The cells were androgen-starved for 3 days and transfected with PSA-Luc and pRL-TK plasmids. NT, cells were transfected with empty vector. Cells were exposed to vehicle control or compounds for 24 h. EPI, EPI-001.

FIG. 22: The analogues of compound 746 and other compounds from this invention inhibit the constitutively activation of AR-v7 (A) and the DHT-induced transactivation of the F876L mutant of full-length AR (B). Experimental details: A) For the NT, HEK293 cells were co-transfected with pIRES vector, PSA-luc reporter and pRL-TK plasmids. For all of the other wells, pIRES-AR-v7, PSA-luc and pRL-TK plasmids were transiently transfected into HEK293 cells. Transfected cells were exposed to DMSO vehicle or compounds in phenol red-free medium and 10% charcoal-stripped FBS (CS-FBS) for 24 h. B) Plasmids expressing F876L AR mutant, PSA-luc and pRL-TK were co-transfected into PC3 cells. Cells were exposed to DMSO vehicle, 10 nM DHT alone or compounds in the presence of 10 nM DHT for 24 h, in triplicate.

FIG. 23: Compound 482 at 1 and 2.5 μM potently suppresses the transcriptional activity of AR-v7 (A), but are inactive against the DHT-induced activation of the NTD-truncated VP16-AR(507-919) fusion protein (B). In contrast, the LBD-targeting compound DHT and Bic has no effect on AR-v7, but are active in VP16-AR(507-919) which retains the AR LBD. See the legend of FIG. 7 for experimental details.

FIG. 24: Effect of 562 analogues in the AR-v7-dependent PSA-luc reporter assay in HEK293 cells. Experimental details: For the NT, empty vector, PSA-luc and pRL-TK plasmids were transiently transfected into HEK293 cells. Cells were exposed to vehicle control or compounds at designated concentrations (μM) for 48 h.

FIG. 25: Effect of compound 746 and its analogues in the AR-v7-dependent PSA-luc reporter assay in HEK293 cells. The experiments were conducted as described above for FIG. 24.

FIG. 26: Effect of 746 analogues with side chain at meta position and other 746 analogues in the AR-v7-dependent PSA-luc reporter assay in HEK293 cells. The experiments were conducted as described above for FIG. 24.

FIG. 27: Effect of 746 analogues with side chain at ortho position in the AR-v7-dependent PSA-luc reporter assay in HEK293 cells. The experiments were conducted as described above for FIG. 24.

FIG. 28: Compounds 410, 428, 558 and 746 potently inhibits full-length AR W741C as well as AR F876L mutant dependent PSA-luc assay in PC3 cells (A and B). However, compounds 410, 428 and 746 are inactive in the VP16-AR(507-919) dependent PSA reporter assay as the AR NTD is absent, indicating that 410, 428 and 746 are targeting the AR NTD. W741C or F876L or VP16-AR(507-919) expressing plasmid as well as PSA-luc and pRL-TK plamids were transiently transfected into PC3 cells. Cells were exposed to DMSO vehicle control, 10 nM DHT or compounds at designated concentration (μM) in the presence of 10 nM DHT for 24 h. Bic, bicalutamide; ENZ, enzalutamide; EPI, EPI-001.

FIG. 29: Compounds 410, 428, 528, 562, 746, 968 and 973 potently inhibit the IRF3-AR(1-547)-dependent ISRE-luc reporter activity. Here, IRF3-AR(1-547) is the fusion of IRF3 DBD with the AR NTD (1-547). In contrast, these compounds are inactive against the wild-type IRF3, suggesting that 410, 428, 528, 562, 746, 968 and 973 are targeting the AR NTD. The plasmids expressing IRF3-AR(1-547) (A) or wild-type IRF3 (B) or IRF3 DBD (for NT) as well as ISRE-luc and pRL-TK were transiently transfected into PC3 cells. Cells were exposed to DMSO vehicle or compounds for 24 h. Bic, bicalutamide; EPI, EPI-001.

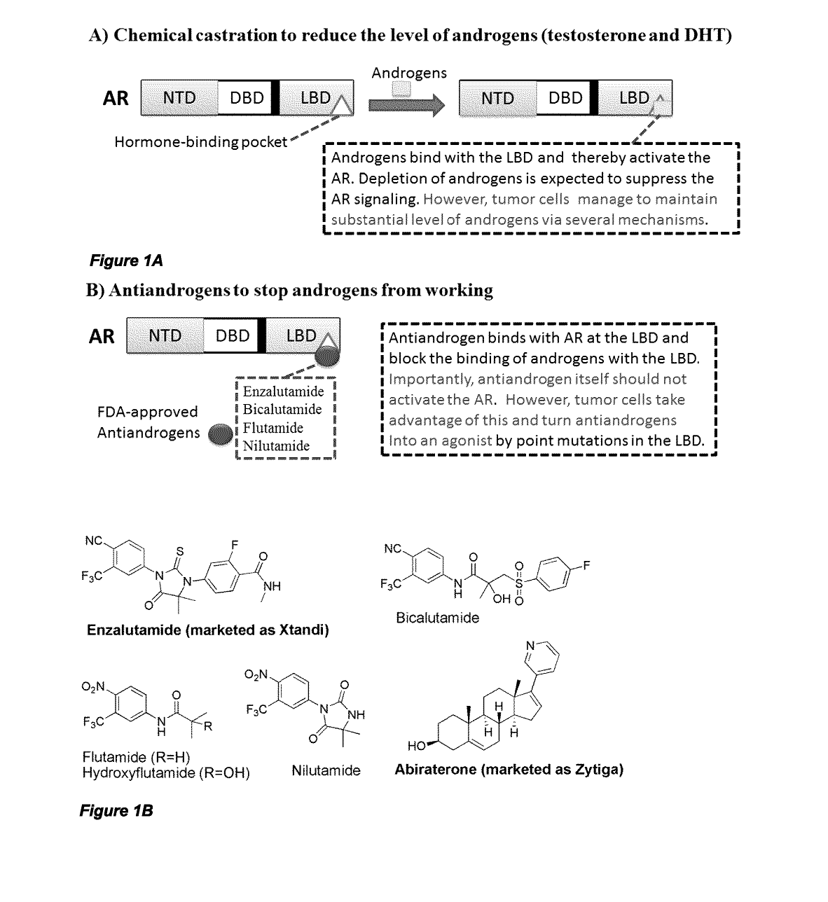

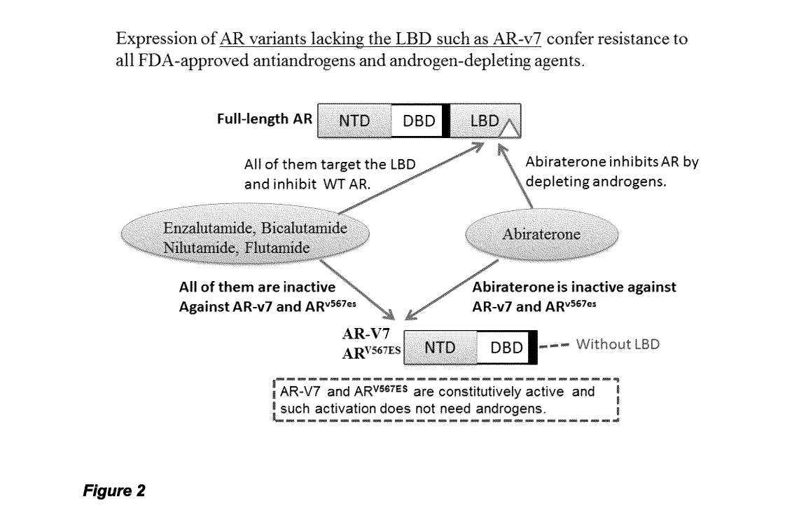

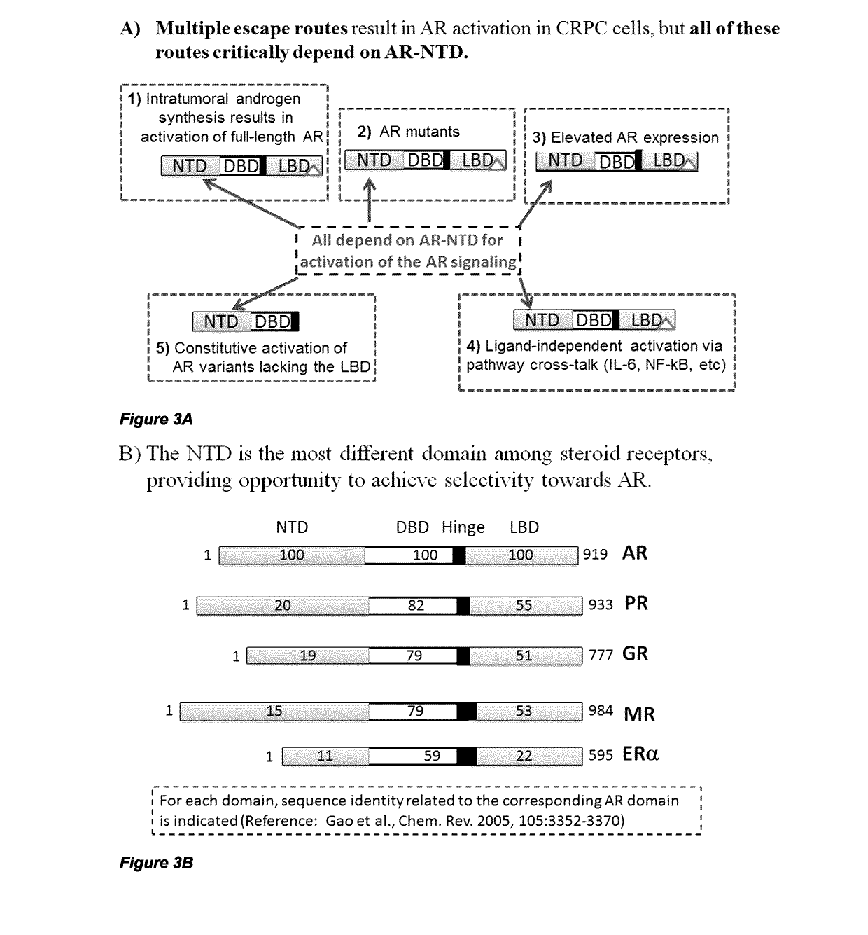

### DESCRIPTION OF ILLUSTRATIVE EMBODIMENTS

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this disclosure pertains.

As used herein, the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

Term “alkyl” or “alk” as used herein, represents a monovalent group derived from a straight or branched chain saturated hydrocarbon comprising, unless otherwise specified, from 1 to 15 carbon atoms and is exemplified by methyl, ethyl, n- and iso-propyl, n-, sec-, iso- and tert-butyl, neopentyl and the like and may be optionally substituted with one, two, three or, in the case of alkyl groups comprising two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)₂R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “alkoxy” or “alkyloxy” as used interchangeably herein, represents an alkyl group attached to the parent molecular group through an oxygen atom.

The term “alkylthio” or “thioalkoxy” as used interchangeably herein, represents an alkyl group attached to the parent molecular group through a sulfur atom.

The term “alkylene” as used herein, represents a saturated divalent hydrocarbon group derived from a straight or branched chain saturated hydrocarbon by the removal of two hydrogen atoms, and is exemplified by methylene, ethylene, isopropylene and the like.

The term “alkenyl” as used herein, represents monovalent straight or branched chain groups of, unless otherwise specified, from 2 to 15 carbons, such as, for example, 2 to 6 carbon atoms or 2 to 4 carbon atoms, containing one or more carbon-carbon double bonds and is exemplified by ethenyl, 1-propenyl, 2-propenyl, 2-methyl-1-propenyl, 1-butenyl, 2-butenyl and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)2R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms; (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “alkynyl” as used herein, represents monovalent straight or branched chain groups of from two to six carbon atoms comprising a carbon-carbon triple bond and is exemplified by ethynyl, 1-propynyl, and the like and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group comprises one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbon atoms; (16) N-protected amino; (17) nitro; (18) oxo or thiooxo; (19) perfluoroalkyl of 1 to 4 carbon atoms; (20) perfluoroalkoxyl of 1 to 4 carbon atoms; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group comprises one to six carbon atoms, (d) substituted or unsubstituted C₁₋₉ heterocyclyl, and (e) substituted or unsubstituted C₂₋₁₅ heterocyclylalkyl, where the alkylene group comprises one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group comprises one to six carbon atoms, (e) substituted or unsubstituted C₁₋₉ heterocyclyl, and (f) substituted or unsubstituted C₂₋₁₅ heterocyclylalkyl, where the alkylene group comprises one to six carbon atoms; (27) C(O)NR^{C}R^{D}, where each of R^{C} and R^{D} is independently selected from the group consisting of (a) hydrogen, (b) alkyl, (c) aryl and (d) arylalkyl, where the alkylene group comprises one to six carbon atoms; (28) S(O)R^{E}, where R^{E} is selected from the group consisting of (a) alkyl, (b) aryl, (c) arylalkyl, where the alkylene group comprises one to six carbon atoms, and (d) hydroxyl; (29) S(O)2R^{E}, where R^{E} is selected from the group consisting of (a) alkyl, (b) aryl, (c) arylalkyl, where the alkylene group comprises one to six carbon atoms, and (d) hydroxyl; (30) S(O)₂NR^{F}R^{G}, where each of R^{F} and R^{G} is independently selected from the group consisting of (a) hydrogen, (b) alkyl, (c) aryl and (d) arylalkyl, where the alkylene group comprises one to six carbon atoms; and (31) —NR^{H}R^{I}, where each of R^{H} and R^{I} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, (i) alkcycloalkyl, where the cycloalkyl group comprises three to eight carbon atoms, and the alkylene group comprises one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The term “aryl” as used herein, represents mono- and/or bicyclic carbocyclic ring systems and/or multiple rings fused together and is exemplified by phenyl, naphthyl, 1,2-dihydronaphthyl, 1,2,3,4-tetrahydronaphthyl, fluorenyl, indanyl, indenyl and the like and may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups independently comprise from one to six carbon atoms; (5) alkylsulfinyl of one to six carbon atoms; (6) alkylsulfinylalkyl, where the alkyl and alkylene groups independently comprise from one to six carbon atoms; (7) alkylsulfonyl of one to six carbon atoms; (8) alkylsulfonylalkyl, where the alkyl and alkylene groups are independently comprised of one to six carbon atoms; (9) aryl; (10) arylalkyl, where the alkyl group comprises one to six carbon atoms; (11) amino; (12) aminoalkyl of one to six carbon atoms; (13) aryl; (14) arylalkyl, where the alkylene group comprises one to six carbon atoms; (15) aryloyl; (16) azido; (17) azidoalkyl of one to six carbon atoms; (18) carboxaldehyde; (19) (carboxaldehyde)alkyl, where the alkylene group comprises one to six carbon atoms; (20) cycloalkyl of three to eight carbon atoms; (21) alkcycloalkyl, where the cycloalkyl group comprises three to eight carbon atoms and the alkylene group comprises one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group comprises one to six carbon atoms; (33) oxo; (34) thioalkoxy of one to six carbon atoms; (35) thioalkoxyalkyl, where the alkyl and alkylene groups independently comprise from one to six carbon atoms; (36) (CH₂)_{q}CO₂R^{A}, where q is an integer ranging from zero to four and R^{A} is selected from the group consisting of (a) alkyl, (b) aryl, and (c) arylalkyl, where the alkylene group comprises one to six carbon atoms; (37) (CH₂)_{q}C(O)NR^{B}R^{C}, where R^{B} and R^{C} are independently selected from the group consisting of (a) hydrogen, (b) alkyl, (c) aryl, and (d) arylalkyl, where the alkylene group comprises one to six carbon atoms; (38) (CH₂)_{q}S(O)₂R^{D}, where R^{D} is selected from the group consisting of (a) alkyl, (b) aryl, and (c) arylalkyl, where the alkylene group comprises one to six carbon atoms; (39) (CH₂)_{q}S(O)₂NR^{E}R^{F}, where each of R^{E} and R^{F} is independently selected from the group consisting of (a) hydrogen, (b) alkyl, (c) aryl, and (d) arylalkyl, where the alkylene group comprises one to six carbon atoms; (40) (CH₂)_{q}NR^{G}R^{H}, where each of R^{G} and R^{H} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms, and (i) alkcycloalkyl, where the cycloalkyl group comprises three to eight carbon atoms, and the alkylene group comprises one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “alkylaryl” as used herein, represents an aryl group attached to the parent molecular group through an alkyl group.

The term “cycloalkyl” as used herein, represents a monovalent saturated or unsaturated non-aromatic cyclic hydrocarbon group of three to eight carbon atoms, unless otherwise specified, and is exemplified by cyclopropyl, cyclobutyl, cyclopentyl, cyclohexyl, cycloheptyl, bicyclo[2.2.1]heptyl and the like. The cycloalkyl groups of the present disclosure can be optionally substituted with: (1) alkanoyl of one to six carbon atoms; (2) alkyl of one to six carbon atoms; (3) alkoxy of one to six carbon atoms; (4) alkoxyalkyl, where the alkyl and alkylene groups independently comprise from one to six carbon atoms; (5) alkylsulfinyl of one to six carbon atoms; (6) alkylsulfinylalkyl, where the alkyl and alkylene groups independently comprise from one to six carbon atoms; (7) alkylsulfonyl of one to six carbon atoms; (8) alkylsulfonylalkyl, where the alkyl and alkylene groups independently comprise from one to six carbon atoms; (9) aryl; (10) arylalkyl, where the alkyl group comprises one to six carbon atoms; (11) amino; (12) aminoalkyl of one to six carbon atoms; (13) aryl; (14) arylalkyl, where the alkylene group comprises one to six carbon atoms; (15) aryloyl; (16) azido; (17) azidoalkyl of one to six carbon atoms; (18) carboxaldehyde; (19) (carboxaldehyde)alkyl, where the alkylene group comprises one to six carbon atoms; (20) cycloalkyl of three to eight carbon atoms; (21) alkcycloalkyl, where the cycloalkyl group comprises three to eight carbon atoms and the alkylene group comprises one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group comprises one to six carbon atoms; (33) oxo; (34) thioalkoxy of one to six carbon atoms; (35) thioalkoxyalkyl, where the alkyl and alkylene groups independently comprise from one to six carbon atoms; (36) (CH₂)_{q}CO₂R^{A}, where q is an integer ranging from zero to four and R^{A} is selected from the group consisting of (a) alkyl, (b) aryl, and (c) arylalkyl, where the alkylene group comprises one to six carbon atoms; (37) (CH₂)_{q}C(O)NR^{B}R^{C}, where each of R^{B} and R^{C} is independently selected from the group consisting of (a) hydrogen, (b) alkyl, (c) aryl, and (d) arylalkyl, where the alkylene group comprises one to six carbon atoms; (38) (CH₂)_{q}S(O)₂R^{D}, where R^{D} is selected from the group consisting of (a) alkyl, (b) aryl, and (c) arylalkyl, where the alkylene group comprises one to six carbon atoms; (39) (CH₂)_{q}S(O)₂NR^{E}R^{F}, where each of R^{E} and R^{F} is independently, selected from the group consisting of (a) hydrogen, (b) alkyl, (c) aryl, and (d) arylalkyl, where the alkylene group comprises one to six carbon atoms; (40) (CH₂)_{q}NR^{G}R^{H}, where each of R^{G} and R^{H} is independently selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group comprises one to six carbon atoms; (h) cycloalkyl of three to eight carbon atoms and (i) alkcycloalkyl, where the cycloalkyl group comprises three to eight carbon atoms, and the alkylene group comprises one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “halogen” or “halo” as used interchangeably herein, represents F, Cl, Br and I.

The term “heteroatom”, as used herein, is understood as being oxygen, sulfur or nitrogen.

The term “carbonyl” as used herein, represents a C(O) group, which can also be represented as C═O.

The term “acyl” or “alkanoyl” as used interchangeably herein, represents an alkyl group, as defined herein, or hydrogen attached to the parent molecular group through a carbonyl group, as defined herein, and is exemplified by formyl, acetyl, propionyl, butanoyl and the like. Exemplary unsubstituted acyl groups comprise from 2 to 10 carbons.

The term “analogue” as used herein, is understood as being a substance similar in structure to another compound but differing in some slight structural detail.

The term “salt(s)” as used herein, is understood as being acidic and/or basic salts formed with inorganic and/or organic acids or bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps. Examples of acid addition salts include but are not limited to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, phosphoric, 2-hydroxyethanesulfonate, lactate, maleate, mandelate, methanesulfonate, 2-naphthalenesulfonate, nicotinate, oxalate, pectinate, persulfate, 3-phenylpropionate, picrate, pivalate, propionate, succinate, tartrate, thiocyanate, tosylate, and undecanoate. Examples of base addition salts include but are not limited to alkali metal salts and alkaline earth metal salts. Non limiting examples of alkali metal salts include lithium, sodium and potassium salts. Non-limiting examples of alkaline earth metal salts include magnesium and calcium salts.

The term “androgen-dependent diseases or disorders” as used herein, refers to diseases or disorders wherein the cells implicated need androgens for survival, proliferation or for maintaining aberrant states.

The “AR-mediated diseases or disorders” as used herein, refers to diseases or disorder that are directly or indirectly driven or maintained by the AR signaling from the wild-type AR, mutants of the full-length AR, the AR variants, or the AR variants that lack certain AR domains or parts of certain AR domains such as the LBD, or a combination of the above ARs.

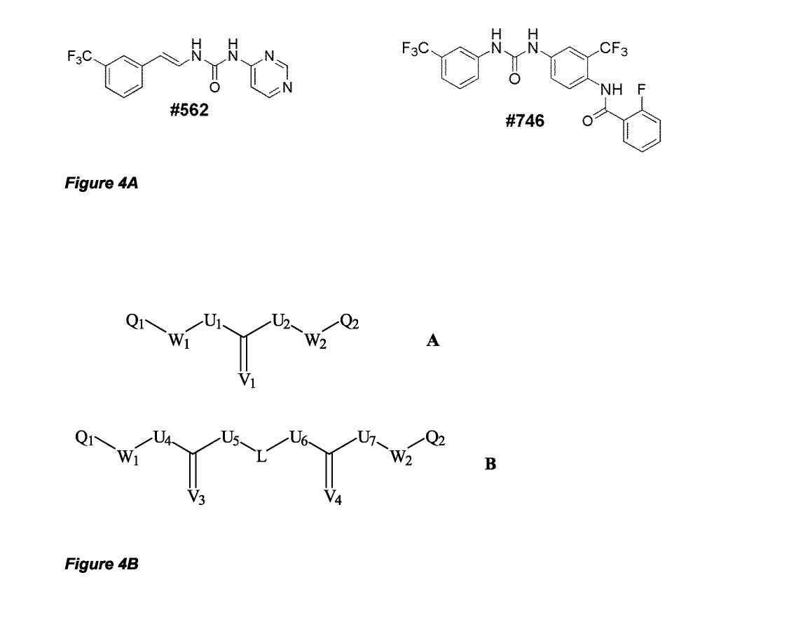

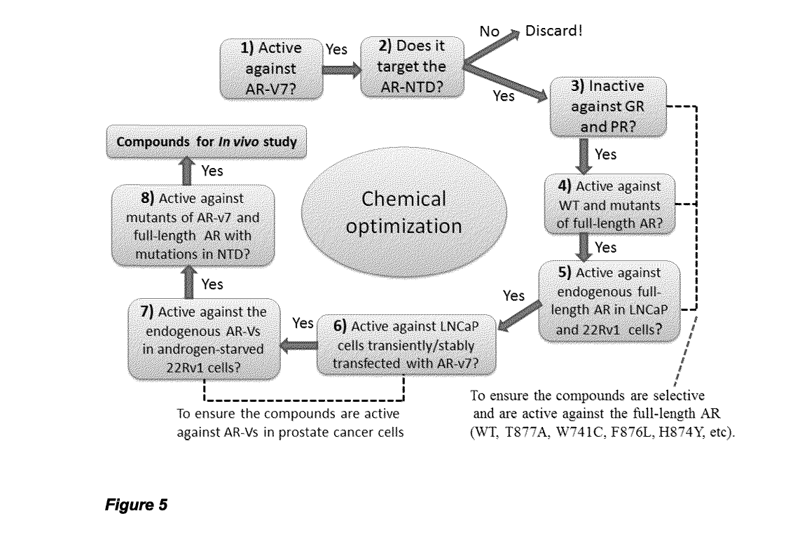

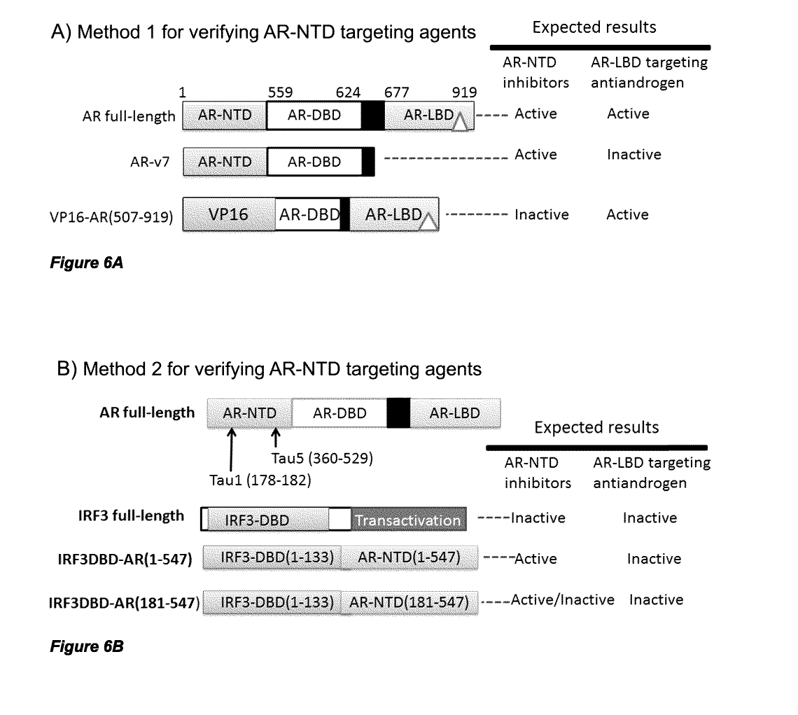

Two compounds according to the invention, namely, compounds 562 and 746, novel AR-NTD inhibitors are outlined in FIG. 4. Our workflow to identify compounds that are AR-NTD inhibitors is shown in FIG. 5. In particular, we have developed two methods for verifying whether a compound targets the AR-NTD (FIG. 6). Specifically, Method 1 utilizes AR-v7 (LBD deleted), full-length AR and the fusion protein VP16-AR(507-919) (NTD deleted). The AR(507-919) is transcriptionally inactive as it lacks the AR-NTD transcriptional domain.²⁵ Fusion of VP16 transactivation domain to AR(507-919) results in fusion protein VP16-AR(507-919) that is transcriptionally active. VP16-AR(509-919) retains the AR-DBD and AR-LBD but lacks the AR-NTD. The AR-NTD inhibitors should be active against AR-v7 and full-length AR, but inactive against VP16-AR(509-919). Method 2 utilizes the DBD of IRF3 (referred to as IRF3DBD) and fusion protein IRF3DBD-AR-NTD, which is the IRF3DBD fused with AR-NTD. The IRF3-DBD alone is transcriptionally inactive as it needs a transactivation domain at the C-terminus.

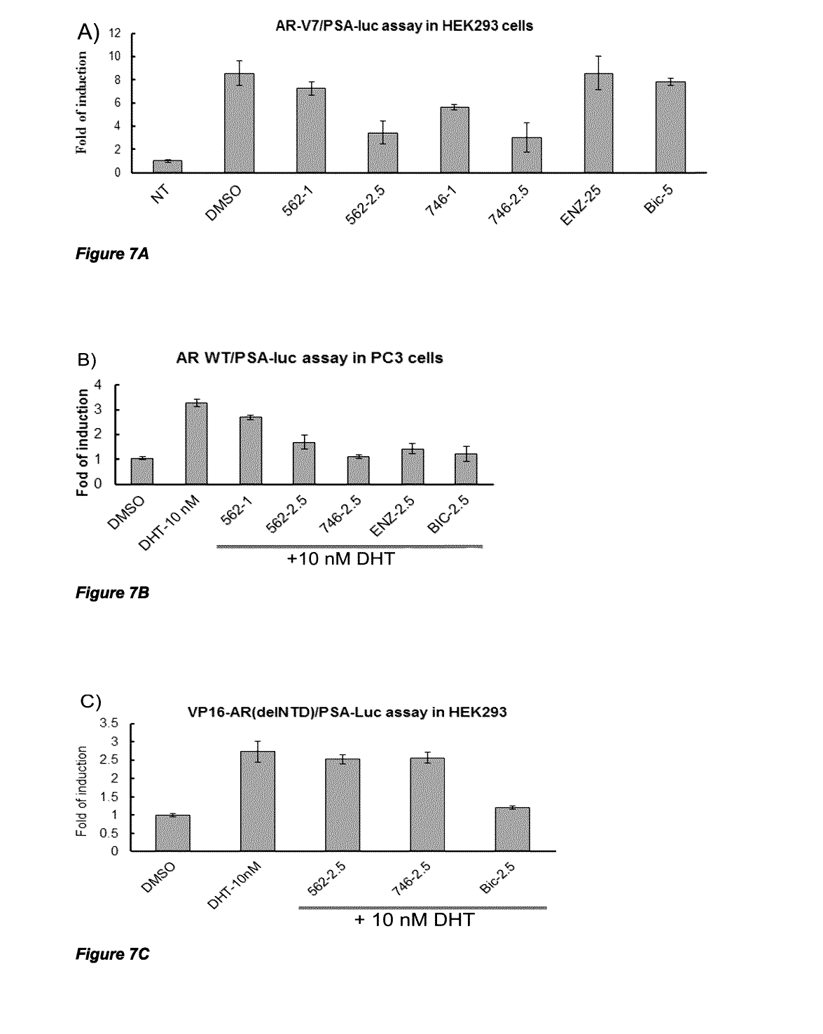

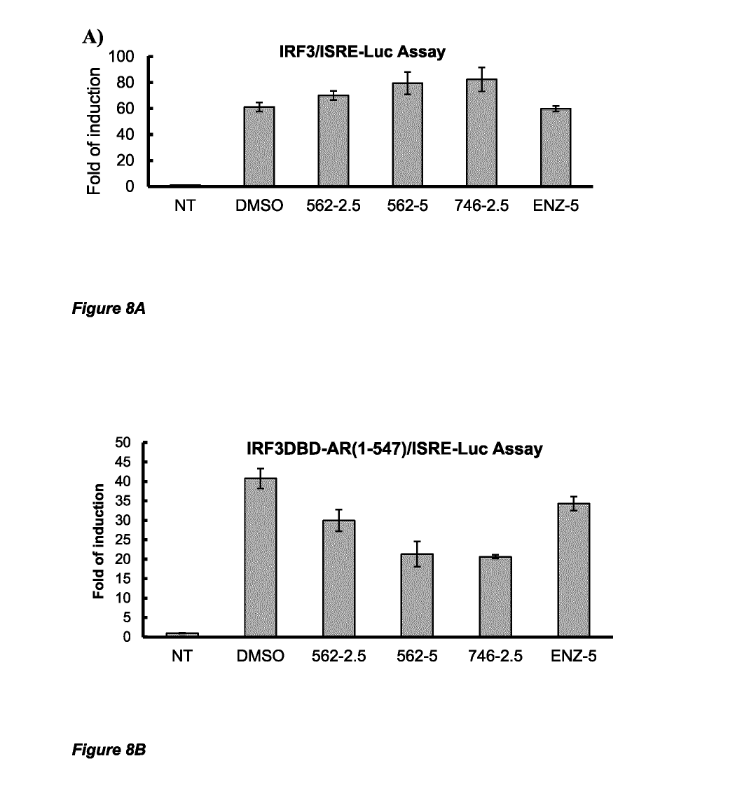

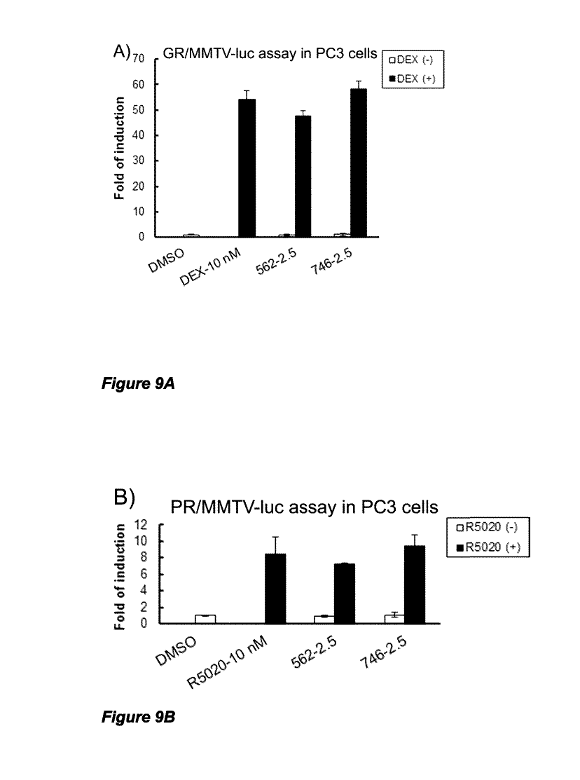

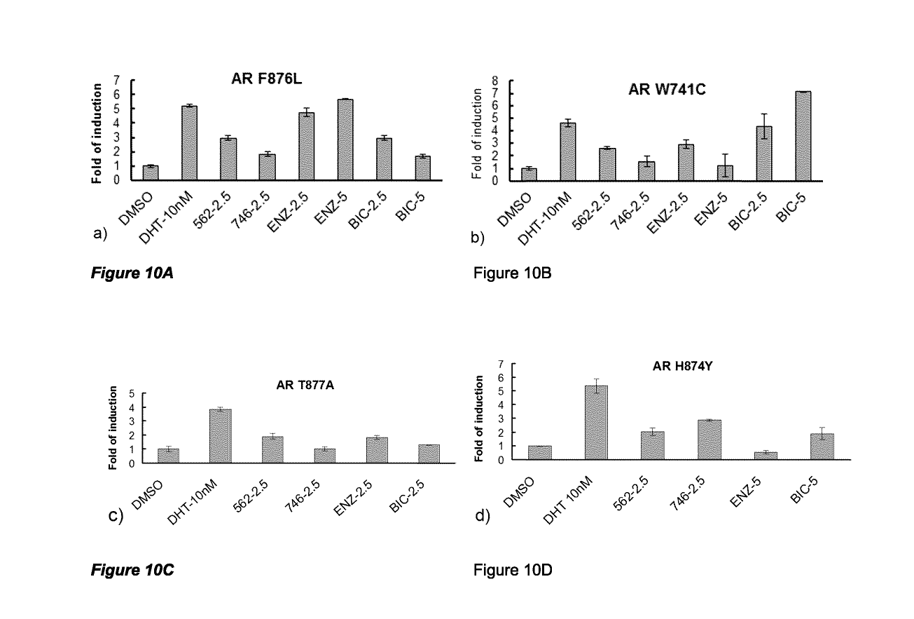

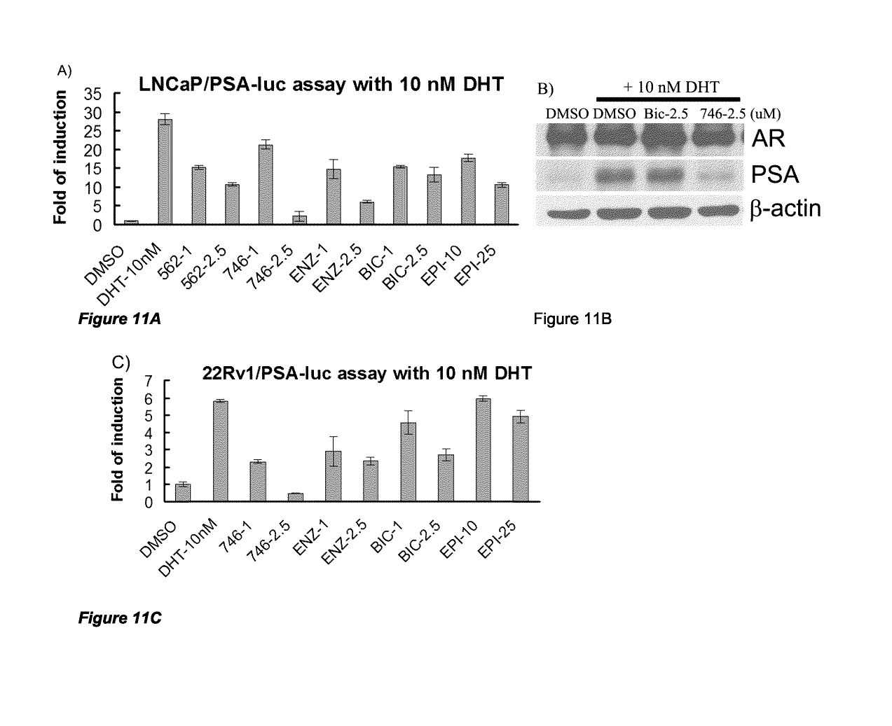

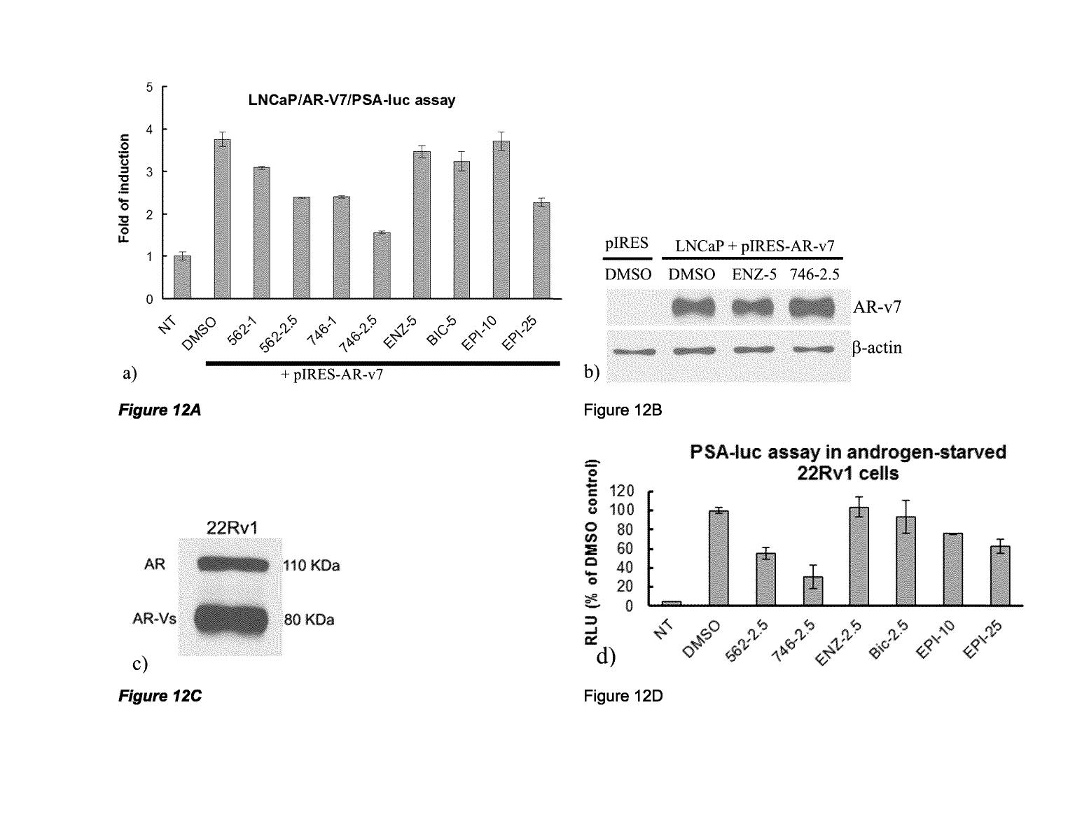

We found that when the AR-NTD is fused with the IRF3-DBD domain, the resulted fusion protein IRF3DBD-AR-NTD has potent transcriptional activity, which could be inhibited by the AR-NTD inhibitors (FIG. 6). The activities of compounds 562 and 746 are summarized as follows:
1) Compounds 562 and 746 are targeting the AR-NTD. As shown in FIG. 7, compounds 562 and 746 dose-dependently inhibit AR-v7 and WT full-length AR, but are inactive against the VP16-AR(507-919). In contrast, LBD-targeting enzalutamide (ENZ) and bicalutamide (BIC) are inactive against AR-v7, but remain active against the full-length AR and VP16-AR(delNTD). This was confirmed by our second method using IRF3DBD-AR-NTD fusion protein (FIG. 8).
2) Compounds 562 and 746 are selective towards the AR when compared with two close homology proteins with the steroid receptor family: glucocorticoid receptor (GR) and Progesterone receptor (PR) (FIG. 9). This presents at least some level of importance. Indeed, a recent unsuccessful phase II clinical trial of antiandrogen mifepristone in CRPC patients revealed that inhibition of GR by mifepristone probably limited its efficacy in CRPC via an increase of adrenal androgens production.²⁶
3) In PC3 cells transiently transfected with AR-expressing plasmids, compounds 562 and 746 inhibit DHT-induced transactivation of the F876L, W741C, T877A and H874Y mutants of full-length ARs. In contrast, enzalutamide cannot inhibit the F876L and bicalutamide cannot inhibit the W741C (FIG. 10).
4) In LNCaP cells which endogenously express the AR T877A mutant, compounds 562 and 746 potently inhibited the DHT-induced AR activation (FIG. 11A) and suppressed PSA expression (FIG. 11B). In 22Rv1 cells which endogenously express the AR H874Y mutant, compound 746 at 1 μM showed inhibitory activity (FIG. 11C). EPI-001 (EPI) is a derivative of bisphenol A diglycidic ether and was discovered by Dr. Sadar to be a novel AR-NTD-targeting agent.¹⁵ To date, EPI-001 is the best characterized compound targeting the AR-NTD.^{15,17} The IC50 of EPI-001 in PSA-luc reporter assay in LNCaP cells was reported to be 12.63±4.33 μM.¹⁷ We have obtained EPI-001 (Sigma-Aldrich catalog number: 92427) and included it in our assay for comparison (FIG. 11). We demonstrated that compound 746 at 2.5 μM presents a greater inhibitory activity than EPI at 25 μM (FIG. 11).
5) In LNCaP cells transiently transfected with AR-v7 expressing plasmids, we demonstrated that expression of AR-v7 confers resistance to enzalutamide and bicalutamide. In contrast, the NTD-targeting agents compound 562, compound 746 and EPI remain active. Again, compounds 562 and 746 present greater inhibitory activities than EPI by at least 10-fold (compared to compounds 562 and 746 at 2.5 μM with EPI at 25 μM) (FIGS. 12A and B).
6) Endogenous expression of the LBD-truncated AR variants in 22Rv1 cells confer resistance to enzalutamide and bicaluamide, but compounds 562 and 746 remain active in such system (FIGS. 12C and D). It should be noted that 22Rv1 cells endogenously express both full-length AR and LBD-truncated AR variants, including AR-v7 (FIG. 12C). To evaluate effect of compounds on the constitutive activation of the endogenous LBD-truncated AR variants in 22Rv1 cells, the cells were cultured in androgen-deleted medium (phenol red-free RPMI 1640 plus 10% CS-FBS) for 3 days to make sure the full-length AR is silenced.

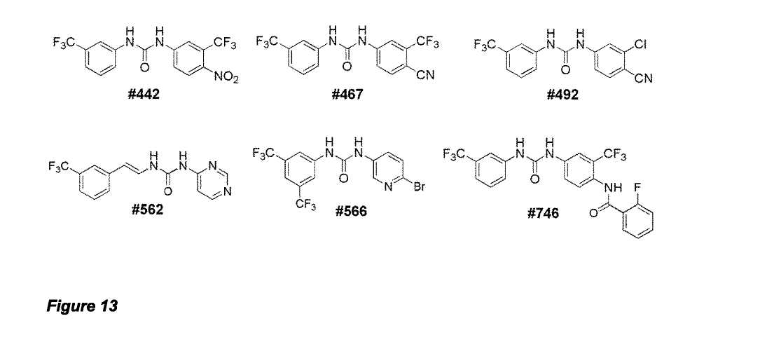

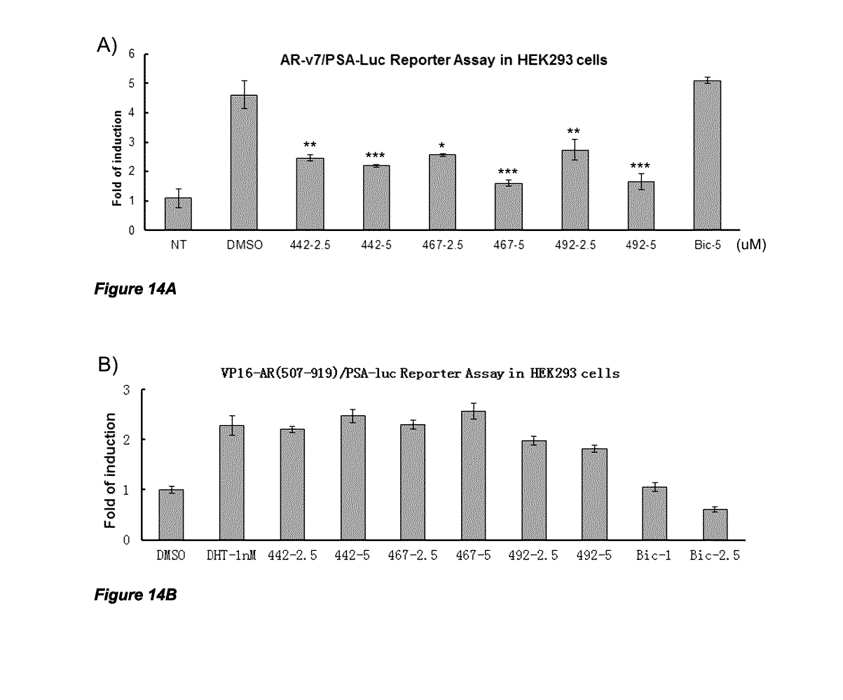

Six compounds according to the invention, AR-NTD inhibitors are outlined in FIG. 13. These AR-NTD inhibitors not only inhibit the constitutive activation of AR-Vs, but also inhibit DHT-induced activation of the wild-type and multiple clinically-relevant mutants of the full-length ARs.

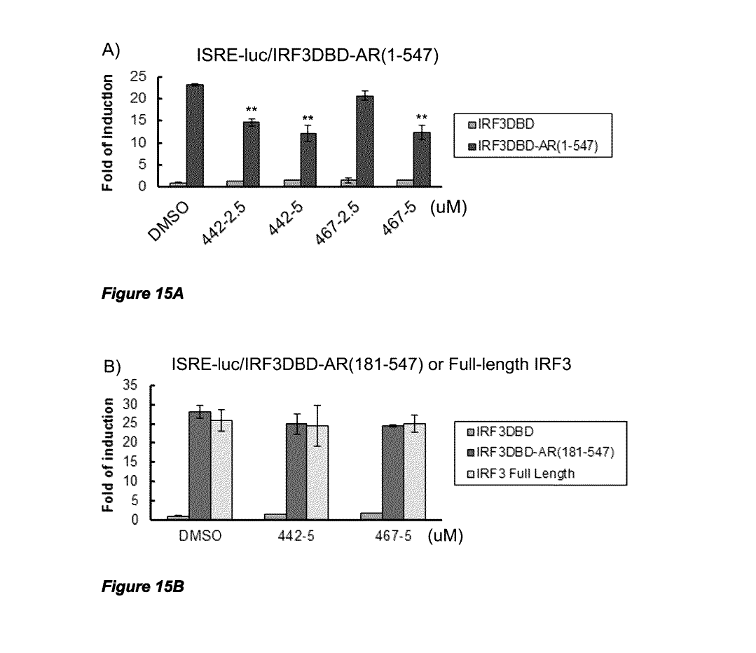

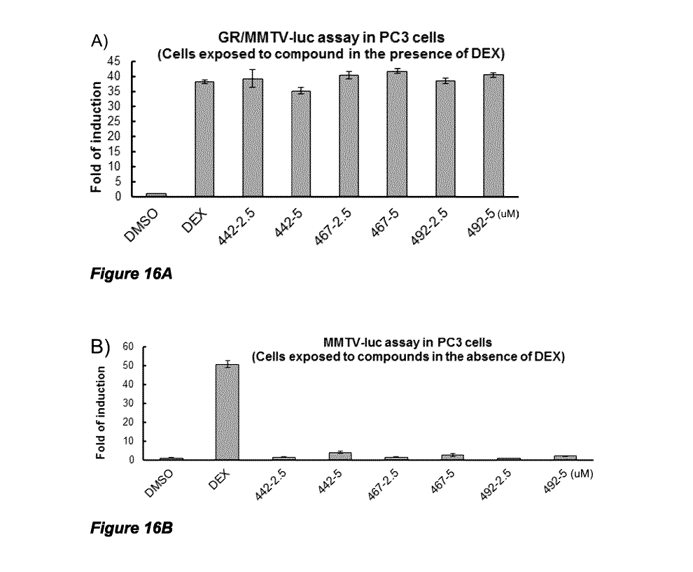

Compounds 442, 467 and 492, but not the LBD-targeting bicalutamide, inhibited constitutive activation of AR-v7 (FIG. 14). Our studies indicated that these compounds target the AR-NTD. By Method 1 (FIG. 6), these compounds are active against the AR activation when the AR-NTD is present (such as AR-V7) and inactive when the AR-NTD is absent (such as VP16-AR(507-919)) (FIG. 14). By Method 2 (FIG. 6), compounds 467 and 442 suppressed the IRF3DBD-AR(1-547)-mediated ISRE-luc activation, but not the IRF3DBD-AR(181-547) or the full-length IRF3 (FIG. 15), suggesting that compounds 467 and 442 target the AR-NTD and their inhibitory activity require the presence of AR residues 1-180.

To evaluate selectivity of our AR-NTD inhibitors, we showed that compounds 467, 442 and 492 at 5 μM were a non-agonist of GR, and were inactive in suppressing GR transactivation induced by 10 nM DEX (FIG. 16). This presents at least some level of importance. Indeed, a recent unsuccessful phase II clinical trial of antiandrogen mifepristone in CRPC patients revealed that inhibition of GR by mifepristone probably limited its efficacy in CRPC via an increase of adrenal androgens production.²⁶

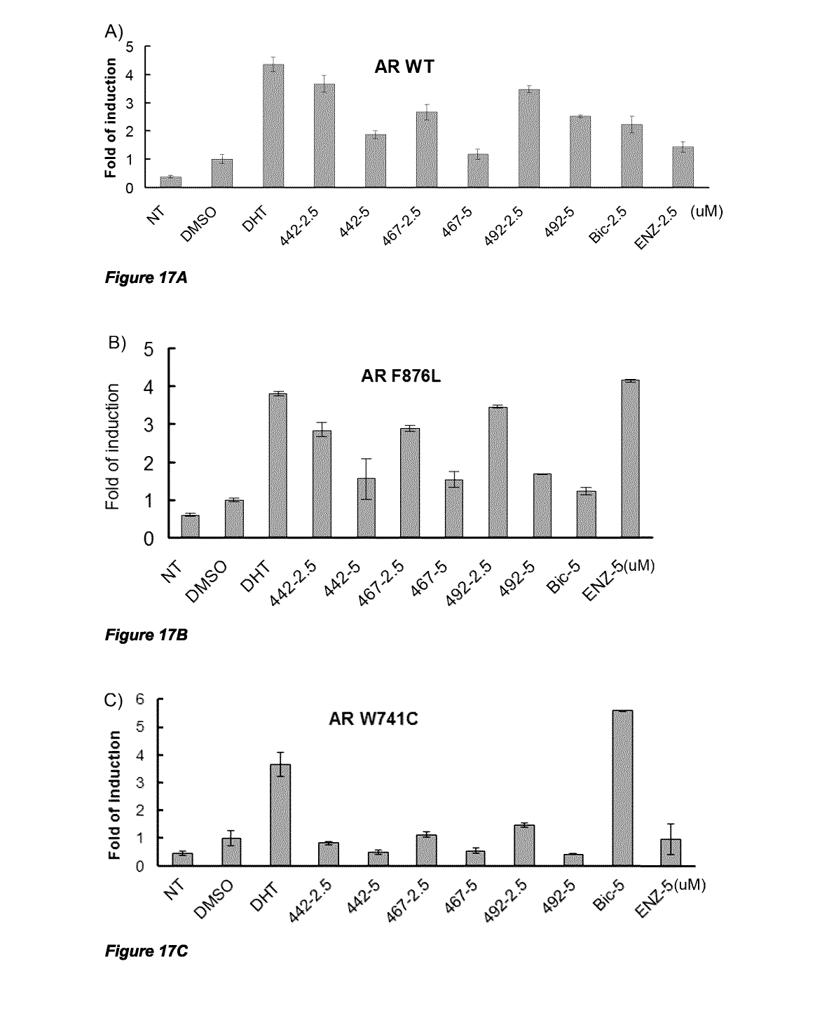

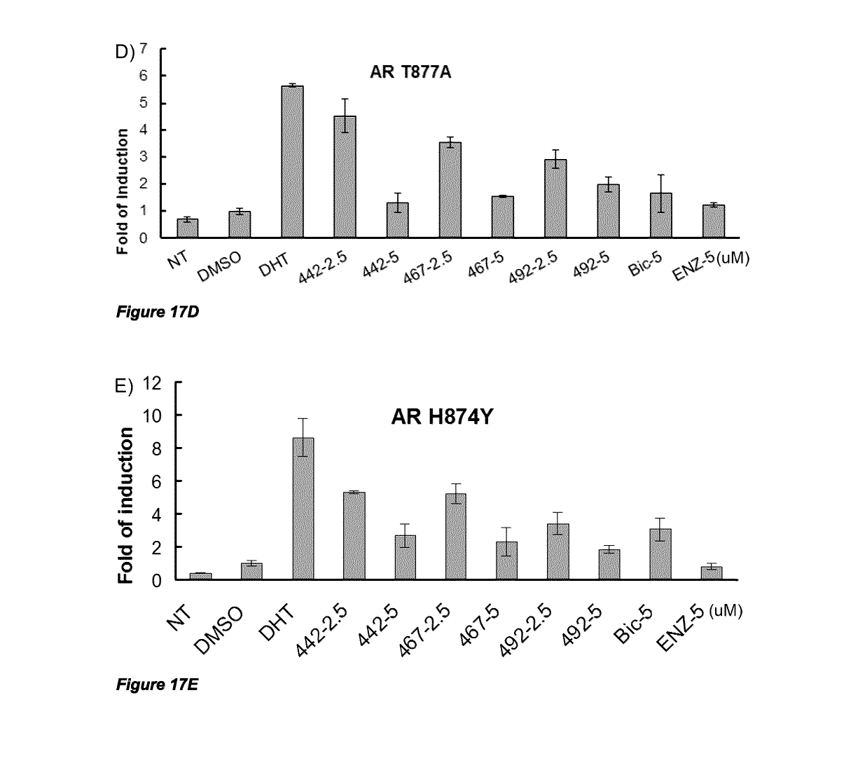

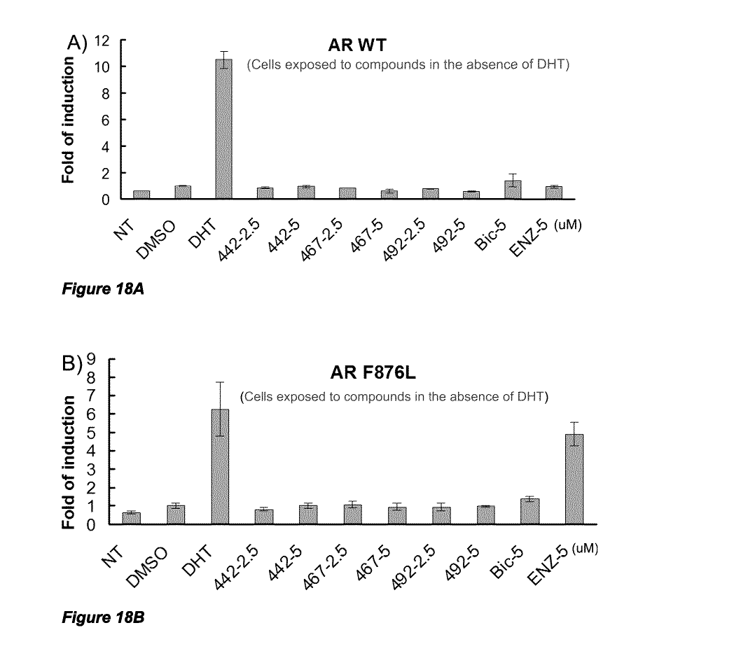

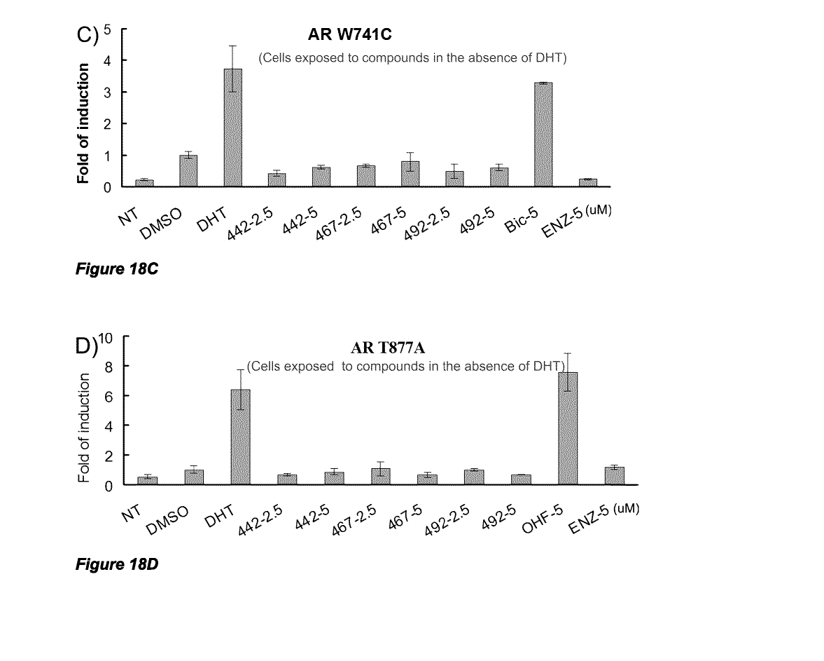

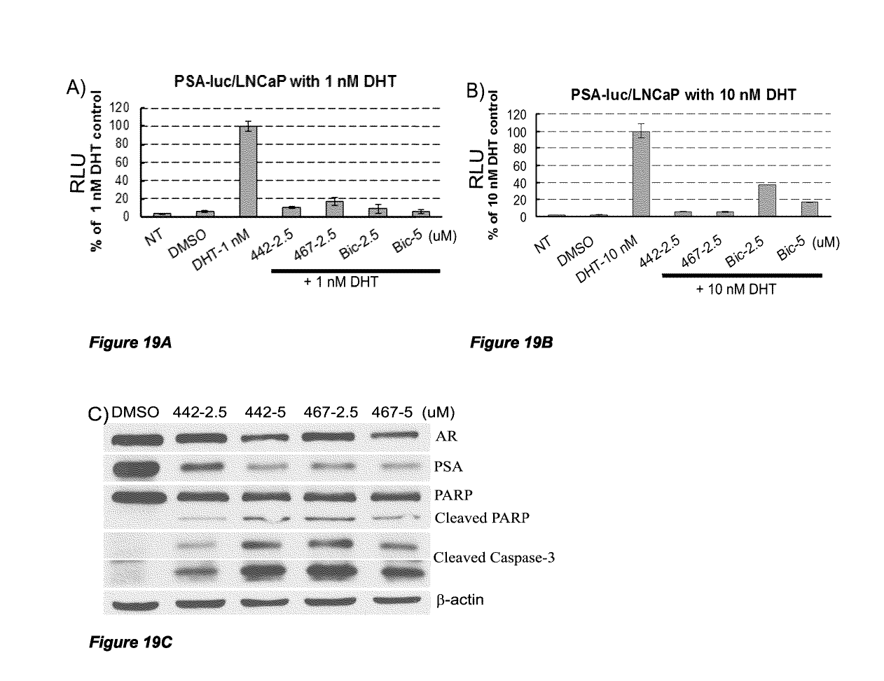

We further demonstrated that compounds 442, 467 and 492 dose-dependently inhibit the wild type and the F876L, W741C, T877A and H874Y mutants of the full-length ARs (FIG. 17) and are non-agonist of these ARs (FIG. 18). Consistent with the literature,^{18,22,24} we showed that enzalutamide (ENZ) activated the F876L mutant, whereas bicalutamide (Bic) and hydroxyflutamide (OHF) activated the W741C and T877A mutants, respectively (FIG. 18). Compounds 442 and 467 suppressed the DHT-induced activation of endogenous AR, suppressed PSA expression and induced apoptosis in LNCaP cells (FIG. 19). Importantly, the inhibitory activity of compounds 442 and 447 was not competed out by increasing DHT from 1 nM to 10 nM (FIG. 19). This is expected for AR-NTD targeting agents. In contrast, activity of Bic was attenuated.

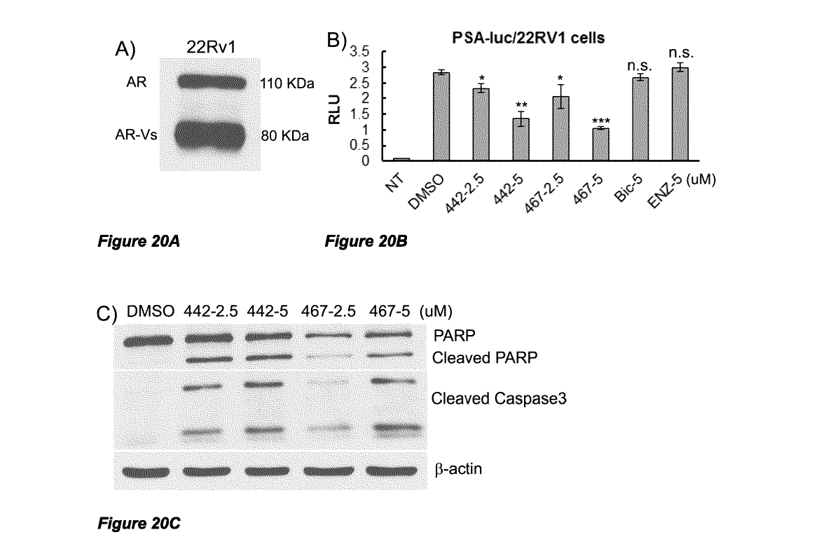

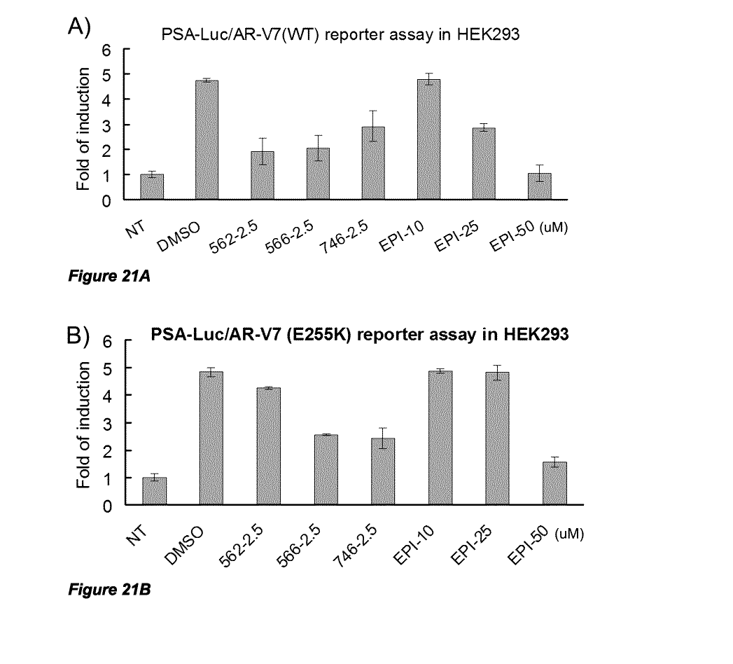

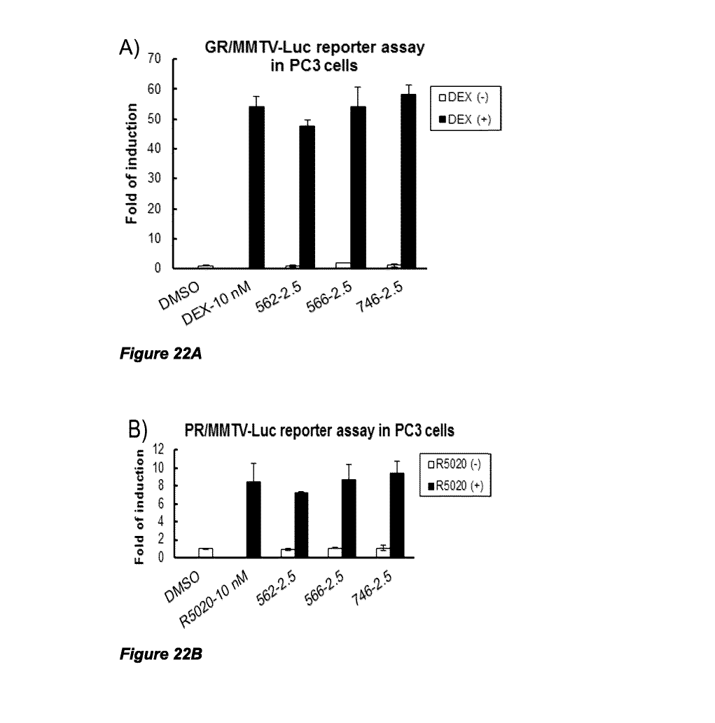

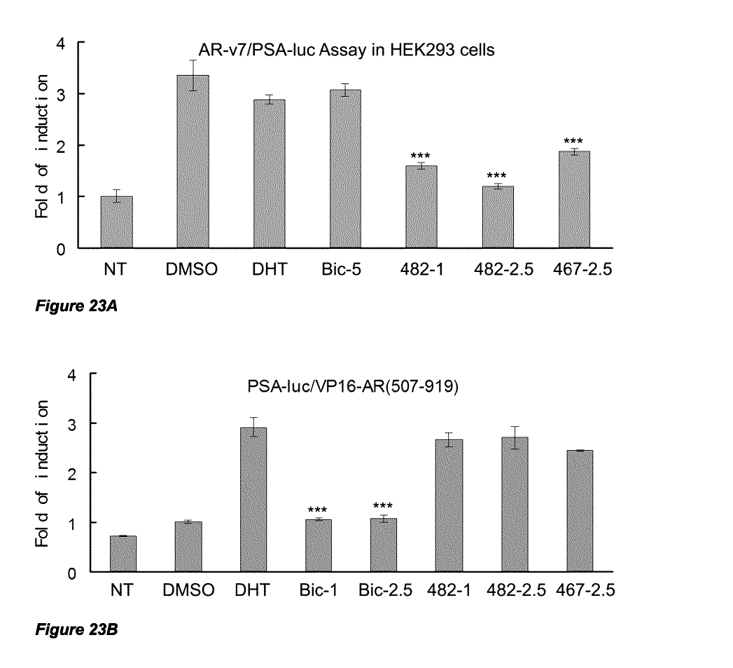

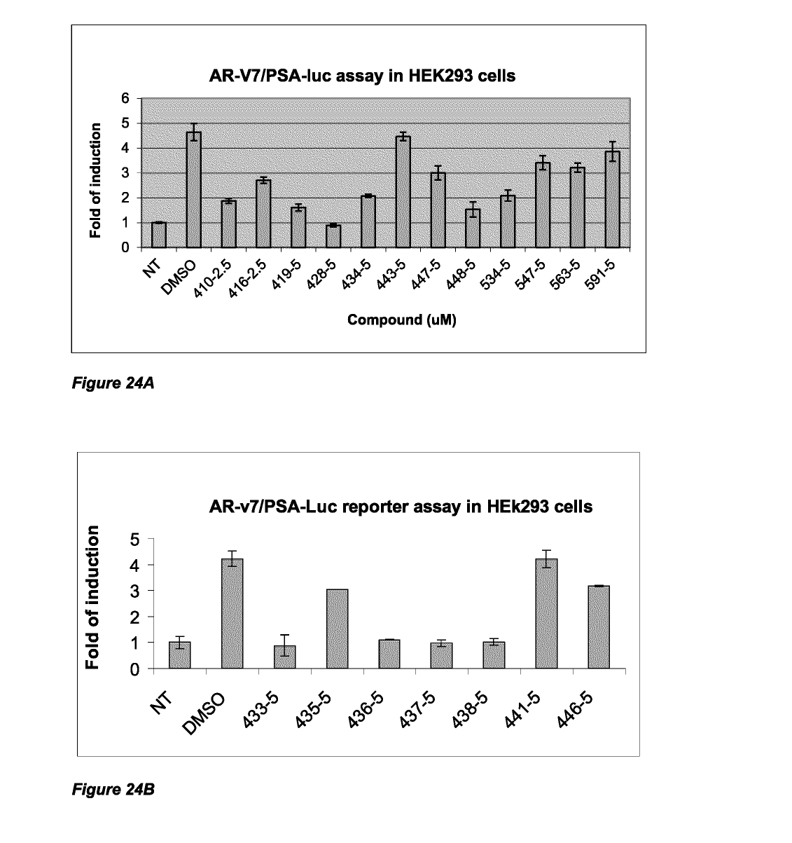

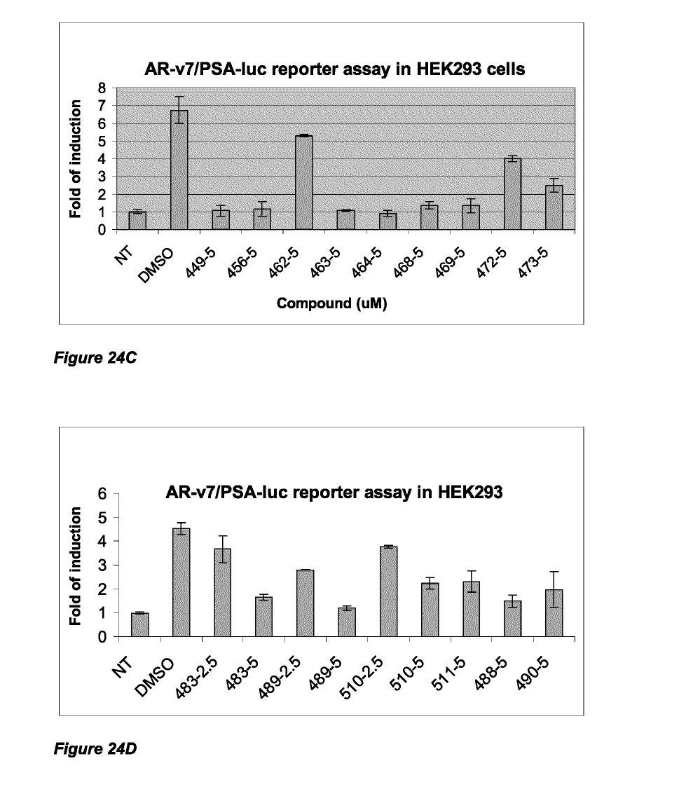

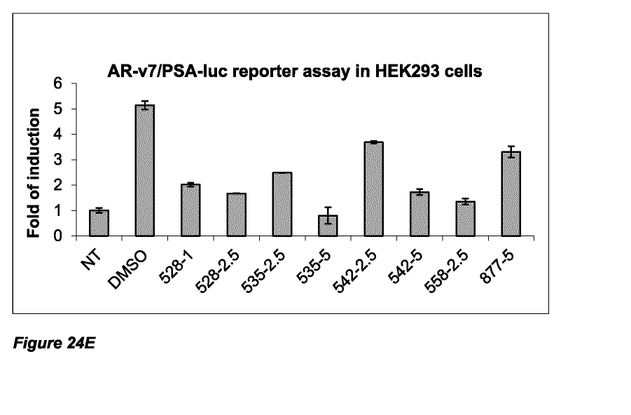

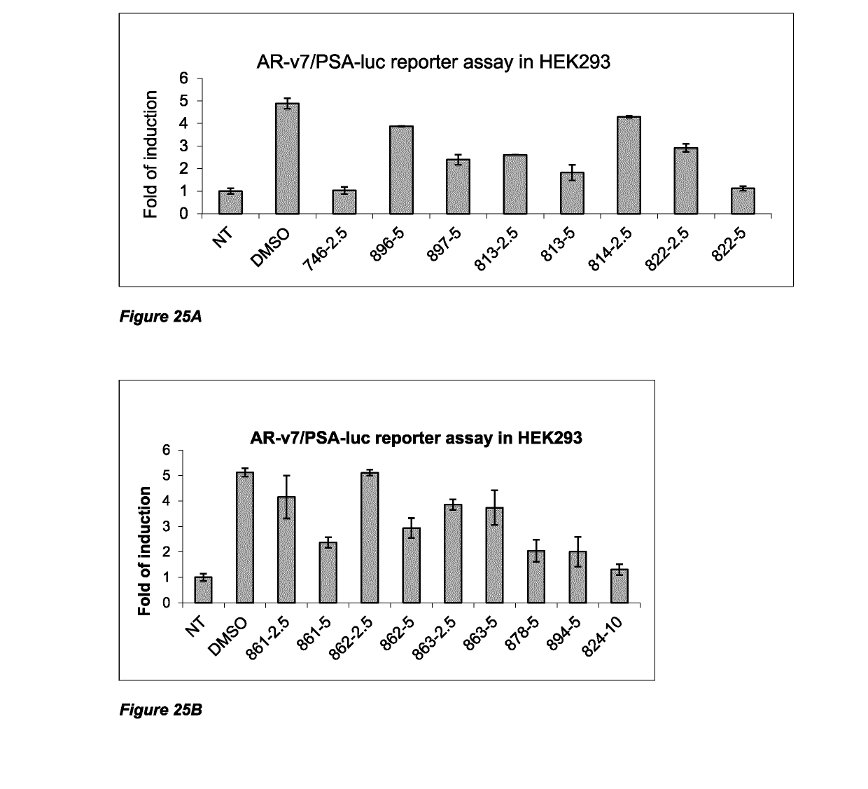

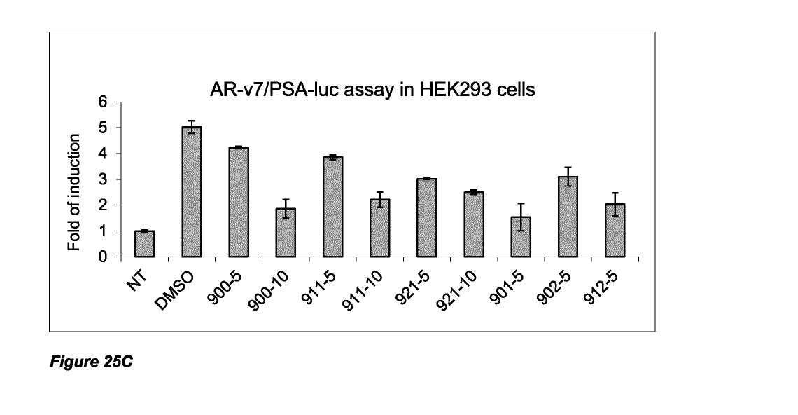

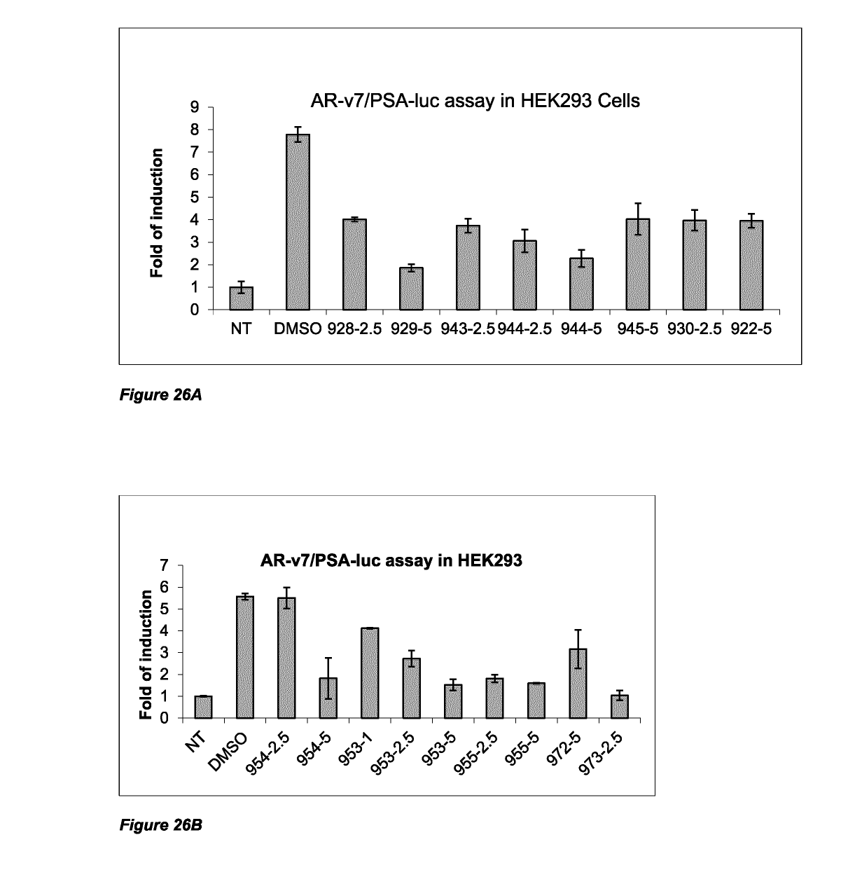

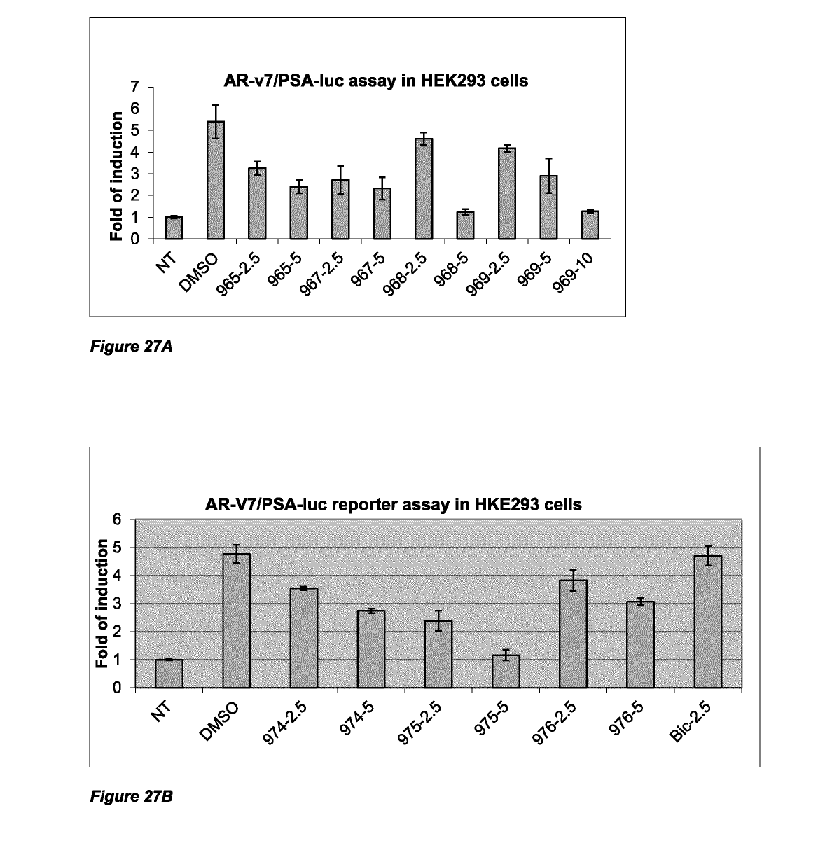

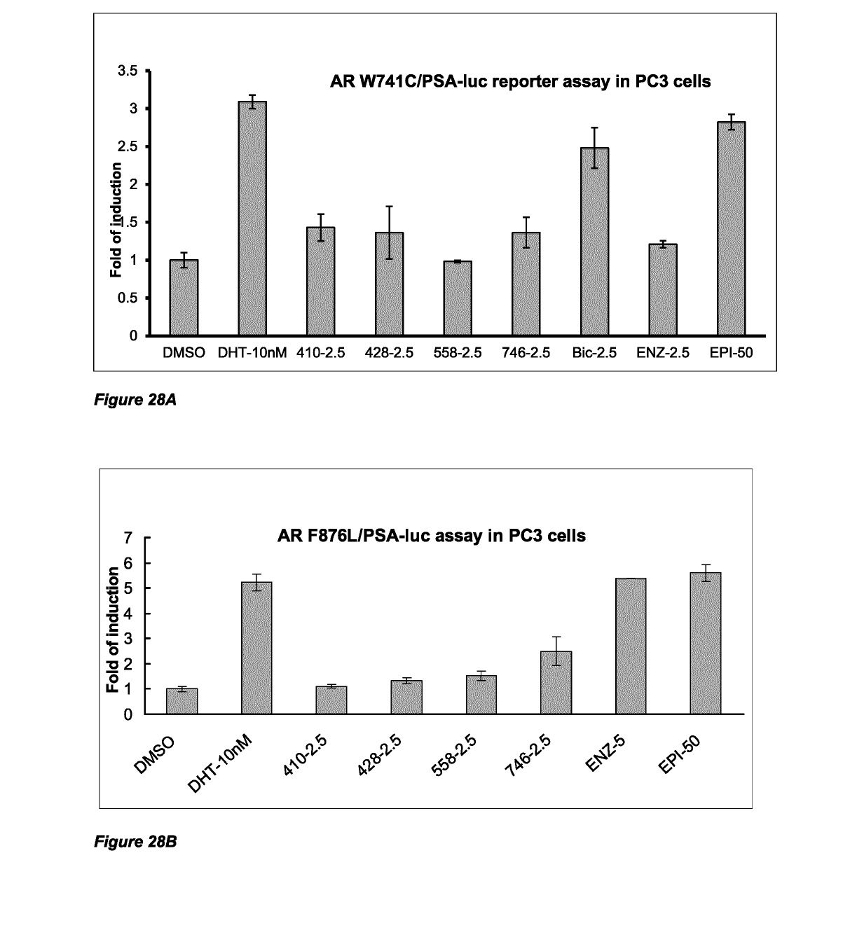

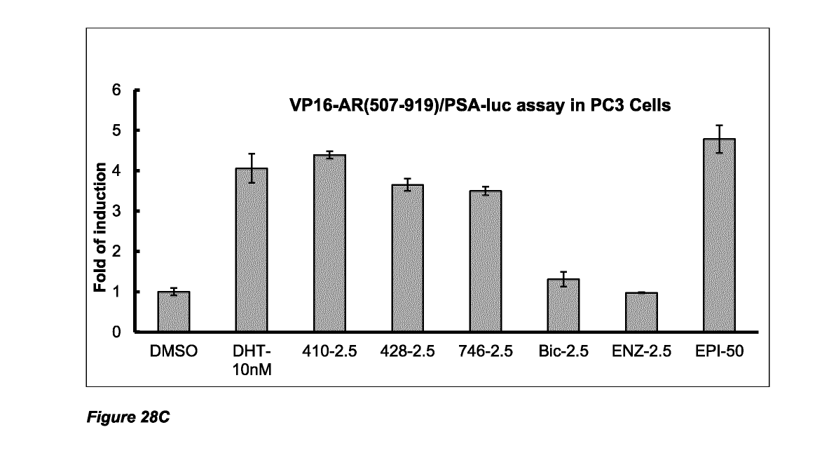

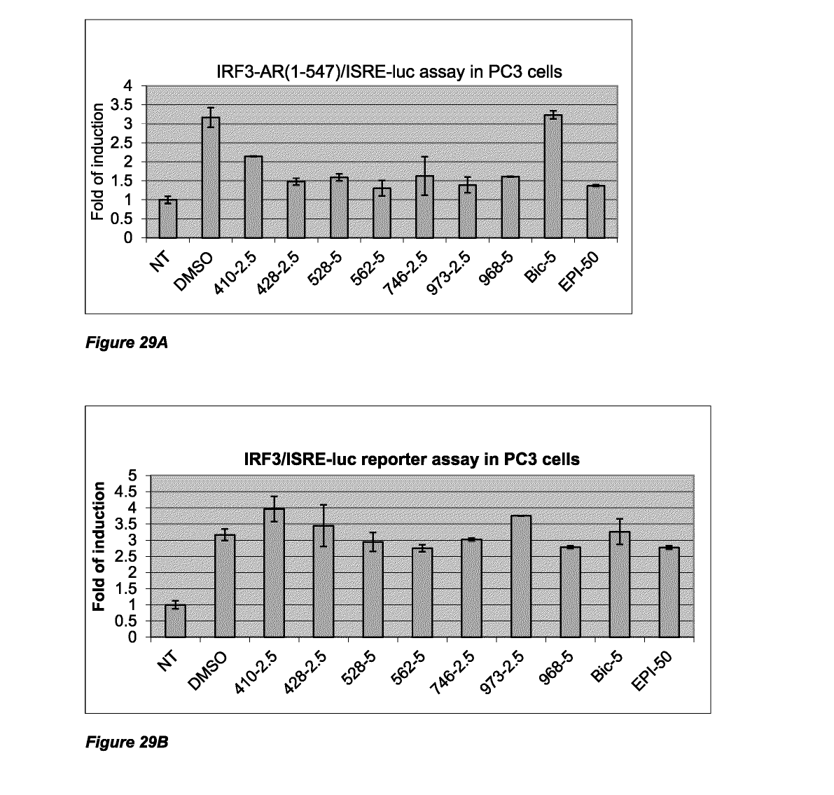

Furthermore, compounds 467 and 442 are active against endogenous AR-Vs (lacking the LBD) in androgen-starved 22Rv1 cells. In contrast, the AR-LBD-directed bicalutamide and enzaluamide are inactive (FIG. 20).

Three additional compounds according to the invention, AR-NTD inhibitors (compounds 562, 566 and 746) inhibit AR-v7 at a dose of 2.5 μM (FIG. 21). Although under other name, compound EPI-001 could be purchased from Sigma-Aldrich (catalog number: 92427. Firstly, we found that EPI-001 at 25 μM is active against AR-V7 (FIG. 21A) and the potency of EPI-001 at this dose is comparable with the result presented in the recent paper of Dr. Sadar et al.,¹⁷ where EPI-001 at 25 μM approximately suppressed constitutive activation of AR^{v567es} by half in PSA-Luc reporter assay in COS-1 cells.¹⁷ Importantly, we showed that compounds 562 and 566 at 2.5 μM are more potent than EPI-001 at 25 μM in suppressing constitutive activation of the wild-type AR-v7 (FIG. 21A).

Furthermore, compounds 562, 566 and 746 present a greater inhibitory activity than EPI-001 against the endogenous AR-Vs in 22Rv1 cells (FIG. 21B). Our ISRE-luc reporter assays in HEK293 cells co-transfected with IRF3DBD-AR(1-547) or IRF3DBD-AR(181-547) or full-length IRF3 suggest that compounds 562, 566 and 746 target the AR-NTD (FIG. 8). Compounds 562, 566 and 746 at 2.5 M do not interfere with transcriptional activity of GR and PR (FIG. 9). For endogenous full-length AR, compounds 562 and 746 showed good activity in PSA-Luc assay in LNCaP and 22Rv1 cells (FIG. 11). The IC₅₀ of compound 562 in PSA-Luc/LNCaP assay is about 1 μM (FIG. 11). In contrast, the IC₅₀ of EPI-001 in PSA-luc assay in LNCaP cells was reported to be 12.63±4.33 μM.¹⁷

### Chemistry

Referring to the reaction schemes provided herein below, Scheme 1 outlines the chemical synthesis of compound 746. Another embodiment of the synthesis of this compound is outlined at Scheme 4. Also, Schemes 2.1, 2.2, 2.3, 2.4 and 2.6 outline chemical syntheses of various analogues of the 562 compound.

Scheme 2 outlines the chemical synthesis of compound 562. Another embodiment of the synthesis of this compound is outlined at Scheme 3. Also, Schemes 1.1, 1.4, 1.5, 1.6, 1.7, 1.8, 1.9, 1.10 and 1.11 outline the chemical synthesis of compound 746 and its analogues.

Scheme 5 outlines the chemical synthesis of compound 566. Also, Schemes 4.1 and 4.2 outline chemical syntheses of various analogues of the 566 compound.

Scheme 1.2 outlines the chemical synthesis of compound 789. Scheme 3.1 outlines the chemical synthesis of compound 804. Scheme 2.5 outlines the chemical synthesis of compound 454. And Scheme 5.1 outlines the chemical synthesis of the bis-urea compounds according to the invention.

More detail information on the various chemical syntheses of the compounds according to the invention is provided herein below.

### Compound 746 and its Analogues

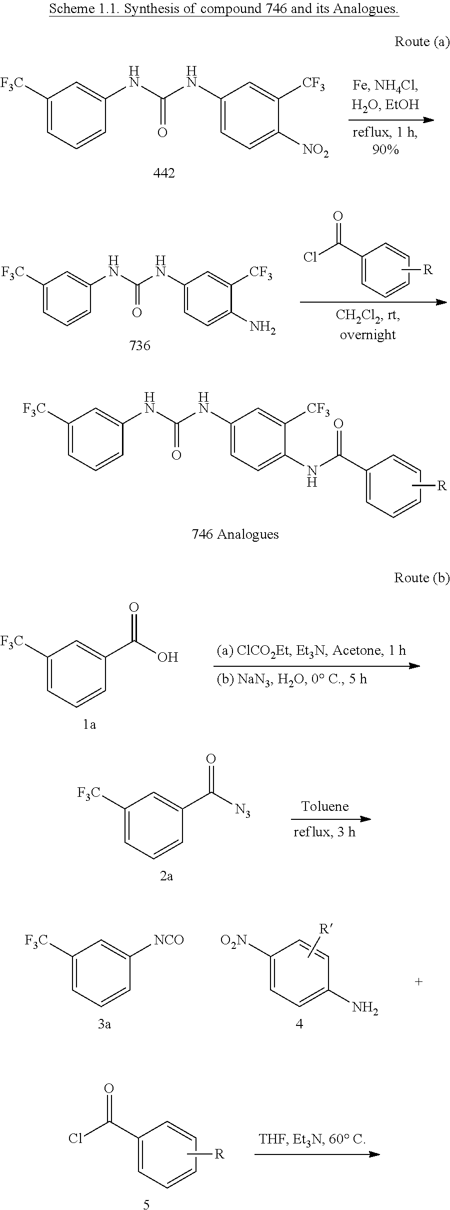

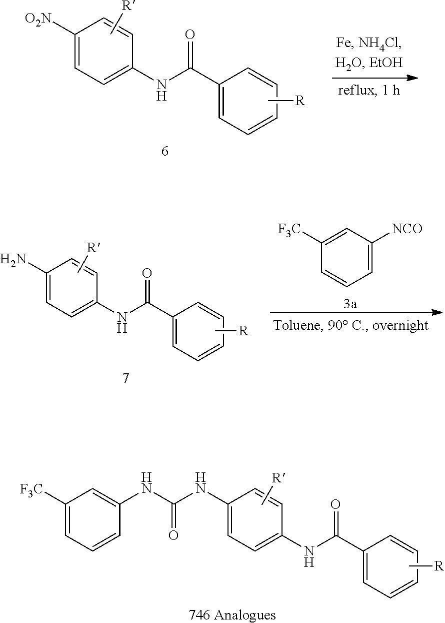

Preparation of Compound 736:

Referring to Scheme 1.1 reproduced above, to a solution of compound 442 (1 mmol) in EtOH (10 mL), iron powder (1.4 g, 25 mmol) was added at reflux. Then 1 mL NH₄Cl solution (0.16 N) was added. The reaction mixture was refluxed for 1 h. The solid was filtered while hot, the filtrate was concentrated under reduced pressure and purified by column chromatography (hexane/EtOAc=4:1) to give compound 736 (0.326 g, 89.8%) as white solid.

General Procedure for the Synthesis of the 746 Analogues Following Route (a)—Scheme 1.1:

To a solution of 736 (0.18 g, 0.5 mmol) and triethylamine (0.1 mL, 1 mmol) in dry THF (10 mL), substituted benzoyl chloride was added dropwise. The reaction mixture was stirred at room temperature overnight. Then water was added to the mixture which was extracted with dichloromethane. The organic phase was washed with water and brine, dried (Na₂SO₄), and concentrated. The obtained crude product was purified by column chromatography.

General Procedure for the Synthesis of the 746 Analogues Following Route (b)—Scheme 1.1:
(i) General procedure for the synthesis of amide 6: To a solution of 4 (1 mmol) and triethylamine (0.1 mL, 1 mmol) in dry THF (10 mL), substituted benzoyl chloride was added dropwise. The reaction mixture was stirred at room temperature for 12 h. Then water was added to the mixture which was extracted with dichloromethane. The organic phase was washed with water and brine, dried (Na₂SO₄), and concentrated. The obtained crude product was purified by column chromatography to give amide 6.
(ii) General procedure for the synthesis of 7: This was performed according to the procedure for the preparation of compound 736 outlined above.
(iii) General procedure for the synthesis of 2a: To a solution of 1a (1 mmol) in dry acetone (10 mL), triethylamine (1.1 mmol) and ethyl chlorocarbamate (1.1 mmol) were added dropwise at 0° C. After stirring at 0° C. for 1 h, sodium azide (1.1 mmol, 0.215 g) dissolved in 5 mL water was added dropwise. Stirring was continued at 0° C. for 5 h. Ice water was added. The mixture was extracted by dichloroform (3×20 mL). The combined organic layers were washed with brine and dried over Na₂SO₄. The organic phase was concentrated under reduced pressure. Colorless oil was obtained and used in the following reaction without further purification.
(iv) General procedure for the synthesis of the 746 Analogues: A solution of aryl azide 2a (0.5 mmol) in toluene (10 mL) was heated at 120° C. for 3 h to give aryl isocyanate 3a, which is not isolated and treated in situ with the respective 7 at 90° C. overnight. The solvent was cooled to room temperature and the precipitate was collected by filtration and washed with toluene.

### Characterization of Compound 746 and its Analogues

746 was prepared from 736 by following route (a): White solid, yield: 28.7%. ¹H NMR (500 MHz, acetone-d₆) δ 9.01 (d, J=11.0 Hz, 1H), 8.58 (d, J=8.0 Hz, 2H), 8.15 (d, J=2.3 Hz, 1H), 8.08 (s, 1H), 8.04-7.99 (m, 2H), 7.78-7.70 (m, 2H), 7.70-7.63 (m, 1H), 7.53 (t, J=8.0 Hz, 1H), 7.44-7.31 (m, 3H). MS (ESI) calculated for C₂₂H₁₅F₇N₃O₂[M+H] 486.1047. Found 486.1056.

743 was prepared from 736 by following route (a): White solid, yield: 36.7%. ¹H NMR (500 MHz, acetone-d₆) δ 9.13 (br, 1H), 8.60-8.57 (m, 2H), 8.16-8.04 (m, 4H), 7.78-7.65 (m, 3H), 7.53 (t, J=8.0 Hz, 1H), 7.39-7.26 (m, 3H). MS (ESI) calculated for C₂₂H₁₅F₇N₃O₂ [M+H] 486.1047. Found 486.1058.

806 was prepared from 736 by following route (b). White solid, yield: 33.5%. ¹H NMR (500 MHz, acetone-d₆) δ 9.35 (br, 1H), 8.38 (br, 1H), 8.16 (br, 1H), 8.09 (s, 1H), 7.85-7.81 (m, 1H), 7.79-7.71 (m, 2H), 7.70-7.65 (m, 1H), 7.62-7.52 (m, 3H), 7.51-7.46 (m, 1H), 7.37-7.23 (m, 3H). MS (ESI) calculated for C₂₁H₁₅F₄N₃O₂[M+H] 417.1100. Found 417.1178.

808 was prepared from 736 by following route (b). White solid, yield: 22.5%. ¹H NMR (500 MHz, acetone-d₆) δ 9.67 (br, 1H), 8.58 (d, J=3.9 Hz, 1H), 8.12-8.05 (m, 2H), 7.90 (d, J=7.8 Hz, 1H), 7.82-7.75 (m, 4H), 7.71 (d, J=8.0 Hz, 1H), 7.65-7.60 (m, 1H), 7.53 (t, J=8.0 Hz, 1H), 7.44-7.40 (m, 1H), 7.35 (d, J=7.8 Hz, 1H). MS (ESI) calculated for C₂₂H₁₅F₄N₄O₂[M+H] 443.1125. Found 443.1135.

814 was prepared from 736 by following route (b). White solid, yield: 36.7%. ¹H NMR (500 MHz, acetone-d₆) δ 9.67 (br, 1H), 8.99-8.99 (m, 1H), 8.34 (d, J=2.0 Hz, 1H), 8.10-8.05 (m, 2H), 8.01-7.98 (m, 1H), 7.86 (td, J=7.6, 1.8 Hz, 1H), 7.74-7.65 (m, 2H), 7.65-7.58 (m, 1H), 7.52 (t, J=8.0 Hz, 1H), 7.41-7.26 (m, 3H). MS (ESI) calculated for C₂₂H₁₅F₇N₃O₂[M+H] 486.1047. Found 486.1056.

815 was prepared from 736 by following route (a): White solid, yield: 23.4%. ¹H NMR (500 MHz, acetone-d₆) δ 9.20 (br, 1H), 8.60 (br, 1H), 8.58 (br, 1H), 8.14 (d, J=2.3 Hz, 1H), 8.08 (s, 1H), 7.87-7.83 (m, 1H), 7.79-7.71 (m, 3H), 7.70-7.64 (m, 1H), 7.63-7.59 (m, 1H), 7.53 (t, J=8.0 Hz, 1H), 7.43-7.38 (m, 1H), 7.35 (d, J=7.8 Hz, 1H). MS (ESI) calculated for C₂₂H₁₅F₇N₃O₂[M+H] 486.1047. Found 486.1057.

820 was prepared from 736 by following route (b). White solid, yield: 42.5%. ¹H NMR (500 MHz, acetone-d₆) δ 9.09 (br, 1H), 8.60 (br, 1H), 8.56 (br, 1H), 8.15 (s, 1H), 8.08 (s, 1H), 7.84-7.74 (m, 2H), 7.71 (d, J=8.0 Hz, 1H), 7.64 (d, J=7.5 Hz, 1H), 7.58-7.43 (m, 4H), 7.35 (d, J=7.7 Hz, 1H). MS (ESI) calculated for C₂₂H₁₅ClF₆N₃O₂ [M+H] 502.0751. Found 502.0761.

813 was prepared from 736 by following route (b). White solid, yield: 38.5%. ¹H NMR (500 MHz, acetone-d₆) δ ¹H NMR (500 MHz, acetone-d₆) δ 8.52 (br, 1H), 8.03 (br, 1H), 7.71-7.66 (m, 3H), 7.54-7.50 (m, 2H), 7.36-7.24 (m, 4H), 7.20-7.15 (m, 1H), 7.16-7.09 (m, 2H). MS (ESI) calculated for C₂₁H₁₅F₄N₃O₂[M+H] 436.1078. Found 436.1082.

789 White solid, yield: 34.7%. ¹H NMR (500 MHz, CDCl₃) δ 8.09 (br, 1H), 7.89 (br, 1H), 7.68 (s, 1H), 7.60 (d, J=8.1 Hz, 1H), 7.52 (s, 1H), 7.45 (d, J=8.5 Hz, 1H), 7.40 (t, J=7.9 Hz, 1H), 7.29 (d, J=7.7 Hz, 1H), 7.23-7.21 (m, 1H), 3.87-3.35 (m, 8H). MS (ESI) calculated for C₂₁H₁₅F₄N₃O₂[M+H] 462.1246. Found 462.1259.

822 White solid, yield: 67.5%.¹H NMR (800 MHz, acetone-d₆) δ 9.84 (br, 1H), 9.33 (br, 1H), 8.74-8.72 (m, 1H), 8.63-8.60 (m, 1H), 8.28 (br, 1H), 8.25-8.22 (m, 1H), 8.09 (d, J=21.0 Hz, 2H), 7.83 (d, J=8.1 Hz, 1H), 7.72 (d, J=8.4 Hz, 1H), 7.54 (t, J=7.9 Hz, 1H), 7.37 (d, J=7.6 Hz, 1H), 7.26-7.17 (m, 2H). MS (ESI) calculated for C₂₁H₁₅F₄N₃O₂[M+H]486.1047. Found 486.1057.

824: White solid. Yield: 47.3%. ¹H NMR (500 MHz, Acetone-de) δ 8.67 (br, 1H), 8.60 (br, 1H), 8.11-8.04 (m, 2H), 7.80-7.78 (m, 1H), 7.71 (d, J=8.0 Hz, 1H), 7.53 (t, J=8.0 Hz, 1H), 7.38 (d, J=8.4 Hz, 1H), 7.36 (d, J=7.7 Hz, 1H), 3.74-3.63 (m, 4H), 3.63-3.49 (m, 2H), 3.31-3.14 (m, 2H).

825: White solid. Yield: 88.2%. ¹H NMR (500 MHz, Acetone-d₆) δ 10.32 (s, 2H), 8.53 (d, J=13.2 Hz, 2H), 8.41 (d, J=8.9 Hz, 1H), 8.24-8.22 (m, 1H), 8.15 (d, J=2.5 Hz, 1H), 8.08 (s, 1H), 7.72 (d, J=8.2 Hz, 1H), 7.69-7.67 (m, 1H), 7.64-7.58 (m, 1H), 7.53 (t, J=8.0 Hz, 1H), 7.34 (d, J=7.8 Hz, 1H), 7.30 (d, J=8.0 Hz, 1H), 7.19-7.14 (m, 1H), 4.13 (s, 3H).

847: White solid. Yield: 45.8%. ¹H NMR (500 MHz, Acetone-d₆) δ 8.54 (br, 1H), 8.38 (br, 1H), 8.03 (s, 1H), 7.87 (s, 1H), 7.68-7.66 (m, 1H), 7.58-7.49 (m, 4H), 7.32 (d, J=7.7 Hz, 1H), 3.74-3.67 (m, 4H), 3.55-3.54 (m, 4H).

850: white solid. Yield: 48.3%. ¹H NMR (500 MHz, Acetone-d₆) δ 8.50 (br, 1H), 8.45 (br, 1H), 8.06 (s, 1H), 7.95 (d, J=2.5 Hz, 1H), 7.75 (dd, J=8.7, 2.5 Hz, 1H), 7.70 (d, J=8.3 Hz, 1H), 7.56-7.49 (m, 2H), 7.33 (d, J=7.8 Hz, 1H), 3.78-3.68 (m, 4H), 2.92-2.82 (m, 4H).

863: White solid. Yield: 87.6%. ¹H NMR (500 MHz, Acetone-d₆) δ 9.02 (d, J=10.9 Hz, 1H), 8.56 (br, 1H), 8.49 (br, 1H), 8.13 (d, J=2.4 Hz, 1H), 8.09-7.93 (m, 2H), 7.73 (dd, J=8.8, 2.4 Hz, 1H), 7.71-7.62 (m, 1H), 7.60-7.57 (m, 1H), 7.41-7.38 (m, 1H), 7.37-7.27 (m, 2H), 7.24-7.17 (m, 1H), 6.81-6.72 (m, 1H).

864: White solid. Yield: 83.5%. ¹H NMR (500 MHz, Acetone-d₆) δ 9.02 (d, J=10.9 Hz, 1H), 8.60 (br, 1H), 8.48 (br, 1H), 8.13 (d, J=2.4 Hz, 1H), 8.09-7.94 (m, 2H), 7.80 (t, J=2.0 Hz, 1H), 7.73 (dd, J=8.8, 2.4 Hz, 1H), 7.69-7.62 (m, 1H), 7.43-7.26 (m, 4H), 7.04-7.02 (m, 1H).

886: White solid. Yield: 91.2%. ¹H NMR (500 MHz, Acetone-d₆) δ 9.03 (d, J=11.0 Hz, 1H), 8.62 (br, 1H), 8.60 (br, 1H), 8.13 (d, J=2.4 Hz, 1H), 8.06-7.96 (m, 2H), 7.75 (dd, J=8.8, 2.4 Hz, 1H), 7.71-7.62 (m, 1H), 7.51 (s, 1H), 7.44 (t, J=2.0 Hz, 1H), 7.43-7.37 (m, 1H), 7.37-7.30 (m, 1H), 6.87 (s, 1H), 3.88 (s, 3H).

The 789 compound was prepared as follows:

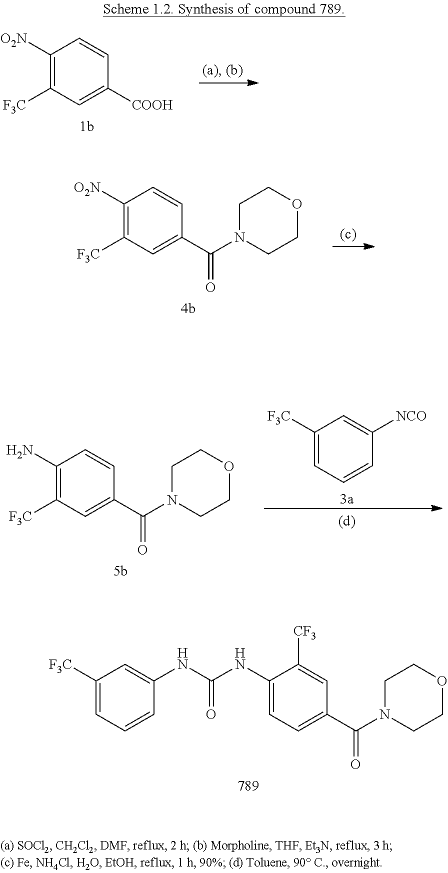

The 746 analogues of Formula (II) were prepared as follows:

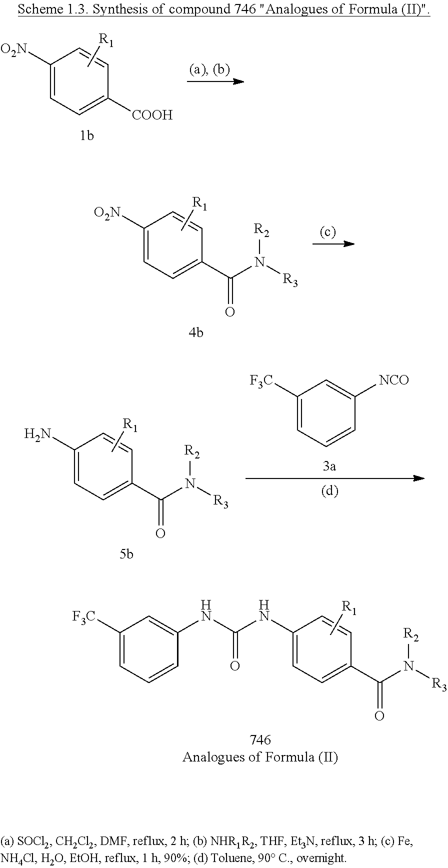

Compound 847 was prepared as follows:

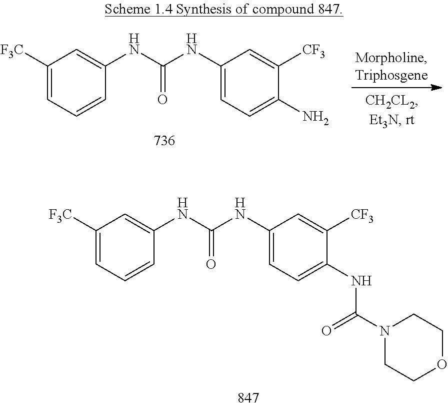

Compound 850 was prepared as follows:

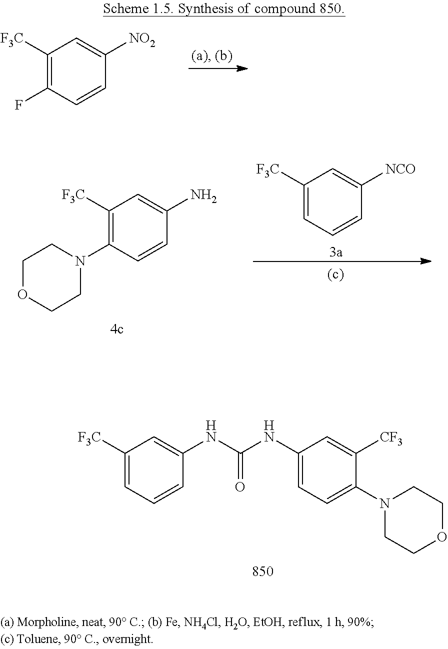

Preparation of Compound 789:

Referring to Schemes 1.2 and 1.3 above: (i) to a suspension of 1b (0.235 g, 1 mmol) in 10 mL of dichloromethane, thionyl chloride (0.15 mL, 2 mmol) and DMF (2 drops) were added dropwise. The mixture was refluxed for 2 h. Excess thionyl chloride was distilled under reduced pressure to give crude chloride, which was dissolved in dry THF (10 mL), morpholone and triethylamine were added. The reaction mixture was refluxed for 3 h. After cooling to room temperature, water was added to the mixture and extracted with dichloromethane. The organic phase was washed with water and brine, dried (Na₂SO₄), and concentrated. The obtained crude product was purified by column chromatography to give amide 4b. (ii) Synthesis of 5b: This was performed according to the procedure for the preparation of compound 736 outlined above. (iii) A mixture of aryl isocyanate 3a (1 mmol) and 5b (1 mmol) in toluene was heated at 90° C. overnight. The solvent was cooled to room temperature and the precipitate was collected by filtration and washed with toluene. Colorless syrup, yield: 56.5%.

Preparation of Compound 847:

Referring to Scheme 1.4 above: To a solution of triphosgene (0.296 g, 1 mmol) in CH₂Cl₂ (5 mL) at rt under N₂ was added 736 (0.36 g, 1 mmol). The reaction mixture was stirred for 30 min at rt. Then Et₃N (2 equiv) in CH₂Cl₂ (1 mL) was added. The mixture was stirred for 30 min. To this mixture was then added morpholine (1 mmol) in CH₂Cl₂ (1 mL). The resulting mixture was stirred for 30 min. Water was added to quench the reaction and extracted with dichloromethane. The organic phase was washed with water and brine, dried (Na₂SO₄), and concentrated. The obtained crude product was purified by column chromatography to give 847.

Preparation of Compound 850:

Referring to Scheme 1.5 above: A mixture of 3a (0.5 mmol) and amine 4c (0.5 mmol) in toluene (10 mL) was heated at 90° C. overnight. The solvent was cooled to room temperature and the precipitate was collected by filtration and washed with toluene to afford 850 as white solid.

The chemical structures of compounds 743, 746, 747, 789, 806, 808, 814, 815, 816, 820, 822, 824, 825, 847, 850, 863, 864 and 886 prepared as described above are depicted in the following Table 1.1.

TABLE 1.1
Compound 746 and its Analogues.

| ID | Structure |
|---|---|
| 746 | 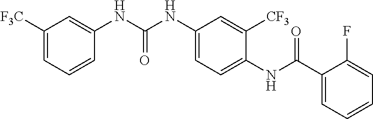 [SMILES: O=C(Nc1cccc(C(F)(F)F)c1)Nc1ccc(NC(=O)c2ccccc2F)c(C(F)(F)F)c1] |
| 743 | 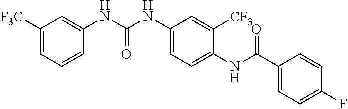 [SMILES: O=C(Nc1cccc(C(F)(F)F)c1)Nc1ccc(NC(=O)c2ccc(F)cc2)c(C(F)(F)F)c1] |
| 747 | 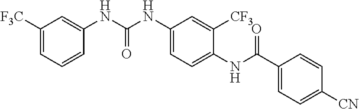 [SMILES: N#Cc1ccc(C(=O)Nc2ccc(NC(=O)Nc3cccc(C(F)(F)F)c3)cc2C(F)(F)F)cc1] |
| 806 | 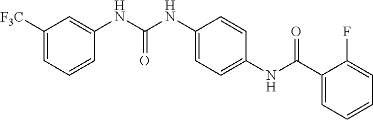 [SMILES: O=C(Nc1ccc(NC(=O)c2ccccc2F)cc1)Nc1cccc(C(F)(F)F)c1] |
| 808 | 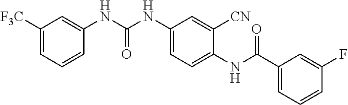 [SMILES: N#Cc1cc(NC(=O)Nc2cccc(C(F)(F)F)c2)ccc1NC(=O)c1cccc(F)c1] |
| 814 | 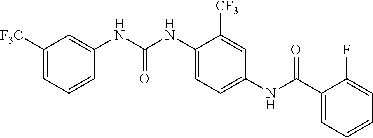 [SMILES: O=C(Nc1cccc(C(F)(F)F)c1)Nc1ccc(NC(=O)c2ccccc2F)cc1C(F)(F)F] |
| 815 | 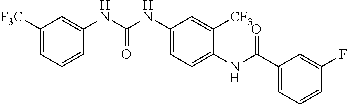 [SMILES: O=C(Nc1cccc(C(F)(F)F)c1)Nc1ccc(NC(=O)c2cccc(F)c2)c(C(F)(F)F)c1] |
| 816 | 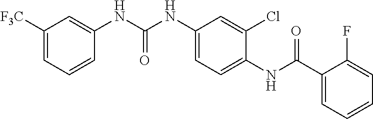 [SMILES: O=C(Nc1cccc(C(F)(F)F)c1)Nc1ccc(NC(=O)c2ccccc2F)c(Cl)c1] |
| 789 | 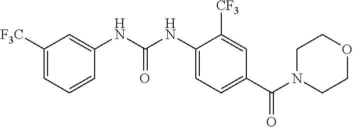 [SMILES: O=C(Nc1cccc(C(F)(F)F)c1)Nc1ccc(C(=O)N2CCOCC2)cc1C(F)(F)F] |
| 820 | 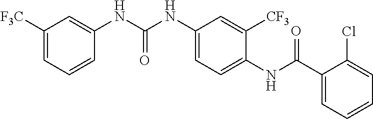 [SMILES: O=C(Nc1cccc(C(F)(F)F)c1)Nc1ccc(NC(=O)c2ccccc2Cl)c(C(F)(F)F)c1] |
| 813 | 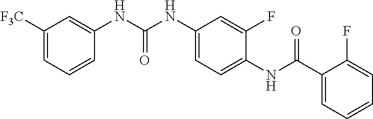 [SMILES: O=C(Nc1cccc(C(F)(F)F)c1)Nc1ccc(NC(=O)c2ccccc2F)c(F)c1] |
| 822 | 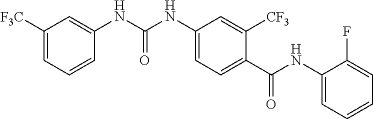 [SMILES: O=C(Nc1cccc(C(F)(F)F)c1)Nc1ccc(C(=O)Nc2ccccc2F)c(C(F)(F)F)c1] |
| 825 | 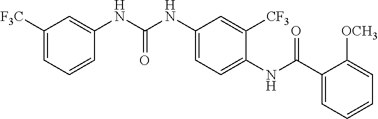 [SMILES: COc1ccccc1C(=O)Nc1ccc(NC(=O)Nc2cccc(C(F)(F)F)c2)cc1C(F)(F)F] |
| 863 | 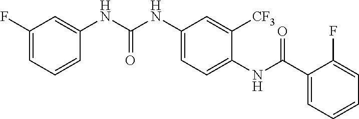 [SMILES: O=C(Nc1cccc(F)c1)Nc1ccc(NC(=O)c2ccccc2F)c(C(F)(F)F)c1] |
| 864 | 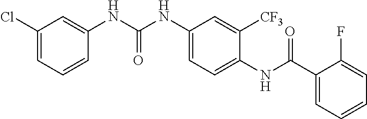 [SMILES: O=C(Nc1cccc(Cl)c1)Nc1ccc(NC(=O)c2ccccc2F)c(C(F)(F)F)c1] |
| 886 | 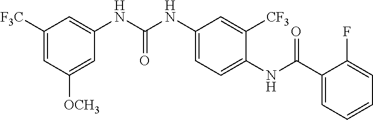 [SMILES: COc1cc(NC(=O)Nc2ccc(NC(=O)c3ccccc3F)c(C(F)(F)F)c2)cc(C(F)(F)F)c1] |
| 824 | 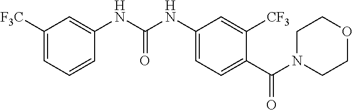 [SMILES: O=C(Nc1cccc(C(F)(F)F)c1)Nc1ccc(C(=O)N2CCOCC2)c(C(F)(F)F)c1] |
| 847 | 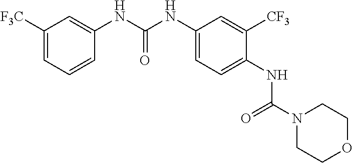 [SMILES: O=C(Nc1cccc(C(F)(F)F)c1)Nc1ccc(NC(=O)N2CCOCC2)c(C(F)(F)F)c1] |
| 850 | 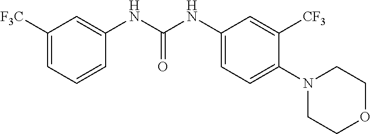 [SMILES: O=C(Nc1cccc(C(F)(F)F)c1)Nc1ccc(N2CCOCC2)c(C(F)(F)F)c1] |

### Synthesis of Additional Analogues of Compound 746

Compound 849 was prepared as follows:

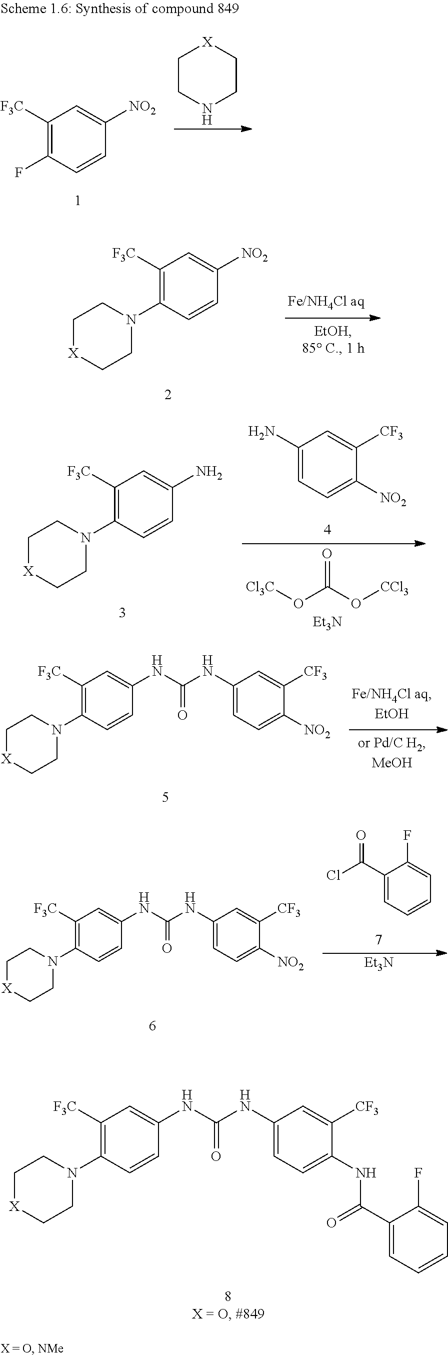

General Procedure for the synthesis of compound 849: referring to the Scheme 1.6 above, morpholine (6.0 mmol) was added to a solution of compound 1 (3.0 mmol) in 20.0 mL DMSO. The mixture was stirred at 100° C. for 4 h. The mixture was diluted with EtOAc and washed with brine. The organic layer was dried over Na₂SO₄. Solvents were removed under reduced pressure to afford the crude products 2, which were purified through flash chromatography on silica gel (Hexane/EtOAc 10:1 to 4:1 as the eluent). Compound 2 (2.0 mmol) was dissolved in EtOH (10.0 mL), Fe powder (200 mg) was added followed by 1.0 mL 5% aqueous solution of NH₄Cl. The mixture was refluxed for 1 h. The solvent was removed in wacuo and the residue was dissolved in acetone. After filtration and concentration in vacuo, the residue was purified by flash chromatography on silica gel (Hexane/EtOAc 3:1 to 1:1 as the eluent) to afford compound 3. To a solution of triphosgene (2.0 mmol) in dry DCM (4.0 mL), amine 4 (2.0 mmol) in DCM (8.0 mL) was added dropwise followed by the dropwise addition of triethylamine (0.6 mL) in DCM (2.0 mL) over 5 min at room temperature. The mixture was stirred for 20 min. Then amine 3 (2.0 mmol) in DCM (4.0 mL) was added dropwise into the mixture. Stirring was continued for 30 min. The reaction was quenched with dilute Na₂CO₃. The organic layer was washed with water and brine, and dried over Na₂SO₄. After filtration and concentration in vacuo, the residues was purified by recrystallization (solvent: DCM) to afford compound 5. Compound 5 (1.0 mmol) was dissolved in EtOH (8.0 mL), Fe powder (100 mg) was added followed by 1.0 mL 5% aqueous solution of NH₄Cl. The mixture was refluxed for 1 h. The solvent was removed in wacuo and the residue was dissolved in acetone. After filtration and concentration in vacuo, the residue was purified by recrystallization (solvent: DCM) to afford compound 6. Compound 6 (0.1 mmol) was dissolved in dry THF (5.0 mL). Triethylamine (0.2 mmol) was added followed by acyl chloride 7 (0.15 mmol) at 0° C. The reaction mixture was stirred at 0° C. for 30 min. Then the reaction was quenched with water and diluted with EtOAc. The organic layer was washed with brine, and dried over Na₂SO₄. After filtration and concentration in vacuo, the residue was purified by flash chromatography in silica gel (Hexane/EtOAc 5:1 to 1:1 as the eluent) to afford compound 849.

Compounds 861 and 862 were prepared as follows:

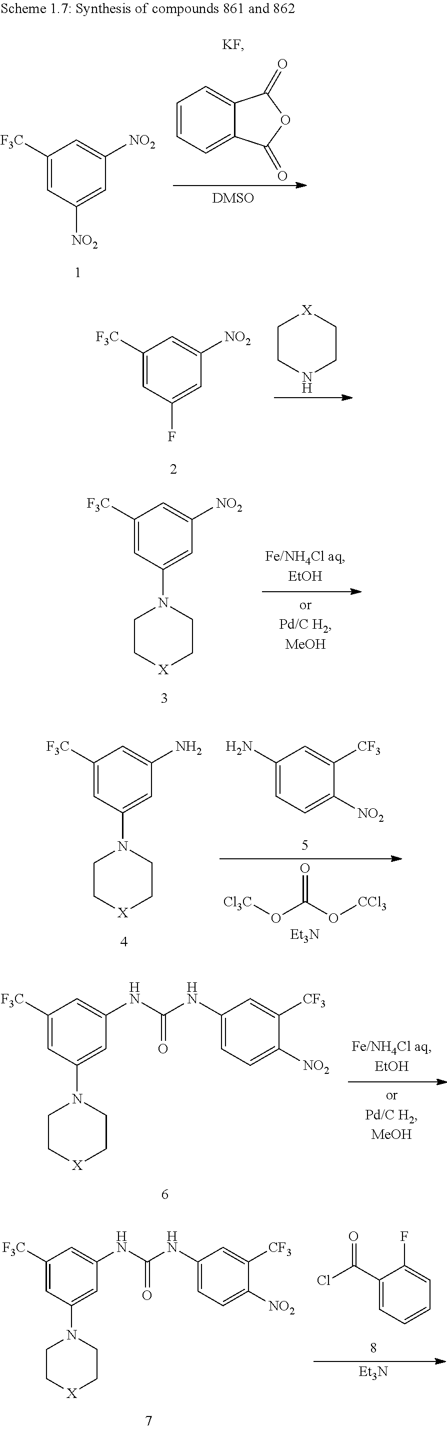

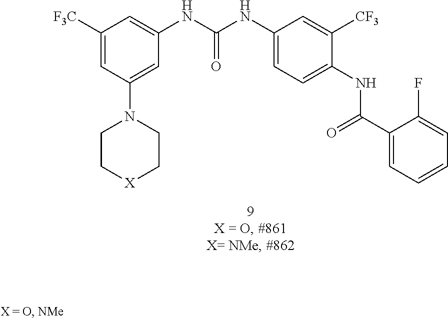

General Procedure for synthesis of compounds 861 and 862: referring to Scheme 1.7 above, a suspension of compound 1 (5.0 mmol), KF (6.0 mmol) and phthalic anhydride (4.0 mmol) in 8.0 mL DMSO. The mixture was stirred at 150° C. for 4 h. The mixture was diluted with EtOAc and washed with brine. The organic layer was dried over Na₂SO₄. Solvents were removed under reduced pressure to afford the crude products 2, which were purified through flash chromatography on silica gel (Hexane/EtOAc 50:1 to 15:1 as the eluent). Marpholine (6.0 mmol) was added to a solution of compound 2 (3.0 mmol) in 20.0 mL DMSO. The mixture was stirred at 100° C. for 4 h. The mixture was diluted with EtOAc and washed with brine. The organic layer was dried over Na₂SO₄. Solvents were removed under reduced pressure to afford the crude products 3, which were purified through flash chromatography on silica gel (Hexane/EtOAc 10:1 to 4:1 as the eluent). Compound 3 (2.0 mmol) was dissolved in EtOH (10.0 mL), Fe powder (200 mg) was added followed by 1.0 mL 5% aqueous solution of NH₄Cl. The mixture was refluxed for 1 h. The solvent was removed in wacuo and the residue was dissolved in acetone. After filtration and concentration in vacuo, the residue was purified by flash chromatography on silica gel (Hexane/EtOAc 3:1 to 1:1 as the eluent) to afford compound 4. To a solution of triphosgene (2.0 mmol) in dry DCM (4.0 mL), amine 5 (2.0 mmol) in DCM (8.0 mL) was added dropwise followed by the dropwise addition of triethylamine (0.6 mL) in DCM (2.0 mL) over 5 min at room temperature. The mixture was stirred for 20 min. Then amine 4 (2.0 mmol) in DCM (4.0 mL) was added dropwise into the mixture. Stirring was continued for 30 min. The reaction was quenched with dilute Na₂CO₃. The organic layer was washed with water and brine, and dried over Na₂SO₄. After filtration and concentration in vacuo, the residue was purified by recrystallization (solvent: DCM) to afford compound 6. Compound 6 (1.0 mmol) was dissolved in EtOH (8.0 mL), Fe powder (100 mg) was added followed by 1.0 mL 5% aqueous solution of NH₄Cl. The mixture was refluxed for 1 h. The solvent was removed in wacuo and the residue was dissolved in acetone. After filtration and concentration in vacuo, the residue was purified by recrystallization (solvent: DCM) to afford compound 7. Compound 7 (0.1 mmol) was dissolved in dry THF (5.0 mL). Triethylamine (0.2 mmol) was added followed by acyl chloride 8 (0.15 mmol) at 0° C. The reaction mixture was stirred at 0° C. for 30 min. Then the reaction was quenched with water and diluted with EtOAc. The organic layer was washed with brine, and dried over Na₂SO₄. After filtration and concentration, the residue was purified by flash chromatography in silica gel (Hexane/EtOAc 5:1 to 1:1 as the eluent) to afford compound 861 or 862.

Characterization of Additional Analogues of 746.

Additional 746 analogues were synthesized according to Schemes 1.1-1.7 above. These compounds were verified by NMR and MS analysis, as outlined below. The structures of these 746 analogues are shown in Table 1.2 below.

849 White solid, 82.1% in yield. ¹H NMR (500 MHz, acetone-d₆) δ 9.01 (d, J=10.9 Hz, 1H), 8.54 (s, 1H), 8.47 (s, 1H), 8.14 (s, 1H), 8.07-7.97 (m, 2H), 7.96 (s, 1H), 7.75 (t, J=9.1 Hz, 2H), 7.66 (d, J=7.1 Hz, 1H), 7.52 (d, J=8.7 Hz, 1H), 7.40 (t, J=7.6 Hz, 1H), 7.34 (dd, J=11.6, 8.1 Hz, 1H), 3.74 (t, J=4.5 Hz, 4H), 2.87 (t, J=4.5 Hz, 4H). TOF MS (ESI), m/z: 571.16 [M+H]⁺.

861 White solid, 76.5% in yield. ¹H NMR (500 MHz, acetone-d₆) δ 9.01 (d, J=11.0 Hz, 1H), 8.54 (s, 1H), 8.43 (s, 1H), 8.14 (d, J=2.3 Hz, 1H), 8.10-7.95 (m, 2H), 7.73 (dd, J=8.8, 2.3 Hz, 1H), 7.70-7.62 (m, 1H), 7.41 (dd, J=12.5, 4.8 Hz, 2H), 7.38-7.28 (m, 2H), 6.90 (s, 1H), 3.80 (t, J=5.0 Hz, 4H), 3.22 (t, J=5.0 Hz, 4H). TOF MS (ESI), m/z: 571.16 [M+H]⁺.

862 White solid, 72.4% in yield. ¹H NMR (500 MHz, acetone-d) δ 9.01 (d, J=11.0 Hz, 1H), 8.61 (d, J=4.4 Hz, 1H), 8.48 (d, J=4.2 Hz, 1H), 8.14 (d, J=2.1 Hz, 1H), 8.07-7.96 (m, 2H), 7.74 (dd, J=8.8, 2.3 Hz, 1H), 7.70-7.62 (m, 1H), 7.44-7.37 (m, 2H), 7.37-7.30 (m, 2H), 6.88 (s, 1H), 3.26 (t, J=5.0 Hz., 4H), 2.86 (s, 3H), 2.52 (t, J=5.0 Hz., 4H). TOF MS (ESI), m/z: 584.19 [M+H]⁺.

878 White solid, 87.0% in yield. ¹H NMR (500 MHz, acetone-d₆) δ 9.03 (d, J=11.0 Hz, 1H), 8.77 (s, 1H), 8.66 (s, 1H), 8.12 (s, 1H), 8.02 (t, J=7.8 Hz, 2H), 7.79-7.70 (m, 3H), 7.70-7.62 (m, 1H), 7.40 (td, J=7.7, 1.0 Hz, 1H), 7.34 (dd, J=11.8, 8.4 Hz, 1H), 7.13 (d, J=8.5 Hz, 1H).

879 White solid, 87.0% in yield. ¹H NMR (500 MHz, acetone-d₆) δ 10.98 (s, 1H), 8.60 (s, 2H), 8.15-8.10 (m, 2H), 8.08 (s, 1H), 7.99 (d, J=9.0 Hz, 1H), 7.73 (t, J=9.2 Hz, 2H), 7.53 (t, J=8.0 Hz, 1H), 7.34 (d, J=7.7 Hz, 1H), 7.25-7.17 (m, 1H), 7.11-7.03 (m, 1H), 3.80-3.74 (m, 4H), 3.15-3.10 (m, 4H).

890 White solid, 93.1% in yield. ¹H NMR (500 MHz, acetone-d₆) δ 9.08 (s, 1H), 8.59 (s, 2H), 8.12 (s, 1H), 8.07 (s, 1H), 7.91 (s, 1H), 7.80 (d, J=4.9 Hz, 1H), 7.73 (t, J=9.9 Hz, 2H), 7.67 (t, J=8.0 Hz, 1H), 7.53 (t, J=8.0 Hz, 1H), 7.35 (d, J=7.8 Hz, 1H), 7.22 (s, 1H).

893 White solid, 52.6% in yield. ¹H NMR (500 MHz, acetone-d₆) δ 9.58 (s, 1H), 8.74 (s, 1H), 8.64 (s, 1H), 8.12-8.04 (m, 2H), 7.87-7.77 (m, 2H), 7.72 (d, J=8.2 Hz, 1H), 7.66 (d, J=8.3 Hz, 1H), 7.54 (t, J=8.0 Hz, 1H), 7.44-7.32 (m, 1H), 7.26-7.16 (m, 1H), 7.16-7.09 (m, 1H), 6.92-6.71 (m, 1H).

894 White solid, 84.9% in yield. ¹H NMR (500 MHz, acetone-d₆) δ 9.10 (s, 1H), 8.60 (d, J=15.5 Hz, 2H), 8.14 (d, J=2.3 Hz, 1H), 8.08 (s, 1H), 7.88 (d, J=8.7 Hz, 1H), 7.78 (d, J=9.0 Hz, 1H), 7.72 (d, J=8.1 Hz, 2H), 7.63-7.48 (m, 2H), 7.44-7.31 (m, 2H). HRMS (ESI) calcd for C₂₂H₁₃F₈N₃O₂[M+H]⁺ 504.0953. Found 504.0952.

896 White solid (hard to dissolve in acetone-d₆), 79.1% in yield. ¹H NMR (500 MHz, acetone-d₆) δ 9.01 (d, J=12.1 Hz, 1H), 8.78 (s, 1H), 8.49 (s, 1H), 8.15 (d, J=8.4 Hz, 1H), 8.07-7.97 (m, 2H), 7.77 (d, J=13.1 Hz, 1H), 7.72 (d, J=9.1 Hz, 1H), 7.69-7.62 (m, 2H), 7.46-7.36 (m, 2H), 7.34 (t, J=8.1 Hz, 1H), 6.88 (d, J=8.4 Hz, 1H). HRMS (ESI) calcd for C₂₂H₁₄F₇N₃O₂[M+H]⁺ 486.1047. Found 486.1046.

897 White solid, 92.7% in yield. ¹H NMR (500 MHz, acetone-d₆) δ 9.02 (d, J=10.8 Hz, 1H), 8.61 (d, J=17.1 Hz, 2H), 8.15 (d, J=2.3 Hz, 1H), 8.02 (dd, J=14.2, 8.5 Hz, 2H), 7.83-7.71 (m, 3H), 7.70-7.59 (m, 3H), 7.45-7.37 (m, 1H), 7.37-7.29 (m, 1H). HRMS (ESI) calcd for C₂₂H₁₄F₇N₃O₂[M+H]⁺ 486.1047. Found 486.1063.

898 White solid, 67.4% in yield. ¹H NMR (500 MHz, acetone-d₆) δ 8.66 (s, 1H), 8.53 (s, 1H), 8.47 (s, 1H), 8.07 (t, J=2.9 Hz, 2H), 7.94 (d, J=8.9 Hz, 1H), 7.71 (d, J=8.2 Hz, 1H), 7.64 (dd, J=8.9, 2.5 Hz, 1H), 7.59-7.48 (m, 4H), 7.34 (d, J=7.7 Hz, 1H), 7.06 (t, J=8.9 Hz, 2H).

900 White solid, 89.0% in yield. ¹H NMR (500 MHz, acetone-d₆) δ 9.36 (s, 1H), 8.62 (s, 1H), 8.58 (s, 1H), 8.16 (s, 1H), 8.08 (s, 1H), 7.80-7.70 (m, 3H), 7.59-7.51 (m, 2H), 7.35 (d, J=7.7 Hz, 1H), 7.16-7.10 (m, 2H). HRMS (ESI) calcd for C₂₂H₁₃F₈N₃O₂[M+H]⁺ 504.0953. Found 504.0965.

901 White solid, 91.3% in yield. ¹H NMR (500 MHz, acetone-d₆) δ 9.09 (d, J=10.2 Hz, 1H), 8.60 (d, J=13.9 Hz, 2H), 8.15 (d, J=2.4 Hz, 1H), 8.07 (s, 1H), 7.95 (d, J=9.2 Hz, 1H), 7.76 (dd, J=8.9, 2.2 Hz, 1H), 7.74-7.65 (m, 2H), 7.53 (t, J=7.9 Hz, 1H), 7.49-7.37 (m, 2H), 7.35 (d, J=7.8 Hz, 1H). HRMS (ESI) calcd for C₂₂H₁₃F₈N₃O₂[M+H]⁺ 504.0953. Found 504.0967.

902 White solid, 85.3% in yield. ¹H NMR (500 MHz, acetone-d₆) δ 8.99 (d, J=10.8 Hz, 1H), 8.58 (d, J=10.3 Hz, 2H), 8.14 (d, J=2.3 Hz, 1H), 8.12-8.03 (m, 2H), 7.95 (d, J=8.9 Hz, 1H), 7.78-7.68 (m, 2H), 7.53 (t, J=7.9 Hz, 1H), 7.35 (d, J=7.7 Hz, 1H), 7.28-7.19 (m, 2H). HRMS (ESI) calcd for C₂₂H₁₃F₈N₃O₂[M+H]⁺ 504.0953. Found 504.0969.

903 White solid, 90.0% in yield. ¹H NMR (500 MHz, acetone-d₆) δ 9.21 (d, J=8.6 Hz, 1H), 8.60 (d, J=16.9 Hz, 2H), 8.27 (s, 1H), 8.16 (s, 1H), 8.08 (s, 1H), 8.02 (s, 1H), 7.92 (d, J=9.3 Hz, 1H), 7.78 (dd, J=8.6, 2.3 Hz, 1H), 7.72 (d, J=8.4 Hz, 1H), 7.65-7.56 (m, 1H), 7.53 (t, J=8.0 Hz, 1H), 7.35 (d, J=8.0 Hz, 1H).

904 White solid, 57.1% in yield. ¹H NMR (500 MHz, acetone-d₆) δ 9.72 (s, 1H), 8.73 (s, 1H), 8.62 (s, 1H), 8.14-8.02 (m, 2H), 7.84 (dd, J=8.1, 2.0 Hz, 1H), 7.78 (d, J=11.6 Hz, 1H), 7.73 (d, J=8.3 Hz, 1H), 7.68 (d, J=8.2 Hz, 1H), 7.54 (t, J=8.0 Hz, 1H), 7.48 (d, J=7.8 Hz, 1H), 7.39 (dd, J=15.0, 8.3 Hz, 2H), 6.90 (td, J=8.7, 2.6 Hz, 1H).

905 White solid, 48.7% in yield. ¹H NMR (500 MHz, acetone-d₆) δ 8.83 (s, 1H), 8.51 (d, J=23.6 Hz, 2H), 8.07 (d, J=2.5 Hz, 2H), 7.92 (t, J=8.2 Hz, 1H), 7.74-7.55 (m, 4H), 7.52 (t, J=8.0 Hz, 1H), 7.34 (d, J=7.7 Hz, 1H), 7.32-7.24 (m, 1H), 7.16 (dd, J=8.2, 1.2 Hz, 1H), 6.75 (td, J=8.4, 2.6 Hz, 1H). HRMS (ESI) calcd for C₂₂H₁₅F₇N₄O₂[M+H] 501.1156. Found 501.1167.

906 White solid, 76.8% in yield. ¹H NMR (500 MHz, acetone-d₆) δ 8.84 (s, 1H), 8.57 (s, 2H), 8.13 (d, J=2.5 Hz, 1H), 8.07 (s, 1H), 7.91 (d, J=8.8 Hz, 1H), 7.84-7.79 (m, 1H), 7.76-7.68 (m, 2H), 7.53 (t, J=8.0 Hz, 1H), 7.35 (d, J=7.8 Hz, 1H), 7.25 (dt, J=3.5, 0.8 Hz, 1H), 6.74-6.66 (m, 1H). HRMS (ESI) calcd for C₂₀H₁₃F₆N₃O₃[M+H]⁺ 458.0934. Found 458.0950.

907 White solid, 94.2% in yield. ¹H NMR (500 MHz, acetone-d₆) δ 9.10 (d, J=9.1 Hz, 1H), 8.61 (d, J=15.1 Hz, 2H), 8.15 (d, J=2.4 Hz, 1H), 8.08 (s, 1H), 7.98-7.90 (m, 2H), 7.76 (dd, J=8.8, 2.4 Hz, 1H), 7.72 (d, J=8.3 Hz, 1H), 7.69-7.64 (m, 1H), 7.53 (t, J=8.0 Hz, 1H), 7.40 (dd, J=10.7, 8.9 Hz, 1H), 7.35 (d, J=7.8 Hz, 1H). HRMS (ESI) calcd for C₂₂H₁₃ClF₇N₃O₂ [M+H]⁺ 520.0657. Found 520.0668.

911 White solid, 69.6% in yield. ¹H NMR (500 MHz, acetone-d₆) δ 9.18 (d, J=8.4 Hz, 1H), 8.61 (d, J=14.6 Hz, 2H), 8.49 (t, J=8.6 Hz, 1H), 8.47-8.33 (m, 1H), 8.14 (d, J=2.5 Hz, 1H), 8.08 (s, 1H), 7.94 (d, J=7.4 Hz, 1H), 7.78 (dd, J=8.8, 2.5 Hz, 1H), 7.72 (d, J=8.2 Hz, 1H), 7.61-7.50 (m, 2H), 7.38-7.33 (m, 1H).

912 White solid, 92.2% in yield. ¹H NMR (500 MHz, acetone-d₆) δ 9.18 (d, J=8.4 Hz, 1H), 8.61 (d, J=14.6 Hz, 2H), 8.49 (t, J=8.6 Hz, 1H), 8.47-8.33 (m, 2H), 8.14 (d, J=2.5 Hz, 1H), 8.08 (s, 1H), 7.94 (d, J=7.4 Hz, 1H), 7.78 (dd, J=8.8, 2.5 Hz, 1H), 7.72 (d, J=8.2 Hz, 1H), 7.61-7.50 (m, 2H), 7.38-7.33 (m, 1H).

921 White solid, 76.1% in yield. ¹H NMR (500 MHz, acetone-d₆) δ 9.25 (d, J=7.3 Hz, 1H), 8.75-8.61 (m, 2H), 8.59 (d, J=4.9 Hz, 1H), 8.15 (d, J=2.4 Hz, 1H), 8.08 (s, 1H), 7.89-7.81 (m, 3H), 7.79 (d, J=9.0 Hz, 1H), 7.72 (d, J=8.0 Hz, 1H), 7.53 (t, J=8.0 Hz, 1H), 7.35 (d, J=7.7 Hz, 1H).

922 White solid, 39.1% in yield. ¹H NMR (500 MHz, acetone-d₆) δ 8.55 (s, 1H), 8.50 (s, 1H), 8.39 (s, 1H), 8.10-8.04 (m, 1H), 8.00 (d, J=15.5 Hz, 1H), 7.91 (d, J=8.8 Hz, 1H), 7.73-7.62 (m, 2H), 7.50 (dt, J=12.5, 8.0 Hz, 2H), 7.40 (dd, J=8.5, 2.2 Hz, 1H), 7.36-7.31 (m, 1H), 7.31-7.27 (m, 1H), 7.19-7.11 (m, 1H), 6.88 (d, J=8.8 Hz, 1H).

930 White solid, 90.4% in yield. ¹H NMR (500 MHz, acetone-d₆) δ 9.08 (d, J=10.3 Hz, 1H), 8.58 (s, 1H), 8.48 (s, 1H), 8.16 (d, J=2.4 Hz, 1H), 7.94 (d, J=8.8 Hz, 1H), 7.90 (s, 1H), 7.74 (dd, J=8.8, 2.4 Hz, 1H), 7.72-7.66 (m, 1H), 7.65-7.60 (m, 1H), 7.48-7.36 (m, 3H), 7.23 (d, J=7.7 Hz, 1H), 6.88 (t, J=56.2 Hz, 1H).

941 White solid, 91.1% in yield. ¹H NMR (500 MHz, acetone-d₆) δ 9.20 (d, J=7.7 Hz, 1H), 8.66 (s, 1H), 8.55 (s, 1H), 8.27 (d, J=4.0 Hz, 1H), 8.18 (d, J=2.4 Hz, 1H), 8.05-7.98 (m, 1H), 7.90 (s, 2H), 7.76 (dd, J=8.8, 2.3 Hz, 1H), 7.66-7.56 (m, 2H), 7.44 (t, J=7.9 Hz, 1H), 7.23 (d, J=7.6 Hz, 1H), 6.89 (t, J=56.2 Hz, 1H).

945 White solid, 83.9% in yield. ¹H NMR (500 MHz, acetone-d₆) δ 8.99 (d, J=9.9 Hz, 1H), 8.56 (s, 1H), 8.47 (s, 1H), 8.15 (d, J=2.5 Hz, 1H), 8.12-8.02 (m, 1H), 7.94 (d, J=8.8 Hz, 1H), 7.89 (s, 1H), 7.74 (dd, J=8.8, 2.4 Hz, 1H), 7.67-7.59 (m, 1H), 7.44 (t, J=7.9 Hz, 1H), 7.30-7.18 (m, 3H), 6.88 (t, J=56.2 Hz, 1H).

952 White solid, 78.9% in yield. ¹H NMR (500 MHz, acetone) δ 9.09 (d, J=8.6 Hz, 1H), 8.63 (d, J=14.2 Hz, 2H), 8.29-8.22 (m, 1H), 8.14 (d, J=2.5 Hz, 1H), 8.07 (s, 1H), 7.91 (d, J=8.8 Hz, 1H), 7.79-7.69 (m, 2H), 7.52 (t, J=8.0 Hz, 1H), 7.34 (dd, J=7.7, 0.8 Hz, 1H), 7.19 (dd, J=11.0, 8.7 Hz, 1H), 7.13 (dd, J=10.6, 8.7 Hz, 1H).

971 White solid, 92.5% in yield. ¹H NMR (500 MHz, acetone) δ 9.43 (s, 1H), 8.68 (s, 1H), 8.64 (s, 1H), 8.17 (s, 1H), 8.08 (s, 1H), 7.78 (s, 2H), 7.74 (d, J=8.2 Hz, 1H), 7.58-7.49 (m, 2H), 7.41-7.34 (m, 2H), 7.27 (t, J=8.6 Hz, 1H).

983 White solid, 72.1% in yield. ¹H NMR (500 MHz, acetone) δ 9.11-9.01 (m, 2H), 8.82 (s, 1H), 8.55 (d, J=5.5 Hz, 1H), 8.13 (d, J=8.7 Hz, 2H), 8.04 (t, J=7.4 Hz, 2H), 7.79 (d, J=8.5 Hz, 1H), 7.72 (d, J=5.5 Hz, 1H), 7.70-7.65 (m, 1H), 7.42 (t, J=7.6 Hz, 1H), 7.36 (dd, J=11.7, 8.4 Hz, 1H).

Synthesis of 746 Analogues with Side Chain at Meta Position

746 analogues with side chain at meta position were prepared acceding to the followings Schemes 1.8 and 1.9:

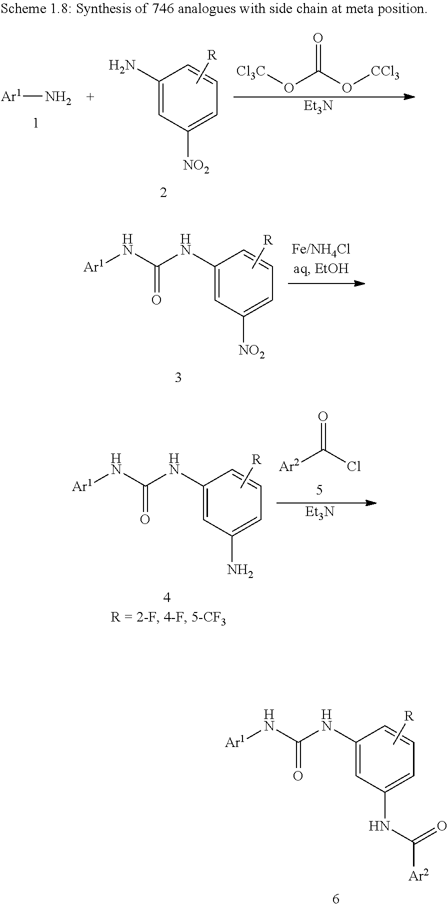

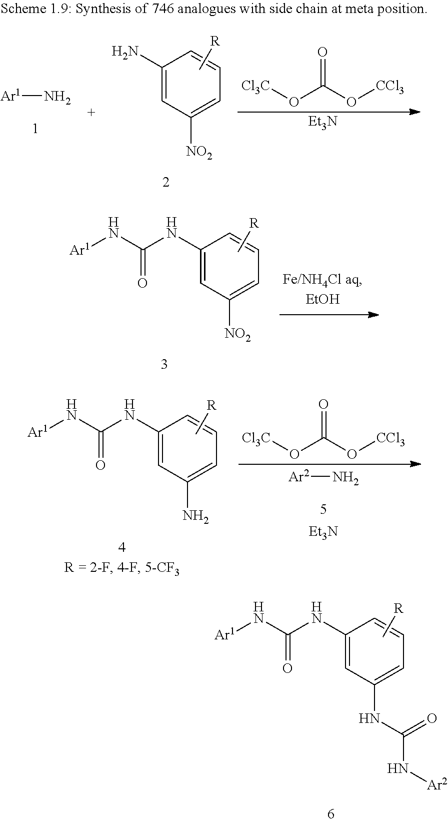

Characterization of 746 Analogues with Side Chain at Meta Position.

746 analogues with side chain at meta position were synthesized according to Schemes 1.8 and 1.9 above. These compounds were verified by NMR analysis as outlined below. The structures of these 746 analogues are shown in Table 1.3 below.

908 White solid, 94.1% in yield. ¹H NMR (500 MHz, acetone-d₆) δ 9.17 (d, J=9.5 Hz, 1H), 8.41 (d, J=37.0 Hz, 3H), 8.10 (s, 1H), 8.03-7.94 (m, 1H), 7.75-7.60 (m, 2H), 7.57-7.46 (m, 2H), 7.42-7.35 (m, 1H), 7.35-7.28 (m, 2H), 7.18 (dd, J=10.6, 9.0 Hz, 1H).

909 White solid, 87.5% in yield. ¹H NMR (500 MHz, acetone-d₆) δ 9.55 (s, 1H), 8.82 (s, 1H), 8.56 (dd, J=7.3, 2.6 Hz, 1H), 8.18-8.13 (m, 2H), 7.84 (td, J=7.5, 1.8 Hz, 1H), 7.76-7.69 (m, 1H), 7.63 (dd, J=8.1, 2.0 Hz, 1H), 7.61-7.55 (m, 1H), 7.52 (t, J=7.9 Hz, 1H), 7.38-7.31 (m, 2H), 7.27 (ddd, J=10.8, 8.3, 1.0 Hz, 1H), 7.17 (dd, J=11.0, 8.9 Hz, 1H).

910 White solid, 89.0% in yield. ¹H NMR (500 MHz, acetone-d₆) δ 9.45 (s, 1H), 8.44 (s, 1H), 8.30 (s, 1H), 8.12 (s, 1H), 8.07 (s, 1H), 7.83 (td, J=7.5, 1.8 Hz, 1H), 7.67 (dd, J=8.2, 2.0 Hz, 1H), 7.62-7.56 (m, 1H), 7.53-7.47 (m, 2H), 7.38-7.23 (m, 5H).

913 White solid, 90.9% in yield. ¹H NMR (500 MHz, acetone-d₆) δ 9.71 (s, 1H), 8.47 (s, 1H), 8.43-8.33 (m, 2H), 8.09 (s, 1H), 7.71 (dd, J=8.1, 1.8 Hz, 1H), 7.64-7.53 (m, 2H), 7.51 (t, J=8.0 Hz, 1H), 7.37-7.27 (m, 1H), 7.23-7.08 (m, 3H).

914 White solid, 86.1% in yield. ¹H NMR (500 MHz, acetone-d₆) δ 9.65 (s, 1H), 8.44 (s, 1H), 8.30 (s, 1H), 8.15 (s, 1H), 8.12-8.06 (m, 1H), 7.88 (ddd, J=7.7, 1.6, 0.9 Hz, 1H), 7.78 (ddd, J=9.7, 2.5, 1.5 Hz, 1H), 7.66 (dd, J=8.2, 1.9 Hz, 1H), 7.62-7.54 (m, 2H), 7.51 (t, J=7.9 Hz, 1H), 7.41-7.29 (m, 2H), 7.29-7.26 (m, 2H).

915 White solid, 84.7% in yield. ¹H NMR (500 MHz, acetone-d₆) δ 9.62 (s, 1H), 8.44 (s, 1H), 8.29 (s, 1H), 8.14 (s, 1H), 8.13-8.05 (m, 3H), 7.65 (d, J=6.3 Hz, 1H), 7.60-7.53 (m, 1H), 7.51 (t, J=8.0 Hz, 1H), 7.38-7.21 (m, 5H).

928 White solid, 35.3% in yield. ¹H NMR (500 MHz, acetone-d₆) δ 9.75 (s, 1H), 8.65 (s, 1H), 8.56 (s, 1H), 8.24 (s, 1H), 8.10 (s, 1H), 7.90 (s, 1H), 7.85 (td, J=7.5, 1.8 Hz, 1H), 7.81 (s, 1H), 7.69 (d, J=8.2 Hz, 1H), 7.65-7.58 (m, 1H), 7.53 (t, J=8.0 Hz, 1H), 7.38-7.33 (m, 2H), 7.29 (ddd, J=10.9, 8.3, 1.0 Hz, 1H).

929 White solid, 92.4% in yield. ¹H NMR (500 MHz, acetone-d₆) δ 9.27 (d, J=5.5 Hz, 1H), 8.46 (s, 1H), 8.40 (s, 2H), 8.10 (s, 1H), 7.92 (dd, J=6.3, 2.7 Hz, 1H), 7.70 (d, J=8.2 Hz, 1H), 7.65 (ddd, J=8.8, 4.3, 2.8 Hz, 1H), 7.57-7.49 (m, 2H), 7.38 (dd, J=10.5, 8.9 Hz, 1H), 7.32 (d, J=7.8 Hz, 1H), 7.18 (dd, J=10.6, 9.0 Hz, 1H).

942 White solid, 92.2% in yield. ¹H NMR (500 MHz, acetone-d₆) δ 9.25 (s, 1H), 8.52 (s, 1H), 8.48-8.35 (m, 2H), 8.10 (s, 1H), 7.74-7.64 (m, 2H), 7.58-7.47 (m, 2H), 7.46-7.35 (m, 2H), 7.32 (d, J=7.7 Hz, 1H), 7.18 (dd, J=10.6, 9.0 Hz, 1H).

944 White solid, 89.5% in yield. ¹H NMR (500 MHz, acetone-d₆) δ 9.40 (s, 1H), 8.49 (s, 1H), 8.45-8.37 (m, 2H), 8.25 (dd, J=6.3, 2.2 Hz, 1H), 8.10 (s, 1H), 8.04-7.96 (m, 1H), 7.70 (d, J=8.4 Hz, 1H), 7.57 (dd, J=16.7, 7.2 Hz, 1H), 7.55-7.47 (m, 2H), 7.32 (d, J=7.8 Hz, 1H), 7.19 (dd, J=10.6, 9.0 Hz, 1H).

946 White solid, 94.1% in yield. ¹H NMR (500 MHz, acetone-d₆) δ 9.32 (s, 1H), 8.48 (s, 1H), 8.36 (s, 1H), 8.18 (d, J=6.2 Hz, 1H), 8.09 (s, 1H), 7.88 (d, J=7.8 Hz, 1H), 7.77 (d, J=9.4 Hz, 1H), 7.69 (d, J=7.8 Hz, 1H), 7.64-7.55 (m, 1H), 7.55-7.45 (m, 2H), 7.39 (t, J=7.5 Hz, 1H), 7.31 (d, J=7.0 Hz, 1H), 7.16 (t, J=9.7 Hz, 1H).

947 White solid, 89.7% in yield. ¹H NMR (500 MHz, acetone-d₆) δ 9.26 (s, 1H), 8.47 (s, 1H), 8.35 (s, 1H), 8.19 (dd, J=6.9, 2.7 Hz, 1H), 8.15-8.06 (m, 3H), 7.68 (d, J=8.2 Hz, 1H), 7.55-7.43 (m, 2H), 7.34-7.25 (m, 3H), 7.15 (dd, J=10.4, 9.0 Hz, 1H).

948 White solid, 92.6% in yield. ¹H NMR (500 MHz, acetone-d₆) δ 9.29 (s, 1H), 8.48 (s, 1H), 8.43-8.34 (m, 2H), 8.09 (s, 1H), 7.70 (d, J=7.9 Hz, 2H), 7.58-7.48 (m, 3H), 7.41-7.34 (m, 1H), 7.32 (d, J=7.8 Hz, 1H), 7.18 (dd, J=10.5, 9.0 Hz, 1H).

949 White solid, 81.4% in yield. ¹H NMR (500 MHz, acetone-d₆) δ 8.84 (s, 1H), 8.71 (s, 1H), 8.37 (s, 1H), 8.31 (s, 1H), 8.07 (s, 1H), 7.92 (s, 1H), 7.74 (d, J=8.2 Hz, 1H), 7.53 (t, J=7.9 Hz, 1H), 7.36 (d, J=7.8 Hz, 1H).

950 White solid, 38.8% in yield. ¹H NMR (500 MHz, acetone-d₆) δ 8.51 (s, 1H), 8.48 (s, 1H), 8.43 (s, 1H), 8.35 (dd, J=7.3, 2.8 Hz, 1H), 8.33-8.28 (m, 2H), 8.09 (s, 1H), 7.70 (dd, J=8.2, 2.0 Hz, 1H), 7.50 (t, J=8.0 Hz, 1H), 7.43 (ddd, J=8.9, 4.4, 2.7 Hz, 1H), 7.34-7.27 (m, 1H), 7.18-7.13 (m, 2H), 7.09 (dd, J=11.1, 8.9 Hz, 1H), 7.05-6.99 (m, 1H).

951 White solid, 83.2% in yield. ¹H NMR (500 MHz, acetone-d₆) δ 9.27 (s, 1H), 8.46 (s, 1H), 8.39 (s, 2H), 8.23 (dd, J=6.9, 2.2 Hz, 1H), 8.09 (s, 1H), 8.01-7.92 (m, 1H), 7.70 (d, J=7.6 Hz, 1H), 7.59-7.46 (m, 2H), 7.32 (d, J=7.7 Hz, 1H), 7.18 (dd, J=10.7, 8.9 Hz, 2H).

953 White solid, 87.2% in yield. ¹H NMR (500 MHz, acetone) δ 8.63 (s, 2H), 8.58 (s, 2H), 8.09 (s, 2H), 7.99 (t, J=1.8 Hz, 1H), 7.69 (dd, J=8.2, 2.0 Hz, 2H), 7.64 (d, J=1.7 Hz, 2H), 7.52 (t, J=8.0 Hz, 2H), 7.34 (d, J=7.7 Hz, 2H).

954 White solid, 74.7% in yield. ¹H NMR (500 MHz, acetone) δ 9.42 (s, 1H), 8.64 (s, 2H), 8.54 (s, 1H), 8.42 (s, 2H), 8.25 (d, J=6.3 Hz, 1H), 8.04-7.95 (m, 1H), 7.76 (d, J=8.6 Hz, 2H), 7.63 (d, J=8.5 Hz, 2H), 7.19 (dd, J=10.5, 9.1 Hz, 1H).

955 White solid, 83.7% in yield. ¹H NMR (500 MHz, acetone) δ 8.61 (s, 4H), 8.01-7.96 (m, 1H), 7.77 (d, J=8.5 Hz, 4H), 7.68-7.59 (m, 6H).

956 White solid, 89.2% in yield. ¹H NMR (500 MHz, acetone) δ 9.17 (d, J=7.0 Hz, 1H), 8.53 (s, 1H), 8.45-8.38 (m, 2H), 8.09-8.01 (m, 1H), 7.77 (d, J=8.5 Hz, 2H), 7.62 (d, J=8.5 Hz, 2H), 7.56-7.48 (m, 1H), 7.27-7.12 (m, 3H).

957 White solid, 92.0% in yield. ¹H NMR (500 MHz, acetone) δ 9.57 (s, 1H), 8.86 (s, 1H), 8.53 (dd, J=7.3, 2.6 Hz, 1H), 8.15 (s, 2H), 7.98-7.88 (m, 1H), 7.76-7.66 (m, 1H), 7.63 (d, J=10.0 Hz, 1H), 7.52 (t, J=8.0 Hz, 1H), 7.38-7.30 (m, 1H), 7.22-7.09 (m, 3H).

958 White solid, 82.2% in yield. ¹H NMR (500 MHz, acetone) δ 9.77 (s, 1H), 8.87 (s, 1H), 8.54 (dd, J=7.3, 2.6 Hz, 1H), 8.24-8.17 (m, 1H), 8.15 (s, 1H), 8.02-7.88 (m, 2H), 7.73 (ddd, J=8.9, 4.4, 2.6 Hz, 1H), 7.63 (d, J=8.1 Hz, 1H), 7.58-7.46 (m, 2H), 7.34 (d, J=7.7 Hz, 1H), 7.18 (dd, J=11.0, 8.9 Hz, 1H).

959 White solid, 87.8% in yield. ¹H NMR (500 MHz, acetone) δ 9.42 (d, J=4.7 Hz, 1H), 8.95 (s, 1H), 8.43 (dd, J=6.7, 2.2 Hz, 1H), 8.26 (d, J=6.2 Hz, 1H), 8.20 (d, J=8.2 Hz, 1H), 8.07-7.99 (m, 1H), 7.73-7.63 (m, 3H), 7.62-7.54 (m, 2H), 7.29 (t, J=8.1 Hz, 1H), 7.20 (dd, J=10.5, 9.1 Hz, 1H).

960 White solid, 90.1% in yield. ¹H NMR (500 MHz, acetone) δ 9.18 (d, J=7.3 Hz, 1H), 8.93 (s, 1H), 8.43 (dd, J=6.8, 2.4 Hz, 1H), 8.19 (d, J=8.2 Hz, 1H), 8.11-8.02 (m, 1H), 7.71-7.63 (m, 3H), 7.60-7.53 (m, 1H), 7.33-7.16 (m, 4H).

963 White solid, 81.2% in yield. ¹H NMR (500 MHz, acetone) δ 9.41 (d, J=4.6 Hz, 1H), 8.40 (d, J=4.5 Hz, 1H), 8.34 (s, 1H), 8.28 (t, J=11.3 Hz, 2H), 8.06-7.98 (m, 1H), 7.59 (t, J=9.6 Hz, 1H), 7.57-7.51 (m, 3H), 7.48-7.41 (m, 2H), 7.19 (dd, J=10.2, 9.3 Hz, 1H).

964 White solid, 74.6% in yield. ¹H NMR (500 MHz, acetone) δ 8.53 (s, 2H), 8.44 (s, 2H), 7.95 (s, 1H), 7.64 (s, 2H), 7.60 (dt, J=11.9, 2.2 Hz, 2H), 7.32 (dd, J=14.9, 8.2 Hz, 2H), 7.20 (dd, J=8.1, 1.4 Hz, 2H), 6.78 (td, J=8.4, 2.4 Hz, 2H).

966 White solid, 87.3% in yield. ¹H NMR (500 MHz, acetone) δ 8.45 (s, 2H), 8.29 (s, 2H), 8.12 (s, 2H), 7.86 (s, 1H), 7.68 (d, J=8.4 Hz, 2H), 7.52 (t, J=8.0 Hz, 2H), 7.33 (d, J=7.7 Hz, 2H), 7.24-7.20 (m, 3H).

970 White solid, 85.7% in yield. ¹H NMR (500 MHz, acetone) δ 9.80 (s, 1H), 8.72 (s, 1H), 8.65 (s, 1H), 8.24 (s, 1H), 8.12 (s, 1H), 8.01-7.87 (m, 2H), 7.79 (s, 1H), 7.70 (d, J=8.4 Hz, 1H), 7.53 (t, J=8.0 Hz, 1H), 7.36 (d, J=7.0 Hz, 1H), 7.26-7.14 (m, 3H).

972 White solid, 71.9% in yield. ¹H NMR (500 MHz, acetone) δ 8.53 (s, 2H), 8.39 (s, 2H), 7.96 (s, 1H), 7.82 (t, J=1.9 Hz, 2H), 7.63 (s, 2H), 7.42-7.35 (m, 2H), 7.31 (t, J=8.1 Hz, 2H), 7.09-7.02 (m, 2H).

973 White solid, 80.2% in yield. ¹H NMR (500 MHz, acetone) δ 8.59 (s, 1H), 8.55 (d, J=3.9 Hz, 2H), 8.38 (s, 1H), 8.11 (s, 1H), 8.01-7.95 (m, 2H), 7.71 (d, J=8.3 Hz, 1H), 7.64 (d, J=13.5 Hz, 2H), 7.54 (t, J=8.0 Hz, 1H), 7.43 (ddd, J=8.1, 1.9, 0.9 Hz, 1H), 7.36 (d, J=7.7 Hz, 1H), 7.26 (t, J=8.0 Hz, 1H), 7.20 (ddd, J=7.9, 1.7, 1.0 Hz, 1H).

Synthesis of 746 Analogues with Side Chain at Ortho Position.

746 analogues with side chain at ortho position were prepared according to the following Schemes 1.10 and 1.11:

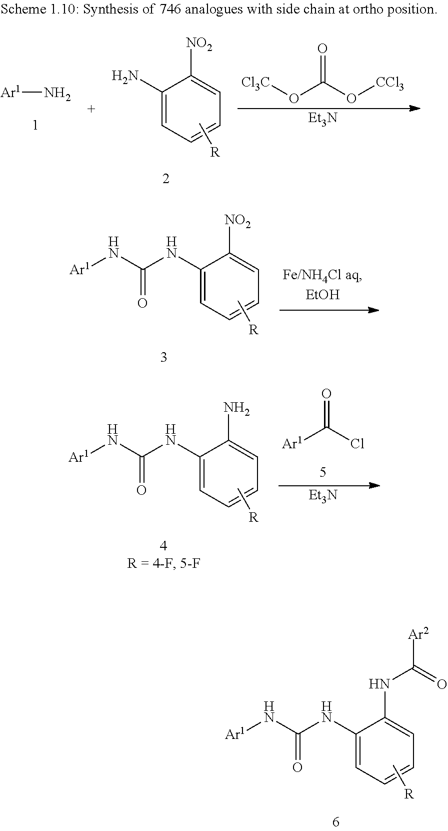

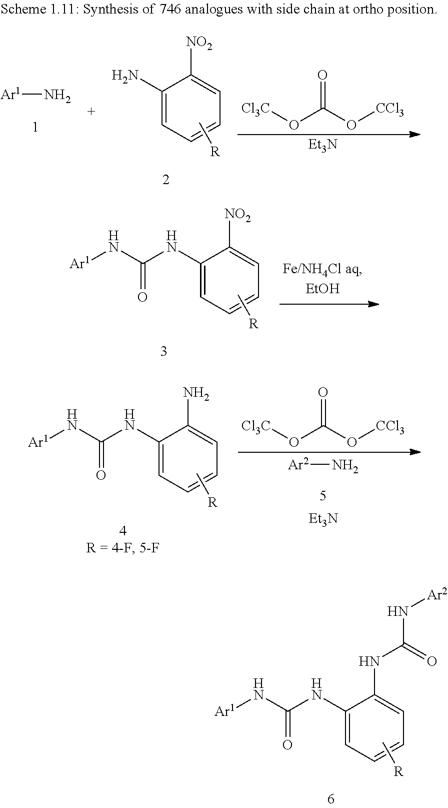

General Procedure for the Synthesis of 746 Analogues with Side Chain at Ortho Position:

Referring to the Scheme 1.10 above, to a solution of triphosgene (1.5 mmol) in dry DCM (4.0 mL), amine 2 (1.5 mmol) in DCM (12.0 mL) was added dropwise followed by the dropwise addition of triethylamine (0.9 mL) in DCM (2.0 mL) over 5 min at room temperature. The mixture was stirred for 20 min. Then amine 1 (1.5 mmol) in DCM (4.0 mL) was added dropwise into the mixture. Stirring was continued for 30 min. The reaction was quenched with dilute Na₂CO₃. The organic layer was washed with water and brine, and dried over Na₂SO₄. After filtration and concentration in vacuo, the residue was purified by recrystallization (solvent: DCM) to afford compound 3. Compound 3 (1.0 mmol) was dissolved in EtOH (8.0 mL), Fe powder (100 mg) was added followed by 1.0 mL 5% aqueous solution of NH₄Cl. The mixture was refluxed for 1 h. The solvent was removed in wacuo and the residue was dissolved in acetone. After filtration and concentration in vacuo, the residues was purified by recrystallization (solvent: DCM) to afford compound 4. Compound 4 (0.05 mmol) was dissolved in dry THF (5.0 mL). Triethylamine (0.2 mmol) was added followed by acyl chloride 5 (0.1 mmol) at rt. The reaction mixture was stirred at rt for 1 h. Then the reaction was quenched with water and diluted with EtOAc. The organic layer was washed with brine, and dried over Na₂SO₄. After filtration and concentration in vacuo, the residue was purified by flash chromatography in silica gel (Hexane/EtOAc 10:1 to 2:1 as the eluent) to afford compound 6, such as 961 or 962.

Referring to the Scheme 1.11 above, to a solution of triphosgene (1.5 mmol) in dry DCM (4.0 mL), amine 2 (1.5 mmol) in DCM (12.0 mL) was added dropwise followed by the dropwise addition of triethylamine (0.9 mL) in DCM (2.0 mL) over 5 min at room temperature. The mixture was stirred for 20 min. Then amine 1 (1.5 mmol) in DCM (4.0 mL) was added dropwise into the mixture. Stirring was continued for 30 min. The reaction was quenched with dilute Na₂CO₃. The organic layer was washed with water and brine, and dried over Na₂SO₄. After filtration and concentration in vacuo, the residue was purified by flash chromatography in silica gel (Hexane/EtOAc 10:1 to 2:1 as the eluent) to afford compound 3. Compound 3 (1.0 mmol) was dissolved in EtOH (8.0 mL), Fe powder (0.5 g) was added followed by 1.0 mL 5% aqueous solution of NH₄Cl. The mixture was refluxed for 1 h. The solvent was removed in wacuo and the residue was dissolved in acetone. After filtration and concentration in vacuo, the residues was purified by recrystallization (solvent: DCM) to afford compound 4. To a solution of triphosgene (0.5 mmol) in dry DCM (4.0 mL), amine 5 (0.5 mmol) in DCM (4.0 mL) was added dropwise followed by the dropwise addition of triethylamine (0.3 mL) in DCM (2.0 mL) over 2 min at room temperature. The mixture was stirred for 20 min. Then compound 4 (0.5 mmol) in DCM (6.0 mL) was added dropwise into the mixture. Stirring was continued for 30 min. The reaction was quenched with dilute Na₂CO₃. The organic layer was washed with water and brine, and dried over Na₂SO₄. After filtration and concentration in vacuo, the residue was purified by flash chromatography in silica gel (Hexane/EtOAc 10:1 to 2:1 as the eluent) to afford compound 6, such as 968.

Characterization of 746 Analogues with Side Chain at Ortho Position.

746 analogues with a side chain at meta position were synthesized according to Schemes 1.10 and 1.11 above. These compounds were verified by NMR analysis as outlined below. The structures of these 746 analogues are shown in Table 1.4 below.

961 White solid, 85.4% in yield. ¹H NMR (500 MHz, acetone) δ 9.66 (d, J=5.9 Hz, 1H), 9.15 (s, 1H), 8.27 (s, 1H), 8.15-8.05 (m, 2H), 7.85 (d, J=8.3 Hz, 1H), 7.69 (d, J=8.3 Hz, 1H), 7.55 (dd, J=8.8, 5.9 Hz, 1H), 7.50 (t, J=8.0 Hz, 1H), 7.32 (d, J=7.7 Hz, 1H), 7.24-7.12 (m, 2H), 7.01 (td, J=8.5, 3.0 Hz, 1H).

962 White solid, 79.5% in yield. ¹H NMR (500 MHz, acetone) δ 9.32 (d, J=5.0 Hz, 1H), 8.79 (s, 1H), 8.17-8.01 (m, 2H), 7.75 (dd, J=11.0, 2.8 Hz, 1H), 7.57 (dd, J=8.8, 6.0 Hz, 1H), 7.50 (d, J=8.9 Hz, 2H), 7.43 (d, J=8.9 Hz, 2H), 7.29-7.15 (m, 2H), 6.93 (td, J=8.5, 2.9 Hz, 1H).

965 White solid, 83.0% in yield. ¹H NMR (500 MHz, acetone) δ 9.31 (d, J=5.6 Hz, 1H), 8.78 (s, 1H), 8.13-8.04 (m, 1H), 7.98 (s, 1H), 7.75 (dd, J=11.0, 2.8 Hz, 1H), 7.61-7.51 (m, 2H), 7.35-7.19 (m, 3H), 7.14 (dd, J=8.2, 1.0 Hz, 1H), 6.94 (td, J=8.5, 2.9 Hz, 1H), 6.76 (td, J=8.3, 2.0 Hz, 1H).

967 White solid, 86.0% in yield. ¹H NMR (500 MHz, acetone) δ 8.80 (s, 2H), 8.06 (s, 2H), 7.97 (s, 2H), 7.71-7.63 (m, 4H), 7.50 (t, J=8.0 Hz, 2H), 7.32 (d, J=7.7 Hz, 2H), 7.22-7.15 (m, 2H).

968 White solid, 79.1% in yield. ¹H NMR (500 MHz, acetone) δ 9.70 (d, J=5.6 Hz, 1H), 8.83 (s, 1H), 8.06 (s, 1H), 8.00-7.90 (m, 2H), 7.81 (dd, J=10.3, 2.6 Hz, 1H), 7.73-7.62 (m, 2H), 7.55 (dd, J=8.8, 5.9 Hz, 1H), 7.51 (t, J=8.0 Hz, 1H), 7.40-7.31 (m, 2H), 7.11-7.00 (m, 1H).

968 White solid, 79.1% in yield. ¹H NMR (500 MHz, acetone) δ 9.70 (d, J=5.6 Hz, 1H), 8.83 (s, 1H), 8.06 (s, 1H), 8.00-7.90 (m, 2H), 7.81 (dd, J=10.3, 2.6 Hz, 1H), 7.73-7.62 (m, 2H), 7.55 (dd, J=8.8, 5.9 Hz, 1H), 7.51 (t, J=8.0 Hz, 1H), 7.40-7.31 (m, 2H), 7.11-7.00 (m, 1H).

974 White solid, 91.2% in yield. ¹H NMR (500 MHz, acetone) δ 9.59 (d, J=7.5 Hz, 1H), 8.71 (s, 1H), 8.17-8.03 (m, 1H), 7.91-7.77 (m, 2H), 7.64-7.49 (m, 2H), 7.29 (td, J=8.2, 6.7 Hz, 1H), 7.26-7.15 (m, 3H), 7.03 (td, J=8.4, 3.0 Hz, 1H), 6.76 (tdd, J=8.6, 2.6, 0.8 Hz, 1H).

975 White solid, 89.3% in yield. ¹H NMR (500 MHz, acetone) δ 9.60 (d, J=7.6 Hz, 1H), 8.72 (s, 1H), 8.02 (td, J=7.8, 1.6 Hz, 1H), 7.84 (s, 2H), 7.71-7.61 (m, 1H), 7.61-7.51 (m, 2H), 7.39 (td, J=7.7, 1.0 Hz, 1H), 7.35-7.24 (m, 2H), 7.18 (ddd, J=8.2, 2.0, 0.8 Hz, 1H), 7.02 (ddd, J=8.8, 8.1, 3.0 Hz, 1H), 6.76 (tdd, J=8.6, 2.6, 0.9 Hz, 1H).

TABLE 1.2
Additional analogues of compound 746

| ID | Structure |
|---|---|
| 896 | 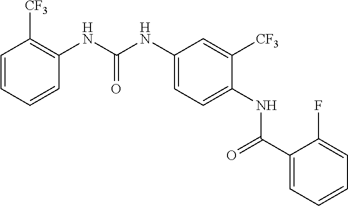 [SMILES: O=C(Nc1ccc(NC(=O)c2ccccc2F)c(C(F)(F)F)c1)Nc1ccccc1C(F)(F)F] |
| 897 | 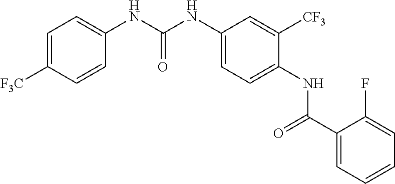 [SMILES: O=C(Nc1ccc(C(F)(F)F)cc1)Nc1ccc(NC(=O)c2ccccc2F)c(C(F)(F)F)c1] |
| 849 | 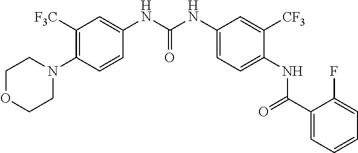 [SMILES: O=C(Nc1ccc(NC(=O)c2ccccc2F)c(C(F)(F)F)c1)Nc1ccc(N2CCOCC2)c(C(F)(F)F)c1] |
| 879 | 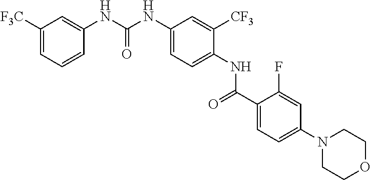 [SMILES: O=C(Nc1cccc(C(F)(F)F)c1)Nc1ccc(NC(=O)c2ccc(N3CCOCC3)cc2F)c(C(F)(F)F)c1] |
| 878 | 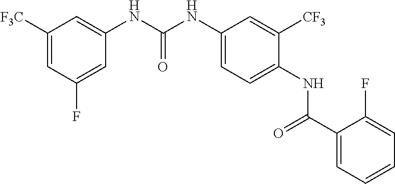 [SMILES: O=C(Nc1cc(F)cc(C(F)(F)F)c1)Nc1ccc(NC(=O)c2ccccc2F)c(C(F)(F)F)c1] |
| 861 | 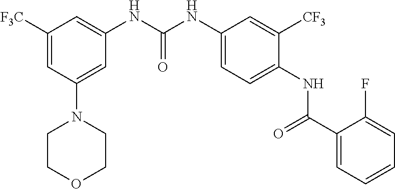 [SMILES: O=C(Nc1cc(N2CCOCC2)cc(C(F)(F)F)c1)Nc1ccc(NC(=O)c2ccccc2F)c(C(F)(F)F)c1] |
| 862 | 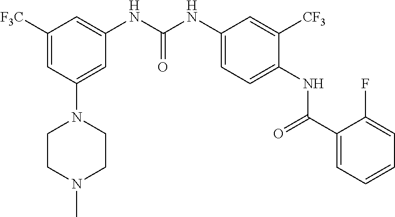 [SMILES: CN1CCN(c2cc(NC(=O)Nc3ccc(NC(=O)c4ccccc4F)c(C(F)(F)F)c3)cc(C(F)(F)F)c2)CC1] |
| 890 | 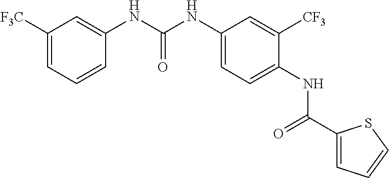 [SMILES: O=C(Nc1cccc(C(F)(F)F)c1)Nc1ccc(NC(=O)c2cccs2)c(C(F)(F)F)c1] |
| 900 | 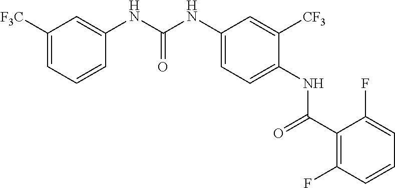 [SMILES: O=C(Nc1cccc(C(F)(F)F)c1)Nc1ccc(NC(=O)c2c(F)cccc2F)c(C(F)(F)F)c1] |
| 906 | 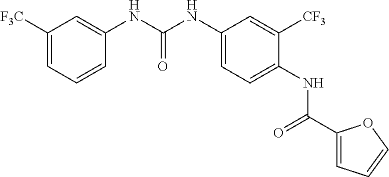 [SMILES: O=C(Nc1cccc(C(F)(F)F)c1)Nc1ccc(NC(=O)c2ccco2)c(C(F)(F)F)c1] |
| 894 | 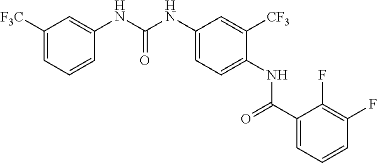 [SMILES: O=C(Nc1cccc(C(F)(F)F)c1)Nc1ccc(NC(=O)c2cccc(F)c2F)c(C(F)(F)F)c1] |
| 901 | 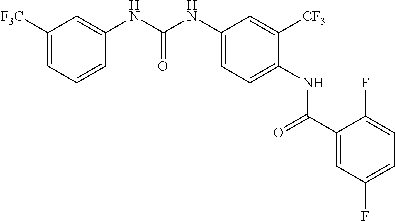 [SMILES: O=C(Nc1cccc(C(F)(F)F)c1)Nc1ccc(NC(=O)c2cc(F)ccc2F)c(C(F)(F)F)c1] |
| 902 | 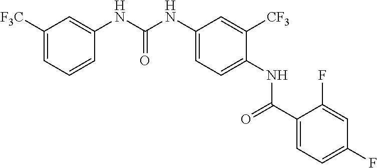 [SMILES: O=C(Nc1cccc(C(F)(F)F)c1)Nc1ccc(NC(=O)c2ccc(F)cc2F)c(C(F)(F)F)c1] |
| 903 | 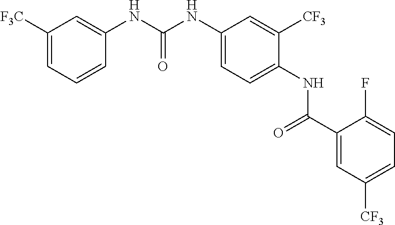 [SMILES: O=C(Nc1cccc(C(F)(F)F)c1)Nc1ccc(NC(=O)c2cc(C(F)(F)F)ccc2F)c(C(F)(F)F)c1] |
| 907 | 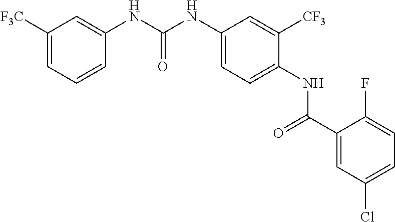 [SMILES: O=C(Nc1cccc(C(F)(F)F)c1)Nc1ccc(NC(=O)c2cc(Cl)ccc2F)c(C(F)(F)F)c1] |
| 911 | 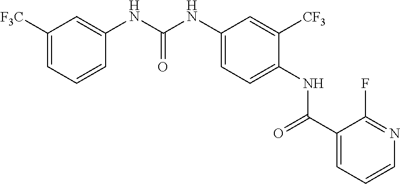 [SMILES: O=C(Nc1cccc(C(F)(F)F)c1)Nc1ccc(NC(=O)c2cccnc2F)c(C(F)(F)F)c1] |
| 952 | 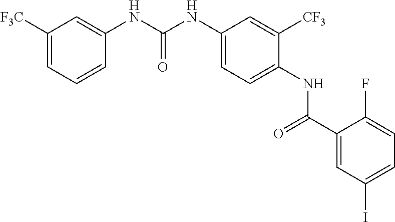 [SMILES: O=C(Nc1cccc(C(F)(F)F)c1)Nc1ccc(NC(=O)c2cc(I)ccc2F)c(C(F)(F)F)c1] |
| 921 | 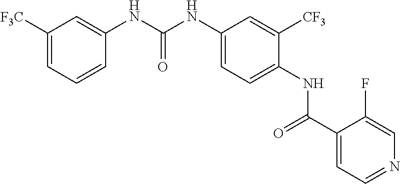 [SMILES: O=C(Nc1cccc(C(F)(F)F)c1)Nc1ccc(NC(=O)c2ccncc2F)c(C(F)(F)F)c1] |
| 904 | 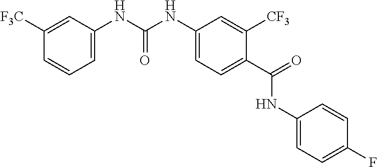 [SMILES: O=C(Nc1cccc(C(F)(F)F)c1)Nc1ccc(C(=O)Nc2ccc(F)cc2)c(C(F)(F)F)c1] |
| 893 | 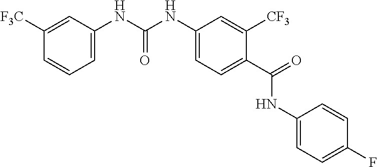 [SMILES: O=C(Nc1cccc(C(F)(F)F)c1)Nc1ccc(C(=O)Nc2ccc(F)cc2)c(C(F)(F)F)c1] |
| 971 | 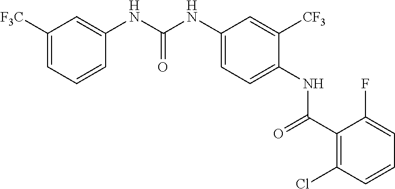 [SMILES: O=C(Nc1cccc(C(F)(F)F)c1)Nc1ccc(NC(=O)c2c(F)cccc2Cl)c(C(F)(F)F)c1] |
| 905 | 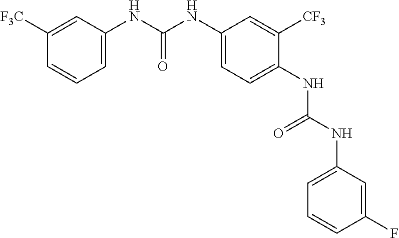 [SMILES: O=C(Nc1cccc(C(F)(F)F)c1)Nc1ccc(NC(=O)Nc2cccc(F)c2)c(C(F)(F)F)c1] |
| 898 | 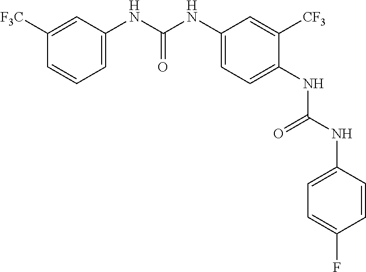 [SMILES: O=C(Nc1cccc(C(F)(F)F)c1)Nc1ccc(NC(=O)Nc2ccc(F)cc2)c(C(F)(F)F)c1] |
| 922 | 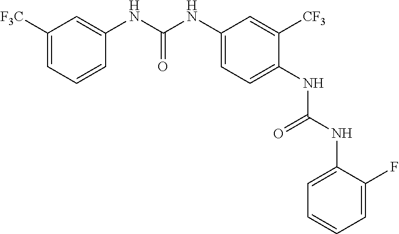 [SMILES: O=C(Nc1cccc(C(F)(F)F)c1)Nc1ccc(NC(=O)Nc2ccccc2F)c(C(F)(F)F)c1] |
| 912 | 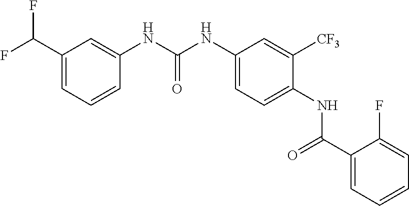 [SMILES: O=C(Nc1cccc(C(F)F)c1)Nc1ccc(NC(=O)c2ccccc2F)c(C(F)(F)F)c1] |
| 923 | 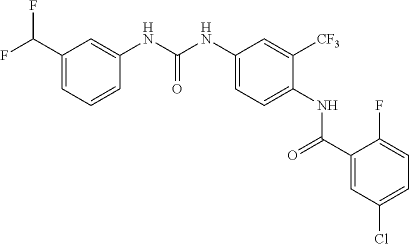 [SMILES: O=C(Nc1cccc(C(F)F)c1)Nc1ccc(NC(=O)c2cc(Cl)ccc2F)c(C(F)(F)F)c1] |
| 930 | 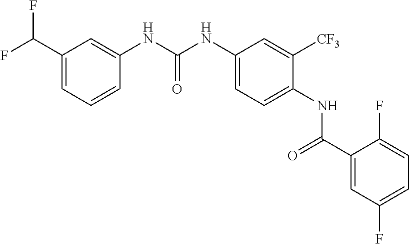 [SMILES: O=C(Nc1cccc(C(F)F)c1)Nc1ccc(NC(=O)c2cc(F)ccc2F)c(C(F)(F)F)c1] |
| 941 | 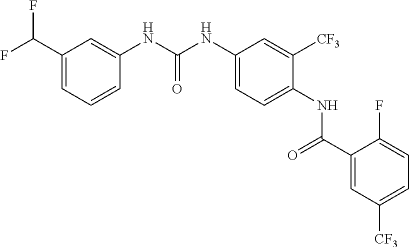 [SMILES: O=C(Nc1cccc(C(F)F)c1)Nc1ccc(NC(=O)c2cc(C(F)(F)F)ccc2F)c(C(F)(F)F)c1] |
| 945 | 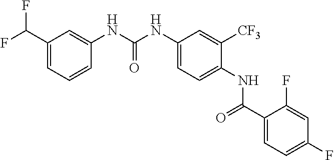 [SMILES: O=C(Nc1cccc(C(F)F)c1)Nc1ccc(NC(=O)c2ccc(F)cc2F)c(C(F)(F)F)c1] |
| 983 | 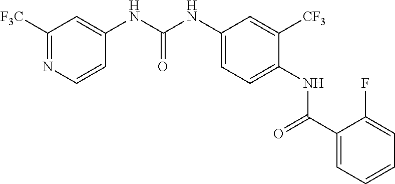 [SMILES: O=C(Nc1ccnc(C(F)(F)F)c1)Nc1ccc(NC(=O)c2ccccc2F)c(C(F)(F)F)c1] |

TABLE 1.3
Analogues of compound 746 with side chain at meta position

| ID | Structure |
|---|---|
| 908 | 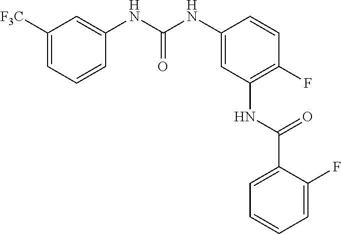 [SMILES: O=C(Nc1cccc(C(F)(F)F)c1)Nc1ccc(F)c(NC(=O)c2ccccc2F)c1] |
| 909 | 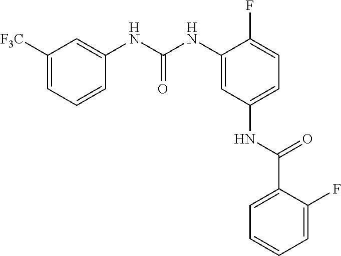 [SMILES: O=C(Nc1cccc(C(F)(F)F)c1)Nc1cc(NC(=O)c2ccccc2F)ccc1F] |
| 910 | 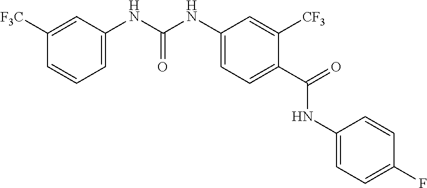 [SMILES: O=C(Nc1cccc(C(F)(F)F)c1)Nc1ccc(C(=O)Nc2ccc(F)cc2)c(C(F)(F)F)c1] |
| 913 | 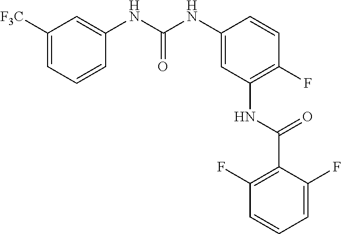 [SMILES: O=C(Nc1cccc(C(F)(F)F)c1)Nc1ccc(F)c(NC(=O)c2c(F)cccc2F)c1] |
| 914 | 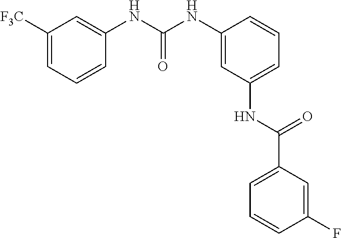 [SMILES: O=C(Nc1cccc(NC(=O)c2cccc(F)c2)c1)Nc1cccc(C(F)(F)F)c1] |
| 915 | 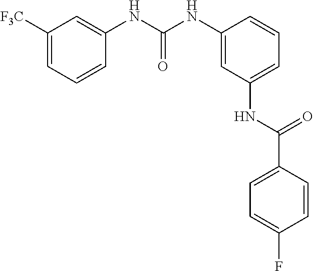 [SMILES: O=C(Nc1cccc(NC(=O)c2ccc(F)cc2)c1)Nc1cccc(C(F)(F)F)c1] |
| 928 | 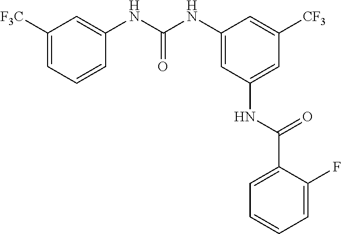 [SMILES: O=C(Nc1cccc(C(F)(F)F)c1)Nc1cc(NC(=O)c2ccccc2F)cc(C(F)(F)F)c1] |
| 929 | 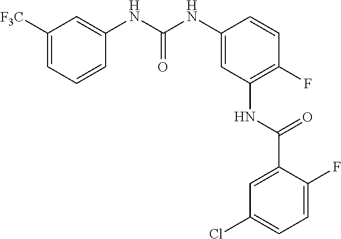 [SMILES: O=C(Nc1cccc(C(F)(F)F)c1)Nc1ccc(F)c(NC(=O)c2cc(Cl)ccc2F)c1] |
| 942 | 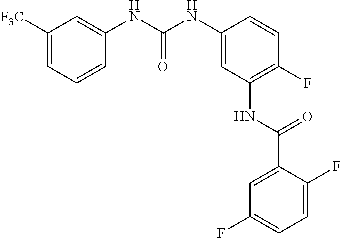 [SMILES: O=C(Nc1cccc(C(F)(F)F)c1)Nc1ccc(F)c(NC(=O)c2cc(F)ccc2F)c1] |
| 943 | 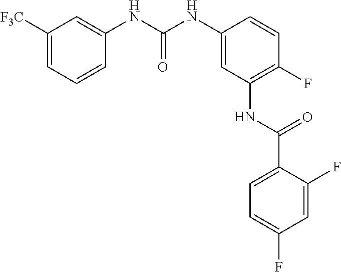 [SMILES: O=C(Nc1cccc(C(F)(F)F)c1)Nc1ccc(F)c(NC(=O)c2ccc(F)cc2F)c1] |
| 944 | 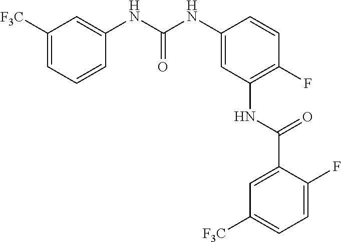 [SMILES: O=C(Nc1cccc(C(F)(F)F)c1)Nc1ccc(F)c(NC(=O)c2cc(C(F)(F)F)ccc2F)c1] |
| 946 | 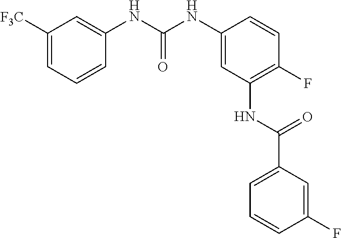 [SMILES: O=C(Nc1cccc(C(F)(F)F)c1)Nc1ccc(F)c(NC(=O)c2cccc(F)c2)c1] |
| 947 | 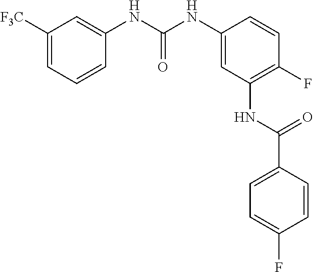 [SMILES: O=C(Nc1cccc(C(F)(F)F)c1)Nc1ccc(F)c(NC(=O)c2ccc(F)cc2)c1] |
| 948 | 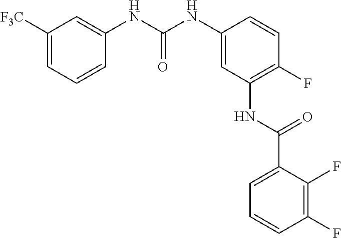 [SMILES: O=C(Nc1cccc(C(F)(F)F)c1)Nc1ccc(F)c(NC(=O)c2cccc(F)c2F)c1] |
| 949 | 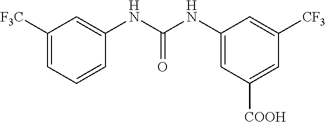 [SMILES: O=C(Nc1cccc(C(F)(F)F)c1)Nc1cc(C(=O)O)cc(C(F)(F)F)c1] |
| 951 | 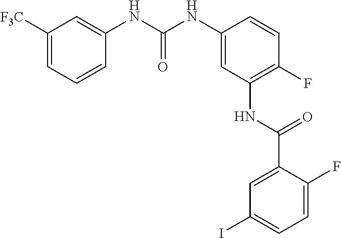 [SMILES: O=C(Nc1cccc(C(F)(F)F)c1)Nc1ccc(F)c(NC(=O)c2cc(I)ccc2F)c1] |
| 954 | 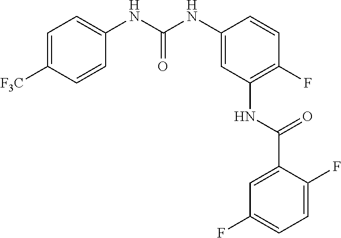 [SMILES: O=C(Nc1ccc(C(F)(F)F)cc1)Nc1ccc(F)c(NC(=O)c2cc(F)ccc2F)c1] |
| 956 | 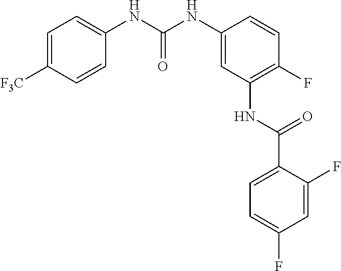 [SMILES: O=C(Nc1ccc(C(F)(F)F)cc1)Nc1ccc(F)c(NC(=O)c2ccc(F)cc2F)c1] |
| 957 | 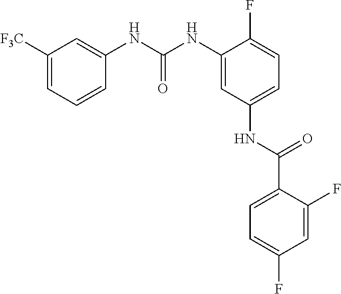 [SMILES: O=C(Nc1cccc(C(F)(F)F)c1)Nc1cc(NC(=O)c2ccc(F)cc2F)ccc1F] |
| 958 | 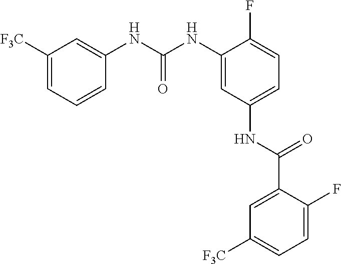 [SMILES: O=C(Nc1cccc(C(F)(F)F)c1)Nc1cc(NC(=O)c2cc(C(F)(F)F)ccc2F)ccc1F] |
| 959 | 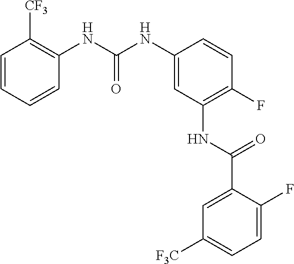 [SMILES: O=C(Nc1ccc(F)c(NC(=O)c2cc(C(F)(F)F)ccc2F)c1)Nc1ccccc1C(F)(F)F] |
| 960 | 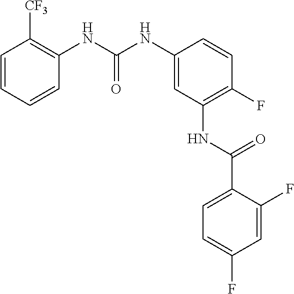 [SMILES: O=C(Nc1ccc(F)c(NC(=O)c2ccc(F)cc2F)c1)Nc1ccccc1C(F)(F)F] |
| 963 | 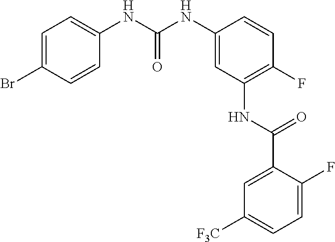 [SMILES: O=C(Nc1ccc(Br)cc1)Nc1ccc(F)c(NC(=O)c2cc(C(F)(F)F)ccc2F)c1] |
| 970 | 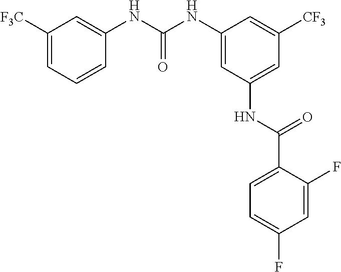 [SMILES: O=C(Nc1cccc(C(F)(F)F)c1)Nc1cc(NC(=O)c2ccc(F)cc2F)cc(C(F)(F)F)c1] |
| 950 | 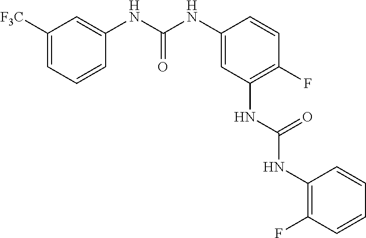 [SMILES: O=C(Nc1cccc(C(F)(F)F)c1)Nc1ccc(F)c(NC(=O)Nc2ccccc2F)c1] |
| 964 | 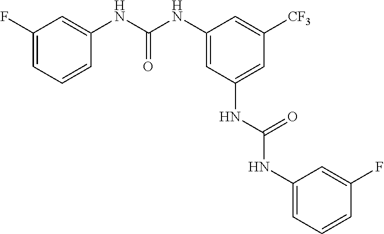 [SMILES: O=C(Nc1cccc(F)c1)Nc1cc(NC(=O)Nc2cccc(F)c2)cc(C(F)(F)F)c1] |
| 953 | 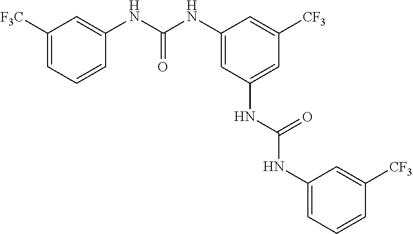 [SMILES: O=C(Nc1cccc(C(F)(F)F)c1)Nc1cc(NC(=O)Nc2cccc(C(F)(F)F)c2)cc(C(F)(F)F)c1] |
| 955 | 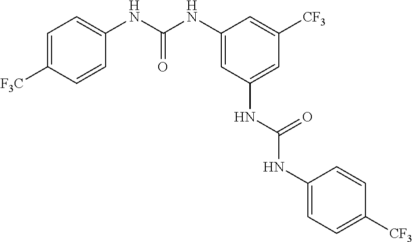 [SMILES: O=C(Nc1ccc(C(F)(F)F)cc1)Nc1cc(NC(=O)Nc2ccc(C(F)(F)F)cc2)cc(C(F)(F)F)c1] |
| 966 | 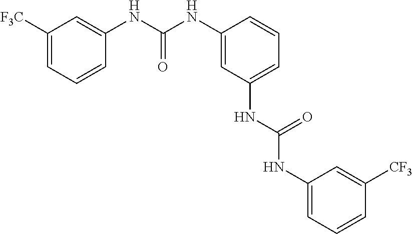 [SMILES: O=C(Nc1cccc(NC(=O)Nc2cccc(C(F)(F)F)c2)c1)Nc1cccc(C(F)(F)F)c1] |
| 972 | 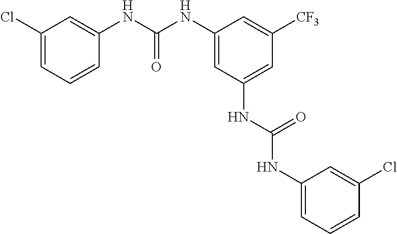 [SMILES: O=C(Nc1cccc(Cl)c1)Nc1cc(NC(=O)Nc2cccc(Cl)c2)cc(C(F)(F)F)c1] |
| 973 | 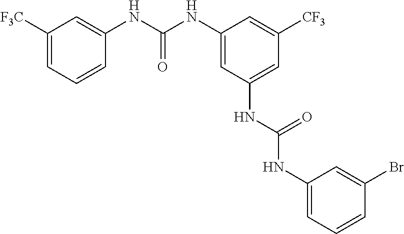 [SMILES: O=C(Nc1cccc(Br)c1)Nc1cc(NC(=O)Nc2cccc(C(F)(F)F)c2)cc(C(F)(F)F)c1] |

TABLE 1.4
Analogues of compound 746 with side chain at ortho position

| ID | Structure |
|---|---|
| 961 | 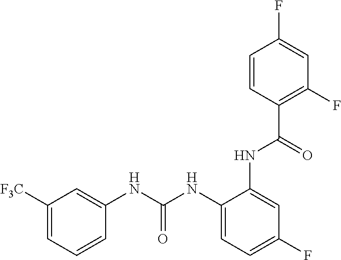 [SMILES: O=C(Nc1cccc(C(F)(F)F)c1)Nc1ccc(F)cc1NC(=O)c1ccc(F)cc1F] |
| 962 | 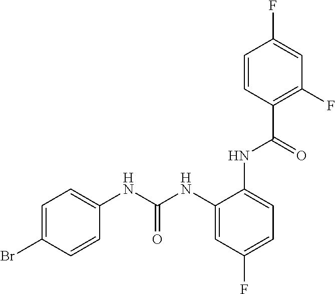 [SMILES: O=C(Nc1ccc(Br)cc1)Nc1cc(F)ccc1NC(=O)c1ccc(F)cc1F] |
| 965 | 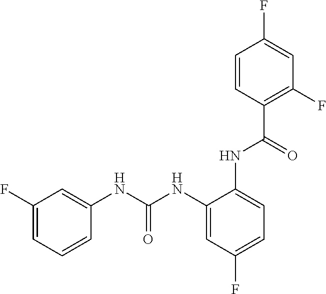 [SMILES: O=C(Nc1cccc(F)c1)Nc1cc(F)ccc1NC(=O)c1ccc(F)cc1F] |
| 968 | 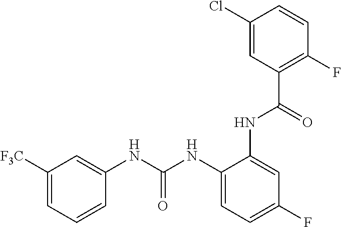 [SMILES: O=C(Nc1cccc(C(F)(F)F)c1)Nc1ccc(F)cc1NC(=O)c1cc(Cl)ccc1F] |
| 969 | 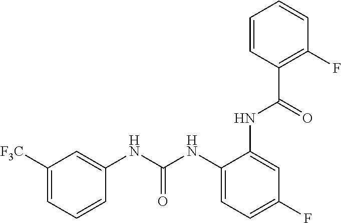 [SMILES: O=C(Nc1cccc(C(F)(F)F)c1)Nc1ccc(F)cc1NC(=O)c1ccccc1F] |
| 974 | 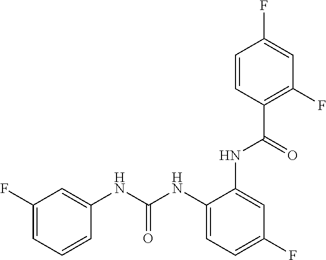 [SMILES: O=C(Nc1cccc(F)c1)Nc1ccc(F)cc1NC(=O)c1ccc(F)cc1F] |
| 975 | 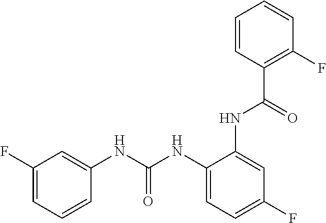 [SMILES: O=C(Nc1cccc(F)c1)Nc1ccc(F)cc1NC(=O)c1ccccc1F] |
| 976 | 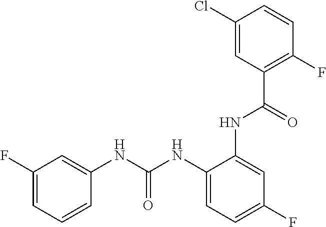 [SMILES: O=C(Nc1cccc(F)c1)Nc1ccc(F)cc1NC(=O)c1cc(Cl)ccc1F] |
| 967 | 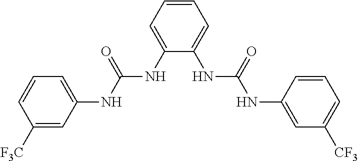 [SMILES: O=C(Nc1cccc(C(F)(F)F)c1)Nc1ccccc1NC(=O)Nc1cccc(C(F)(F)F)c1] |

### Compound 562 and its “Analogues of Formula (I)”

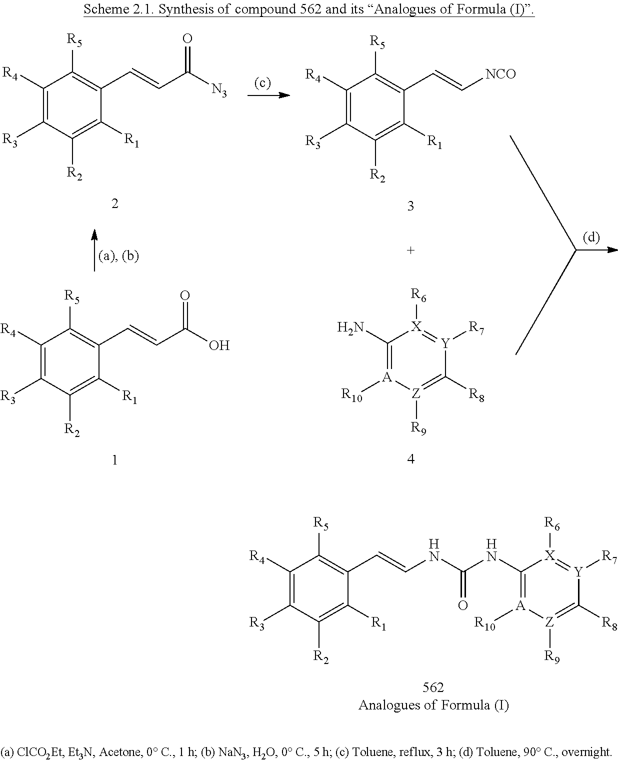

General Procedure for the Synthesis of Aryl Azid 2—Scheme 2.1:

To a solution of 1 (1 mmol) in dry acetone (10 mL), triethylamine (1.1 mmol) and ethyl chlorocarbamate (1.1 mmol) were added dropwise at 0° C. After stirring at 00° C. for 1 h, sodium azide (1.1 mmol, 0.215 g) dissolved in 5 mL water was added dropwise. Stirring was continued at 00° C. for 5 h. Ice water was added. The mixture was extracted by dichloromethane (3×20 mL). The combined organic layers were washed with brine and dried over Na₂SO₄. The organic phase was concentrated under reduced pressure. Colorless oil was obtained and used in the following reaction without further purification.

General Procedure for the Synthesis of the 562 “Analogues of Formula (I)”-Scheme 2.1:

A solution of aryl azide 2 (0.5 mmol) in toluene (10 mL) was heated at 120° C. for 3 h to give aryl isocyanate 3, which is not isolated and treated in situ with the respective 4 at 90° C. overnight. The solvent was cooled to room temperature and the precipitate was collected by filtration and washed with toluene.

### Characterization of Compound 562 and its “Analogues of Formula (I)”

480: White solid, mp. 236-238° C., yield: 26.9%. ¹H NMR (500 MHz, acetone-d₆) δ 8.97 (br. d, J=10.4 Hz, 1H), 8.95 (br, 1H), 8.30 (dd, J₁=2.4 Hz, J₂=2.4 Hz, 1H), 8.14 (s, 1H), 7.88 (d, J=8.8 Hz, 1H), 7.84 (d, J=8.8 Hz, 1H), 7.74-7.71 (m, 1H), 7.66 (d, J=8.0 Hz, 1H), 6.46 (d, J=14.4 Hz, 1H).

481: White solid, mp. 223-225° C., yield: 58.8%. ¹H NMR (500 MHz, acetone-d₆) δ 10.3 (br, 1H), 9.32 (br, 1H), 8.67 (s, 1H), 8.13 (d, J=8.0 Hz, 2H), 7.79 (d, J=8.8 Hz, 1H), 7.79-7.76 (m, 1H), 7.74-7.72 (m, 1H), 7.68 (d, J=8.0 Hz, 1H), 6.57 (d, J=14.4 Hz, 1H).

482: White solid, mp. 214-216° C., yield: 48.1%. ¹H NMR (500 MHz, acetone-d₆) δ 8.88 (br, 1H), 8.79 (br. d, J=12.0 Hz, 1H), 8.74 (s, 1H), 8.30 (dd, J₁=2.4 Hz, J₂=2.4 Hz, 1H), 7.99 (s, 2H), 7.84 (d, J=8.8 Hz, 1H), 7.80-7.77 (m, 1H), 7.73 (s, 1H), 6.32 (d, J=14.4 Hz, 1H). HRMS-ESI calcd for [M+H]⁺ 401.0832. Found: 400.085.

483: White solid, mp. 231-233° C., yield: 48.1%. ¹H NMR (500 MHz, acetone-d₆) δ 10.27 (br, 1H), 9.32 (br, 1H), 8.66 (s, 1H), 8.13 (dd, J₁=2.4 Hz, J₂=2.4 Hz, 1H), 8.02 (s, 2H), 7.83-7.79 (m, 1H), 7.75 (s, 2H), 6.45 (d, J=14.4 Hz, 1H). HRMS-ESI calcd for [M+H]⁺ 401.0832. Found: 400.0849.

487: White solid, mp. 247-249° C., yield: 82.1%. ¹H NMR (500 MHz, acetone-d₆) δ 10.34 (br, 1H), 9.51 (br, 1H), 9.14 (s, 1H), 8.56 (dd, J₁=2.4 Hz, J₂=2.4 Hz, 1H), 8.15 (s, 1H), 7.91 (d, J=8.0 Hz, 1H), 7.85-7.83 (m, 1H), 7.75-7.72 (m, 1H), 7.69 (d, J=8.8 Hz, 1H), 6.62 (d, J=14.4 Hz, 1H).

489: White solid, mp. 247-248° C., yield: 73.2%. ¹H NMR (500 MHz, acetone-d₆) δ 10.29 (br, 1H), 9.52 (br, 1H), 9.13 (s, 1H), 8.56 (dd, J₁=2.4 Hz, J₂=2.4 Hz, 1H), 8.02 (s, 2H), 7.84-7.80 (m, 2H), 7.75 (s, 1H), 6.49 (d, J=14.4 Hz, 1H).

503: White solid, mp. 206-208° C., yield: 76.7%. ¹H NMR (500 MHz, acetone-d₆) δ 8.67 (br, 1H), 8.66 (d, J=2.0 Hz, 1H), 8.44 (br, 1H), 8.26 (dd, J₁=1.5 Hz, J₂=1.5 Hz, 1H), 8.09-8.07 (m, 1H), 8.01 (s, 2H), 7.87-7.82 (m, 1H), 7.75 (s, 1H), 7.33 (dd, J₁=8.5 Hz, J₂=8.0 Hz, 1H), 6.29 (d, J=15.0 Hz, 1H).

504: White solid, mp. 246-247° C., yield: 85.3%. ¹H NMR (500 MHz, acetone-d₆) δ 8.84 (br. d, J=9.5 Hz, 1H), 8.68 (d, J=2.5 Hz, 1H), 8.49 (br, 1H), 8.27 (d, J=3.5 Hz, 1H), 8.17 (s, 1H), 8.09-8.07 (m, 1H), 7.92 (d, J=8.5 Hz, 1H), 7.8-7.76 (m, 1H), 7.68 (d, J=8.0 Hz, 1H), 7.33 (dd, J₁=8.0 Hz, J₂=8.0 Hz, 1H), 6.44 (dd, J₁=2.5 Hz, J₂=2.0 Hz, 1H).

510: White solid, mp. 208-210° C., yield: 33.9%. ¹H NMR (500 MHz, acetone-d₆) δ 8.89 (br, 1H), 8.78-8.77 (m, 1), 8.69 (br. d, J=10.0 Hz, 1H), 8.34 (dd, J₁=2.5 Hz, J₂=2.5 Hz, 1H), 7.87 (d, J=8.5 Hz, 1H), 7.72-7.63 (m, 3H), 7.55 (t, J=7.5 Hz, 1H), 7.49 (d, J=7.5 Hz, 1H), 6.26 (d, J=14.5 Hz, 1H).

511: White solid, mp. 215-217° C., yield: 70.0%. ¹H NMR (500 MHz, acetone-d₆) δ 10.24 (br, 1H), 9.32 (br, 1H), 8.70 (d, J=2.0 Hz, 1H), 8.16 (dd, J₁=2.0 Hz, J₂=2.0 Hz, 1H), 7.79-7.66 (m, 4H), 7.56 (t, J=7.5 Hz, 1H), 7.50 (d, J=7.5 Hz, 1H), 6.39 (d, J=14.5 Hz, 1H).

512: White solid, mp. 203-205° C., yield: 38.8%. ¹H NMR (500 MHz, acetone-d₆) δ 8.84 (br, 1H), 8.80-8.76 (m, 1H), 8.53 (br. d, J=10.0 Hz, 1H), 8.34 (dd, J₁=2.5 Hz, J₂=3.0 Hz, 1H), 7.86 (d, J=10.0 Hz, 1H), 7.54-7.49 (m, 1H), 7.37 (dd, J₁=1.0 Hz, J₂=1.0 Hz, 2H), 7.33-7.29 (m, 2H), 7.19-7.16 (m, 1H), 6.16 (d, J=14.5 Hz, 1H).

527: White solid, mp. 202-204° C., yield: 50.6%. ¹H NMR (500 MHz, acetone-d₆) δ 9.33 (br.d, J=10.0 Hz, 1H), 8.62 (d, J=8.0 Hz, 1H), 8.09-8.08 (m, 1H), 8.03 (s, 2H), 7.93 (br, 1H), 7.86-7.82 (m, 1H), 7.76 (s, 1H), 7.45-7.42 (m, 1H), 6.32 (d, J=15.0 Hz, 1H).

528: White solid, mp. 243-245° C., yield: 67.8%. ¹H NMR (500 MHz, acetone-d₆) δ 10.20 (br, 1H), 9.15 (br, 1H), 8.96 (s, 1H), 8.30 (s, 2H), 8.06 (s, 2H), 7.86 (d, J=15.0 Hz, 1H), 7.78 (s, 1H), 6.47 (d, J=15.0 Hz, 1H). HRMS-ESI calcd for [M+Na]⁺ 399.0651. Found: 399.0665.

531: White solid, mp. 266-268° C., yield: 62.5%. ¹H NMR (500 MHz, acetone-d₆) δ 11.54 (br, 1H), 9.20 (br, 1H), 8.70 (s, 2H), 8.07 (s, 2H), 7.86 (d, J=9.0 Hz, 1H), 7.78 (s, 1H), 7.20 (d, J=4.0 Hz, 1H), 6.54 (d, J=14.5 Hz, 1H).

533: White solid, mp. 188-190° C., yield: 57.4%. ¹H NMR (500 MHz, acetone-d₆) δ 9.43 (d, J=3.0 Hz, 1H), 9.14 (br. d, J=9.0 Hz, 1H), 8.27-8.25 (m, 1H), 8.09 (br, 1H), 8.04 (s, 2H), 7.87-7.83 (m, 1H), 7.76 (s, 1H), 7.51 (dt, J₁=2.5 Hz, J₂=5.0 Hz, 1H), 6.32 (dd, J₁=2.5 Hz, J₂=2.5 Hz, 1H).

535: White solid, mp. 181-183° C., yield: 38.8%. ¹H NMR (500 MHz, acetone-d₆) δ 8.76 (br, 1H), 8.73 (br, 1H), 8.43 (s, 1H), 8.18 (d, J=2.0 Hz, 1H), 8.09 (dd, J₁=2.0 Hz, J₂=2.0 Hz, 1H), 8.02 (s, 2H), 7.86-7.81 (m, 1H), 7.76 (s, 1H), 6.33 (d, J=14.5 Hz, 1H).

536: White solid, mp. 209-211° C., yield: 78.4%. ¹H NMR (500 MHz, acetone-d₆) δ 8.63 (br.d, J=10.0 Hz, 1H), 8.53 (s, 1H), 8.34 (br, 1H), 7.99 (s, 2H), 7.94 (d, J=8.5 Hz, 1H), 7.87-7.82 (m, 1H), 7.74 (s, 1H), 7.18 (d, J=8.5 Hz, 1H), 6.27 (d, J=15.0 Hz, 1H).

537: White solid, mp. 199-201° C., yield: 60.4%. ¹H NMR (500 MHz, acetone-d₆) δ 8.86 (br.d, J=10.0 Hz, 1H), 8.29 (dt, J₁=6.5 Hz, J₂=8.0 Hz, 1H), 8.20 (d, J=4.5 Hz, 1H), 8.00 (s, 2H), 7.88-7.83 (m, 1H), 7.78 (br, 1H), 7.75 (s, 1H), 7.21 (dt, J₁=5.0 Hz, J₂=7.5 Hz, 1H), 6.27 (d, J=15.0 Hz, 1H).

538: White solid, mp. 223-224° C., yield: 52.3%. ¹H NMR (500 MHz, acetone-d₆) δ 9.65 (br, 1H), 9.07 (dd, J₁=1.5 Hz, J₂=2.0 Hz, 1H), 8.09 (s, 2H), 7.90-7.85 (m, 1H), 7.81 (s, 1H), 7.61 (dd, J₁=1.5 Hz, J₂=2.5 Hz, 1H), 7.27-7.17 (m, 1H), 6.55 (dd, J₁=1.5 Hz, J₂=2.0 Hz, 1H).

539: White solid, mp. 185-186° C., yield: 68.8%. ¹H NMR (500 MHz, acetone-d₆) δ 8.79 (br.d, J=9.5 Hz, 1H), 8.68 (br, 1H), 8.58 (s, 1H), 8.43 (s, 1H), 8.34 (s, 1H), 8.01 (s, 2H), 7.83 (dd, J₁=8.5 Hz, J₂=9.5 Hz, 1H), 7.76 (s, 1H), 6.33 (d, J=14.5 Hz, 1H).

540: White solid, mp. 204-205° C., yield: 71.6%. ¹H NMR (500 MHz, acetone-d₆) δ 8.73 (br.d, J=9.5 Hz, 1H), 8.59 (br, 1H), 8.51 (d, J=2.5 Hz, 1H), 8.04 (dd, J₁=2.0 Hz, J₂=2.0 Hz, 1H), 8.01 (s, 2H), 7.83 (dd, J₁=14.5 Hz, J₂=14.5 Hz, 1H), 7.76 (s, 1H), 7.55 (d, J=9.0 Hz, 1H), 6.31 (d, J=15.0 Hz, 1H).

541: White solid, mp. 191-193° C., yield: 71.9%. ¹H NMR (500 MHz, acetone-d₆) δ 8.96 (s, 1H), 8.84 (br.d, J=10.5 Hz, 1H), 8.22 (d, J=5.0 Hz, 1H), 8.00 (s, 2H), 7.88-7.81 (m, 2H), 7.74 (s, 1H), 7.22 (d, J=4.5 Hz, 1H), 6.27 (d, J=14.5 Hz, 1H), 2.33 (s, 3H).

543: White solid, mp. 244-245° C., yield: 59.2%. ¹H NMR (500 MHz, acetone-d₆) δ 11.70 (br, 1H), 9.26 (br, 1H), 8.76 (s, 1H), 8.26 (s, 2H), 8.10 (s, 2H), 7.90 (d, J=13.5 Hz, 1H), 7.80 (s, 1H), 7.73 (s, 1H), 7.66 (s, 3H), 6.55 (d, J=14.0 Hz, 1H).

546: White solid, mp. 216-218° C., yield: 64.2%. ¹H NMR (500 MHz, acetone-d₆) δ 8.67 (br.d, J=10.0 Hz, 1H), 8.48 (s, 1H), 8.39 (br, 1H), 8.12 (s, 1H), 7.99 (s, 2H), 7.89 (s, 1H), 7.87-7.82 (m, 1H), 7.74 (s, 1H), 6.29 (d, J=14.0 Hz, 1H), 2.34 (s, 3H).

548: White solid, mp. 245-247° C., yield: 65.4%. ¹H NMR (500 MHz, acetone-d₆) δ 10.57 (br, 1H), 9.34 (br, 1H), 8.66 (s, 1H), 8.15 (d, J=7.5 Hz, 1H), 8.07 (s, 2H), 7.86 (d, J=8.0 Hz, 1H), 7.78 (d, J=11.5 Hz, 2H), 6.50 (d, J=14.0 Hz, 1H).

549: White solid, mp. 244-246° C., yield: 70.5%. ¹H NMR (500 MHz, acetone-d₆) δ 10.29 (br, 1H), 9.25 (br, 1H), 8.58 (s, 1H), 8.06 (s, 2H), 7.98 (s, 1H), 7.87 (dt, J₁=9.0 Hz, J₂=8.0 Hz, 1H), 7.78 (s, 1H), 7.38 (s, 1H), 6.48 (d, J=14.0 Hz, 1H).

550: White solid, mp. 184-186° C., yield: 51.5%. ¹H NMR (500 MHz, acetone-d₆) δ 8.68 (br, 1H), 8.58 (br.d, J=10.0 Hz, 1H), 8.42 (s, 1H), 8.17 (d, J=3.0 Hz, 1H), 8.11-8.08 (m, 1H), 7.71-7.69 (m, 1H), 7.67-7.64 (m, 2H), 7.54 (dt, J₁=8.0 Hz, J₂=7.5 Hz, 1H), 7.48 (d, J=7.5 Hz, 1H), 6.22 (d, J=14.5 Hz, 1H).

551: White solid, mp. 179-181° C., yield: 68.0%. ¹H NMR (500 MHz, acetone-d₆) δ 9.46 (d, J=3.5 Hz, 1H), 9.02 (br.d, J=10.0 Hz, 1H), 8.25 (d, J=5.0 Hz, 1H), 8.06 (br, 1H), 7.73-7.70 (m, 1H), 7.68-7.65 (m, 2H), 7.54 (dt, J₁=7.5 Hz, J₂=7.5 Hz, 1H), 7.50-7.48 (m, 2H), 6.21 (d, J=15.0 Hz, 1H).

552: White solid, mp. 185-187° C., yield: 40.8%. ¹H NMR (500 MHz, acetone-d₆) δ 8.65 (br, 2H), 8.57 (d, J=2.0 Hz, 1H), 8.44 (dt, J₁=2.0 Hz, J₂=2.0 Hz, 1H), 8.33 (d, J=2.0 Hz, 1H), 7.70-7.63 (m, 3H), 7.54 (dt, J₁=8.0 Hz, J₂=8.0 Hz, 1H), 7.48 (d, J=8.0 Hz, 1H), 6.23 (d, J=14.5 Hz, 1H).

553: White solid, mp. 183-185° C., yield: 65.4%. ¹H NMR (500 MHz, acetone-d₆) δ 8.57 (br.d, J=10.0 Hz, 1H), 8.55 (br, 1H), 8.50 (d, J=3.0 Hz, 1H), 8.05-8.03 (m, 1H), 7.69-7.63 (m, 3H), 7.55-7.52 (m, 2H), 7.48 (d, J=7.5 Hz, 1H), 6.21 (d, J=14.5 Hz, 1H).

554: White solid, mp. 190-192° C., yield: 17.3%. ¹H NMR (500 MHz, acetone-d₆) δ 8.53 (d, J=2.5 Hz, 1H), 8.47 (br.d, J=11.0 Hz, 1H), 8.30 (br, 1H), 7.96-7.94 (m, 1H), 7.70-7.64 (m, 3H), 7.53 (dt, J₁=7.5 Hz, J₂=8.0 Hz, 1H), 7.46 (d, J=7.5 Hz, 1H), 7.18 (d, J=8.0 Hz, 1H), 6.17 (d, J=15.0 Hz, 1H), 2.58 (s, 3H).

555: White solid, mp. 174-176° C., yield: 73.9%. ¹H NMR (500 MHz, acetone-d₆) δ 8.98 (d, J=7.5 Hz, 1H), 8.68 (br.d, J=10.0 Hz, 1H), 8.21 (d, J=4.5 Hz, 1H), 7.76 (br, 1H), 7.71-7.66 (m, 3H), 7.53 (dt, J₁=7.5 Hz, J₂=8.0 Hz, 1H), 7.46 (d, J=7.5 Hz, 1H), 7.21 (d, J=5.0 Hz, 1H), 6.16 (d, J=14.5 Hz, 1H), 2.34 (s, 3H).

556: White solid, mp. 181-183° C., yield: 71.6%. ¹H NMR (500 MHz, acetone-d₆) δ 8.49 (br.d, J=10.5 Hz, 1H), 8.47 (d, J=2.0 Hz, 1H), 8.34 (br, 1H), 8.11 (s, 1H), 7.90 (s, 1H), 7.71-7.64 (m, 3H), 7.53 (dt, J₁=7.5 Hz, J₂=8.0 Hz, 1H), 7.47 (d, J=7.5 Hz, 1H), 6.18 (d, J=15.0 Hz, 1H), 2.33 (s, 3H).

557: White solid, mp. 166-168° C., yield: 60.7%. ¹H NMR (500 MHz, acetone-d₆) δ 8.72 (br.d, J=10.5 Hz, 1H), 8.32-8.28 (m, 1H), 8.19 (dd, J₁=1.0 Hz, J₂=1.0 Hz, 1H), 7.73 (br, 1H), 7.71-7.64 (m, 3H), 7.53 (dt, J₁=7.5 Hz, J₂=8.0 Hz, 1H), 7.47 (d, J=7.5 Hz, 1H), 7.20 (dd, J₁=8.0 Hz, J₂=8.0 Hz, 1H), 6.16 (d, J=15.0 Hz, 1H), 2.50 (s, 3H).

558: White solid, mp. 203-205° C., yield: 17.3%. ¹H NMR (500 MHz, acetone-d₆) δ 10.13 (br, 1H), 9.13 (br, 1H), 8.94 (s, 1H), 8.30-8.28 (m, 2H), 7.73-7.68 (m, 3H), 7.56 (dt, J₁=7.5 Hz, J₂=8.0 Hz, 1H), 7.50 (d, J=7.5 Hz, 1H), 6.37 (d, J=14.5 Hz, 1H).

559: White solid, mp. 242-244° C., yield: 60.0%. ¹H NMR (500 MHz, acetone-d₆) δ 11.42 (br, 1H), 9.08 (br, 1H), 8.71 (d, J=7.0 Hz, 2H), 7.74-7.69 (m, 3H), 7.56 (dt, J₁=7.5 Hz, J₂=8.0 Hz, 1H), 7.51 (d, J=8.0 Hz, 1H), 7.19 (dt, J₁=4.5 Hz, J₂=5.0 Hz, 1H), 6.44 (d, J=15.0 Hz, 1H).

560: White solid, mp. 201-203° C., yield: 17.3%. ¹H NMR (500 MHz, acetone-d₆) δ 11.07 (br.d, J=8.5 Hz, 1H), 9.58 (br, 1H), 9.07 (d, J=5.0 Hz, 2H), 7.76-7.69 (m, 3H), 7.61-7.52 (m, 3H), 6.44 (d, J=15.0 Hz, 1H).

561: White solid, mp. 227-228° C., yield: 71.6%. ¹H NMR (500 MHz, acetone-d₆) δ 11.59 (br.d, J=10.0 Hz, 1H), 9.14 (br, 1H), 8.75 (d, J=5.5 Hz, 1H), 8.27-8.24 (m, 2H), 7.78-7.71 (m, 4H), 7.68-7.64 (m, 3H), 7.57 (t, J=8.0 Hz, 1H), 7.52 (d, J=8.0 Hz, 1H), 6.43 (d, J=14.5 Hz, 1H).

564: White solid, mp. 208-210° C., yield: 56.7%. ¹H NMR (500 MHz, acetone-d₆) δ 10.33 (br, 1H), 9.12 (br, 1H), 8.49 (s, 1H), 7.96 (dd, J₁=2.5 Hz, J₂=2.5 Hz, 1H), 7.59-7.51 (m, 4H), 7.39 (t, J=7.5 Hz, 1H), 7.34 (d, J=7.5 Hz, 1H), 6.23 (d, J=15.0 Hz, 1H).

583: White solid, mp. 213-21° C., yield: 77.1%. ¹H NMR (500 MHz, acetone-d₆) δ 10.25 (br, 1H), 9.16 (br, 1H), 8.98 (s, 1H), 8.32-8.29 (m, 2H), 8.19 (s, 1H), 7.94 (d, J=8.0 Hz, 1H), 7.79 (d, J=9.5 Hz, 1H), 7.72 (d, J=8.5 Hz, 1H), 6.62-6.57 (m, 1H).

542: White solid, mp. 209-212° C., yield: 29.8%. ¹H NMR (500 MHz, acetone-d₆) δ 8.89 (br, 1H), 8.62 (s, 1H), 8.12 (br, 1H), 7.87 (s, 2H), 7.64-7.60 (m, 2H), 6.18 (d, J=14.5 Hz, 1H).

544: White solid, mp. 223-225° C., yield: 58.5%. ¹H NMR (500 MHz, acetone-d₆) δ 10.53 (br, 1H), 9.26 (br, 1H), 8.83 (s, 1H), 8.60 (dd, J₁=1.5 Hz, J₂=1.0 Hz, 1H), 8.07 (s, 2H), 7.85 (dd, J₁=14.5 Hz, J₂=15.0 Hz, 1H), 7.79 (s, 1H), 7.55 (d, J=5.0 Hz, 1H), 6.51 (d, J=14.5 Hz, 1H).

545: White solid, mp. 224-226° C., yield: 23.1%. ¹H NMR (500 MHz, acetone-d₆) δ 11.15 (br, 1H), 9.09 (s, 2H), 8.10 (s, 2H), 7.83 (s, 2H), 6.65 (d, J=14.5 Hz, 1H).

562: White solid, mp. 207-209° C., yield: 60.0%. ¹H NMR (500 MHz, acetone-d₆) δ 10.43 (br, 1H), 9.21 (br, 1H), 8.83 (s, 1H), 8.59 (d, J=6.0 Hz, 1H), 7.74-7.65 (m, 3H), 7.58-7.50 (m, 3H), 6.41 (d, J=14.5 Hz, 1H).

766: White solid, yield: 83.2%. ¹H NMR (500 MHz, acetone-d₆) δ 10.08 (br, 1H), 9.54 (br, 1H), 8.18 (d, J=9.4 Hz, 1H), 7.85 (d, J=9.4 Hz, 1H), 7.78-7.67 (m, 2H), 7.54-7.45 (m, 3H), 6.26 (d, J=14.7 Hz, 1H).

875: White solid. Yield: 67.8%. ¹H NMR (500 MHz, Acetone-de) δ 10.40 (br, 1H), 9.21 (br, 1H), 8.79 (d, J=1.1 Hz, 1H), 8.55 (d, J=5.8 Hz, 1H), 7.59 (d, J=14.7 Hz, 1H), 7.52 (d, J=5.8 Hz, 1H), 7.45-7.39 (m, 2H), 7.32 (s, 1H), 7.17-7.05 (m, 1H), 6.32 (d, J=14.7 Hz, 1H).

The chemical structures of compounds 480, 481, 483, 487, 489, 503, 504, 510, 511, 512, 527, 528, 531, 533, 535, 536, 537, 538, 539, 540, 541, 543, 546, 548, 549, 550, 551, 552, 553, 554, 555, 556, 557, 558, 559, 560, 561, 564, 583, 542, 544, 545, 562, 766 and 875 prepared as described above are provided in Table 2.1 herein below.

### The 562 “Analogues of Formula (II)”

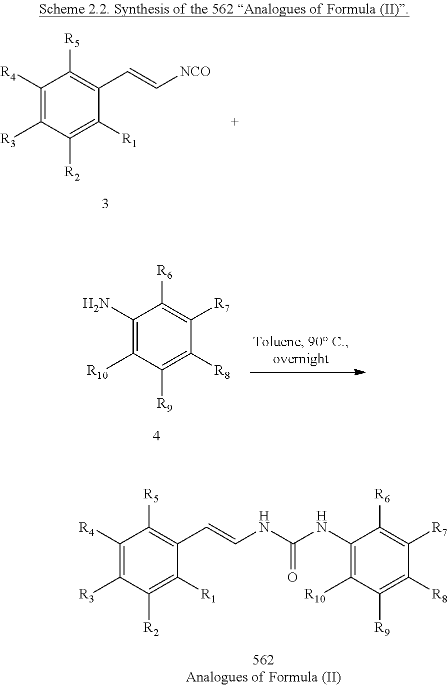

General Procedure for the Synthesis of the 562 “Analogues of Formula (II)”-Scheme 2.2:

An equimolar mixture of aryl isocyanate 3 and aryl amine 4 in toluene was heated at 90° C. overnight. After cooling to room temperature, white solid was precipitated, which was collected by filtration and washed with toluene.

### Characterization of the 562 “Analogues of Formula (II)”

403: White solid, mp. 129-131° C., yield: 38.8%. ¹H NMR (500 MHz, acetone-d₆) δ 8.61 (br, 1H), 8.54 (br. d, J=10.0 Hz, 1H), 8.10 (s, 1H), 7.73-7.65 (m, 4H), 7.56-7.52 (m, 2H), 7.47 (d, J=9.0 Hz, 1H), 7.36 (d, J=8.0 Hz, 1H), 6.20 (d, J=14.5 Hz, 1H).

404: White solid, mp. 208-210° C., yield: 4.8%. ¹H NMR (500 MHz, acetone-d₆) δ M.P. 208-210° C. 9.19 (br, 1H), 8.93 (br. d, J=10.0 Hz, 1H), 8.30 (d, J=2.5 Hz, 1H), 8.15 (d, J=9.0 Hz, 1H), 8.02 (dd, J₁=2.0 Hz, J₂=2.0 Hz, 1H), 7.84 (d, J=9.0 Hz, 1H), 7.68 (d, J=8.0 Hz, 1H), 7.65-7.59 (m, 2H), 7.39 (t, J=8.0 Hz, 1H), 6.49-6.44 (m, 1H).

405: White solid, mp. 233-235° C., yield: 73.7%. ¹H NMR (500 MHz, acetone-d₆) δ 9.09 (br, 1H), 8.90 (d, J=10.5 Hz, 1H), 8.29 (d, J=2.0 Hz, 1H), 7.97 (m, 2H), 7.84 (d, J=9.0 Hz, 1H), 7.68 (d, J=7.5 Hz, 1H), 7.65-7.59 (m, 2H), 7.39 (t, J=7.5 Hz, 1H), 6.46 (dd, J₁=2.0 Hz, J₂=2.5 Hz, 1H). LHMS-ESI, m/z [M+H]⁺ 400.09.

406: White solid, mp. 158-160° C., yield: 53.5%. ¹H NMR (500 MHz, acetone-d₆) δ 8.72 (br. d, J=10.5 Hz, 1H), 8.62 (br, 1H), 8.10 (s, 1H), 7.82 (d, J=8.5 Hz, 1H), 7.73 (d, J=8.0 Hz, 1H), 7.68-7.60 (m, 3H), 7.54 (t, J=8.0 Hz, 1H), 7.38-7.35 (m, 2H), 6.41-6.38 (m, 1H).

407: White solid, mp. 213-215° C., yield: 64.4%. ¹H NMR (500 MHz, acetone-d₆) δ 9.20 (br, 1H), 8.79 (br. d, J=10.5 Hz, 1H), 8.29 (d, J=2.5 Hz, 1H), 8.15 (d, J=9.0 Hz, 1H) 8.02 (dd, J₁=2.0 Hz, J₂=2.5 Hz, 1H), 7.69 (dd, J₁=14.5 Hz, J₂=14.5 Hz, 1H), 7.65-7.59 (m, 4H), 6.25 (d, J=14.5 Hz, 1H).

408: White solid, mp. 178-180° C., yield: 70.1%. ¹H NMR (500 MHz, acetone-d₆) δ 8.66 (br, 1H), 8.57 (br. d, J=10.5 Hz, 1H), 8.09 (s, 1H), 7.74-7.69 (m, 2H), 7.64-7.53 (m, 5H), 7.69 (d, J=7.5 Hz, 1H), 6.18 (d, J=14.5 Hz, 1H).

409: White solid, mp. 175-177° C., yield: 47.2%. ¹H NMR (500 MHz, acetone-d₆) δ M 9.09 (br, 1H), 8.57 (br. d, J=10.0 Hz, 1H), 8.31-8.28 (m, 1H), 8.14 (d, J=9.0 Hz, 1H), 7.99 (dd, J₁=2.5 Hz, J₂=2.0 Hz, 1H), 7.52 (dd, J₁=10.5 Hz, J₂=10.5 Hz, 1H), 7.22 (t, J=8.0 Hz, 1H), 6.96-6.94 (m, 2H), 6.77-6.74 (m, 1H), 6.14 (d, J=14.5 Hz, 1H), 3.83 (s, 3H). LHMS-ESI, m/z [M+H]⁺ 382.10.

410: White solid, mp. 181-183° C., yield: 55.1%. ¹H NMR (500 MHz, acetone-d₆) δ 9.12 (br, 1H), 8.74 (br. d, J=10.0 Hz, 1H), 8.29 (d, J=2.0 Hz, 1H), 8.00-7.94 (m, 2H), 7.72-7.63 (m, 3H), 7.57-7.54 (m, 1H), 7.49 (d, J=8.0 Hz, 1H), 6.27 (d, J=14.5 Hz, 1H). LHMS-ESI, m/z [M+H]⁺ 400.09.

411: White solid, mp. 145-147° C., yield: 32.7%. ¹H NMR (500 MHz, acetone-de) δ 8.55 (br, 1H), 8.36 (br. d, J=10.5 Hz, 1H), 8.11 (s, 1H), 7.71 (d, J=8.5 Hz, 1H), 7.57-7.52 (m, 2H), 7.35 (d, J=8.0 Hz, 1H), 7.23-7.19 (m, 1H), 6.94-6.92 (m, 2H), 6.75-6.72 (m, 1H), 6.07 (d, J=14.5 Hz, 1H), 3.83 (s, 3H).

412: White solid, mp. 213-215° C., yield: 32.0%. ¹H NMR (500 MHz, acetone-d₆) δ 9.14 (br, 1H), 8.67 (br. d, J=10.5 Hz, 1H), 8.29 (d, J=2.5 Hz, 1H), 8.15 (d, J=8.5 Hz, 1H), 8.00 (dd, J₁=2.5 Hz, J₂=2.5 Hz, 1H), 7.57 (dd, J₁=14.5 Hz, J₂=14.5 Hz, 1H), 7.37-7.32 (m, 1H), 7.21 (d, J=8.5 Hz, 1H), 7.18-7.15 (m, 1H), 6.93 (ddd, J₁=3.0 Hz, J₂=2.5 Hz, J₃=2.5 Hz, 1H), 6.17 (d, J=14.5 Hz, 1H). LHMS-ESI, m/z [M+H]⁺ 307.08.

413: White solid, mp. 179-181° C., yield: 78.4%. ¹H NMR (500 MHz, acetone-d₆) δ 8.41 (br. d, J=10.5 Hz, 1H), 8.28 (br, 1H), 7.72-7.67 (m, 2H), 7.64 (s, 1H), 7.58-7.51 (m, 3H), 7.45 (d, J=7.5 Hz, 1H), 7.33-7.29 (m, 2H), 7.05-7.01 (m, 1H), 6.14 (d, J=15.0 Hz, 1H).

414: White solid, mp. 171-173° C., yield: 65.4%. ¹H NMR (500 MHz, acetone-d₆) δ 8.59 (br, 1H), 8.46 (br. d, J=10.5 Hz, 1H), 8.09 (s, 1H), 7.73-7.71 (m, 1H), 7.60 (dd, J₁=14.5 Hz, J₂=14.5 Hz, 1H), 7.55-7.52 (m, 1H), 7.37-7.30 (m, 2H), 7.19 (d, J=7.5 Hz, 1H), 7.13 (dd, J₁=1.5 Hz, J₂=1.5 Hz, 1H), 6.92-6.88 (m, 1H), 6.11 (d, J=15.0 Hz, 1H).

415: White solid, mp. 159-161° C., yield: 51.6%. ¹H NMR (500 MHz, acetone-d₆) δ 9.56 (br, 1H), 8.37 (br. d, J=10.0 Hz, 1H), 8.10 (s, 1H), 7.72 (d, J=10.0 Hz, 1H), 7.57-7.52 (m, 2H), 7.36-7.34 (m, 3H), 7.32-7.29 (m, 2H), 7.17-7.14 (m, 1H), 6.10 (d, J=14.5 Hz, 1H).

416: White solid, mp. 195-197° C., yield: 60.4%. ¹H NMR (500 MHz, acetone-d₆) δ 8.76 (br, 1H), 8.57 (br. d, J=10.5 Hz, 1H), 7.79-7.76 (m, 2H), 7.73-7.69 (m, 3H), 7.67-7.64 (m, 2H), 7.56-7.53 (m, 1H), 7.48 (d, J=8.0 Hz, 1H), 6.22 (d, J=14.5 Hz, 1H). LHMS-ESI, m/z [M+H]⁺ 332.10.

417: White solid, mp. 144-146° C., yield: 78.6%. ¹H NMR (500 MHz, acetone-d₆) δ 8.39 (br. d, J=6.5 Hz, 1H), 8.28 (br, 1H), 7.71-7.66 (m, 2H), 7.64 (s, 1H), 7.54-7.50 (m, 1H), 7.46 (d, J=8.0 Hz, 1H), 7.34 (t, J=2.0 Hz, 1H), 7.04-7.02 (m, 1H), 6.63-6.60 (m, 1H), 6.15 (d, J=15.0 Hz, 1H).

421: White solid, mp. 199-201° C., yield: 71.2%. ¹H NMR (500 MHz, acetone-d₆) δ 9.06 (br, 1H), 8.71 (br. d, J=10.0 Hz, 1H), 8.29 (s, 1H), 8.00-7.94 (m, 2H), 7.71-7.58 (m, 5H), 6.25 (d, J=14.5 Hz, 1H). LHMS-ESI, m/z [M+H]⁺ 400.09.

429: White solid, mp. 166-168° C., yield: 16.1%. ¹H NMR (500 MHz, acetone-d₆) δ 8.57 (br. d, J=10.5 Hz, 1H), 8.26 (b, 1H), 7.81 (d, J=8.5 Hz, 1H), 7.67-7.59 (m, 3H), 7.37-7.33 (m, 2H), 7.21 (t, J=8.0 Hz, 1H), 7.05-7.03 (m, 1H), 6.62 (dd, J₁=3.0 Hz, J₂=2.5 Hz, 1H), 6.35 (dd, J₁=2.0 Hz, J₂=2.5 Hz, 1H), 3.80 (s, 3H).

430: White solid, mp. 154-156° C., yield: 67.1%. ¹H NMR (500 MHz, acetone-d₆) δ 8.23 (br, 1H), 8.20 (br, 1H), 7.55 (dd, J₁=15.0 Hz, J₂=15.0 Hz, 1H), 7.35-7.33 (m, 1H), 7.21-7.18 (m, 2H), 7.03-7.00 (m, 1H), 6.93-6.91 (m, 2H), 6.73-6.71 (m, 1H), 6.62-6.59 (m, 1H), 6.02 (d, J=15.0 Hz, 1H), 3.83 (s, 3H), 3.80 (s, 3H).

433: White solid, mp. 193-196° C., yield: 44.7%. ¹H NMR (500 MHz, acetone-d₆) δ 9.04 (br, 1H), 8.63 (br. d, J=10.0 Hz, 1H), 8.29-8.28 (m, 1H), 8.02-7.94 (m, 2H), 7.58 (dd, J₁=14.5 Hz, J₂=14.5 Hz, 1H), 7.36-7.32 (m, 1H), 7.21 (d, J=8.0 Hz, 1H), 7.17-7.14 (m, 1H), 6.95-6.90 (m, 1H), 6.17 (d, J=15.0 Hz, 1H). LHMS-ESI, m/z [M+H]⁺ 350.09.

435: White solid, mp. 193-195° C., yield: 82.5%. ¹H NMR (500 MHz, acetone-d₆) δ 8.28 (br. d, J=10.5 Hz, 1H), 7.92 (b, 1H), 7.74-7.67 (m, 2H), 7.63 (s, 1H), 7.53 (t, J=3.0 Hz, 1H), 7.45 (d, J=7.5 Hz, 1H), 7.37 (d, J=9.0 Hz, 1H), 6.76 (d, J=9.0 Hz, 1H), 6.11 (d, J=14.5 Hz, 1H), 2.93 (s, 6H).

436: White solid, mp. 228-230° C., yield: 36.5%. ¹H NMR (500 MHz, acetone-d₆) δ 9.25 (br, 1H), 9.07 (br. d, J=10.0 Hz, 1H), 8.31 (d, J=3.0 Hz, 1H), 8.20-8.17 (m, 2H), 8.04 (dd, J₁=2.0 Hz, J₂=2.0 Hz, 1H), 7.94 (d, J=8.0 Hz, 1H), 7.81-7.72 (m, 2H), 6.52 (dd, J₁=2.0 Hz, J₂=2.0 Hz, 1H). HRMS-ESI calcd for [M+H]⁺ 488.06512. Found: 488.06578.

437: White solid, mp. 202-204° C., yield: 25.5%. ¹H NMR (500 MHz, acetone-d₆) δ 9.15 (br, 1H), 8.72 (br. d, J=10.0 Hz, 1H), 8.31 (s, 1H), 8.17 (d, J=9.0 Hz, 1H), 8.02 (dd, J₁=2.0 Hz, J₂=2.0 Hz, 1H), 7.66-7.62 (m, 1H), 7.47-7.45 (m, 2H), 7.35 (s, 1H), 7.14 (d, J=5.0 Hz, 1H), 6.24 (d, J=15.0 Hz, 1H). LHMS-ESI, m/z [M+H]⁺ 436.07.

438: White solid, mp. 196-198° C., yield: 14.0%. ¹H NMR (500 MHz, acetone-d₆) δ 9.16 (br, 1H), 8.87 (br. d, J=10.5 Hz, 1H), 8.29 (d, J=2.5 Hz, 1H), 8.15 (d, J=9.0 Hz, 1H), 8.04-8.00 (m, 3H), 7.84 (dd, J₁=14.5 Hz, J₂=14.5 Hz, 1H), 7.78 (s, 1H), 6.38 (d, J=15.0 Hz, 1H). HRMS-ESI calcd for [M+H]⁺ 488.06512. Found: 488.06599.

441: White solid, mp. 215-217° C., yield: 48.9%. ¹H NMR (500 MHz, acetone-d₆) δ 9.09 (br. d, J=10.5 Hz, 1H), 8.57 (d, J=9.0 Hz, 1H), 8.47 (b, 1H), 7.95 (dd, J₁=2.5 Hz, J₂=2.5 Hz, 1H), 7.83 (d, J=2.0 Hz, 1H), 7.72-7.65 (m, 3H), 7.55 (t, J=7.5 Hz, 1H), 7.48 (d, J=7.5 Hz, 1H), 6.20 (d, J=15.0 Hz, 1H), 4.09 (s, 3H).

445: White solid, mp. 202-204° C., yield: 27.0%. ¹H NMR (500 MHz, acetone-d₆) δ 8.96 (br. d, J=10.5 Hz, 1H), 8.49-8.47 (m, 1H), 8.14-8.11 (m, 2H), 8.04 (b, 1H), 7.72-7.65 (m, 3H), 7.54 (t, J=7.5 Hz, 1H), 7.48 (d, J=7.5 Hz, 1H), 6.21 (d, J=15.0 Hz, 1H), 2.45 (s, 3H).

446: White solid, mp. 163-166° C., yield: 42.4%. ¹H NMR (500 MHz, acetone-d) δ 8.53 (br, 1H), 7.74-7.67 (m, 5H), 7.65 (s, 1H), 7.63-7.61 (m, 2H), 7.56-7.52 (m, 3H), 7.47 (d, J=8.0 Hz, 1H), 6.18 (d, J=14.5 Hz, 1H).

449: White solid, mp. 165-167° C., yield: 30.7%. ¹H NMR (500 MHz, acetone-d₆) δ 9.08 (br, 1H), 8.66 (br. d, J=10.5 Hz, 1H), 8.23 (d, J=9.5 Hz, 2H), 8.83 (d, J=3.0 Hz, 2H), 7.71-7.65 (m, 3H), 7.55 (t, J=7.5 Hz, 1H), 7.49 (d, J=8.0 Hz, 1H), 6.25 (d, J=14.5 Hz, 1H).

456: White solid, mp. 195-197° C., yield: 29.3%. ¹H NMR (500 MHz, acetone-d₆) δ 9.11 (br, 1H), 8.77 (br. d, J=10.5 Hz, 1H), 8.28 (d, J=2.5 Hz, 1H), 8.18-8.14 (m, 2H), 8.02-8.00 (m, 2H), 7.86 (t, J=5.5 Hz, 1H), 7.75-7.69 (m, 1H), 7.62-7.58 (m, 1H), 6.32 (dd, J₁=4.0 Hz, J₂=4.0 Hz, 1H). LHMS-ESI, m/z [M+H]⁺ 397.08.

462: White solid, mp.>300° C., yield: 51.1%. ¹H NMR (500 MHz, acetone-d₆) δ 8.80 (br. d, J=10.5 Hz, 1H), 8.32 (br, 1H), 7.57-7.48 (m, 5H), 7.07 (d, J=8.5 Hz, 2H), 6.51 (d, J=14.5 Hz, 2H), 6.04-5.97 (m, 1H), 4.83 (br, 2H).

463: White solid, mp. 233-235° C., yield: 29.9%. ¹H NMR (500 MHz, acetone-d₆) δ 9.08 (br, 1H), 8.84 (br. d, J=10.5 Hz, 1H), 8.29 (s, 1H), 8.03 (s, 2H), 8.00-7.94 (m, 2H), 7.86-7.81 (m, 1H), 7.77 (s, 1H), 6.37 (d, J=14.5 Hz, 1H). HRMS-ESI calcd for [M+H]⁺ 468.05729. Found: 468.07602.

464: White solid, mp. 228-230° C., yield: 50.9%. ¹H NMR (500 MHz, acetone-d₆) δ 9.12 (br, 1H), 9.00 (br. d, J=10.0 Hz, 1H), 8.29 (s, 1H), 8.17 (s, 1H), 7.99-7.95 (m, 2H), 7.92 (d, J=8.5 Hz, 1H), 7.78-7.69 (m, 2H), 6.52-6.48 (m, 1H). HRMS-ESI calcd for [M+H]⁺ 468.05729. Found: 468.07611.

468: White solid, mp. 256-258° C., yield: 53.9%. ¹H NMR (500 MHz, acetone-d₆) δ 8.86 (br. d, J=10.5 Hz, 1H), 8.79 (br, 1H), 8.17 (s, 1H), 7.92 (d, J=8.0 Hz, 1H), 7.79-7.77 (m, 3H), 7.75-7.69 (m, 3H), 6.49-6.44 (m, 1H). HRMS-ESI calcd for [M+H]⁺ 400.08791. Found: 400.08927.

469: White solid, mp. 212-214° C., yield: 43.2%. ¹H NMR (500 MHz, acetone-d₆) δ 8.75 (br, 1H), 8.69 (br. d, J=10.5 Hz, 1H), 8.02 (s, 2H), 7.86-7.77 (m, 6H), 6.32 (d, J=14.5 Hz, 1H). HRMS-ESI calcd for [M+H]⁺ 400.08791. Found: 400.08976.

472: White solid, mp. 257-259° C., yield: 22.3%. ¹H NMR (500 MHz, acetone-d₆) δ 9.36 (br. d, J=11.0 Hz, 1H), 8.57 (d, J=9.0 Hz, 1H), 8.50 (br, 1H), 8.19 (s, 1H), 7.97-7.92 (m, 2H), 7.85 (d, J=2.0 Hz, 1H), 7.79-7.76 (m, 1H), 7.70 (d, J=8.5 Hz, 1H), 6.47-6.43 (m, 1H), 4.11 (s, 3H).

473: White solid, mp. 253-255° C., yield: 26.8%. ¹H NMR (500 MHz, acetone-d₆) δ 8.98 (br, 1H), 8.91 (br. d, J=10.5 Hz, 1H), 8.24 (d, J=9.0 Hz, 2H), 8.18 (s, 1H), 7.93 (d, J=8.5 Hz, 1H), 7.84 (d, J=9.0 Hz, 2H), 7.78 (dd, J₁=14.0 Hz, J₂=14.0 Hz, 1H), 7.70 (d, J=8.0 Hz, 1H), 6.49 (dd, J₁=3.0 Hz, J₂=3.0 Hz, 1H).

474: White solid, mp. 251-253° C., yield: 69.8%. ¹H NMR (500 MHz, acetone-d₆) δ 9.19 (br. d, J=10.5 Hz, 1H), 8.48-8.46 (m, 1H), 8.19 (s, 1H), 8.15-8.13 (m, 2H), 8.05 (br, 1H), 7.93 (d, J=8.0 Hz, 1H), 7.79 (dd, J₁=14.0 Hz, J₂=14.0 Hz, 1H), 7.70 (d, J=8.0 Hz, 1H), 6.44 (dd, J₁=2.0 Hz, J₂=2.0 Hz, 1H), 2.48 (s, 3H).

488: White solid, mp. 249-251° C., yield: 60.5%. ¹H NMR (500 MHz, acetone-d₆) δ 8.91 (br, 1H), 8.89 (br, 1H), 8.12 (s, 1H), 8.01 (d, J=2.4 Hz, 1H), 7.88 (d, J=8.0 Hz, 1H), 7.75 (d, J=8.8 Hz, 1H), 7.71-7.69 (m, 1H), 7.67 (d, J=8.8 Hz, 1H), 7.58-7.57 (m, 1H), 6.45 (dd, J₁=1.6 Hz, J₂=1.6 Hz, 1H).

490: White solid, mp. 231-233° C., yield: 58.2%. ¹H NMR (500 MHz, acetone-d₆) δ 8.86 (br, 1H), 8.73 (br. d, J=10.4 Hz, 1H), 8.00 (s, 1H), 7.97 (s, 2H), 7.79-7.74 (m, 2H), 7.72 (s, 1H), 7.56 (dd, J₁=1.6 Hz, J₂=1.6 Hz, 1H), 6.31 (d, J=14.4 Hz, 1H).

723: White solid, yield: 91.4%. ¹H NMR (500 MHz, acetone-d₆) δ 8.39 (d, J=10.5 Hz, 1H), 7.93 (s, 2H), 7.91 (br, 1H), 7.85-7.79 (m, 1H), 7.68 (s, 1H), 7.37-7.28 (m, 2H), 6.76-6.67 (m, 2H), 6.16 (d, J=14.6 Hz, 1H), 2.88 (s, 6H).

The chemical structures of compounds 403, 404, 405, 406, 407, 408, 409, 410, 411, 412, 413, 414, 415, 416, 417, 421, 429, 430, 433, 435, 436, 437, 438, 441, 445, 446, 449, 456, 462, 463, 464, 468, 469, 472, 473, 474, 488, 490 and 723 prepared as described above are provided in Table 2.2 herein below.

### The 562 “Analogues of Formula (III)”

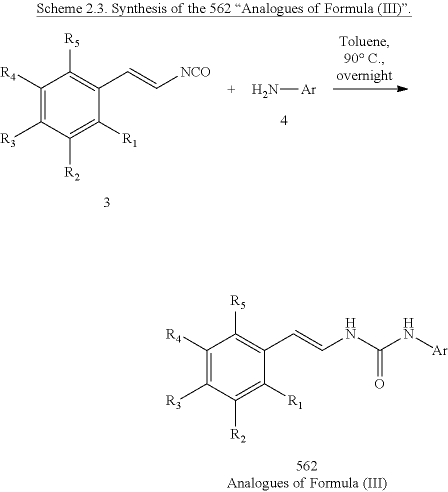

General Procedure for the Synthesis of the 562 “Analogues of Formula (III)”-Scheme 2.3:

An equimolar mixture of aryl isocyanate 3 and aryl amine 4 in toluene was heated at 90° C. overnight. After cooling to room temperature, white solid was precipitated, which was collected by filtration and washed with toluene.

### Characterization of the 562 “Analogues of Formula (III)”

418: White solid, mp. 177-179° C., yield: 88.0%. ¹H NMR (500 MHz, acetone-d₆) δ 8.34 (br. d, J=10.5 Hz, 1H), 8.18 (br, 1H), 7.70-7.65 (m, 2H), 7.62 (s, 1H), 7.52 (t, J=8.0 Hz, 1H), 7.45 (d, J=8.0 Hz, 1H), 7.32-7.31 (m, 1H), 6.85 (dd, J₁=2.0 Hz, J₂=2.0 Hz, 1H), 6.78 (d, J=8.0 Hz, 1H), 6.13 (d, J=15.0 Hz, 1H), 6.99 (s, 2H).

427: White solid, mp. 192-194° C., yield: 77.1%. ¹H NMR (500 MHz, acetone-d₆) δ M.P. 192-194° C. ¹H NMR (500 MHz, CD₃COCD₃): δ 8.50 (br. d, J=10.5 Hz, 1H), 8.14 (b, 1H), 7.80 (d, J=8.5 Hz, 1H), 7.66-7.58 (m, 3H), 7.36-7.31 (m, 2H), 6.86 (dd, J₁=2.5 Hz, J₂=2.0 Hz, 1H), 6.78 (d, J=8.5 Hz, 1H), 6.35-6.31 (m, 1H), 5.99 (s, 2H).

431: White solid, mp. 178-180° C., yield: 67.9%. ¹H NMR (500 MHz, acetone-d₆) δ 8.15 (br. d, J=10.5 Hz, 1H), 8.10 (b, 1H), 7.54 (dd, J₁=14.5 Hz, J₂=14.5 Hz, 1H), 7.32 (d, J=2.0 Hz, 1H), 7.20 (t, J=8.0 Hz, 1H), 6.92-6.89 (m, 2H), 6.84 (dd, J₁=2.0 Hz, J₂=2.0 Hz, 1H), 6.77 (d, J=8.5 Hz, 1H), 6.73-6.70 (m, 1H), 6.00 (d, J=15.0 Hz, 1H), 5.99 (s, 2H), 3.82 (s, 3H).

432: White solid, mp. 180-182° C., yield: 71.0%. ¹H NMR (500 MHz, acetone-d₆) δ 8.36 (br. d, J=10.5 Hz, 1H), 8.19 (b, 1H), 7.71 (dd, J₁=14.5 Hz, J₂=14.5 Hz, 1H), 7.62-7.54 (m, 4H), 7.32 (t, J=2.0 Hz, 1H), 6.85 (dd, J₁=2.0 Hz, J₂=2.0 Hz, 1H), 6.78 (d, J=8.0 Hz, 1H), 6.11 (d, J=14.5 Hz, 1H), 5.99 (s, 2H).

515: White solid, mp. 199-201° C., yield: 75.3%. ¹H NMR (500 MHz, acetone-d₆) δ 8.80 (dd, J₁=1.5 Hz, J₂=1.0 Hz, 1H), 8.67 (br. d, J=10.5 Hz, 1H), 8.68 (br, 1H), 8.28 (d, J=2.0 Hz, 1H), 8.23 (d, J=8.0 Hz, 1H), 8.01-7.98 (m, 3H), 7.90 (ddd, J₁=14.5 Hz, J₂=1.5 Hz, J₃=1.5 Hz, 1H), 7.80 (dd, J₁=2.5 Hz, J₂=2.0 Hz, 1H), 7.75 (s, 1H), 7.47 (dd, J₁=8.0 Hz, J₂=8.5 Hz, 1H), 6.31 (d, J=14.5 Hz, 1H).

516: White solid, mp. 214-216° C., yield: 77.5%. ¹H NMR (500 MHz, acetone-d₆) δ 10.21 (br, 1H), 8.45 (br. d, J=10.5 Hz, 1H), 8.07 (br, 1H), 7.96 (s, 2H), 7.89 (dd, J₁=14.5 Hz, J₂=15.0 Hz, 1H), 7.80 (s, 1H), 7.71 (s, 1H), 7.38 (d, J=10.5 Hz, 1H), 7.35 (d, J=2.5 Hz, 1H), 7.21 (d, J=8.5 Hz, 1H), 6.45 (d, J=3.0 Hz, 1H), 6.21 (d, J=14.5 Hz, 1H).

517: White solid, mp. 226-228° C., yield: 83.5%. ¹H NMR (500 MHz, acetone-d₆) δ 8.84 (br.d, J=10.0 Hz, 1H), 8.80 (dd, J₁=1.5 Hz, J₂=1.5 Hz, 1H), 8.66 (br, 1H), 8.28 (d, J=1.5 Hz, 1H), 8.24 (d, J=7.5 Hz, 1H), 8.17 (s, 1H), 8.00 (d, J=9.0 Hz, 1H), 7.92 (d, J=8.5 Hz, 1H), 7.86-7.79 (m, 2H), 7.68 (d, J=8.0 Hz, 1H), 7.48 (dd, J₁=8.0 Hz, J₂=8.5 Hz, 1H), 6.45 (dd, J₁=2.0 Hz, J₂=2.0 Hz, 1H).

518: White solid, mp. 216-218° C., yield: 72.3%. ¹H NMR (500 MHz, acetone-d₆) δ 10.21 (br, 1H), 8.64 (br. d, J=10.5 Hz, 1H), 8.14 (s, 1H), 8.08 (br, 1H), 7.89 (d, J=8.5 Hz, 1H), 7.85 (dd, J₁=14.0 Hz, J₂=14.5 Hz, 1H), 7.80 (s, 1H), 7.64 (d, J=8.0 Hz, 1H), 7.38 (d, J=8.5 Hz, 1H), 7.35 (s, 1H), 7.22 (d, J=8.5 Hz, 1H), 6.46 (d, J=3.0 Hz, 1H), 6.37 (dd, J₁=1.5 Hz, J₂=2.0 Hz, 1H).

519: White solid, mp. 197-199° C., yield: 86.8%. ¹H NMR (500 MHz, acetone-d₆) δ 8.79 (d, J=1.0 Hz, 1H), 8.59 (br, 1H), 8.51 (br. d, J=9.5 Hz, 1H), 8.28 (s, 1H), 8.23 (d, J=8.0 Hz, 1H), 7.99 (d, J=9.0 Hz, 1H), 7.79 (dd, J₁=2.0 Hz, J₂=2.0 Hz, 1H), 7.74-7.69 (m, 2H), 7.66 (s, 1H), 7.53 (t, J=8.0 Hz, 1H), 7.49-7.45 (m, 2H), 6.21 (d, J=15.0 Hz, 1H).

520: White solid, mp. 215-217° C., yield: 76.8%. ¹H NMR (500 MHz, acetone-d₆) δ 10.19 (br, 1H), 8.29 (br. d, J=10.5 Hz, 1H), 8.04 (br, 1H), 7.80 (d, J=2.0 Hz, 1H), 7.76-7.71 (m, 1H), 7.64 (d, J=8.0 Hz, 1H), 7.61 (s, 1H), 7.50 (t, J=8.0 Hz, 1H), 7.43 (d, J=7.5 Hz, 1H), 7.38 (d, J=8.5 Hz, 1H), 7.34 (t, J=2.5 Hz, 1H), 7.21 (dd, J₁=2.0 Hz, J₂=2.0 Hz, 1H), 6.46-6.44 (m, 1H), 6.09 (d, J=14.5 Hz, 1H).

523: White solid, mp. 208-209° C., yield: 81.7%. ¹H NMR (500 MHz, acetone-d₆) δ 8.88 (s, 1H), 8.76 (br.d, J=10.0 Hz, 1H), 8.71 (br, 1H), 8.63 (s, 1H), 8.02-7.99 (m, 3H), 7.91-7.87 (m, 2H), 7.76 (s, 1H), 7.65 (t, J=7.5 Hz, 1H), 7.59 (t, J=7.0 Hz, 1H), 6.34 (d, J=14.5 Hz, 1H).

524: White solid, mp. 220-221° C., yield: 61.7%. ¹H NMR (500 MHz, acetone-d₆) δ 8.93 (br.d, J=10.0 Hz, 1H), 8.89 (s, 1H), 8.75 (br, 1H), 8.63 (s, 1H), 8.17 (s, 1H), 8.00 (d, J=8.0 Hz, 1H), 7.92 (t, J=9.0 Hz, 2H), 7.84 (t, J=9.0 Hz, 1H), 7.69 (d, J=8.0 Hz, 1H), 7.66-7.63 (m, 1H), 7.58 (t, J=6.0 Hz, 1H), 6.48 (d, J=14.0 Hz, 1H).

525: White solid, mp. 203-205° C., yield: 81.2%. ¹H NMR (500 MHz, acetone-d₆) δ 8.88 (s, 1H), 8.67 (br, 1H), 8.63 (s, 1H), 8.60 (br, 1H), 7.99 (d, J=8.5 Hz, 1H), 7.90 (d, J=7.5 Hz, 1H), 7.75-7.61 (m, 4H), 7.59-7.53 (m, 2H), 7.48 (d, J=7.5 Hz, 1H), 6.24 (d, J=15.0 Hz, 1H).

The chemical structures of compounds 418, 427, 431, 432, 515, 516, 517, 518, 519, 520, 523, 524 and 525 prepared as described above are provided in Table 2.3 herein below.

### The 562 “Analogues of Formula (IV)”

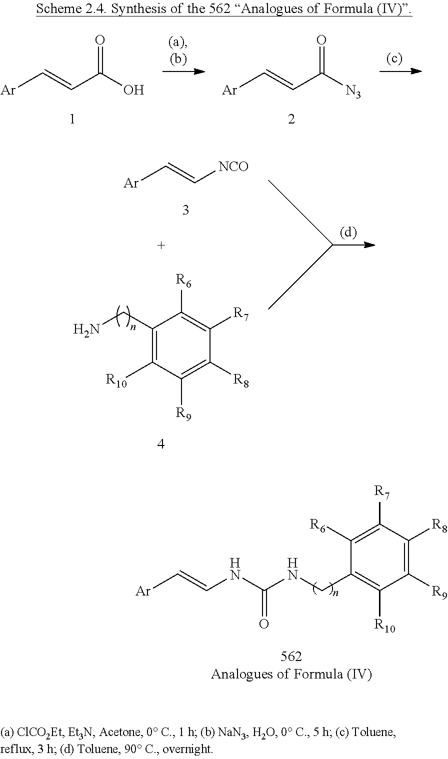

General Procedure for the Synthesis of Aryl Azid 2—Scheme 2.4:

To a solution of 1 (1 mmol) in dry acetone (10 mL), triethylamine (1.1 mmol) and ethyl chlorocarbamate (1.1 mmol) were added dropwise at 0° C. After stirring at 0° C. for 1 h, sodium azide (1.1 mmol, 0.215 g) dissolved in 5 mL water was added dropwise. Stirring was continued at 0° C. for 5 h. Ice water was added. The mixture was extracted by dichloromethane (3×20 mL). The combined organic layers were washed with brine and dried over Na₂SO₄. The organic phase was concentrated under reduced pressure. Colorless oil was obtained and used in the following reaction without further purification.

General Procedure for the Synthesis of the 562 “Analogues of Formula (IV)”-Scheme 2.4:

A solution of aryl azide 2 (0.5 mmol) in toluene (10 mL) was heated at 120° C. for 3 h to give aryl isocyanate 3, which is not isolated and treated in situ with the respective 4 at 90° C. overnight. The solvent was cooled to room temperature and the precipitate was collected by filtration and washed with toluene.

### Characterization of the 562 “Analogues of Formula (IV)”

419: White solid, mp. 189-190° C., yield: 19.0%. ¹H NMR (500 MHz, acetone-d₆) δ 9.12 (br, 1H), 8.58 (br. d, J=10.0 Hz, 1H), 8.28 (d, J=2.0 Hz, 1H), 8.14 (d, J=9.0 Hz, 1H), 8.00 (dd, J₁=2.0 Hz, J₂=2.0 Hz, 1H), 7.35 (dd, J₁=14.5.0 Hz, J₂=14.5 Hz, 1H), 7.22 (d, J=5.0 Hz, 1H), 6.98 (dd, J₁=5.0 Hz, J₂=5.0 Hz, 1H), 6.93 (d, J=3.5 Hz, 1H), 6.39 (d, J=14.0 Hz, 1H). LHMS-ESI, m/z [M+H]⁺ 358.05.

420 White solid, mp. 172-174° C., yield: 74.3%. ¹H NMR (500 MHz, acetone-d₆) δ 8.58 (br, 1H), 8.36 (br. d, J=10.0 Hz, 1H), 8.09 (s, 1H), 7.71 (dd, J₁=1.5 Hz, J₂=2.0 Hz, 1H), 7.53 (t, J=8.0 Hz, 1H), 7.41-7.34 (m, 2H), 7.19 (d, J=5.0 Hz, 1H), 6.96 (dd, J₁=5.0 Hz, J₂=5.0 Hz, 1H), 6.89 (d, J=3.5 Hz, 1H), 6.32 (d, J=14.5 Hz, 1H).

424 White solid, mp. 183-185° C., yield: 73.3%. ¹H NMR (500 MHz, acetone-d₆) δ 8.68 (br, 1H), 8.36 (br. d, J=10.0 Hz, 1H), 7.77-7.75 (m, 2H), 7.69 (d, J=9.0 Hz, 2H), 7.36 (dd, J₁=14.5 Hz, J₂=14.5 Hz, 1H), 7.20 (d, J=5.0 Hz, 1H), 6.97-6.95 (m, 1H), 6.90 (d, J=3.5 Hz, 1H), 6.34 (d, J=14.5 Hz, 1H). LHMS-ESI, m/z [M+H]⁺ 270.07.

425 White solid, mp. 181-183° C., yield: 83.9%. ¹H NMR (500 MHz, acetone-d₆) δ 8.19 (br, 1H), 8.18 (br, 1H), 7.39 (dd, J₁=14.5 Hz, J₂=14.5 Hz, 1H), 7.33-7.32 (m, 1H), 7.20-7.16 (m, 2H), 7.02-7.00 (m, 1H), 6.96-6.94 (m, 1H), 6.87 (d, J=3.0 Hz, 1H), 6.61-6.59 (m, 1H), 6.27 (d, J=14.5 Hz, 1H), 3.79 (s, 3H).

426: White solid, mp. 203-205° C., yield: 81.9%. ¹H NMR (500 MHz, acetone-d₆) δ 8.14 (br. d, J=10.5 Hz, 1H), 8.10 (b, 1H), 7.38 (dd, J₁=14.5 Hz, J₂=14.5 Hz, 1H), 7.31-7.29 (m, 1H), 7.16 (d, J=5.5 Hz, 1H), 6.94 (dd, J₁=5.5 Hz, J₂=5.0 Hz, 1H), 6.86-6.82 (m, 2H), 6.77 (d, J=8.5 Hz, 1H), 6.24 (d, J=14.5 Hz, 1H), 5.98 (s, 2H).

428: White solid, mp. 199-201° C., yield: 45.7%. ¹H NMR (500 MHz, acetone-d₆) δ M.P. 199-201° C. 9.01 (br, 1H), 8.52 (br. d, J=10.0 Hz, 1H), 8.28 (d, J=2.0 Hz, 1H), 7.98-7.92 (m, 2H), 7.34 (dd, J₁=14.5 Hz, J₂=14.5 Hz, 1H), 7.22 (d, J=5.0 Hz, 1H), 6.97 (dd, J₁=5.0 Hz, J₂=5.0 Hz, 1H), 6.93 (d, J=3.5 Hz, 1H), 6.38 (d, J=14.5 Hz, 1H). LHMS-ESI, m/z [M+H]⁺ 338.06.

434: White solid, mp. 163-165° C., yield: 17.6%. ¹H NMR (500 MHz, acetone-d₆) δ 9.11 (br, 1H), 8.58 (br. d, J=10.0 Hz, 1H), 8.31-8.28 (m, 1H), 8.21-8.16 (m, 1H), 8.03-8.01 (m, 1H), 7.48 (s, 1H), 7.45-7.40 (m, 1H), 6.44 (d, J=1.5 Hz, 1H), 6.35 (d, J=3.5 Hz, 1H), 6.11 (d, J=15.0 Hz, 1H). LHMS-ESI, m/z [M+H]⁺ 342.07.

443: White solid, mp. 129-131° C., yield: 21.5%. ¹H NMR (500 MHz, acetone-d₆) δ 9.03 (br, 1H), 8.42 (br. d, J=10.0 Hz, 1H), 8.28 (d, J=2.5 Hz, 1H), 8.14 (d, J=9.0 Hz, 1H), 7.97 (dd, J₁=3.0 Hz, J₂=3.0 Hz, 1H), 7.53-7.51 (m, 2H), 7.24 (dd, J₁=14.5 Hz, J₂=14.5 Hz, 1H), 6.71 (d, J=1.0 Hz, 1H), 6.04 (d, J=14.5 Hz, 1H).

444: White solid, mp. 187-189° C., yield: 33.3%. ¹H NMR (500 MHz, acetone-d₆) δ 9.04 (br, 1H), 8.47 (br. d, J=10.0 Hz, 1H), 8.28 (d, J=2.0 Hz, 1H), 8.13 (d, J=8.5 Hz, 1H), 7.98 (dd, J₁=2.5 Hz, J₂=2.0 Hz, 1H), 7.37 (dd, J₁=14.5 Hz, J₂=14.5 Hz, 1H), 6.98 (s, 1H), 6.79 (s, 2H), 6.11 (d, J=14.5 Hz, 1H), 5.99 (s, 2H). LHMS-ESI, m/z [M+H]⁺ 396.08.

447: White solid, mp. 118-120° C., yield: 21.6%. ¹H NMR (500 MHz, acetone-d₆) δ 8.48 (br. d, J=10.5 Hz, 1H), 8.26-8.23 (m, 2H), 7.66-7.63 (m, 3H), 7.63 (s, 1H), 7.51 (t, J=8.0 Hz, 1H), 7.43 (d, J=7.5 Hz, 1H), 6.68 (t, J=7.5 Hz, 1H), 6.06 (d, J=15.0 Hz, 1H), 4.61 (d, J=6.0 Hz, 2H).

448: White solid, mp. 118-120° C., yield: 44.2%. ¹H NMR (500 MHz, acetone-d₆) δ 8.93 (br, 1H), 8.27 (d, J=2.0 Hz, 1H), 8.09 (d, J=9.0 Hz, 1H), 7.88 (dd, J₁=2.5 Hz, J₂=2.0 Hz, 1H), 7.64 (s, 1), 7.62-7.56 (m, 3H), 6.34 (br, 1H), 3.61-3.57 (m, 2H), 3.02 ((t, J=7.0 Hz, 2H). LHMS-ESI, m/z [M+H]⁺ 422.09.

450: White solid, mp. 191-193° C., yield: 23.3%. ¹H NMR (500 MHz, acetone-d₆) δ 9.14 (br, 1H), 8.64 (br. d, J=10.0 Hz, 1H), 8.33 (d, J=2.5 Hz, 1H), 8.22 (d, J=8.0 Hz, 1H), 8.15 (d, J=8.5 Hz, 1H), 8.01 (dd, J₁=2.5 Hz, J₂=2.5 Hz, 1H), 7.79 (d, J=7.5 Hz, 1H), 7.71 (s, 1H), 7.60 (dd, J₁=14.5 Hz, J₂=15.0 Hz, 1H), 7.42-7.30 (m, 2H), 6.32 (d, J=14.5 Hz, 1H), 1.71 (s, 9H). LHMS-ESI, m/z [M+H]⁺ 491.15.

453: White solid, mp. 121-123° C., yield: 52.4%. ¹H NMR (500 MHz, acetone-d₆) δ 9.09 (br, 1H), 8.55 (br. d, J=10.5 Hz, 1H), 8.26 (d, J=2.0 Hz, 1H), 8.14 (d, J=9.0 Hz, 1H), 8.07 (s, 1H), 8.01 (dd, J₁=2.5 Hz, J₂=2.5 Hz, 1H), 7.71 (dd, J₁=14.5 Hz, J₂=14.5 Hz, 1H), 7.26 (s, 1H), 6.05 (d, J=14.5 Hz, 1H), 1.65 (s, 9H). LHMS-ESI, m/z [M+Na]⁺ 464.12.

459: White solid, mp. 130-132° C., yield: 66.2%. ¹H NMR (500 MHz, acetone-d₆) δ 8.13 (br, 1H), 7.66-7.54 (m, 7H), 7.49 (t, J=7.5 Hz, 1H), 7.41 (d, J=8.0 Hz, 1H), 6.08-6.04 (br. m, 1H), 5.99 (d, J=14.5 Hz, 1H), 3.57-3.53 (m, 2H), 2.98 (t, J=7.0 Hz, 2H).

460: White solid, mp. 80-82° C., yield: 29.4%. ¹H NMR (500 MHz, acetone-d₆) δ 8.40 (br. d, J=8.5 Hz, 1H), 7.70-7.51 (m, 7H), 7.50 (t, J=8.0 Hz, 1H), 7.42 (d, J=8.0 Hz, 1H), 6.60 (br, 1H), 6.04 (d, J=14.5 Hz, 1H), 4.55 (d, J=5.5 Hz, 2H).

461: White solid, mp. 152-153° C., yield: 60.4%. ¹H NMR (500 MHz, acetone-d₆) δ M 9.03 (br, 1H), 8.26 (d, J=2.0 Hz, 1H), 8.10 (d, J=8.5 Hz, 1H), 7.93 (dd, J₁=2.0 Hz, J₂=2.5 Hz, 1H), 7.73-7.70 (m, 2H), 7.64-7.59 (m, 2H), 6.85 (br.d, J=5.0 Hz, 1H), 4.59 (d, J=6.0 Hz, 2H). LHMS-ESI, m/z [M+H]⁺ 408.08.

633: White solid, yield: 63.1%. ¹H NMR (500 MHz, acetone-d₆) δ 8.22 (br, 1H), 7.69-7.52 (m, 7H), 7.47 (d, J=8.3 Hz, 2H), 6.03 (br, 1H), 5.94 (d, J=14.6 Hz, 1H), 3.53 (dt, J=13.2, 7.1 Hz, 2H), 2.95 (t, J=7.1 Hz, 2H).

634: White solid, yield: 66.7%. ¹H NMR (500 MHz, acetone-d₆) δ 7.57 (t, J=7.7 Hz, 2H), 7.49 (d, J=7.7 Hz, 1H), 7.46-7.35 (m, 5H), 7.21 (t, J=7.7 Hz, 1H), 6.48 (d, J=11.0 Hz, 1H), 6.16 (d, J=11.0 Hz, 1H), 4.58 (t, J=5.6 Hz, 1H), 3.55 (dd, J=5.6, 7.0 Hz, 2H), 2.91 (t, J=7.0 Hz, 2H).

635: White solid, yield: 66.2%. ¹H NMR (500 MHz, acetone-d₆) δ 7.99 (d, J=10.4 Hz, 1H), 7.59-7.50 (m, 4H), 7.41-7.36 (m, 1H), 7.35-7.34 (m, 1H), 6.33-6.32 (m, 1H), 6.01-6.00 (m, 1H), 5.94 (br, 1H), 5.76 (d, J=14.6 Hz, 1H), 3.49 (dd, J=10.4, 7.1 Hz, 2H), 2.93 (t, J=7.1 Hz, 2H).

642: White solid, yield: 55.4%. ¹H NMR (500 MHz, acetone-d₆) δ 7.98 (d, J=10.7 Hz, 1H), 7.62-7.50 (m, 4H), 7.50-7.42 (m, 1H), 7.28-7.18 (m, 4H), 7.07-7.03 (m, 1H), 5.91 (br, 1H), 5.84 (d, J=14.7 Hz, 1H), 3.52-3.44 (m, 2H), 2.93 (t, J=7.1 Hz, 2H).

982 White solid, 72.3% in yield. ¹H NMR (500 MHz, acetone) δ 9.25 (s, 1H), 8.27 (s, 1H), 8.20-8.14 (m, 2H), 7.91 (d, J=8.6 Hz, 1H), 7.86 (d, J=8.6 Hz, 1H), 7.41-7.27 (m, 2H), 6.61 (s, 1H), 4.82 (d, J=4.9 Hz, 2H).

Compound 454 was prepared according to the following scheme:

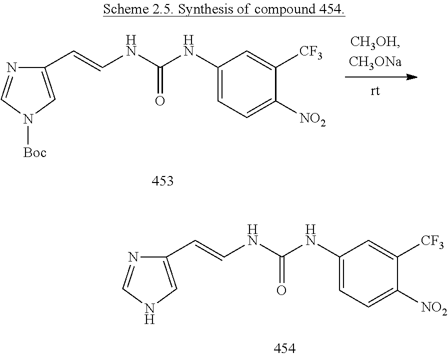 [SMILES: CC(C)(C)OC(=O)n1cnc(/C=C/NC(=O)Nc2ccc([N+](=O)[O-])c(C(F)(F)F)c2)c1 ; O=C(N/C=C/c1c[nH]cn1)Nc1ccc([N+](=O)[O-])c(C(F)(F)F)c1]

Preparation of Compound 454:

Referring to Scheme 2.5 reproduced above, to a solution of 453 (50 mg, 0.113 mmol) in 4 mL methanol, sodium methoxide (13 mg, 0.24 mmol) dissolved in 3 mL methanol was added. The mixture was stirred at room temperature for 1 h. Then 12 mL water was added to the mixture when the reaction was completed (detected by TLC. Yellow solid precipitated from the reaction mixture and was collected by filtration. The product was dried under reduced pressure. 37 mg (96% in yield) of 454 was obtained as yellow solid. mp. 145-147° C., yield: 96%. ¹H NMR (500 MHz, acetone-d₆) δ 9.06 (br, 1H), 8.39 (br, 1H), 8.26 (s, 1H), 8.13 (d, J=8.5 Hz, 1H), 7.99 (d, J=9.0 Hz, 1H), 7.60 (s, 1H), 7.51 (dd, J₁=14.5 Hz, J₂=14.5 Hz, 1H), 6.97 (s, 1H), 6.11 (d, J=14.5 Hz, 1H). LHMS-ESI, m/z [M+H]⁺ 342.08.

The chemical structures of compounds 419, 420, 424, 425, 426, 428, 434, 443, 444, 447, 448, 450, 453, 454, 459, 460, 461, 633, 634, 635 and 642 prepared as described above are provided in Table 2.4 herein below.

### The 562 “Analogues of Formula (V)”

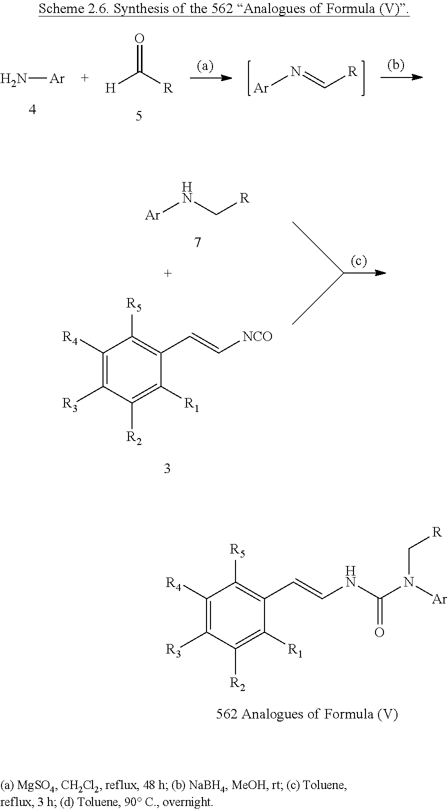

General Procedure for the Synthesis of Intermediate 7:

Referring to Scheme 2.6 reproduced above, to a solution of arylamine 4 (2.1 mmol) and aldehyde 5 (2.3 mmol) in dichloromethane (20 mL), magnesium sulfate (4.2 mmol, 0.5 g) was added. The mixture was refluxed for 24 h. The crude product was obtained after filtering the solid and distilling off the solvent, which was used directly in the following step. Then the residue was dissolved in 15 mL of methanol. Sodium borohydride was added and the resulting mixture was stirred at room temperature for 5 h. Ammonium chloride (2M, 20 mL) was then added to quench the reaction. The solution was extracted with ethyl acetate (3×20 mL). The organic layer was dried over MgSO₄ and then removed in vacuo. The residue was purified by column chromatography.

General Procedure for the Synthesis of the 562 “Analogues of Formula (V)”—Scheme 2.6:

A mixture of aryl isocyanate 3 and amine 7 in toluene was heated at 90° C. overnight. After cooling to room temperature, white solid was precipitated, which was collected by filtration and washed with toluene.

### Characterization of the 562 “Analogues of Formula (V)”

534: White solid, mp. 181-183° C., yield: 38.8%. ¹H NMR (500 MHz, acetone-d₆) δ 8.76 (s, 1H), 8.67 (d, J=9.0 Hz, 1H), 8.05-8.00 (m, 2H), 7.94 (s, 1H), 7.82 (d, J=14.5 Hz, 1H), 7.74 (s, 1H), 6.16 (d, J=14.5 Hz, 1H), 5.31 (s, 1H), 4.48 (s, 2H), 2.02-1.95 (m, 2H), 1.68 (s, 3H), 0.87-0.84 (m, 3H).

547: White solid, mp. 179-180° C., yield: 60.8%. ¹H NMR (500 MHz, acetone-d₆) δ 8.38 (br.d, J=10.0 Hz, 1H), 7.92-7.86 (m, 3H), 7.68 (s, 1H), 6.20 (d, J=15.0 Hz, 1H), 3.92-3.87 (m, 2H), 1.34 (s, 6H), 1.33 (s, 6H).

563: White solid, mp. 112-114° C., yield: 10.8%. ¹H NMR (500 MHz, acetone-d₆) δ 8.76 (d, J=2.5 Hz, 1H), 8.51 (br.d, J=10.0 Hz, 1H), 8.08-7.99 (m, 2H), 7.68-7.59 (m, 3H), 7.51 (t, J=7.5 Hz, 1H), 7.46 (d, J=8.0 Hz, 1H), 6.08 (d, J=14.5 Hz, 1H), 5.33-5.29 (m, 1H), 4.48 (s, 2H), 2.04-1.99 (m, 2H), 1.68 (s, 3H), 0.89-0.83 (m, 3H).

591: White solid, mp. 56-58° C., yield: 21.3%. ¹H NMR (500 MHz, acetone-d₆) δ 11.93 (br.d, J=9.5 Hz, 1H), 9.71 (t, J=1.5 Hz, 1H), 7.98 (t, J=1.0 Hz, 2H), 7.96 (s, 2H), 7.88-7.83 (m, 1H), 7.74 (s, 1H), 7.38-7.28 (m, 5H), 6.14 (d, J=15.0 Hz, 1H), 5.16 (s, 2H).

620: White solid. Yield: 56.7%. ¹H NMR (500 MHz, CDCl₃) δ 7.66-7.61 (m, 1H), 7.58 (s, 2H), 7.54 (s, 1H), 7.45-7.33 (m, 3H), 7.20-7.14 (m, 1H), 7.11 (d, J=6.9 Hz, 2H), 6.82-6.74 (m, 3H), 6.32 (d, J=10.9 Hz, 1H), 5.70 (d, J=14.6 Hz, 1H), 4.86 (s, 2H), 3.73 (s, 3H).

621: White solid. Yield: 53.5%. ¹H NMR (500 MHz, CDCl₃) δ 7.65-7.57 (m, 3H), 7.55 (s, 1H), 7.24-7.22 (m, 1H), 7.18 (t, J=7.8 Hz, 1H), 7.14 (d, J=2.0 Hz, 1H), 6.87 (dd, J=8.0, 2.1 Hz, 1H), 6.78 (d, J=8.0 Hz, 3H), 6.30 (d, J=10.8 Hz, 1H), 5.75 (d, J=14.6 Hz, 1H), 4.82 (s, 2H), 3.75 (s, 3H), 2.37 (s, 3H).

622: White solid. Yield: 49.5%. ¹H NMR (800 MHz, CDCl₃) δ 7.63 (m, 2H), 7.60 (s, 2H), 7.56 (s, 1H), 7.53 (t, J=7.9 Hz, 1H), 7.39 (s, 1H), 7.28 (d, J=7.8 Hz, 1H), 7.19 (t, J=7.9 Hz, 1H), 6.82-6.72 (m, 3H), 6.20 (d, J=10.7 Hz, 1H), 5.76 (d, J=14.6 Hz, 1H), 4.87 (s, 2H), 3.74 (s, 3H).

623: White solid. Yield: 60.1%. ¹H NMR (800 MHz, CDCl₃) δ 7.69 (d, J=8.4 Hz, 2H), 7.64-7.56 (m, 4H), 7.29-7.25 (m, 5H), 7.19 (d, J=6.9 Hz, 2H), 6.29 (d, J=10.7 Hz, 1H), 5.79 (d, J=14.6 Hz, 1H), 4.93 (s, 2H).

The chemical structures of compounds 534, 547, 563, 591, 620, 621, 622 and 623 prepared as described above are outlined in Table 2.5 below.

### Compound 804 and its Analogues

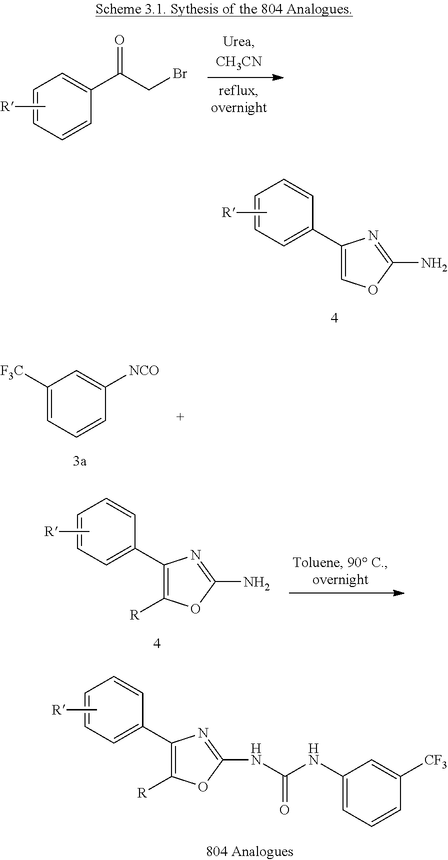

General Procedure for the Synthesis of Compound 2-Amino-Oxazoles 4:

A mixture of substituted 2-bromoacetonphenone (2 mmol) and urea (20 mmol, 10 eq) were reflux overnight in acetonitrile (25 mL). After cooling to room temperature, the reaction mixture was concentrated and purified by column chromatography.

General Procedure for the Synthesis of Compound 804 and its Analogues—Scheme 3.1:

A mixture of 3-(trifluoromethyl)benzyl isocyanate 3a, and amine 4 in toluene was heated at 90° C. for overnight. The solvent was cooled to room temperature and the precipitate was collected by filtration and washed with toluene.

### Characterization of Compound 804 and its Analogues

804: White solid, yield: 69.4%. ¹H NMR (800 MHz, acetone-d₆) δ 11.15 (br, 1H), 10.13 (br, 1H), 8.15 (s, 1H), 7.93-7.90 (m, 4H), 7.77-7.74 (m, 2H), 7.26-7.19 (m, 2H). MS (ESI) calculated for C₁₇H₁₂FN₄O₂[M+H] 323.0938. Found 323.0944.

790: White solid, yield: 45.5%. ¹H NMR (500 MHz, acetone-d₆) δ 11.14 (br, 1H), 9.87 (br, 1H), 8.23 (s, 1H), 7.76-7.73 (m, 3H), 7.60-7.57 (m, 1H), 7.50-7.47 (m, 2H), 7.43-7.41 (m, 1H), 7.38-7.35 (m, 1H), 2.94 (q, J=7.5 Hz, 2H), 1.31 (t, J=7.5 Hz, 3H).

791: White solid, yield: 76.5%. ¹H NMR (500 MHz, acetone-d₆) δ 11.09 (br, 1H), 8.23 (br, 1H), 7.79-7.72 (m, 3H), 7.63-7.55 (m, 3H), 7.51-7.36 (m, 8H).

797: White solid, yield: 77.5%. ¹H NMR (500 MHz, acetone-d₆) δ 10.89 (br, 1H), 10.15 (br, 1H), 8.37 (s, 1H), 8.28 (s, 1H), 8.14-8.06 (m, 2H), 7.90-7.84 (m, 2H), 7.80 (d, J=8.1 Hz, 1H), 7.61 (t, J=8.1 Hz, 1H), 7.45 (d, J=8.1 Hz, 1H).

798: White solid, yield: 74.5%. ¹H NMR (500 MHz, acetone-d₆) δ 10.99 (br, 1H), 10.09 (br, 1H), 8.27 (s, 1H), 8.19 (s, 1H), 7.92-7.86 (m, 2H), 7.78 (d, J=8.2 Hz, 1H), 7.59 (t, J=8.2 Hz, 1H), 7.50-7.46 (m, 2H), 7.44 (d, J=8.2 Hz, 1H).

799: White solid, yield: 69.7%. ¹H NMR (800 MHz, acetone-d₆) δ 11.02 (br, 1H), 10.06 (br, 1H), 8.28 (s, 1H), 8.15 (s, 1H), 7.97-7.89 (m, 2H), 7.80-7.78 (m, 1H), 7.60 (t, J=7.9 Hz, 1H), 7.45 (d, J=7.9 Hz, 1H), 7.27-7.20 (m, 2H).

803: White solid, yield: 53.9%. ¹H NMR (800 MHz, acetone-d₆) δ 11.12 (s, 1H), 10.15 (s, 1H), 8.22 (s, 1H), 7.94-7.88 (m, 4H), 7.77 (d, J=8.6 Hz, 2H), 7.49 (d, J=8.6 Hz, 2H).

805: White solid, yield: 76.5%. ¹H NMR (500 MHz, acetone-d₆) δ 11.11 (br, 1H), 10.05 (br, 1H), 8.28 (s, 1H), 8.16 (s, 1H), 7.88-7.87 (m, 2H), 7.80 (d, J=8.1 Hz, 1H), 7.61 (t, J=8.1 Hz, 1H), 7.48-7.44 (m, 3H), 7.40-7.35 (m, 1H).

783:16-113-E157F98 White solid. Yield, 57.1%. ¹H NMR (500 MHz, acetone-d₆) δ 8.59 (br, 1H), 8.07 (s, 1H), 7.69-7.56 (m, 5H), 7.53-7.33 (m, 7H), 7.26 (d, J=7.7 Hz, 1H), 6.59 (br, 1H), 4.66 (d, J=5.5 Hz, 2H).

885: White solid. Yield: 37.7%. ¹H NMR (500 MHz, Acetone-d₆) δ 11.33 (br, 1H), 10.50 (br, 1H), 8.88 (d, J=1.0 Hz, 1H), 8.66 (d, J=5.8 Hz, 1H), 8.17 (s, 1H), 8.08 (d, J=5.8 Hz, 1H), 7.93-7.84 (m, 2H), 7.32-7.21 (m, 2H).

The chemical structures of compounds 804, 790, 791, 798, 803, 802, 805, 797, 799, 803, 805, 783, 788 and 885 prepared as described above are depicted in the following Table 3.1.

TABLE 3.1
Compound 804 and its Analogues

| ID. | Structure |
|---|---|
| 804 | 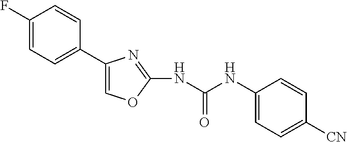 [SMILES: N#Cc1ccc(NC(=O)Nc2nc(-c3ccc(F)cc3)co2)cc1] |
| 790 | 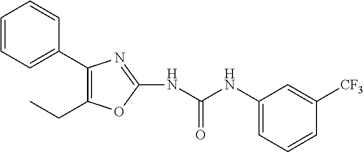 [SMILES: CCc1oc(NC(=O)Nc2cccc(C(F)(F)F)c2)nc1-c1ccccc1] |
| 791 | 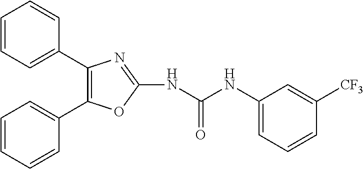 [SMILES: O=C(Nc1cccc(C(F)(F)F)c1)Nc1nc(-c2ccccc2)c(-c2ccccc2)o1] |
| 797 | 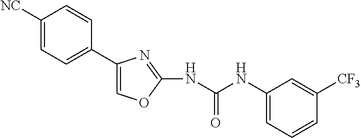 [SMILES: N#Cc1ccc(-c2coc(NC(=O)Nc3cccc(C(F)(F)F)c3)n2)cc1] |
| 798 | 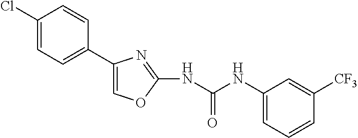 [SMILES: O=C(Nc1cccc(C(F)(F)F)c1)Nc1nc(-c2ccc(Cl)cc2)co1] |
| 799 | 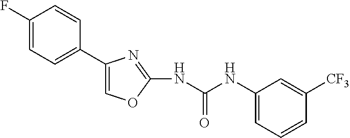 [SMILES: O=C(Nc1cccc(C(F)(F)F)c1)Nc1nc(-c2ccc(F)cc2)co1] |
| 803 | 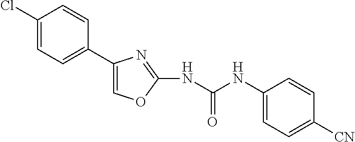 [SMILES: N#Cc1ccc(NC(=O)Nc2nc(-c3ccc(Cl)cc3)co2)cc1] |
| 805 | 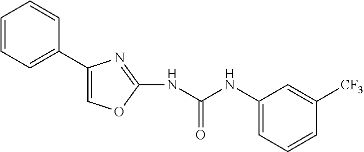 [SMILES: O=C(Nc1cccc(C(F)(F)F)c1)Nc1nc(-c2ccccc2)co1] |
| 802 | 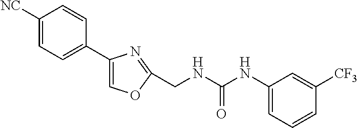 [SMILES: N#Cc1ccc(-c2coc(CNC(=O)Nc3cccc(C(F)(F)F)c3)n2)cc1] |
| 783 | 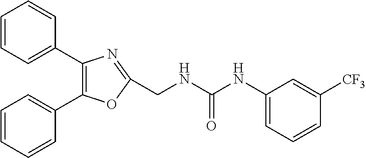 [SMILES: O=C(NCc1nc(-c2ccccc2)c(-c2ccccc2)o1)Nc1cccc(C(F)(F)F)c1] |
| 788 | 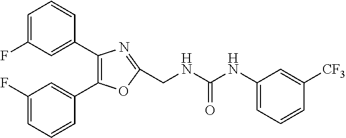 [SMILES: O=C(NCc1nc(-c2cccc(F)c2)c(-c2cccc(F)c2)o1)Nc1cccc(C(F)(F)F)c1] |
| 885 | 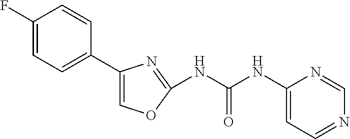 [SMILES: O=C(Nc1ccncn1)Nc1nc(-c2ccc(F)cc2)co1] |

### Compound 566 and its “Analogues of Formula (I)”

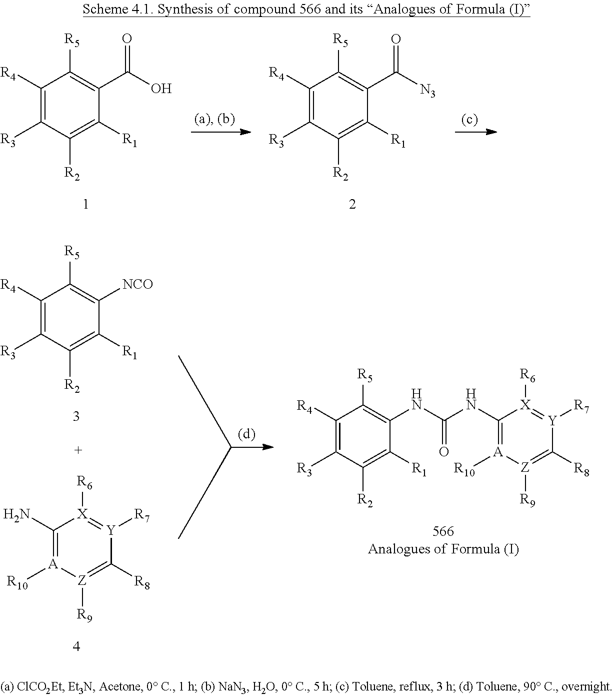

General Procedure for the Synthesis of Aryl Azid 2:

Referring to Scheme 4.1 reproduced above, to a solution of 1 (1 mmol) in dry acetone (10 mL), triethylamine (1.1 mmol) and ethyl chlorocarbamate (1.1 mmol) were added dropwise at 0° C. After the mixture was stirred at 0° C. for 1 h, sodium azide (1.1 mmol, 0.215 g) dissolved in 5 mL water was added dropwise. Stirring was continued at 00° C. for 5 h. Ice water was added. The mixture was extracted by dichloromethane (3×20 mL). The combined organic layers were washed with brine and dried over Na₂SO₄. The organic phase was concentrated under reduced pressure. Colorless oil was obtained and used in the following reaction without further purification.

General Procedure for the Synthesis of Compound 566 and its “Analogues of formula (I)”—Scheme 4.1:

A solution of aryl azide 2 (0.5 mmol) in toluene (10 mL) was heated at 120° C. for 3 h to give aryl isocyanate 3, which is not isolated and treated in situ with the respective 4 at 90° C. overnight. The solvent was cooled to room temperature and the precipitate was collected by filtration and washed with toluene.

### Characterization of Compound 566 and its “Analogues of Formula (I)”

484: White solid, mp. 239-241° C., yield: 39.9%. ¹H NMR (500 MHz, acetone-d₆) δ 10.85 (br, 1H), 9.32 (br, 1H), 8.72 (s, 1H), 8.13 (s, 2H), 7.79 (d, J=7.2 Hz, 1H), 7.65 (t, J=9.6 Hz, 1H), 7.55 (t, J=8.0 Hz, 1H), 7.39 (d, J=7.2 Hz, 1H).

486: White solid, mp. 238-240° C., yield: 7.8%. ¹H NMR (500 MHz, acetone-d₆) δ 8.86 (br, 1H), 8.74 (d, J=1.6 Hz, 2H), 8.32 (dd, J₁=2.4 Hz, J₂=2.4 Hz, 1H), 8.05 (s, 1H), 7.84 (d, J=8.8 Hz, 1H), 7.54 (t, J=8.0 Hz, 1H), 7.37 (d, J=8.0 Hz, 1H).

491: White solid, mp. 249-251° C., yield: 18.4%. ¹H NMR (500 MHz, acetone-d₆) δ) 10.77 (br, 1H), 9.39 (br, 1H), 9.10 (d, J=3.0 Hz, 1H), 8.43 (dd, J₁=2.5 Hz, J₂=3.0 Hz, 1H), 8.05 (s, 1H), 7.71 (d, J=8.0 Hz, 1H), 7.59 (t, J=8.0 Hz, 1H), 7.45 (t, J=8.0 Hz, 1H), 7.28 (d, J=7.5 Hz, 1H).

495: White solid, mp.>300° C., yield: 32.6%. ¹H NMR (500 MHz, acetone-d₆) δ M.P.>300° C. 11.39 (br, 1H), 9.52 (br, 1H), 8.78 (d, J=2.0 Hz, 1H), 8.36 (s, 2H), 8.20 (dd, J₁=2.0 Hz, J₂=2.0 Hz, 1H), 7.73 (s, 1H), 7.65 (t, J=7.0 Hz, 1H).

496: White solid, mp. 239-241° C., yield: 28.4%. ¹H NMR (500 MHz, acetone-d₆) δ 11.49 (br, 1H), 9.72 (br, 1H), 9.28 (d, J=3.0 Hz, 1H), 8.63 (dd, J₁=2.5 Hz, J₂=3.0 Hz, 1H), 8.41 (s, 2H), 7.75 (s, 1H), 7.71-7.68 (m, 1H).

498: White solid, mp. 232-234° C., yield: 41.8%. ¹H NMR (500 MHz, acetone-d₆) δ 8.88 (br, 1H), 8.81 (br, 1H), 8.78 (dd, J₁=0.5 Hz, J₂=0.5 Hz, 1H), 8.35 (dd, J₁=3.0 Hz, J₂=2.5 Hz, 1H), 7.88 (d, J=9.0 Hz, 1H), 7.80 (d, J=9.0 Hz, 2H), 7.68 (d, J=8.5 Hz, 2H).

499: White solid, mp. 255-257° C., yield: 79.1%. ¹H NMR (500 MHz, acetone-d₆) δ 10.84 (br, 1H), 9.36 (br, 1H), 8.77 (d, J=1.5 Hz, 1H), 8.18 (dd, J₁=2.5 Hz, J₂=2.0 Hz, 1H), 7.88 (d, J=7.5 Hz, 2H), 7.74-7.69 (m, 3H).

501: White solid, mp. 255-257° C., yield: 92.6%. ¹H NMR (500 MHz, acetone-d₆) δ 10.89 (br, 1H), 9.55 (br, 1H), 9.26 (s, 1H), 8.61 (dd, J₁=2.5 Hz, J₂=2.5 Hz, 1H), 7.92 (d, J=8.5 Hz, 2H), 7.80-7.76 (m, 1H), 7.72 (d, J=9.0 Hz, 2H).

506: White solid, mp. 214-216° C., yield: 49.8%. ¹H NMR (500 MHz, acetone-d₆) δ 8.91 (br, 1H), 8.67 (d, J=2.5 Hz, 1H), 8.57 (br, 1H), 8.29 (dd, J₁=1.0 Hz, J₂=1.5 Hz, 1H), 8.24 (s, 2H), 8.12-8.09 (m, 1H), 7.67 (s, 1H), 7.36-7.33 (m, 1H).

507: White solid, mp. 206-207° C., yield: 88.2%. ¹H NMR (500 MHz, acetone-d₆) δ 8.67 (d, J=1.5 Hz, 1H), 8.65 (br, 1H), 8.41 (br, 1H), 8.27 (d, J=4.0 Hz, 1H), 8.11-8.08 (m, 1H), 7.79 (d, J=8.5 Hz, 2H), 7.66 (d, J=8.5 Hz, 2H), 7.33 (dd, J₁=8.0 Hz, J₂=8.0 Hz, 1H).

565: White solid, mp. 158-160° C., yield: 7.8%. ¹H NMR (500 MHz, acetone-d₆) δ 9.55 (br, 1H), 8.58 (s, 2H), 8.37 (s, 1H), 8.29 (br, 1H), 8.24 (s, 2H), 7.69 (s, 1H).

566: White solid, mp.>300° C., yield: 26.3%. ¹H NMR (500 MHz, acetone-d₆) δ 8.81 (br, 1H), 8.56 (br, 1H), 8.35 (s, 1H), 8.07 (s, 2H), 7.90 (d, J=9.0 Hz, 1H), 7.52 (s, 1H), 7.40 (d, J=8.5 Hz, 1H).

567: White solid, mp. 216-218° C., yield: 41.8%. ¹H NMR (500 MHz, acetone-d₆) δ 8.69 (br, 1H), 8.67 (br, 1H), 8.42 (t, J=2.0 Hz, 1H), 8.17 (d, J=2.5 Hz, 1H), 8.12-8.09 (m, 2H), 7.73 (dd, J₁=1.5 Hz, J₂=1.5 Hz, 1H), 7.56 (t, J=8.0 Hz, 1H), 7.39 (d, J=7.5 Hz, 1H).

568: White solid, mp. 162-164° C., yield: 32.5%. ¹H NMR (500 MHz, acetone-d₆) δ 9.28 (d, J=4.0 Hz, 1H), 8.97 (br, 1H), 8.10 (d, J=5.0 Hz, 1H), 7.94 (s, 1H), 7.91 (br, 1H), 7.57 (dd, J₁=1.0 Hz, J₂=1.0 Hz, 1H), 7.41 (t, J=8.0 Hz, 1H), 7.34 (d, J=5.0 Hz, 1H), 7.23 (d, J=8.0 Hz, 1H).

569: White solid, mp. 206-208° C., yield: 43.7%. ¹H NMR (500 MHz, acetone-d₆) δ 8.66 (br, 1H), 8.56 (br, 1H), 8.50 (d, J=2.5 Hz, 1H), 8.08 (s, 1H), 8.06 (dd, J₁=2.5 Hz, J₂=3.0 Hz, 1H), 7.72 (d, J=8.0 Hz, 1H), 7.57-7.53 (m, 2H), 7.38 (d, J=7.5 Hz, 1H).

570: White solid, mp. 172-174° C., yield: 29.7%. ¹H NMR (500 MHz, acetone-d₆) δ 8.96 (d, J=7.0 Hz, 1H), 8.76 (br, 1H), 8.22 (d, J=4.5 Hz, 1H), 8.09 (s, 1H), 7.76 (br, 1H), 7.72 (dd, J₁=1.5 Hz, J₂=2.0 Hz, 1H), 7.54 (t, J=8.0 Hz, 1H), 7.35 (d, J=8.0 Hz, 1H), 7.22 (d, J=5.0 Hz, 1H), 2.35 (s, 3H).

571: White solid, mp. 206-208° C., yield: 77.1%. ¹H NMR (500 MHz, acetone-d₆) δ 10.68 (br, 1H), 9.14 (br, 1H), 8.88 (d, J=4.5 Hz, 1H), 8.34 (s, 1H), 8.30 (d, J=3.0 Hz, 1H), 8.18 (s, 1H), 7.83 (d, J=8.5 Hz, 1H), 7.59 (t, J=8.0 Hz, 1H), 7.42 (d, J=8.0 Hz, 1H).

572: White solid, mp. 218-219° C., yield: 39.8%. ¹H NMR (500 MHz, acetone-d₆) δ 12.07 (br, 1H), 9.16 (br, 1H), 8.79 (d, J=5.0 Hz, 1H), 8.28-8.25 (m, 2H), 8.20 (s, 1H), 7.92 (d, J=8.0 Hz, 1H), 7.72 (d, J=5.5 Hz, 1H), 7.68-7.60 (m, 4H), 7.43 (d, J=8.0 Hz, 1H).

573: White solid, mp. 213-215° C., yield: 24.5%. ¹H NMR (500 MHz, acetone-d₆) δ 8.98 (br, 1H), 8.85 (br, 1H), 8.45 (t, J=1.5 Hz, 1H), 8.24 (s, 2H), 8.20 (d, J=3.0 Hz, 1H), 8.12-8.08 (m, 1H), 7.69 (s, 1H).

575: White solid, mp. 202-204° C., yield: 56.6%. ¹H NMR (500 MHz, acetone-d₆) δ 11.19 (br, 1H), 9.29 (br, 1H), 8.74 (s, 1H), 8.20 (s, 1H), 8.14 (dd, J₁=2.5 Hz, J₂=2.5 Hz, 1H), 7.86 (d, J=8.5 Hz, 1H), 7.66 (t, J=7.5 Hz, 1H), 7.59 (t, J=8.0 Hz, 1H), 7.42 (d, J=8.0 Hz, 1H).

576: White solid, mp. 219-211° C., yield: 29.3%. ¹H NMR (500 MHz, acetone-d₆) δ 11.14 (br, 1H), 9.31 (br, 1H), 8.86 (s, 1H), 8.36 (s, 3H), 8.33 (d, J=2.5 Hz, 1H), 7.72 (s, 1H).

579: White solid, mp. 216-218° C., yield: 33.1%. ¹H NMR (500 MHz, acetone-d₆) δ 8.85 (br, 1H), 8.53 (d, J=2.5 Hz, 1H), 8.48 (br, 1H), 8.24 (s, 2H), 7.97 (dd, J₁=2.5 Hz, J₂=2.5 Hz, 1H), 7.65 (s, 1H), 7.21 (d, J=8.5 Hz, 1H), 2.47 (s, 3H).

580: White solid, mp. 164-166° C., yield: 47.1%. ¹H NMR (500 MHz, acetone-d₆) δ 11.54 (br, 1H), 9.62 (br, 1H), 9.11 (d, J=5.0 Hz, 1H), 8.19 (s, 1H), 7.85 (d, J=8.0 Hz, 1H), 7.62 (d, J=4.5 Hz, 2H), 7.45 (d, J=7.5 Hz, 1H).

584: White solid, mp. 210-212° C., yield: 10.8%. ¹H NMR (500 MHz, acetone-d₆) δ 11.93 (br, 1H), 9.84 (br, 1H), 9.12 (d, J=5.5 Hz, 1H), 8.38 (s, 2H), 7.76 (s, 1H), 7.65 (d, J=5.5 Hz, 1H).

739: White solid, yield: 84.6%. ¹H NMR (500 MHz, acetone-d₆) δ 8.63 (br, 1H), 8.48 (br, 1H), 8.35-8.29 (m, 1H), 8.26-8.18 (m, 1H), 8.09 (s, 1H), 7.74-7.69 (m, 1H), 7.55 (t, J=8.0 Hz, 1H), 7.37 (d, J=7.8 Hz, 1H), 7.07 (dd, J=8.8, 3.4 Hz, 1H).

740: White solid, yield: 75.6%. ¹H NMR (500 MHz, acetone-d₆) δ 8.79 (br, 1H), 8.70 (br, 1H), 8.49 (d, J=2.8 Hz, 1H), 8.13-8.11 (m, 1H), 8.07 (s, 1H), 7.70 (d, J=8.0 Hz, 1H), 7.51 (t, J=8.0 Hz, 1H), 7.41-7.31 (m, 2H).

741: White solid, yield: 84.6%. ¹H NMR (500 MHz, acetone-d₆) δ 8.79 (br, 1H), 8.70 (br, 1H), 8.49 (d, J=2.8 Hz, 1H), 8.13-8.11 (m, 1H), 8.07 (s, 1H), 7.70 (d, J=8.1 Hz, 1H), 7.51 (t, J=8.0 Hz, 1H), 7.41-7.31 (m, 2H).

754: White solid. Yield, 83.2%. ¹H NMR (500 MHz, acetone-d₆) δ 8.90 (br, 1H), 8.60 (br, 1H), 8.29 (s, 1H), 8.23-8.15 (m, 3H), 7.64 (s, 1H), 7.07-7.04 (m, 1H).

755: White solid. Yield, 88.4%. ¹H NMR (500 MHz, acetone-d₆) δ 8.93 (br, 1H), 8.69 (br, 1H), 8.49 (d, J=2.8 Hz, 1H), 8.20 (s, 2H), 8.13 (dd, J=8.7, 2.8 Hz, 1H), 7.65 (s, 1H), 7.40 (d, J=8.7 Hz, 1H).

758: White solid, yield: 65.3%. ¹H NMR (500 MHz, acetone-d₆) δ 10.50 (br, 1H), 8.67 (s, 2H), 8.51 (s, 2H), 8.35 (s, 1H), 7.84 (s, 1H).

763: White solid, yield: 63.2%. ¹H NMR (500 MHz, acetone-d₆) δ 9.13 (br, 1H), 8.21 (s, 2H), 8.17 (d, J=4.9 Hz, 1H), 7.90 (br, 1H), 7.62 (s, 1H), 7.33 (d, J=4.9 Hz, 1H).

764: White solid, yield: 54.5%. ¹H NMR (500 MHz, acetone-d₆) δ 9.07 (br, 1H), 8.42-8.40 (m, 1H), 8.20 (s, 2H), 7.93 (s, 1H), 7.62 (br, 1H), 6.99 (d, J=2.5 Hz, 1H), 2.39 (s, 3H).

773: White solid, yield: 88.5%. ¹H NMR (500 MHz, acetone-d₆) δ 8.62 (br, 1H), 8.53 (br, 1H), 8.47 (d, J=2.6 Hz, 1H), 8.03 (s, 1H), 8.01 (d, J=2.6 Hz, 1H), 7.78-7.75 (m, 1H), 7.53-7.49 (m, 2H), 7.41 (d, J=7.6 Hz, 1H).

522: White solid, mp. 299-301° C., yield: 31.9%. ¹H NMR (500 MHz, acetone-d₆) δ 10.18 (br, 1H), 8.34 (br, 1H), 8.15 (s, 1H), 8.00 (br, 1H), 7.82 (d, J=2.0 Hz, 1H), 7.71 (d, J=8.0 Hz, 1H), 7.51 (t, J=7.5 Hz, 1H), 7.38 (d, J=9.0 Hz, 1H), 7.34 (t, J=2.5 Hz, 1H), 7.30 (d, J=8.0 Hz, 1H), 7.21 (dd, J₁=2.0 Hz, J₂=2.0 Hz, 1H), 6.45 (d, J=2.0 Hz, 1H).

530: White solid, mp. 192-194° C., yield: 9.6%. ¹H NMR (500 MHz, acetone-d₆) δ 10.22 (br, 1H), 8.69 (br, 1H), 8.26 (s, 2H), 8.17 (br, 1H), 7.82 (s, 1H), 7.59 (s, 1H), 7.39 (d, J=9.0 Hz, 1H), 7.36 (s, 1H), 7.22 (d, J=8.5 Hz, 1H), 6.47 (s, 1H).

574: White solid, mp. 200-202° C., yield: 73.6%. ¹H NMR (500 MHz, acetone-d₆) δ 11.06 (br, 1H), 9.24 (br, 1H), 8.89 (s, 1H), 8.60 (d, J=6.0 Hz, 1H), 8.18 (s, 1H), 7.84 (d, J=8.0 Hz, 1H), 7.60 (t, J=8.0 Hz, 1H), 7.49-7.43 (m, 2H).

578: White solid, mp. 215-217° C., yield: 27.1%. ¹H NMR (500 MHz, acetone-d₆) δ 11.56 (br, 1H), 9.40 (br, 1H), 8.90 (s, 1H), 8.62 (d, J=5.5 Hz, 1H), 8.37 (s, 2H), 7.74 (s, 1H), 7.45 (t, J=6.0 Hz, 1H).

737: White solid, yield: 38.6%. ¹H NMR (500 MHz, acetone-d₆) δ 8.82 (br, 1H), 8.77 (d, J=2.4 Hz, 1H), 8.65 (s, 1H), 8.37 (dd, J=8.6, 2.4 Hz, 1H), 8.23 (d, J=8.2 Hz, 1H), 8.09 (s, 1H), 7.85 (d, J=8.6 Hz, 1H), 7.64 (d, J=7.8 Hz, 1H), 7.40 (t, J=7.6 Hz, 1H), 7.30 (t, J=7.4 Hz, 1H), 4.51 (q, J=7.1 Hz, 2H), 1.47 (t, J=7.1 Hz, 3H).

738: White solid, yield: 51.6%. ¹H NMR (500 MHz, acetone-d₆) δ 9.07 (s, 1H), 8.37 (s, 1H), 8.01 (s, 1H), 7.28-7.22 (m, 1H), 7.02-6.99 (m, 3H), 6.95 (d, J=8.2 Hz, 1H), 6.92-6.82 (m, 3H), 6.56 (t, J=7.4 Hz, 1H).

744: White solid, yield: 38.6%. ¹H NMR (500 MHz, acetone-d₆) δ 10.08 (br, 1H), 8.76 (d, J=2.6 Hz, 1H), 8.68 (br, 1H), 8.35 (dd, J=8.6, 2.6 Hz, 1H), 8.19 (br, 1H), 7.81 (d, J=8.6 Hz, 1H), 7.65 (s, 1H), 7.55 (d, J=8.0 Hz, 1H), 7.42 (d, J=8.2 Hz, 1H), 7.20-7.11 (m, 1H), 7.08-7.01 (m, 1H).

753: White solid. Yield, 86.4%. ¹H NMR (500 MHz, acetone-d₆) δ 8.75 (d, J=2.2 Hz, 1H), 8.69 (br, 1H), 8.34 (dd, J=8.6, 2.2 Hz, 1H), 8.24 (br, 1H), 7.81 (d, J=8.6 Hz, 1H), 7.56-7.54 (m, 2H), 7.40 (d, J=8.6 Hz, 1H), 7.20 (t, J=7.6 Hz, 1H), 7.05 (t, J=7.6 Hz, 1H), 3.83 (s, 3H).

The chemical structures of compounds 484, 486, 491, 495, 496, 498, 499, 501, 506, 507, 565, 566, 567, 568, 569, 570, 571, 572, 573, 575, 576, 579, 580, 584, 739, 740, 741, 754, 755, 758, 763, 764, 773, 522, 530, 574, 578, 737, 738, 744 and 753 prepared as described above are provided in Table 4.1 herein below.

### The 566 “Analogues of Formula (II)”

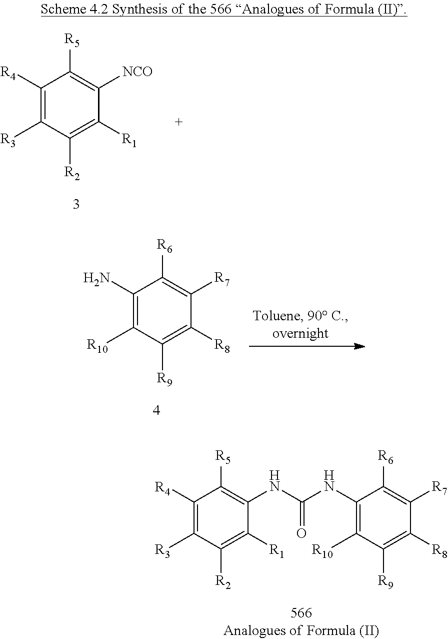

General Procedure for the Synthesis of the 566 “Analogues of Formula (II)”-Scheme 4.2:

A mixture of aryl isocyanate 3 and amine 4 in toluene was heated at 90° C. overnight. The solvent was cooled to room temperature and the precipitate was collected by filtration and washed with toluene.

### Characterization of the 566 “Analogues of Formula (II)”

442: White solid, mp. 198-200° C., yield: 23.4%. ¹H NMR (500 MHz, acetone-d₆) δ 9.17 (br, 1H), 8.82 (br, 1H), 8.28 (d, J=2.5 Hz, 1H), 8.15 (d, J=9.0 Hz, 1H), 8.04 (s, 1H), 8.02 (dd, J₁=2.5 Hz, J₂=2.5 Hz, 1H), 7.77-7.75 (m, 1H), 7.58 (t, J=8.0 Hz, 1H), 7.41 (d, J=7.5 Hz, 1H). LHMS-ESI, m/z [M+H]⁺ 394.06.

465: White solid, mp. 286-289° C., yield: 47.8%. ¹H NMR (500 MHz, acetone-d₆): δ 9.29 (br, 1H), 9.25 (br, 1H), 8.29-8.27 (m, 2H), 8.17 (d, J=9.0 Hz, 1H), 8.06-7.99 (m, 3H). LHMS-ESI, m/z [M+H]⁺ 419.06.

467: White solid, mp. 235-237° C., yield: 47.8%. ¹H NMR (500 MHz, acetone-d₆): δ 9.05 (br, 1H), 8.76 (br, 1H), 8.29 (d, J=1.5 Hz, 1H), 8.08 (s, 1H), 7.99-7.96 (m, 2H), 7.75 (d, J=8.0 Hz, 1H), 7.59 (t, J=8.0 Hz, 1H), 7.43-7.41 (m, 1H).

492: White solid, mp. 285-287° C., yield: 10.0%. ¹H NMR (800 MHz, acetone-d₆): δ 8.83 (br, 1H), 8.66 (br, 1H), 8.03 (s, 1H), 8.02 (d, J=1.6 Hz, 1H), 7.75 (d, J=9.6 Hz, 1H), 7.69 (d, J=8.0 Hz, 1H), 7.55 (dd, J₁=1.6 Hz, J₂=1.6 Hz, 1H), 7.53 (t, J=8.0 Hz, 1H), 7.36 (d, J=8.0 Hz, 1H).

494: White solid, mp. 279-281° C., yield: 12.7%. ¹H NMR (500 MHz, acetone-d₆): δ 9.22 (br, 1H), 9.08 (br, 1H), 8.29 (d, J=2.0 Hz, 1H), 8.25 (s, 2H), 8.03-7.98 (m, 2H), 7.72 (1H).

500: White solid, mp. 291-294° C., yield: 14.7%. ¹H NMR (500 MHz, acetone-d₆): δ 9.05 (br, 1H), 9.01 (br, 1H), 8.23 (s, 2H), 8.06 (d, J=2.5 Hz, 1H), 7.81 (d, J=8.5 Hz, 1H), 7.71 (s, 1H), 7.63 (dd, J₁=2.0 Hz, J₂=2.5 Hz, 1H).

502: White solid, mp. 257-259° C., yield: 48.3%. ¹H NMR (500 MHz, acetone-d₆): δ 8.87 (br, 1H), 8.74 (br, 1H), 8.06 (d, J=2.0 Hz, 1H), 7.80-7.77 (m, 3H), 7.68 (d, J=8.5 Hz, 2H), 7.60 (dd, J₁=2.5 Hz, J₂=2.5 Hz, 1H).

509: White solid, mp. 228-230° C., yield: 14.5%. ¹H NMR (500 MHz, acetone-d₆): δ 9.04 (br, 1H), 8.81 (br, 1H), 8.30 (d, J=1.5 Hz, 1H), 7.97 (m, 2H), 7.81-7.95 (d, J=8.5 Hz, 2H), 7.69 (d, J=9.0 Hz, 2H).

646: White solid, yield: 83.2%. ¹H NMR (500 MHz, acetone-d₆) δ 8.70 (br, 1H), 8.33 (br, 1H), 8.03 (d, J=2.1 Hz, 1H), 7.73 (d, J=8.6 Hz, 1H), 7.53 (dd, J=8.6, 2.1 Hz, 1H), 7.27 (t, J=2.1 Hz, 1H), 7.19 (t, J=8.2 Hz, 1H), 7.01-6.99 (m, 1H), 6.63-6.60 (m, 1H), 3.77 (s, 3H).

647: White solid, yield: 87.3%. ¹H NMR (500 MHz, acetone-d₆) δ 8.77 (br, 1H), 8.50 (br, 1H), 8.01 (d, J=2.1 Hz, 1H), 7.77-7.75 (m, 1H), 7.74 (d, J=8.6 Hz, 1H), 7.54 (dd, J=8.6, 2.1 Hz, 1H), 7.37-7.35 (m, 1H), 7.30 (t, J=8.0 Hz, 1H), 7.07-7.04 (m, 1H).

680: White solid, yield: 77.9%. ¹H NMR (500 MHz, acetone-d₆) δ 8.72 (br, s, 1H), 8.34 (br, s, 1H), 8.04 (d, J=2.0 Hz, 1H), 7.74 (t, J=6.8 Hz, 1H), 7.55-7.52 (m, 3H), 7.32-7.25 (m, 2H), 7.06-7.03 (m, 1H).

701: White solid, yield: 87.3%. ¹H NMR (500 MHz, acetone-d₆) δ 8.96 (br, 1H), 8.57 (br, 1H), 8.25 (d, J=1.9 Hz, 1H), 7.95 (d, J=8.6 Hz, 1H), 7.91 (dd, J=8.6, 1.9 Hz, 1H), 7.77 (s, 1H), 7.43-7.35 (m, 1H), 7.31 (t, J=8.1 Hz, 1H), 7.08-7.06 (m, 1H).

702: White solid, yield: 84.4%. ¹H NMR (500 MHz, acetone-d₆) δ 8.89 (br, 1H), 8.38 (br, 1H), 8.27 (d, J=1.8 Hz, 1H), 7.94 (d, J=8.6 Hz, 1H), 7.90 (dd, J=8.6, 1.8 Hz, 1H), 7.54 (d, J=8.3 Hz, 2H), 7.31 (t, J=7.9 Hz, 2H), 7.05 (t, J=7.4 Hz, 1H).

703: White solid, yield: 86.5%. ¹H NMR (500 MHz, acetone-d₆) δ 8.86 (br, 1H), 8.30 (br, 1H), 8.27 (d, J=2.1 Hz, 1H), 7.93 (d, J=8.6 Hz, 1H), 7.88 (dd, J=8.6, 2.1 Hz, 1H), 7.38 (s, 1H), 7.31 (d, J=8.3 Hz, 1H), 7.18 (t, J=7.8 Hz, 1H), 6.87 (d, J=7.5 Hz, 1H), 2.30 (s, 3H).

704: White solid, yield: 88.5%. ¹H NMR (500 MHz, acetone-d₆) δ 8.94 (br, 1H), 8.60 (br, 1H), 8.25 (d, J=2.0 Hz, 1H), 7.95 (d, J=8.6 Hz, 1H), 7.91 (dd, J=8.6, 2.0 Hz, 1H), 7.56-7.53 (m, 1H), 7.37-7.28 (m, 1H), 7.21-7.20 (m, 1H), 6.82-6.78 (m, 1H).

705: White solid, yield: 89.7%. ¹H NMR (500 MHz, acetone-d₆) δ 8.87 (br, 1H), 8.39 (br, 1H), 8.25 (d, J=2.0 Hz, 1H), 7.94 (d, J=8.6 Hz, 1H), 7.90 (dd, J=8.6, 2.0 Hz, 1H), 7.28 (s, 1H), 7.20 (t, J=8.2 Hz, 1H), 7.02-7.01 (m, 1H), 6.63-7.61 (m, 1H).

706: White solid, yield: 86.5%. ¹H NMR (500 MHz, acetone-d₆) δ 9.62 (br, 1H), 8.58-8.56 (m, 1H), 8.24 (d, J=1.9 Hz, 1H), 8.12 (br, 1H), 7.98 (t, J=7.8 Hz, 2H), 7.92 (dd, J=8.6, 1.9 Hz, 1H), 7.68 (d, J=8.2 Hz, 1H).

736: White solid, yield: 90%. ¹H NMR (500 MHz, acetone-d₆) δ 8.37 (br, 1H), 8.06 (s, 1H), 8.00 (br, 1H), 7.68-7.65 (m, 2H), 7.48 (t, J=8.0 Hz, 1H), 7.40 (dd, J=8.7, 2.3 Hz, 1H), 7.29 (d, J=7.7 Hz, 1H), 6.88 (d, J=8.7 Hz, 1H), 4.93 (br, 2H).

745: White solid, yield: 83.4%. ¹H NMR (500 MHz, acetone-d₆) δ 8.26 (br, 1H), 8.08 (s, 1H), 7.77 (br, 1H), 7.67-7.62 (m, 1H), 7.48-7.44 (m, 1H), 7.26 (d, J=7.7 Hz, 1H), 7.21-7.16 (m, 2H), 6.65-6.60 (m, 2H), 4.45 (br, 2H).

772: White solid, yield: 79.5%. ¹H NMR (500 MHz, acetone-d₆) δ 8.87 (br, 1H), 8.68 (br, 1H), 8.03 (s, 2H), 7.76 (d, J=8.6 Hz, 2H), 7.58-7.49 (m, 2H), 7.44-7.39 (m, 1H).

774: White solid, yield: 63.1%. ¹H NMR (800 MHz, acetone-d₆) δ 8.62 (br, 1H), 8.04 (br, 1H), 7.77 (m, 2H), 7.51 (t, J=8.0 Hz, 3H), 7.41 (d, J=8.0 Hz, 2H).

792: White solid. Yield, 43.5%. ¹H NMR (500 MHz, acetone-d₆) δ 9.04 (br, 1H), 8.20 (s, 1H), 8.12-7.97 (m, 2H), 7.89-7.78 (m, 1H), 7.76-7.66 (m, 2H), 7.47-7.35 (m, 1H).

829: White solid. Yield: 57.5%. ¹H NMR (500 MHz, Acetone-d₆) δ 9.10 (br, 1H), 8.74 (br, 1H), 8.25 (d, J=2.3 Hz, 1H), 8.13 (d, J=9.0 Hz, 1H), 8.04-8.02 (m, 1H), 7.99 (dd, J=9.0, 2.4 Hz, 1H), 7.84-7.77 (m, 1H), 7.38 (t, J=9.7 Hz, 1H).

887: White solid. Yield: 77.8%. ¹H NMR (500 MHz, Acetone-de) δ 9.13 (br, 1H), 8.77 (br, 1H), 8.25-8.24 (m, 1H), 8.12 (d, J=9.0 Hz, 1H), 8.03-7.95 (m, 1H), 7.51 (s, 1H), 7.43 (t, J=2.0 Hz, 1H), 6.91 (s, 1H), 3.87 (d, J=5.8 Hz, 3H).

The chemical structures of compounds 442, 465, 467, 492, 494, 500, 502, 509, 646, 647, 680, 701, 702, 703, 704, 705, 706, 736, 745, 772, 774, 792, 829 and 887 prepared as described above are outlined in Table 4.1 below.

The bis-urea Compounds

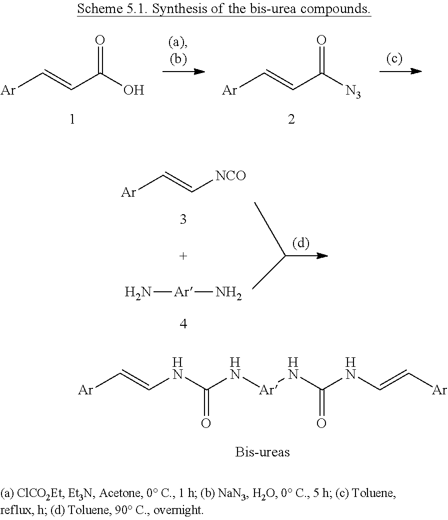

General Procedure for the Synthesis of aryl azid 2:

Referring to Scheme 5.1 reproduced above, to a solution of 1 (1 mmol) in dry acetone (10 mL), triethylamine (1.1 mmol) and ethyl chlorocarbamate (1.1 mmol) were added dropwise at 0° C. After stirring at 0° C. for 1 h, sodium azide (1.1 mmol, 0.215 g) dissolved in 5 mL water was added dropwise. Stirring was continued at 0° C. for 5 h. Ice water was added. The mixture was extracted by dichloromethane (3×20 mL). The combined organic layers were washed with brine and dried over Na₂SO₄. The organic phase was concentrated under reduced pressure. Colorless oil was obtained and used in the following reaction without further purification.

General Procedure for the Synthesis of the bis-ureas of the Invention—Scheme 5.1:

A solution of aryl azide 2 (0.5 mmol) in toluene (10 mL) was heated at 120° C. for 3 h to give aryl isocyanate 3, which is not isolated and treated in situ with the respective diamine 4 at 90° C. overnight. After cooling to room temperature, white solid was precipitated, which was collected by filtration and washed with toluene.

Characterization of the bis-urea Compounds

439: White solid, mp.>300° C., yield: 66.3%. ¹H NMR (500 MHz, DMSO-d₆) δ 8.81 (d, J=11.0 Hz, 2H), 8.63 (b, 2H), 7.58 (d, J=8.0 Hz, 2H), 7.55 (s, 2H), 7.49-7.41 (m, 4H), 7.35 (d, J=9.0 Hz, 2H), 7.31 (s, 4H), 6.02 (d, J=14.5 Hz, 2H).

440: White solid, mp.=214-216° C., yield: 78.1%. ¹H NMR (500 MHz, acetone-d₆) δ 8.36 (d, J=11.5 Hz, 2H), 8.28 (b, 2H), 7.82 (d, J=1.5 Hz, 1H), 7.71-7.63 (m, 6H), 7.52 (t, J=8.0 Hz, 2H), 7.46 (d, J=7.5 Hz, 2H), 7.26-7.19 (m, 3H), 6.16 (d, J=14.5 Hz, 2H).

451: White solid, mp.=162-165° C., yield: 82.7%. ¹H NMR (500 MHz, acetone-d₆) δ 8.78 (d, J=10.5 Hz, 2H), 8.04 (br, 2H), 7.69-7.62 (m, 8H), 7.52 (t, J=7.5 Hz, 2H), 7.46 (d, J=8.0 Hz, 2H), 7.19-7.16 (m, 2H), 6.14 (d, J=14.5 Hz, 2H).

452: White solid, mp.>300° C., yield: 58.5%. ¹H NMR (500 MHz, acetone-d₆) 8.26 (br. d, J=10.5 Hz, 2H), 8.21 (br. d, J=8.5 Hz, 2H), 8.16 (s, 2H), 7.78 (d, J=8.0 Hz, 2H), 7.66-7.61 (m, 4H), 7.39 (s, 3H) 7.39-7.30 (m, 5H), 6.18 (d, J=14.5 Hz, 2H), 1.70 (s, 18H).

455: White solid, mp.>300° C., yield: 53.0%. ¹H NMR (500 MHz, DMSO-d₆) δ 8.72 (d, J=10.5 Hz, 2H), 8.64 (s, 2H), 7.41-7.39 (m, 2H), 7.37 (s, 4H), 7.31 (d, J=7.5 Hz, 4H), 7.27 (t, J=8.0 Hz, 4H), 7.27 (t, J=7.5 Hz, 2H), 5.99 (d, J=14.5 Hz, 2H).

457: solid, mp.>300° C., yield: 69.1%. ¹H NMR (500 MHz, DMSO-d₆) δ 8.48 (br. d, J=10.5 Hz, 1H), 8.18 (s, 1H), 7.35 (s, 2H), 7.25-7.19 (m, 2H), 7.05 (d, J=8.0 Hz, 2H), 6.95 (d, J=8.5 Hz, 2H), 6.82-6.79 (m, 2H), 6.74-6.70 (m, 2H), 6.50 (t, J=3.5 Hz, 2H), 5.95 (d, J=5.0 Hz, 4H), 5.92-5.85 (m, 2H).

458: White solid, mp.>300° C., yield: 53.3%. ¹H NMR (500 MHz, DMSO-d₆) δ 8.76 (s, 1H), 8.33 (s, 1H), 8.09 (d, J=12.5 Hz, 2H), 7.93-7.89 (m, 2H), 7.82-7.77 (m, 2H), 7.62-7.50 (m, 4H), 7.39 (s, 2H), 7.08 (d, J=8.5 Hz, 2H), 6.52 (d, J=8.5 Hz, 2H), 6.10 (dd, J₁=15.0 Hz, J₂=15.0 Hz, 2H).

466: White solid, mp.>300° C., yield: 46.4%. ¹H NMR (500 MHz, acetone-d₆) δ 8.86 (d, J=10.5 Hz, 2H), 8.67 (br, 2H), 7.53-7.51 (m, 5H), 7.49-7.45 (m, 5H), 7.32 (s, 4H), 6.01 (d, J=14.5 Hz, 2H).

532: White solid, mp. 228-230° C., yield: 59.4%. ¹H NMR (500 MHz, DMSO-d₆) δ 11.38 (br, 1H), 10.24 (br, 1H), 8.13 (s, 1H), 8.04 (s, 1H), 7.77-7.74 (m, 3H), 7.64 (s, 2H), 7.34 (s, 1H), 7.27 (d, J=11.5 Hz, 1H), 6.24 (d, J=12.0 Hz, 1H), 5.77 (d, J=8.0 Hz, 1H), 5.41 (d, J=13.5 Hz, 1H).

The chemical structures of compounds 439, 440, 451, 452, 455, 457, 458, 466 and 532 prepared as described above are outlined in Table 4.1 below.

MTT assays: LNCaP, 22Rv1, Du145, H1975, A549, MB231 and MCF-7 cells are maintained in RPMI 1640 supplemented with 10% FBS. Cells were seeded at a density of 6-7×10³ cells per well in 96-well plates. After overnight incubation, cells in fresh RPMI 1640 supplemented with 10% FBS were exposed to DMSO vehicle control or test compounds at designated concentrations for 72 h. Viable cells were evaluated by MTT assays. Experiments were performed in triplicate and repeated at least twice. The results are outlined in the tables below.

TABLE 2.1
Compound 562 and its “Analogues of Formula (I)”.

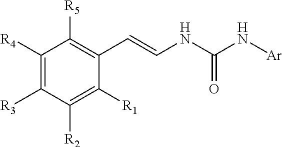

| | Substituents | | | | | | | | | | | |
|---|---|---|---|---|---|---|---|---|---|---|---|---|
| | | | | Ar | | | | | | Cytotoxicity (IC_{50,} (μM) | | |
| ID | R₁ | R₂ | R₄ | | R₆ | R₇ | R₈ | R₉ | R₁₀ | LNCaP | 22Rv1 | DU145 |
| 480 | CF₃ | | CF₃ | B | | | CN | | | | 1.8 | 1.7 |
| 481 | CF₃ | | CF₃ | A | | | CN | | | | 2.6 | 0.8 |
| 482 | | CF₃ | CF₃ | B | | | CN | | | | 1.7 | 2.3 |
| 483 | | CF₃ | CF₃ | A | | | CN | | | | 2.3 | 2.0 |
| 487 | CF₃ | | CF₃ | A | | | NO₂ | | | | | |
| 489 | | CF₃ | CF₃ | B | | | NO₂ | | | | | |
| 503 | | CF₃ | CF₃ | B | | | | | | 7.5 | 11.3 | 9.5 |
| 504 | CF₃ | | CF₃ | B | | | | | | | | |
| 510 | | | CF₃ | B | | | CN | | | 3.3 | 6.6 | 5.7 |
| 511 | | | CF₃ | A | | | CN | | | | | |
| 512 | | | | B | | | CN | | | | | |
| 527 | | CF₃ | CF₃ | A | | | | | Br | 5.3 | 7.6 | 6.8 |
| 528 | | CF₃ | CF₃ | D | | | | | | 0.12 | <1 | <1 |
| 531 | | CF₃ | CF₃ | E | | | | | | <1 | I.A. | <1 |
| 533 | | CF₃ | CF₃ | B | Cl | | | | | 2.0 | 10.9 | 6.5 |
| 535 | | CF₃ | CF₃ | B | | F | | | | 2.6 | 3.1 | 3.0 |
| 536 | | CF₃ | CF₃ | B | | | CH₃ | | | | | |
| 537 | | CF₃ | CF₃ | B | | | | | CH₃ | | | |
| 538 | | CF₃ | CF₃ | E | | CF₃ | | | | <1 | | 1.2 |
| 539 | | CF₃ | CF₃ | B | | Br | | | | | | |
| 540 | | CF₃ | CF₃ | B | | | Br | | | | | |
| 541 | | CF₃ | CF₃ | B | CH₃ | | | | | | | |
| 543 | | CF₃ | CF₃ | E | | Ph | | | | | | |
| 546 | | CF₃ | CF₃ | B | | CH₃ | | | | | | |
| 548 | | CF₃ | CF₃ | C | | | CF₃ | | | | | |
| 549 | | CF₃ | CF₃ | C | | CF₃ | | | | | | |
| 550 | | | CF₃ | B | | F | | | | | | |
| 551 | | | CF₃ | B | Cl | | | | | | | |
| 552 | | | CF₃ | B | | Br | | | | | | |
| 553 | | | CF₃ | B | | | Br | | | | | |
| 554 | | | CF₃ | B | | | CH₃ | | | | | |
| 555 | | | CF₃ | B | CH₃ | | | | | | | |
| 556 | | | CF₃ | B | | CH₃ | | | | | | |
| 557 | | | CF₃ | B | | | | | CH₃ | | | |
| 558 | | | CF₃ | D | | | | | | 0.06 | 0.10 | |
| 559 | | | CF₃ | E | | | | | | | | |
| 560 | | | CF₃ | E | | CF₃ | | | | | | |
| 561 | | | CF₃ | E | | Ph | | | | | | |
| 564 | | | CF₃ | C | | | CF₃ | | | | | |
| 583 | CF₃ | | CF₃ | D | | | | | | | | |

| | | |
|---|---|---|
| 542 | 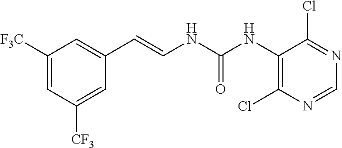 [SMILES: O=C(N/C=C/c1cc(C(F)(F)F)cc(C(F)(F)F)c1)Nc1c(Cl)ncnc1Cl] | |
| 544 | 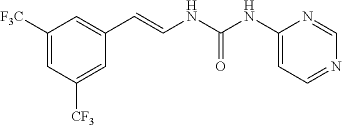 [SMILES: O=C(N/C=C/c1cc(C(F)(F)F)cc(C(F)(F)F)c1)Nc1ccncn1] | |
| 545 | 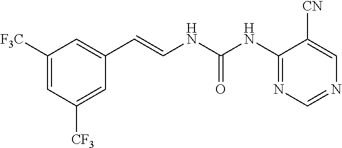 [SMILES: N#Cc1cncnc1NC(=O)N/C=C/c1cc(C(F)(F)F)cc(C(F)(F)F)c1] | |
| 562 | 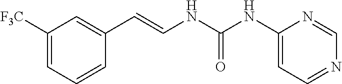 [SMILES: O=C(N/C=C/c1cccc(C(F)(F)F)c1)Nc1ccncn1] | 4.4 |
| 766 | 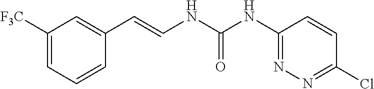 [SMILES: O=C(N/C=C/c1cccc(C(F)(F)F)c1)Nc1ccc(Cl)nn1] | |

Note:
I.A. = Inactive; Cytotoxicity was evaluated by MTT assays.
R₃ = R₅ = H

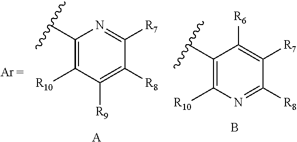

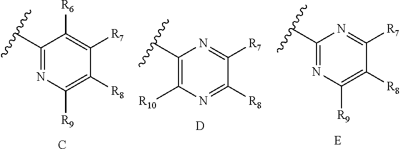

TABLE 2.2
Compound 562 and its “Analogues of Formula (II)”.

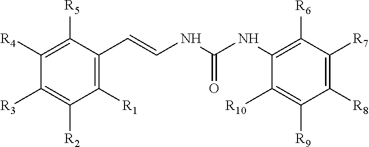

| | Substituents | | | | | | | | | | Cytotoxicity (IC_{50,} (μM) | | |
|---|---|---|---|---|---|---|---|---|---|---|---|---|---|
| ID | R₁ | R₂ | R₃ | R₄ | R₅ | R₆ | R₇ | R₈ | R₉ | R₁₀ | LNCaP | 22Rv1 | DU145 |
| 403 | | | | CF₃ | | | CF₃ | | | | 12.3 | | >20 |
| 404 | | | | | CF₃ | | CF₃ | NO₂ | | | 9.3 | | 16.4 |
| 405 | | | | | CF₃ | | | CN | | | 3.4 | | 8.3 |
| 406 | | | | | CF₃ | | CF₃ | | | | 14.4 | I.A. | I.A. |
| 407 | | | CF₃ | | | | CF₃ | NO₂ | | | 9.6 | | 15.3 |
| 408 | | | CF₃ | | | | CF₃ | | | | 15.6 | | I.A. |
| 409 | | | | CH₃O | | | CF₃ | NO₂ | | | I.A. | I.A. | I.A. |
| 410 | | | | CF₃ | | | CF₃ | CN | | | 4.4 | | 10.4 |
| 411 | | | | CH₃O | | | CF₃ | | | | I.A. | I.A. | I.A. |
| 412 | | | | F | | | CF₃ | NO₂ | | | 18.8 | >20 | I.A. |
| 413 | | | | CF₃ | | | | | | | >20 | I.A. | I.A. |
| 414 | | | | F | | | CF₃ | | | | 3.8 | 8.2 | 7.8 |
| 415 | | | | | | | CF₃ | | | | 6.6 | 11 | 14 |
| 416 | | | | CF₃ | | | | CN | | | 1.6 | 3.6 | 2.6 |
| 417 | | | | CF₃ | | | CH₃O | | | | 14.6 | 10.8 | 12.2 |
| 421 | | | CF₃ | | | | CF₃ | CN | | | 1.4 | 7.2 | 3.4 |
| 429 | CF₃ | | | | | | CH₃O | | | | 9.1 | 16.4 | 14.9 |
| 430 | | | | CH₃O | | | CH₃O | | | | >20 | >20 | I.A. |
| 433 | | | | F | | | CF₃ | CN | | | 1.9 | 3.2 | 3.9 |
| 435 | | | | CF₃ | | | | N(CH₃)₂ | | | 13.1 | >20 | 20 |
| 436 | CF₃ | | | CF₃ | | | CF₃ | NO₂ | | | 0.5 | 0.8 | 0.7 |
| 437 | | | | CF₃O | | | CF₃ | NO₂ | | | 1.6 | 2.7 | 3.0 |
| 438 | | CF₃ | | CF₃ | | | CF₃ | NO₂ | | | 0.5 | 0.6 | 0.9 |
| 441 | | | | CF₃ | | CH₃O | | NO₂ | | | 4.8 | 8.3 | 6.7 |
| 445 | | | | CF₃ | | CH₃ | | | | | 4.1 | 5.2 | 4.8 |
| 446 | | | | CF₃ | | | | 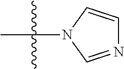 [SMILES: *n1ccnc1] | | | 11.1 | 18.4 | >20 |
| 449 | | | | CF₃ | | | | NO₂ | | | 2.1 | 5.3 | 3.9 |
| 456 | | | | NO₂ | | | CF₃ | NO₂ | | | 2.0 | 4.1 | 5.2 |
| 462 | | | CF₃ | | | | | NH₂ | | | 3.5 | 6.3 | 3.2 |
| 463 | | CF₃ | | CF₃ | | | CF₃ | CN | | | 1.2 | 1.0 | 1.2 |
| 464 | CF₃ | | | CF₃ | | | CF₃ | CN | | | 0.4 | 0.7 | 0.4 |
| 468 | CF₃ | | | CF₃ | | | | CN | | | 0.9 | 1.9 | 1.5 |
| 469 | | CF₃ | | CF₃ | | | | CN | | | 1.0 | 1.9 | 1.7 |
| 472 | CF₃ | | | CF₃ | | CH₃O | | NO₂ | | | | | |
| 473 | CF₃ | | | CF₃ | | | | NO₂ | | | | | |
| 474 | CF₃ | | | CF₃ | | CH₃ | | NO₂ | | | | | |
| 488 | CF₃ | | | CF₃ | | | Cl | CN | | | | | |
| 490 | | CF₃ | | CF₃ | | | Cl | CN | | | | | |
| 723 | | CF₃ | | CF₃ | | | | N(CH₃)₂ | | | | | |

TABLE 2.3
The 562 “Analogues of Formula (III)”.

| | | Cytotoxicity (IC50 μM) | | |
|---|---|---|---|---|
| ID | Structures | LNCaP | 22Rv1 | DU145 |
| 418 | 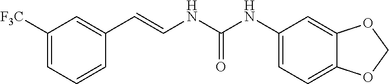 [SMILES: O=C(N/C=C/c1cccc(C(F)(F)F)c1)Nc1ccc2c(c1)OCO2] | >20 | >20 | >20 |
| 427 | 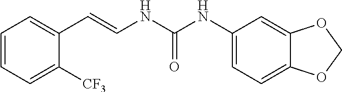 [SMILES: O=C(N/C=C/c1ccccc1C(F)(F)F)Nc1ccc2c(c1)OCO2] | 9.5 | I.A. | 19.2 |
| 431 | 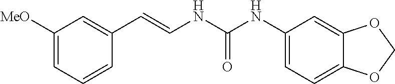 [SMILES: COc1cccc(/C=C/NC(=O)Nc2ccc3c(c2)OCO3)c1] | 17.3 | >20 | >20 |
| 432 | 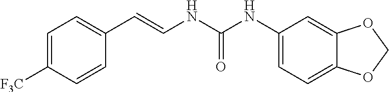 [SMILES: O=C(N/C=C/c1ccc(C(F)(F)F)cc1)Nc1ccc2c(c1)OCO2] | >20 | >20 | >20 |
| 515 | 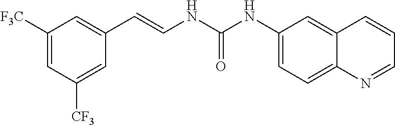 [SMILES: O=C(N/C=C/c1cc(C(F)(F)F)cc(C(F)(F)F)c1)Nc1ccc2ncccc2c1] | >10 | >10 | >10 |
| 516 | 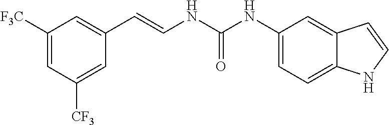 [SMILES: O=C(N/C=C/c1cc(C(F)(F)F)cc(C(F)(F)F)c1)Nc1ccc2[nH]ccc2c1] | >10 | >10 | I.A. |
| 517 | 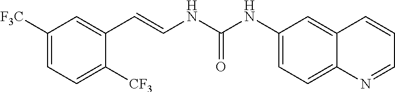 [SMILES: O=C(N/C=C/c1cc(C(F)(F)F)ccc1C(F)(F)F)Nc1ccc2ncccc2c1] | 3.4 | >10 | 6.2 |
| 518 | 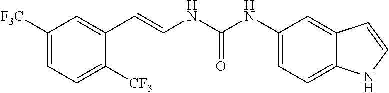 [SMILES: O=C(N/C=C/c1cc(C(F)(F)F)ccc1C(F)(F)F)Nc1ccc2[nH]ccc2c1] | | | |
| 519 | 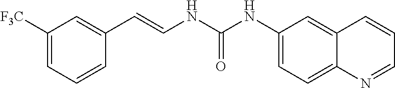 [SMILES: O=C(N/C=C/c1cccc(C(F)(F)F)c1)Nc1ccc2ncccc2c1] | | | |
| 520 | 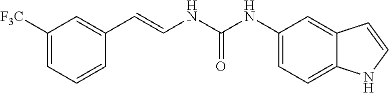 [SMILES: O=C(N/C=C/c1cccc(C(F)(F)F)c1)Nc1ccc2[nH]ccc2c1] | 6.5 | >10 | >10 |
| 523 | 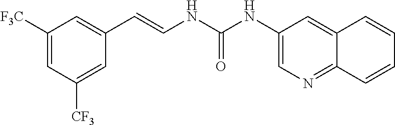 [SMILES: O=C(N/C=C/c1cc(C(F)(F)F)cc(C(F)(F)F)c1)Nc1cnc2ccccc2c1] | 8.0 | 8.4 | 10 |
| 524 | 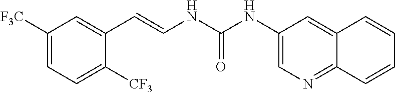 [SMILES: O=C(N/C=C/c1cc(C(F)(F)F)ccc1C(F)(F)F)Nc1cnc2ccccc2c1] | 3.4 | 8.4 | 7.4 |
| 525 | 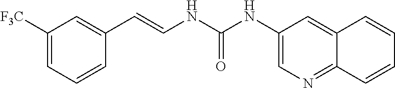 [SMILES: O=C(N/C=C/c1cccc(C(F)(F)F)c1)Nc1cnc2ccccc2c1] | >10 | >10 | >10 |

TABLE 2.4
The 562 “Analogues of Formula (IV)”.

| | | Cytotoxicity (IC_{50,} μM) | | |
|---|---|---|---|---|
| ID | Structures | LNCaP | 22Rv1 | DU145 |
| 419 | 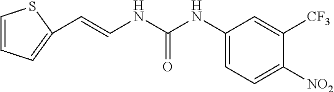 [SMILES: O=C(N/C=C/c1cccs1)Nc1ccc([N+](=O)[O-])c(C(F)(F)F)c1] | 3.2 | 4.1 | 6.9 |
| 420 | 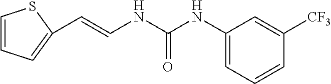 [SMILES: O=C(N/C=C/c1cccs1)Nc1cccc(C(F)(F)F)c1] | 6.4 | 12.3 | 12.3 |
| 424 | 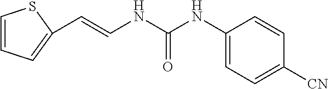 [SMILES: N#Cc1ccc(NC(=O)N/C=C/c2cccs2)cc1] | 5.0 | 16.7 | 14.4 |
| 425 | 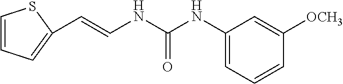 [SMILES: COc1cccc(NC(=O)N/C=C/c2cccs2)c1] | >20 | I.A. | I.A. |
| 426 | 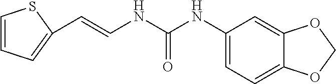 [SMILES: O=C(N/C=C/c1cccs1)Nc1ccc2c(c1)OCO2] | >20 | I.A. | I.A. |
| 428 | 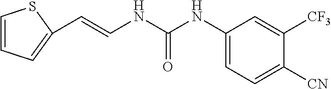 [SMILES: N#Cc1ccc(NC(=O)N/C=C/c2cccs2)cc1C(F)(F)F] | 2.1 | 3.7 | 5.1 |
| 434 | 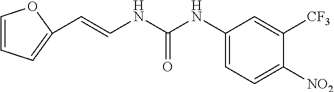 [SMILES: O=C(N/C=C/c1ccco1)Nc1ccc([N+](=O)[O-])c(C(F)(F)F)c1] | 2.0 | 5.6 | 7.8 |
| 443 | 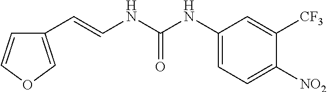 [SMILES: O=C(N/C=C/c1ccoc1)Nc1ccc([N+](=O)[O-])c(C(F)(F)F)c1] | I.A. | I.A. | I.A. |
| 444 | 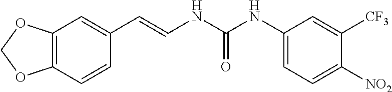 [SMILES: O=C(N/C=C/c1ccc2c(c1)OCO2)Nc1ccc([N+](=O)[O-])c(C(F)(F)F)c1] | 14.6 | 17.5 | 14.0 |
| 447 | 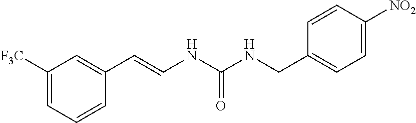 [SMILES: O=C(N/C=C/c1cccc(C(F)(F)F)c1)NCc1ccc([N+](=O)[O-])cc1] | 7.5 | 7.5 | 8.0 |
| 448 | 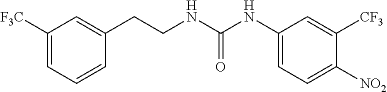 [SMILES: O=C(NCCc1cccc(C(F)(F)F)c1)Nc1ccc([N+](=O)[O-])c(C(F)(F)F)c1] | 1.8 | 4.5 | 7.5 |
| 450 | 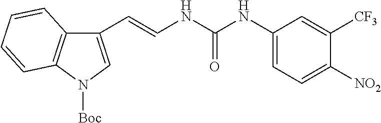 [SMILES: CC(C)(C)OC(=O)n1cc(/C=C/NC(=O)Nc2ccc([N+](=O)[O-])c(C(F)(F)F)c2)c2ccccc21] | 1.8 | 4.0 | 4.9 |
| 453 | 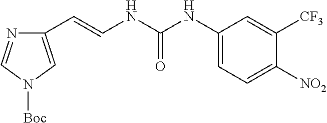 [SMILES: CC(C)(C)OC(=O)n1cnc(/C=C/NC(=O)Nc2ccc([N+](=O)[O-])c(C(F)(F)F)c2)c1] | 4.0 | >20 | >20 |
| 454 | 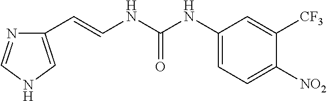 [SMILES: O=C(N/C=C/c1c[nH]cn1)Nc1ccc([N+](=O)[O-])c(C(F)(F)F)c1] | >10 | I.A. | I.A. |
| 459 | 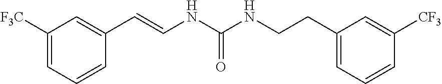 [SMILES: O=C(N/C=C/c1cccc(C(F)(F)F)c1)NCCc1cccc(C(F)(F)F)c1] | 7.1 | I.A. | >10 |
| 460 | 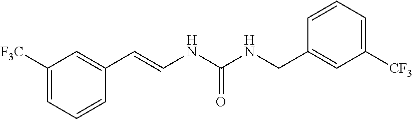 [SMILES: O=C(N/C=C/c1cccc(C(F)(F)F)c1)NCc1cccc(C(F)(F)F)c1] | 7.9 | I.A. | I.A. |
| 461 | 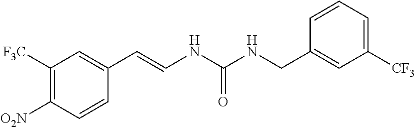 [SMILES: O=C(N/C=C/c1ccc([N+](=O)[O-])c(C(F)(F)F)c1)NCc1cccc(C(F)(F)F)c1] | 2.4 | 3.4 | 3.4 |
| 633 | 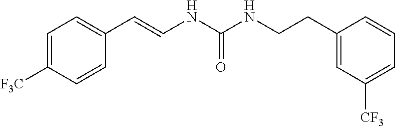 [SMILES: O=C(N/C=C/c1ccc(C(F)(F)F)cc1)NCCc1cccc(C(F)(F)F)c1] | | | |
| 634 | 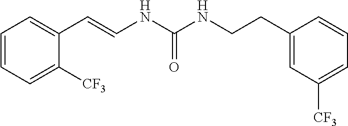 [SMILES: O=C(N/C=C/c1ccccc1C(F)(F)F)NCCc1cccc(C(F)(F)F)c1] | | | |
| 635 | 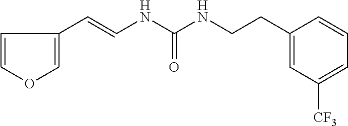 [SMILES: O=C(N/C=C/c1ccoc1)NCCc1cccc(C(F)(F)F)c1] | | | |
| 642 | 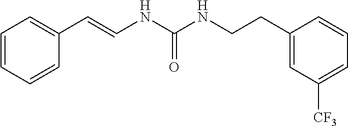 [SMILES: O=C(N/C=C/c1ccccc1)NCCc1cccc(C(F)(F)F)c1] | | | |
| 937 | 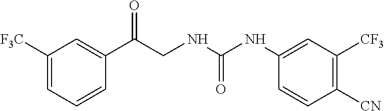 [SMILES: N#Cc1ccc(NC(=O)NCC(=O)c2cccc(C(F)(F)F)c2)cc1C(F)(F)F] | 7.3 | | |
| 982 | 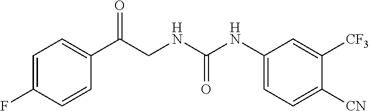 [SMILES: N#Cc1ccc(NC(=O)NCC(=O)c2ccc(F)cc2)cc1C(F)(F)F] | 4.6 | | |

TABLE 2.5
The 562 “Analogues of Formula (V)”.

| | | Cytotoxicity (IC_{50,} μM) | | |
|---|---|---|---|---|
| ID | Structures | LNCaP | 22Rv1 | DU145 |
| 534 | 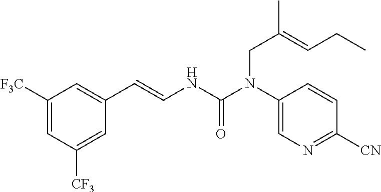 [SMILES: CC/C=C(\C)CN(C(=O)N/C=C/c1cc(C(F)(F)F)cc(C(F)(F)F)c1)c1ccc(C#N)nc1] | 6.0 | 7.3 | 7.1 |
| 547 | 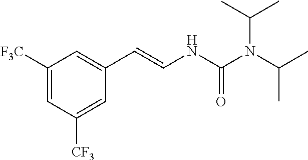 [SMILES: CC(C)N(C(=O)N/C=C/c1cc(C(F)(F)F)cc(C(F)(F)F)c1)C(C)C] | | | |
| 563 | 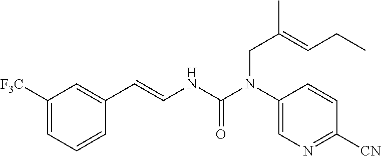 [SMILES: CC/C=C(\C)CN(C(=O)N/C=C/c1cccc(C(F)(F)F)c1)c1ccc(C#N)nc1] | | | |
| 591 | 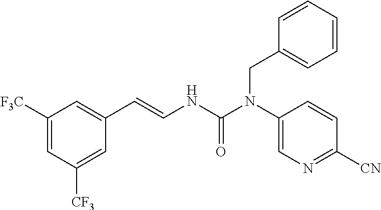 [SMILES: N#Cc1ccc(N(Cc2ccccc2)C(=O)N/C=C/c2cc(C(F)(F)F)cc(C(F)(F)F)c2)cn1] | | | |
| 620 | 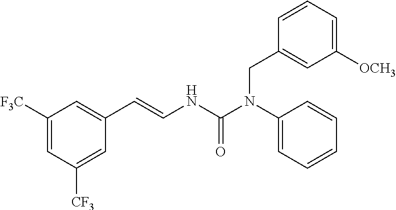 [SMILES: COc1cccc(CN(C(=O)N/C=C/c2cc(C(F)(F)F)cc(C(F)(F)F)c2)c2ccccc2)c1] | | | |
| 621 | 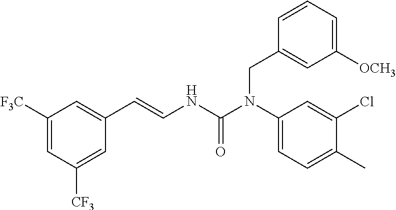 [SMILES: COc1cccc(CN(C(=O)N/C=C/c2cc(C(F)(F)F)cc(C(F)(F)F)c2)c2ccc(C)c(Cl)c2)c1] | | | |
| 622 | 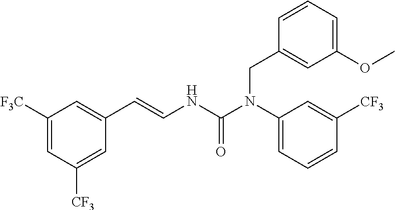 [SMILES: COc1cccc(CN(C(=O)N/C=C/c2cc(C(F)(F)F)cc(C(F)(F)F)c2)c2cccc(C(F)(F)F)c2)c1] | | | |
| 623 | 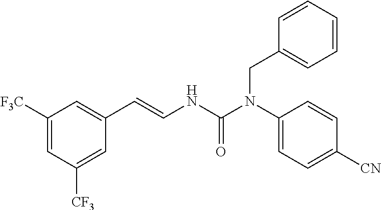 [SMILES: N#Cc1ccc(N(Cc2ccccc2)C(=O)N/C=C/c2cc(C(F)(F)F)cc(C(F)(F)F)c2)cc1] | | | |

TABLE 3.1
Compound 804 and its Analogues.

| ID. | Structure |
|---|---|
| 804 | 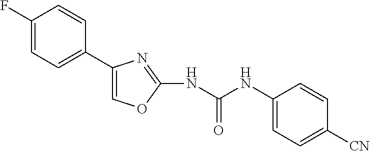 [SMILES: N#Cc1ccc(NC(=O)Nc2nc(-c3ccc(F)cc3)co2)cc1] |
| 790 | 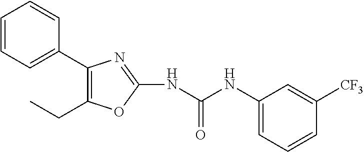 [SMILES: CCc1oc(NC(=O)Nc2cccc(C(F)(F)F)c2)nc1-c1ccccc1] |
| 791 | 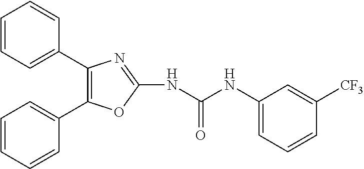 [SMILES: O=C(Nc1cccc(C(F)(F)F)c1)Nc1nc(-c2ccccc2)c(-c2ccccc2)o1] |
| 797 | 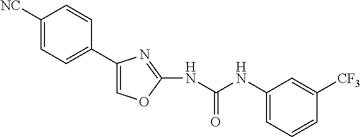 [SMILES: N#Cc1ccc(-c2coc(NC(=O)Nc3cccc(C(F)(F)F)c3)n2)cc1] |
| 798 | 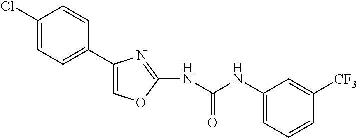 [SMILES: O=C(Nc1cccc(C(F)(F)F)c1)Nc1nc(-c2ccc(Cl)cc2)co1] |
| 799 | 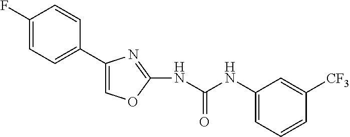 [SMILES: O=C(Nc1cccc(C(F)(F)F)c1)Nc1nc(-c2ccc(F)cc2)co1] |
| 803 | 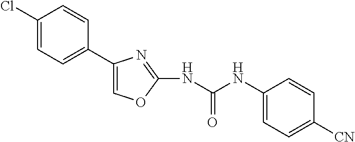 [SMILES: N#Cc1ccc(NC(=O)Nc2nc(-c3ccc(Cl)cc3)co2)cc1] |
| 805 | 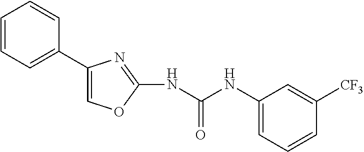 [SMILES: O=C(Nc1cccc(C(F)(F)F)c1)Nc1nc(-c2ccccc2)co1] |
| 802 | 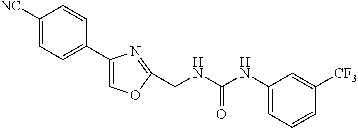 [SMILES: N#Cc1ccc(-c2coc(CNC(=O)Nc3cccc(C(F)(F)F)c3)n2)cc1] |
| 783 | 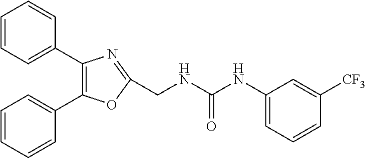 [SMILES: O=C(NCc1nc(-c2ccccc2)c(-c2ccccc2)o1)Nc1cccc(C(F)(F)F)c1] |
| 788 | 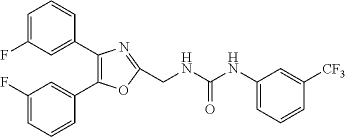 [SMILES: O=C(NCc1nc(-c2cccc(F)c2)c(-c2cccc(F)c2)o1)Nc1cccc(C(F)(F)F)c1] |
| 885 | 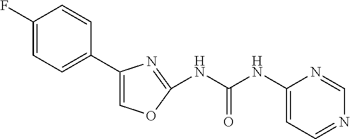 [SMILES: O=C(Nc1ccncn1)Nc1nc(-c2ccc(F)cc2)co1] |

TABLE 4.1
Compound 566 and its “Analogues of Formula (I)”.
566

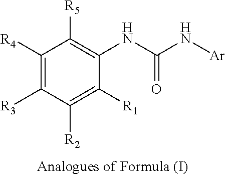

| | | | | | | | | | | Cytotoxicity | | |
|---|---|---|---|---|---|---|---|---|---|---|---|---|
| | Substituents | | | | | | | | | (IC_{50,} μM) | | |
| | | | | Ar | | | | | | LNC- | 22R- | DU- |
| ID | R₂ | R₃ | R₄ | | R₆ | R₇ | R₈ | R₉ | R₁₀ | aP | v1 | 145 |
| 484 | | | CF₃ | A | | | CN | | | 1.6 | 6.4 | 3.7 |
| 486 | | | CF₃ | B | | | CN | | | 3.4 | 8.0 | 3.0 |
| 491 | | | CF₃ | A | | | NO₂ | | | 17.3 | >20 | 1.9 |
| 495 | CF₃ | | CF₃ | A | | | CN | | | 0.3 | 1.1 | 0.1 |
| 496 | CF₃ | | CF₃ | A | | | NO₂ | | | 0.4 | 1.3 | 0.1 |
| 498 | | CF₃ | | B | | | CN | | | 3.6 | 6.3 | 10.4 |
| 499 | | CF₃ | | A | | | CN | | | 4.2 | >20 | 6.6 |
| 501 | | CF₃ | | A | | | NO₂ | | | 1.4 | >20 | >20 |
| 506 | CF₃ | | CF₃ | B | | | | | | 4.5 | 9.2 | 11.9 |
| 507 | | CF₃ | | B | | | | | | 18.8 | >20 | >20 |
| 565 | CF₃ | | CF₃ | B | Cl | | | | | 3.8 | >20 | >20 |
| 566 | CF₃ | | CF₃ | B | | | Br | | | 0.8 | 1.1 | 1.7 |
| 567 | | | CF₃ | B | | F | | | | 6.1 | 8.2 | 12.9 |
| 568 | | | CF₃ | B | Cl | | | | | 10.9 | >20 | >20 |
| 569 | | | CF₃ | B | | | Br | | | 4.1 | 3.8 | 7.8 |
| 570 | | | CF₃ | B | CH₃ | | | | | 12.9 | >20 | Inac- |
| | | | | | | | | | | | | tive |
| 571 | | | CF₃ | D | | | | | | 15.8 | >20 | >20 |
| 572 | | | CF₃ | E | | Ph | | | | 1.5 | 3.3 | 4.1 |
| 573 | CF₃ | | CF₃ | B | | F | | | | | | |
| 575 | | | CF₃ | C | | | CF₃ | | | 4.4 | 15 | 9.2 |
| 576 | CF₃ | | CF₃ | D | | | | | | 4.4 | 3.4 | 14.1 |
| 579 | CF₃ | | CF₃ | B | | | CH₃ | | | 4.5 | 4.9 | 8.7 |
| 580 | | | CF₃ | E | | CF₃ | | | | 0.9 | 2.4 | 2.3 |
| 584 | CF₃ | | CF₃ | E | | CF₃ | | | | | | |
| 739 | CF₃ | | | B | | | F | | | | | |
| 740 | CF₃ | | | B | | | Cl | | | | | |
| 741 | CF₃ | | | B | | Cl | Cl | | | | | |
| 754 | CF₃ | | CF₃ | B | | | F | | | | | |
| 755 | CF₃ | | CF₃ | B | | | Cl | | | | | |
| 758 | CF₃ | | CF₃ | B | | Cl | Cl | | | | | |
| 763 | CF₃ | | CF₃ | B | CH₃ | | | | Cl | | | |
| 764 | CF₃ | | CF₃ | B | CH₃ | | F | | | | | |
| 773 | CN | | | | | | Br | | | | | |

| | | | | |
|---|---|---|---|---|
| 522 | 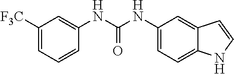 [SMILES: O=C(Nc1cccc(C(F)(F)F)c1)Nc1ccc2[nH]ccc2c1] | >10 | >10 | >10 |
| 530 | 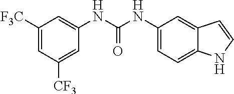 [SMILES: O=C(Nc1cc(C(F)(F)F)cc(C(F)(F)F)c1)Nc1ccc2[nH]ccc2c1] | 6 | 7.1 | 8.3 |
| 574 | 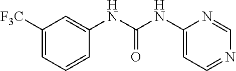 [SMILES: O=C(Nc1cccc(C(F)(F)F)c1)Nc1ccncn1] | 13.8 | >20 | >20 |
| 578 | 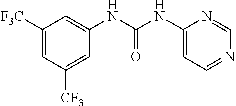 [SMILES: O=C(Nc1cc(C(F)(F)F)cc(C(F)(F)F)c1)Nc1ccncn1] | 1.3 | 5.5 | 2.4 |
| 737 | 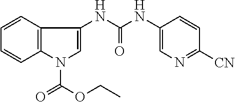 [SMILES: CCOC(=O)n1cc(NC(=O)Nc2ccc(C#N)nc2)c2ccccc21] | | | |
| 738 | 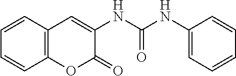 [SMILES: O=C(Nc1ccccc1)Nc1cc2ccccc2oc1=O] | | | |
| 744 | 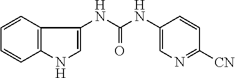 [SMILES: N#Cc1ccc(NC(=O)Nc2c[nH]c3ccccc23)cn1] | | | |
| 753 | 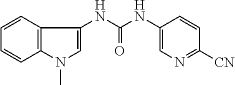 [SMILES: Cn1cc(NC(=O)Nc2ccc(C#N)nc2)c2ccccc21] | | | |

R₁ = R₅ = H

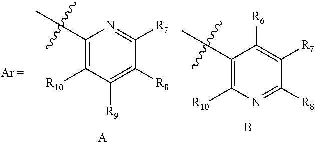

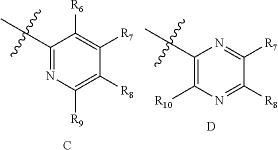

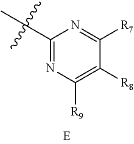

TABLE 4.2
Compound 566 and its “Analogues of Formula (II)”.

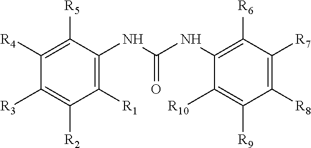

| | Substituents | | | | | | | | | |
|---|---|---|---|---|---|---|---|---|---|---|
| ID | R₁ | R₂ | R₃ | R₄ | R₅ | R₆ | R₇ | R₈ | R₉ | R₁₀ |
| 442 | | | | CF₃ | | | CF₃ | NO₂ | | |
| 465 | | | NO₂ | CF₃ | | | CF₃ | CN | | |
| 467 | | | | CF₃ | | | CF₃ | CN | | |
| 492 | | | | CF₃ | | | Cl | CN | | |
| 494 | | CF₃ | | CF₃ | | | CF₃ | CN | | |
| 500 | | CF₃ | | CF₃ | | | Cl | CN | | |
| 502 | | | CF₃ | | | | Cl | CN | | |
| 509 | | | CF₃ | | | | CF₃ | CN | | |
| 646 | | OCH₃ | | | | | Cl | CN | | |
| 647 | | Cl | | | | | Cl | CN | | |
| 680 | | | | | | | Cl | CN | | |
| 701 | | OCH₃ | | | | | CF₃ | CN | | |
| 702 | | | | | | | CF₃ | CN | | |
| 703 | | CH₃ | | | | | CF₃ | CN | | |
| 704 | | F | | | | | CF₃ | CN | | |
| 705 | | Cl | | | | | CF₃ | CN | | |
| 706 | CF₃ | | | CF₃ | | | CF₃ | CN | | |
| 736 | | CF₃ | | | | | CF₃ | NH₂ | | |
| 745 | | CF₃ | | | | | | NH₂ | | |
| 772 | | CN | | | | | Cl | CN | | |
| 774 | | CN | | | | | CN | CN | | |
| 792 | | CF₃ | | CF₃ | | | F | CN | | |
| 829 | | CF₃ | F | | | | CF₃ | NO₂ | | |
| 887 | | CF₃ | | OCH₃ | | | CF₃ | | | |

| | Cytotoxicity (IC_{50,} μM) | | |
|---|---|---|---|
| ID | LNCaP | 22Rv1 | DU145 |
| 442 | 0.5 | 1.5 | 1.5 |
| 465 | 4.1 | 5.2 | 5.3 |
| 467 | 0.9 | 1.7 | 1.6 |
| 492 | 0.8 | 1.9 | 5.1 |
| 494 | 0.3 | 1.6 | 1.5 |
| 500 | 0.2 | 0.4 | 1.3 |
| 502 | 1.2 | 2.2 | 4.1 |
| 509 | 0.7 | 1.6 | 5.4 |
| 646 | | | |
| 647 | | | |
| 680 | | | |
| 701 | | | |
| 702 | | | |
| 703 | | | |
| 704 | | | |
| 705 | | | |
| 706 | | | |
| 736 | | | |
| 745 | | | |
| 772 | | | |
| 774 | | | |
| 792 | | | |
| 829 | | | |
| 887 | | | |

TABLE 5.1
Bis-urea compounds.

| | | Cytotoxicity (IC₅₀, μM) | | |
|---|---|---|---|---|
| ID | Structures | LNCaP | 22Rv1 | DU145 |
| 439 | 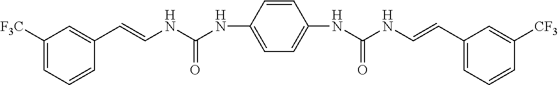 [SMILES: O=C(N/C=C/c1cccc(C(F)(F)F)c1)Nc1ccc(NC(=O)N/C=C/c2cccc(C(F)(F)F)c2)cc1] | | | |
| 440 | 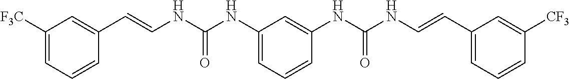 [SMILES: O=C(N/C=C/c1cccc(C(F)(F)F)c1)Nc1cccc(NC(=O)N/C=C/c2cccc(C(F)(F)F)c2)c1] | 1.7 | 4.9 | 5.1 |
| 451 | 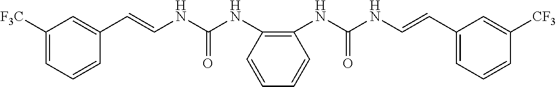 [SMILES: O=C(N/C=C/c1cccc(C(F)(F)F)c1)Nc1ccccc1NC(=O)N/C=C/c1cccc(C(F)(F)F)c1] | 2.4 | 4.4 | 5.4 |
| 452 | 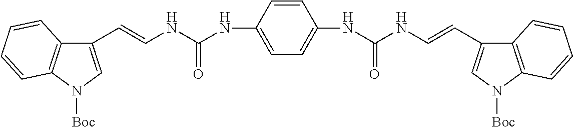 [SMILES: CC(C)(C)OC(=O)n1cc(/C=C/NC(=O)Nc2ccc(NC(=O)N/C=C/c3cn(C(=O)OC(C)(C)C)c4ccccc34)cc2)c2ccccc21] | >20 | >20 | 3.4 |
| 455 | 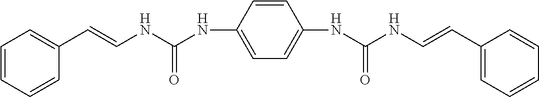 [SMILES: O=C(N/C=C/c1ccccc1)Nc1ccc(NC(=O)N/C=C/c2ccccc2)cc1] | | | |
| 457 | 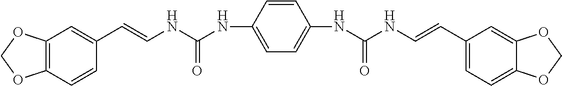 [SMILES: O=C(N/C=C/c1ccc2c(c1)OCO2)Nc1ccc(NC(=O)N/C=C/c2ccc3c(c2)OCO3)cc1] | | | |
| 458 | 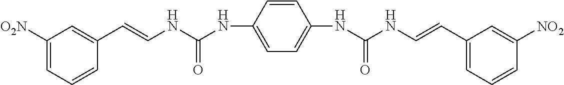 [SMILES: O=C(N/C=C/c1cccc([N+](=O)[O-])c1)Nc1ccc(NC(=O)N/C=C/c2cccc([N+](=O)[O-])c2)cc1] | | | |
| 466 | 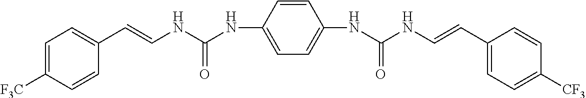 [SMILES: O=C(N/C=C/c1ccc(C(F)(F)F)cc1)Nc1ccc(NC(=O)N/C=C/c2ccc(C(F)(F)F)cc2)cc1] | | | |
| 532 | 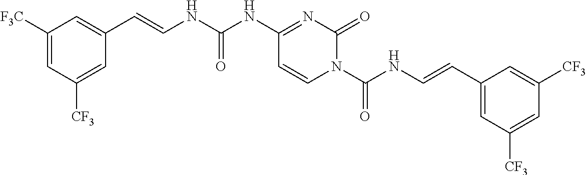 [SMILES: O=C(N/C=C/c1cc(C(F)(F)F)cc(C(F)(F)F)c1)Nc1ccn(C(=O)N/C=C/c2cc(C(F)(F)F)cc(C(F)(F)F)c2)c(=O)n1] | 2.4 | 4.3 | 5.1 |

TABLE 6.1
Effect of selected compounds against a panel of cancer cell lines, including
prostate cancer, lung cancer, breast cancer, liver hepatocellular carcinoma
and ovarian cancer, as evaluated by MTT assays (72 h treatment).

| | Cytotoxicity (IC₅₀, μM) | | | | | | | |
|---|---|---|---|---|---|---|---|---|
| ID | LNCaP | 22Rv1 | H1975 | A549 | MB231 | MCF-7 | HepG2 | OVCAR-3 |
| 410 | 1.8 | 2.5 | 2.5 | 2.7 | 4.0 | 2.6 | 0.92 | |
| 428 | 2.1 | 3.7 | 7.7 | 4.7 | 7.6 | 5.2 | 2.4 | |
| 528 | 0.12 | <1 | | | | | | |
| 558 | 0.06 | 0.10 | 2.4 | 0.09 | 0.30 | <0.5 | 0.14 | |
| 746 | 1.0 | 2.2 | 2.5 | 2.4 | 4.6 | 2.5 | 1.2 | |
| 822 | 3.0 | 4.0 | 6.3 | 6.0 | 8.1 | 4.5 | 3.2 | |
| 861 | 9.7 | | | | | | | |
| 862 | 3.4 | | | | | | | |
| 875 | 4.6 | 8.8 | | | | | | |
| 877 | 4.4 | 23.5 | | | | | | |
| 878 | 3.4 | 3.2 | | | | | | |
| 879 | 6.3 | 6.8 | | | | | | |
| 896 | 5.2 | 18.9 | | | | | | |
| 897 | 1.9 | 7.0 | | | | | | |
| 898 | 1.4 | 5.1 | | | | | | |
| 899 | 6.7 | 15.0 | | | | | | |
| 900 | >10 | 10.4 | | | | | | |
| 901 | 6.6 | 4.7 | | | | | | |
| 902 | 7.4 | 4.5 | | | | | | |
| 903 | 6.8 | 5.0 | | | | | | |
| 904 | 4.1 | 3.2 | | | | | | |
| 905 | 5.5 | 11.4 | | | | | | |
| 907 | 4.2 | 4.2 | | | | | | |
| 911 | 13.8 | 10.0 | | | | | | |
| 912 | 11.6 | 16.0 | | | | | | |
| 913 | 12.3 | 14.2 | | | | | | |
| 914 | 7.3 | 8.7 | | | | | | |
| 915 | 9.0 | 10.6 | | | | | | |
| 928 | 2.4 | 2.0 | | | | | | |
| 929 | 5.5 | 5.7 | | | | | | |
| 930 | 8.2 | 5.6 | | | | | | |
| 937 | 7.3 | | | | | | | |
| 941 | 5.4 | 3.6 | | | | | | |
| 942 | 5.7 | 7.1 | | | | | | |
| 943 | 6.3 | 13.0 | | | | | | |
| 944 | 5.5 | 4.5 | | | | | | |
| 945 | 7.3 | 7.1 | | | | | | |
| 946 | 8.4 | 9.5 | | | | | | |
| 947 | 12.7 | 15.9 | | | | | | |
| 948 | 8.0 | 17.1 | | | | | | |
| 949 | 2.9 | 32.9 | | | | | | |
| 950 | 3.8 | 7.8 | | | | | | |
| 951 | 4.0 | 9.8 | | | | | | |
| 952 | 5.2 | 7.8 | | | | | | |
| 953 | 1.4 | 0.9 | | | | | | |
| 954 | 3.7 | 5.7 | | | | | | |
| 955 | 1.5 | 0.9 | | | | | | |
| 956 | 3.5 | 9.5 | | | | | | |
| 959 | 13.5 | 8.7 | | | | | | |
| 960 | | 5.7 | | | | | | |
| 961 | 6.2 | 10.1 | | | | | | |
| 962 | 5.3 | 4.9 | | | | | | |
| 963 | 2.4 | 4.7 | | | | | | |
| 964 | 2.4 | 1.8 | | | | | | |
| 965 | 4.3 | 7.7 | | | | | | |
| 966 | | 6.6 | | | | | | |
| 967 | 2.7 | 3.1 | | | | | | |

As will be understood by a skilled person considering the present specification, in certain embodiments, compounds according to the invention present activities against the LNCaP and 22Rv1 AR positive prostate cancer cells. Also, in other embodiments, compounds according to the invention present activities against DU145 AR negative prostate cancer cells, suggesting that such compounds can modulate other target(s) different from the AR.

Although the present invention has been described hereinabove by way of specific embodiments thereof, it may be modified, without departing from the spirit and nature of the subject invention as defined in the appended claims.

The present description refers to a number of documents, the content of which is herein incorporated by reference in their entirety.

### REFERENCES

1. Sadar M D. Small molecule inhibitors targeting the “Achilles' heel” of androgen receptor activity. Cancer Res. 2011; 71(4):1208-1213.
2. Taplin M E, Ho S M. The endocrinology of prostate cancer. J. Clin. Endocrinol. Metab. 2001; 86(8):3467-3477.
3. Tannock I F, de Wit R, Berry W R et al. Docetaxel plus prednisone or mitoxantrone plus prednisone for advanced prostate cancer. New Engl. J. Med. 2004; 351(15):1502-1512.
4. Petrylak D P, Tangen C M, Hussain M H et al. Docetaxel and estramustine compared with mitoxantrone and prednisone for advanced refractory prostate cancer. New Engl. J. Med. 2004; 351(15):1513-1520.
5. Agoulnik I U, Weigel N L. Androgen receptor action in hormone-dependent and recurrent prostate cancer. J. Cell. Biochem. 2006; 99(2):362-372.
6. Attard G, Swennenhuis J F, Olmos D et al. Characterization of ERG, AR and PTEN gene status in circulating tumor cells from patients with castration-resistant prostate cancer. Cancer Res. 2009; 69(7):2912-2918.
7. Krishnan A V, Zhao X Y, Swami S et al. A glucocorticoid-responsive mutant androgen receptor exhibits unique ligand specificity: therapeutic implications for androgen-independent prostate cancer. Endocrinology 2002; 143(5):1889-1900.
8. Eder I E, Haag P, Bartsch G, Klocker H. Targeting the androgen receptor in hormone-refractory prostate cancer—new concepts. Future Oncol. 2005; 1(1):93-101.
9. Chen C D, Welsbie D S, Tran C et al. Molecular determinants of resistance to antiandrogen therapy. Nat. Med. 2004; 10(1):33-39.
10. Hu R, Dunn T A, Wei S et al. Ligand-independent androgen receptor variants derived from splicing of cryptic exons signify hormone-refractory prostate cancer. Cancer Res. 2009; 69(1):16-22.
11. Guo Z, Yang X, Sun F et al. A novel androgen receptor splice variant is up-regulated during prostate cancer progression and promotes androgen depletion-resistant growth. Cancer Res. 2009; 69(6):2305-2313.
12. Sun S, Sprenger C C T, Vessella R L et al. Castration resistance in human prostate cancer is conferred by a frequently occurring androgen receptor splice variant. J. Clin. Invest. 2010; 120(8):2715-2730.
13. Zhang X, Morrissey C, Sun S et al. Androgen receptor variants occur frequently in castration resistant prostate cancer metastases. PLoS One 2011; 6(11):e27970.
14. Hornberg E, Ylitalo E B, Crnalic S et al. Expression of androgen receptor splice variants in prostate cancer bone metastases is associated with castration-resistance and short survival. PLoS ONE 2011; 6(4):e19059.
15. Andersen R J, Mawji N R, Wang J et al. Regression of Castrate-Recurrent Prostate Cancer by a Small-Molecule Inhibitor of the Amino-Terminus Domain of the Androgen Receptor. Cancer Cell 2010; 17(6):535-546.
16. Biles J E, White K D, McNeal T P, Begley T H. Determination of the diglycidyl ether of bisphenol A and its derivatives in canned foods. J. Agric. Food Chem. 1999; 47(5): 1965-1969.
17. Myung J K, Banuelos C A, Fernandez J G et al. An androgen receptor N-terminal domain antagonist for treating prostate cancer. J. Clin. Invest. 2013; 123(7):2948-2960.
18. Korpal M, Korn J M, Gao X et al. An F876L Mutation in Androgen Receptor Confers Genetic and Phenotypic Resistance to MDV3100 (Enzalutamide). Cancer Discov. 2013; 3(9):1030-1043.
19. Joseph J D, Lu N, Qian J et al. A Clinically Relevant Androgen Receptor Mutation Confers Resistance to Second-Generation Antiandrogens Enzalutamide and ARN-509. Cancer Discov. 2013; 3(9):1020-1029.
20. Culig Z, Hobisch A, Cronauer M V et al. Mutant androgen receptor detected in an advanced-stage prostatic carcinoma is activated by adrenal androgens and progesterone. Mol. Endocrinol. 1993; 7(12):1541-1550.
21. Chang Cy, Walther P J, McDonnell D P. Glucocorticoids Manifest Androgenic Activity in a Cell Line Derived from a Metastatic Prostate Cancer. Cancer Res. 2001; 61(24):8712-8717.
22. Steketee K, Timmerman L, Ziel-van der Made A C J, Doesburg P, Brinkmann A O, Trapman J. Broadened ligand responsiveness of androgen receptor mutants obtained by random amino acid substitution of H874 and mutation hot spot T877 in prostate cancer. Int. J. Cancer 2002; 100(3):309-317.
23 Urushibara M, Ishioka J, Hyochi N et al. Effects of steroidal and non-steroidal antiandrogens on wild-type and mutant androgen receptors. Prostate 2007; 67(8):799-807.
24. Hara T, Miyazaki J, Araki H et al. Novel mutations of androgen receptor: A possible mechanism of bicalutamide withdrawal syndrome. Cancer Res. 2003; 63(1):149-153.
25. Jenster G, van der Korput H A, Trapman J, Brinkmann A O. Identification of two transcription activation units in the N-terminal domain of the human androgen receptor. J. Biol. Chem. 1995; 270(13):7341-7346.
26. Taplin M E, Manola J, Oh W K et al. A phase II study of mifepristone (RU-486) in castration-resistant prostate cancer, with a correlative assessment of androgen-related hormones. BJU Int. 2008; 101(9):1084-1089.

1. A compound of general formula A or B below, or a pharmaceutically acceptable salt thereof, or a solvate or hydrate thereof, 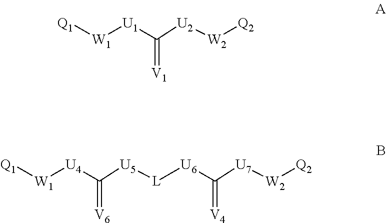
wherein:
U₁, U₂, U₄, U₅, U₆ and U₇ are each independently selected from a heteroatom and NR₁R₂ wherein R₁ and R₂ are each independently selected from H, alkyl, cycloalkyl, alkene, alkyne, aryl and alkylaryl, a 5 to 8-member ring comprising one or more heteroatom which are the same or different, or R₁ and R₂ together form a 5 to 8-member ring comprising one or more heteroatom; optionally, the ring is substituted with a substituent selected from alkyl, cycloalkyl alkoxy, alkoxy, thioalkoxy, OH, SH, NH₂, a halogen atom, a halogeno alkyl, a halogeno alkoxy, a halogeno thioalkoxy, CN, NO₂, SO₂ and COOH;
V₁, V₃ and V₄ are each independently selected from a heteroatom and carbon atom;
W₁ and W₂ are each independently present of absent, and are each independently selected from alkylene, alkenyl, alkynyl, a 5 to 20-member ring or bicycle ring comprising one or more heteroatom which are the same or different; optionally, the ring or bicycle ring is substituted with a group selected from alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, CN, NO₂, SO₂, COOH and NR₃R₄ wherein R₃ and R₄ are each independently selected from H, alkyl, cycloalkyl, alkene, alkyne, aryl and alkylaryl, or R₃ and R₄ together form a 5 to 8-member ring optionally comprising one or more heteroatom which are the same or different;
Q₁ is selected from alkyl, cycloalkyl, alkene, alkyne, aryl and alkylaryl, a 5 to 20-member ring or bicycle ring optionally comprising one or more heteroatom which are the same or different; optionally, the ring or bicycle ring is substituted with a substituent selected from alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, CN, NO₂, SO₂, COOH, acyloxycarbonyl, NR₃R₄ and C(═O)NR₃R₄ wherein R₃ and R₄ are each independently selected from H, alkyl, cycloalkyl, alkene, alkyne, aryl and alkylaryl, or R₃ and R₄ together form a 5 to 8-member ring optionally comprising one or more heteroatom which are the same or different; optionally, the 5 to 8-member ring is attached to an alkyl, a cycloalkyl, an alkene, an alkynyl, an aryl, aralkylryl or an acyloxycarbonyl; optionally, two consecutive substituents on the 5 to 20-member ring or bicycle ring together form a 5 to 8-member ring optionally comprising one or more heteroatom which are the same or different;
Q₂ is as defined above for Q₁, or is -Q′₂-U₃—C(═V₂)Q₃, wherein: U₃ is as defined above for U₁, U₂, U₄, U₅, U₆ and U₇; V₂ is as defined above for V₁, V₃ and V₄; and Q′₂ and Q₃ are each independently as defined above for Q₁;
L is selected from alkylene, alkenyl, alkynyl, a 5 to 20-member ring or bicycle ring comprising one or more heteroatom which are the same or different; optionally, the ring or bicycle ring is substituted with a group selected from alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, CN, NO₂, SO₂, COOH and NR₃R₄ wherein R₃ and R₄ are each independently selected from H, alkyl, cycloalkyl, alkene, alkyne, aryl and alkylaryl, or R₃ and R₄ together form a 5 to 8-member ring optionally comprising one or more heteroatom which are the same or different;
optionally L together with either U₅ or U₆ or both U₅ and U₆ form a 5 to 20-member ring or bicycle ring optionally comprising one or more heteroatom which are the same or different; optionally, the ring or bicycle ring is substituted with a substituent selected from alkyl, cycloalkyl, alkene, alkyne, aryl and alkylaryl, alkoxy, thioalkoxy, OH, SH, NH₂, a halogen atom, a halogeno alkyl, a halogeno alkoxy, a halogeno thioalkoxy, CN, NO₂, SO₂, COOH, acyloxycarbonyl, NR₃R₄ and C(═O)NR₃R₄ wherein R₃ and R₄ are each independently selected from H, alkyl, cycloalkyl, alkene, alkyne, aryl and alkylaryl, or R₃ and R₄ together form a 5 to 8-member ring optionally comprising one or more heteroatom which are the same or different; optionally, the 5 to 8-member ring is attached to an alkyl, a cycloalkyl, an alkene, an alkynyl, an aryl, analkylryl or an acyloxycarbonyl; optionally, two consecutive substituents on the 5 to 20-member ring or bicycle ring together form a 5 to 8-member ring optionally comprising one or more heteroatom which are the same or different;
the heteroatom is selected from O, N and S.

2. A compound according to claim 1 having the general formula A1, A2, A2′, A3, A3′, A3″, A4, A5, A6, A7, A8, A9, A10, A11, A12, A13, A14, A15, A16, A17, A18, A19, B1 or B2 outlined below 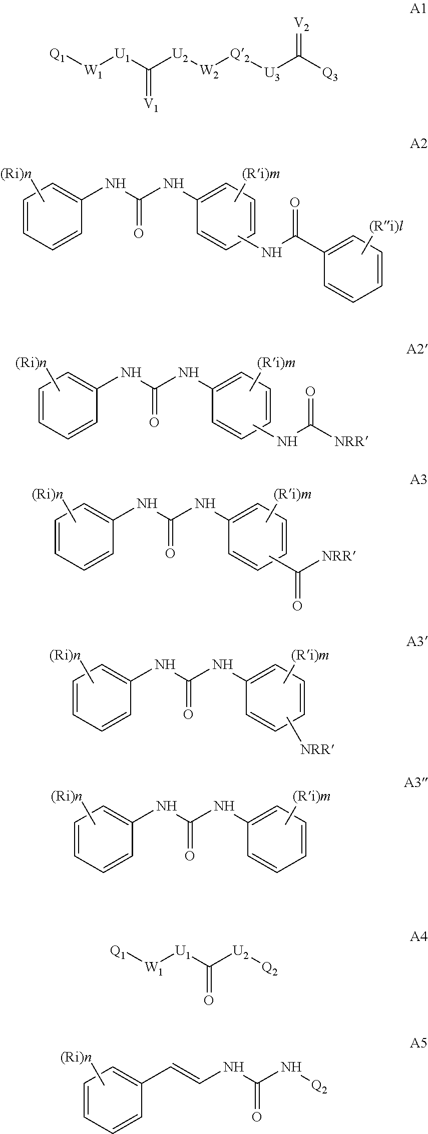 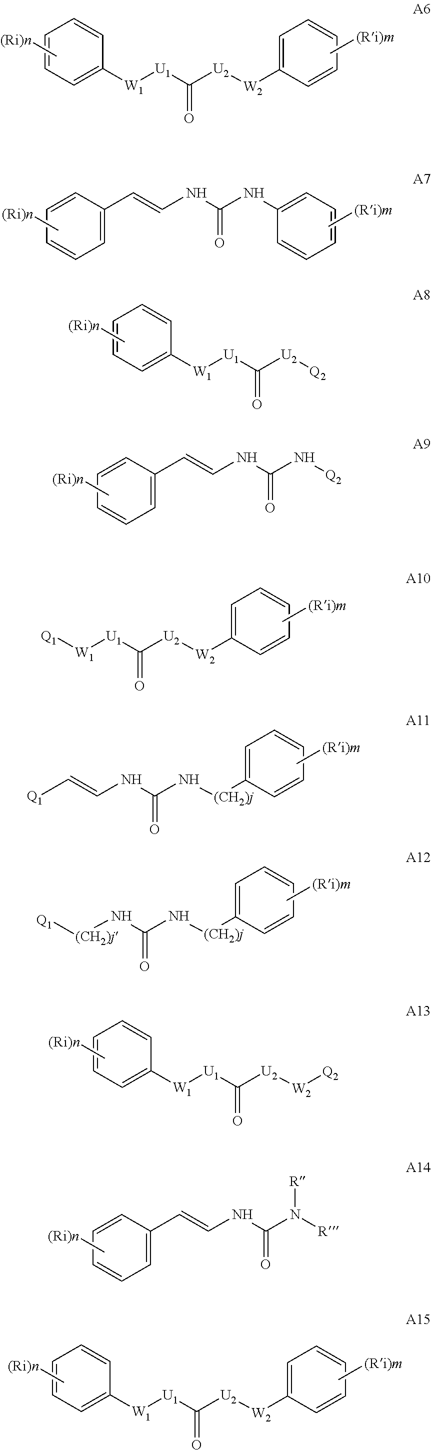 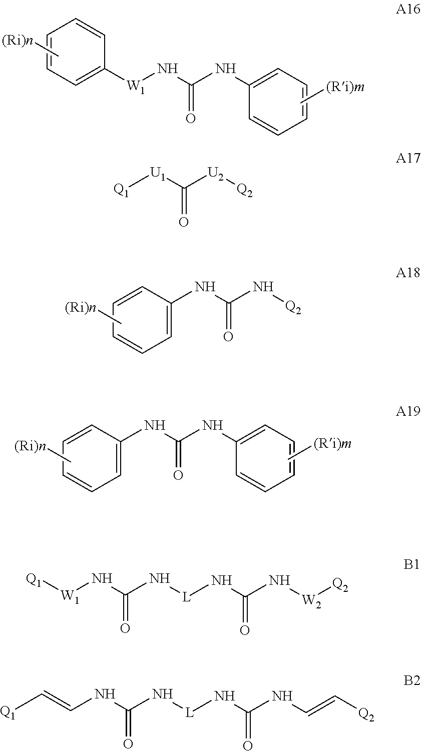
wherein:
n is an integer selected from 0 to 5, and each Ri is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH; optionally, two consecutive Ri together form a 5 to 8-member ring which optionally comprises one or more heteroatom which are the same or different;
m is an integer selected from 0 to 4, and each R′i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH: optionally, two consecutive R′i together form a 5 to 8-member ring which optionally comprises one or more heteroatom which are the same or different;
l is an integer selected from 0 to 5, and each R″i is independently selected from alkyl, cycloalkyl, alkoxy, thioalkoxy, OH, SH, NH₂, a halogen atom, a halogeno alkyl, a halogeno alkoxy, a halogeno thioalkoxy, CN, NO₂, SO₂ and COOH: optionally, two consecutive R″i together form a 5 to 8-member ring which optionally comprises one or more heteroatom which are the same or different; and
at least one of R and R′ is as Q₁, or R and R′ are as R₃ and R₄.

3. (canceled)

4. A compound according to claim 2 and having the general formula A2, which is selected from the group of compounds depicted below

| ID | Structure |
|---|---|
| 746 | 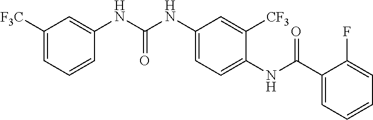 [SMILES: O=C(Nc1cccc(C(F)(F)F)c1)Nc1ccc(NC(=O)c2ccccc2F)c(C(F)(F)F)c1] |
| 743 | 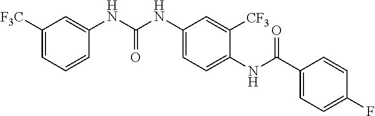 [SMILES: O=C(Nc1cccc(C(F)(F)F)c1)Nc1ccc(NC(=O)c2ccc(F)cc2)c(C(F)(F)F)c1] |
| 747 | 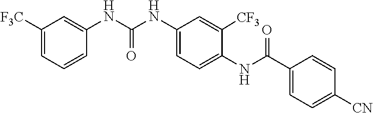 [SMILES: N#Cc1ccc(C(=O)Nc2ccc(NC(=O)Nc3cccc(C(F)(F)F)c3)cc2C(F)(F)F)cc1] |
| 806 | 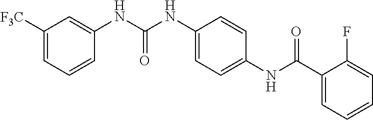 [SMILES: O=C(Nc1ccc(NC(=O)c2ccccc2F)cc1)Nc1cccc(C(F)(F)F)c1] |
| 808 | 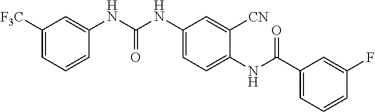 [SMILES: N#Cc1cc(NC(=O)Nc2cccc(C(F)(F)F)c2)ccc1NC(=O)c1cccc(F)c1] |
| 814 | 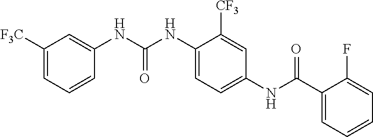 [SMILES: O=C(Nc1cccc(C(F)(F)F)c1)Nc1ccc(NC(=O)c2ccccc2F)cc1C(F)(F)F] |
| 815 | 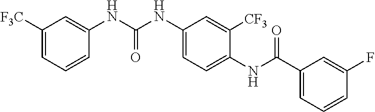 [SMILES: O=C(Nc1cccc(C(F)(F)F)c1)Nc1ccc(NC(=O)c2cccc(F)c2)c(C(F)(F)F)c1] |
| 816 | 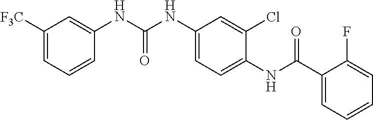 [SMILES: O=C(Nc1cccc(C(F)(F)F)c1)Nc1ccc(NC(=O)c2ccccc2F)c(Cl)c1] |
| 820 | 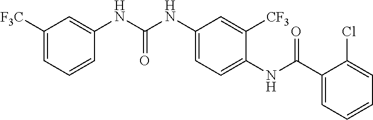 [SMILES: O=C(Nc1cccc(C(F)(F)F)c1)Nc1ccc(NC(=O)c2ccccc2Cl)c(C(F)(F)F)c1] |
| 813 | 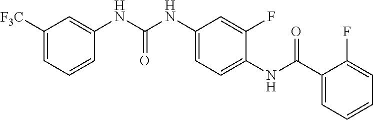 [SMILES: O=C(Nc1cccc(C(F)(F)F)c1)Nc1ccc(NC(=O)c2ccccc2F)c(F)c1] |
| 825 | 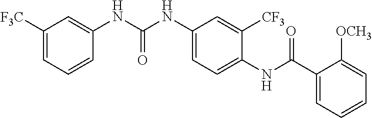 [SMILES: COc1ccccc1C(=O)Nc1ccc(NC(=O)Nc2cccc(C(F)(F)F)c2)cc1C(F)(F)F] |
| 863 | 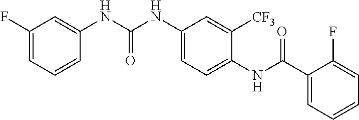 [SMILES: O=C(Nc1cccc(F)c1)Nc1ccc(NC(=O)c2ccccc2F)c(C(F)(F)F)c1] |
| 864 | 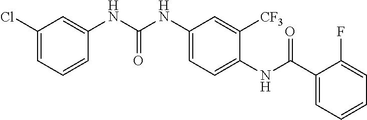 [SMILES: O=C(Nc1cccc(Cl)c1)Nc1ccc(NC(=O)c2ccccc2F)c(C(F)(F)F)c1] |
| 886 | 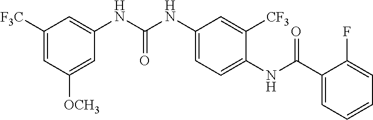 [SMILES: COc1cc(NC(=O)Nc2ccc(NC(=O)c3ccccc3F)c(C(F)(F)F)c2)cc(C(F)(F)F)c1] |
| 896 | 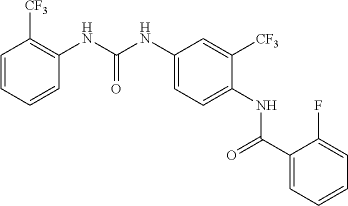 [SMILES: O=C(Nc1ccc(NC(=O)c2ccccc2F)c(C(F)(F)F)c1)Nc1ccccc1C(F)(F)F] |
| 897 | 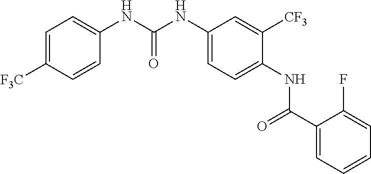 [SMILES: O=C(Nc1ccc(C(F)(F)F)cc1)Nc1ccc(NC(=O)c2ccccc2F)c(C(F)(F)F)c1] |
| 849 | 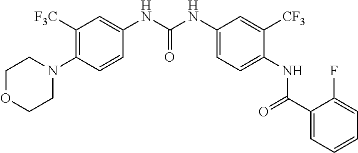 [SMILES: O=C(Nc1ccc(NC(=O)c2ccccc2F)c(C(F)(F)F)c1)Nc1ccc(N2CCOCC2)c(C(F)(F)F)c1] |
| 879 | 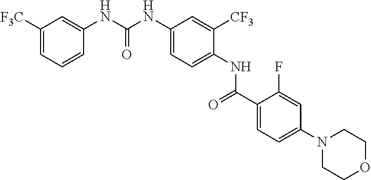 [SMILES: O=C(Nc1cccc(C(F)(F)F)c1)Nc1ccc(NC(=O)c2ccc(N3CCOCC3)cc2F)c(C(F)(F)F)c1] |
| 878 | 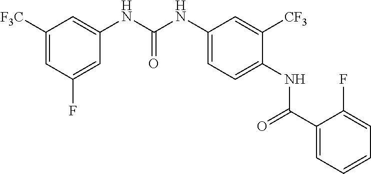 [SMILES: O=C(Nc1cc(F)cc(C(F)(F)F)c1)Nc1ccc(NC(=O)c2ccccc2F)c(C(F)(F)F)c1] |
| 861 | 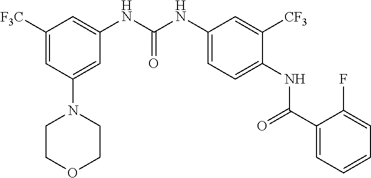 [SMILES: O=C(Nc1cc(N2CCOCC2)cc(C(F)(F)F)c1)Nc1ccc(NC(=O)c2ccccc2F)c(C(F)(F)F)c1] |
| 862 | 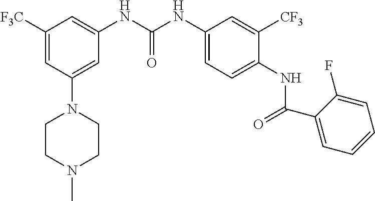 [SMILES: CN1CCN(c2cc(NC(=O)Nc3ccc(NC(=O)c4ccccc4F)c(C(F)(F)F)c3)cc(C(F)(F)F)c2)CC1] |
| 890 | 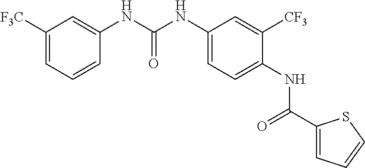 [SMILES: O=C(Nc1cccc(C(F)(F)F)c1)Nc1ccc(NC(=O)c2cccs2)c(C(F)(F)F)c1] |
| 900 | 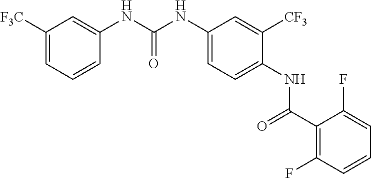 [SMILES: O=C(Nc1cccc(C(F)(F)F)c1)Nc1ccc(NC(=O)c2c(F)cccc2F)c(C(F)(F)F)c1] |
| 906 | 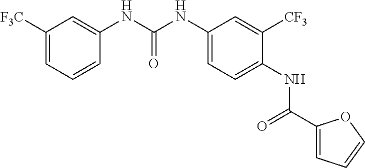 [SMILES: O=C(Nc1cccc(C(F)(F)F)c1)Nc1ccc(NC(=O)c2ccco2)c(C(F)(F)F)c1] |
| 894 | 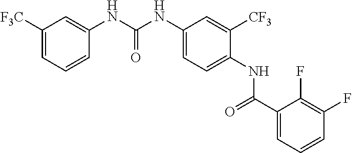 [SMILES: O=C(Nc1cccc(C(F)(F)F)c1)Nc1ccc(NC(=O)c2cccc(F)c2F)c(C(F)(F)F)c1] |
| 901 | 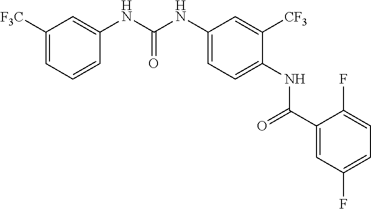 [SMILES: O=C(Nc1cccc(C(F)(F)F)c1)Nc1ccc(NC(=O)c2cc(F)ccc2F)c(C(F)(F)F)c1] |
| 902 | 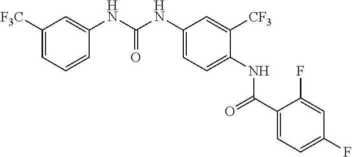 [SMILES: O=C(Nc1cccc(C(F)(F)F)c1)Nc1ccc(NC(=O)c2ccc(F)cc2F)c(C(F)(F)F)c1] |
| 903 | 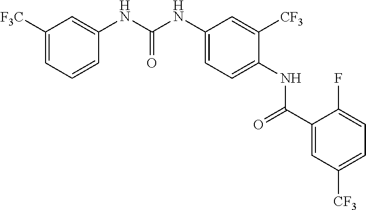 [SMILES: O=C(Nc1cccc(C(F)(F)F)c1)Nc1ccc(NC(=O)c2cc(C(F)(F)F)ccc2F)c(C(F)(F)F)c1] |
| 907 | 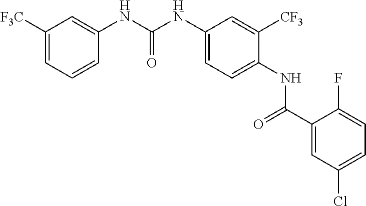 [SMILES: O=C(Nc1cccc(C(F)(F)F)c1)Nc1ccc(NC(=O)c2cc(Cl)ccc2F)c(C(F)(F)F)c1] |
| 911 | 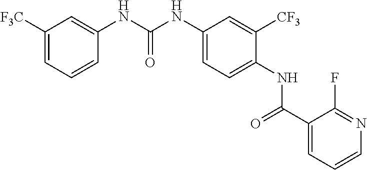 [SMILES: O=C(Nc1cccc(C(F)(F)F)c1)Nc1ccc(NC(=O)c2cccnc2F)c(C(F)(F)F)c1] |
| 952 | 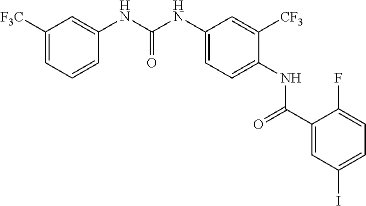 [SMILES: O=C(Nc1cccc(C(F)(F)F)c1)Nc1ccc(NC(=O)c2cc(I)ccc2F)c(C(F)(F)F)c1] |
| 921 | 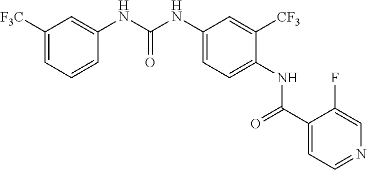 [SMILES: O=C(Nc1cccc(C(F)(F)F)c1)Nc1ccc(NC(=O)c2ccncc2F)c(C(F)(F)F)c1] |
| 971 | 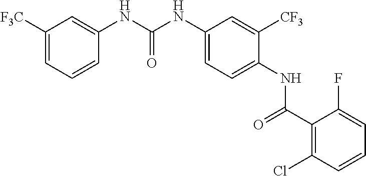 [SMILES: O=C(Nc1cccc(C(F)(F)F)c1)Nc1ccc(NC(=O)c2c(F)cccc2Cl)c(C(F)(F)F)c1] |
| 912 | 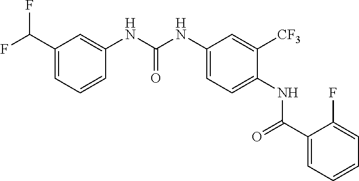 [SMILES: O=C(Nc1cccc(C(F)F)c1)Nc1ccc(NC(=O)c2ccccc2F)c(C(F)(F)F)c1] |
| 923 | 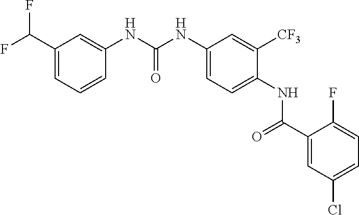 [SMILES: O=C(Nc1cccc(C(F)F)c1)Nc1ccc(NC(=O)c2cc(Cl)ccc2F)c(C(F)(F)F)c1] |
| 930 | 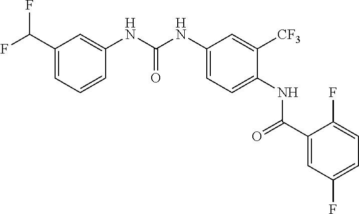 [SMILES: O=C(Nc1cccc(C(F)F)c1)Nc1ccc(NC(=O)c2cc(F)ccc2F)c(C(F)(F)F)c1] |
| 941 | 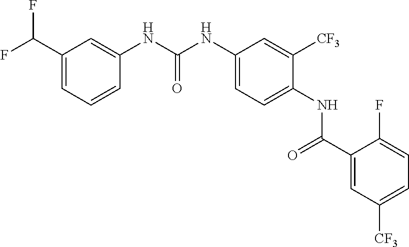 [SMILES: O=C(Nc1cccc(C(F)F)c1)Nc1ccc(NC(=O)c2cc(C(F)(F)F)ccc2F)c(C(F)(F)F)c1] |
| 945 | 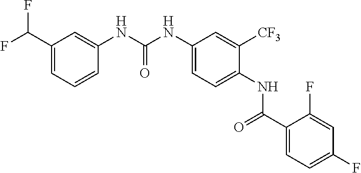 [SMILES: O=C(Nc1cccc(C(F)F)c1)Nc1ccc(NC(=O)c2ccc(F)cc2F)c(C(F)(F)F)c1] |
| 983 | 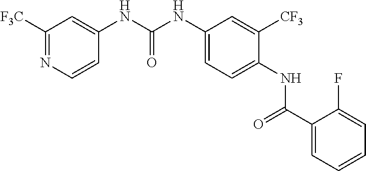 [SMILES: O=C(Nc1ccnc(C(F)(F)F)c1)Nc1ccc(NC(=O)c2ccccc2F)c(C(F)(F)F)c1] |
| 908 | 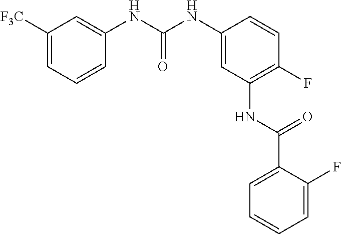 [SMILES: O=C(Nc1cccc(C(F)(F)F)c1)Nc1ccc(F)c(NC(=O)c2ccccc2F)c1] |
| 909 | 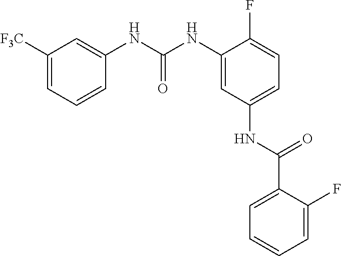 [SMILES: O=C(Nc1cccc(C(F)(F)F)c1)Nc1cc(NC(=O)c2ccccc2F)ccc1F] |
| 910 | 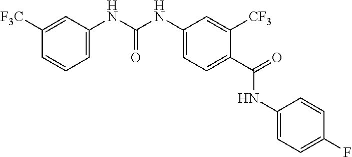 [SMILES: O=C(Nc1cccc(C(F)(F)F)c1)Nc1ccc(C(=O)Nc2ccc(F)cc2)c(C(F)(F)F)c1] |
| 913 | 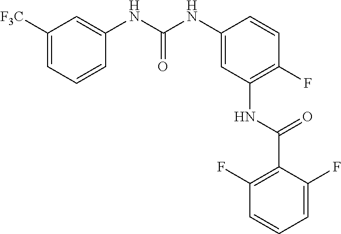 [SMILES: O=C(Nc1cccc(C(F)(F)F)c1)Nc1ccc(F)c(NC(=O)c2c(F)cccc2F)c1] |
| 914 | 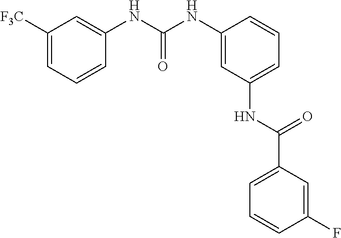 [SMILES: O=C(Nc1cccc(NC(=O)c2cccc(F)c2)c1)Nc1cccc(C(F)(F)F)c1] |
| 915 | 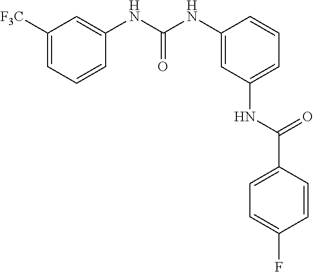 [SMILES: O=C(Nc1cccc(NC(=O)c2ccc(F)cc2)c1)Nc1cccc(C(F)(F)F)c1] |
| 928 | 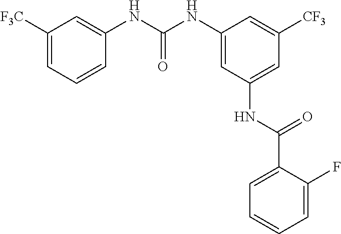 [SMILES: O=C(Nc1cccc(C(F)(F)F)c1)Nc1cc(NC(=O)c2ccccc2F)cc(C(F)(F)F)c1] |
| 929 | 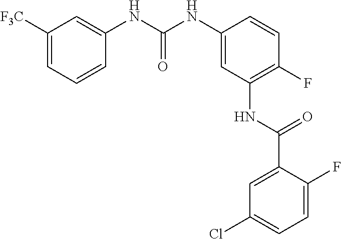 [SMILES: O=C(Nc1cccc(C(F)(F)F)c1)Nc1ccc(F)c(NC(=O)c2cc(Cl)ccc2F)c1] |
| 942 | 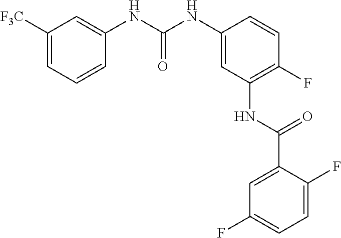 [SMILES: O=C(Nc1cccc(C(F)(F)F)c1)Nc1ccc(F)c(NC(=O)c2cc(F)ccc2F)c1] |
| 943 | 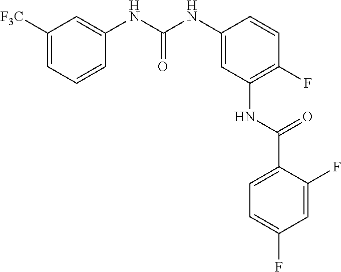 [SMILES: O=C(Nc1cccc(C(F)(F)F)c1)Nc1ccc(F)c(NC(=O)c2ccc(F)cc2F)c1] |
| 944 | 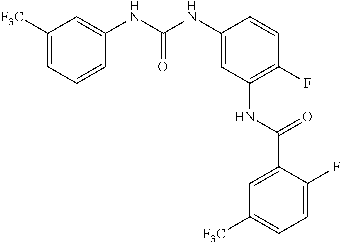 [SMILES: O=C(Nc1cccc(C(F)(F)F)c1)Nc1ccc(F)c(NC(=O)c2cc(C(F)(F)F)ccc2F)c1] |
| 946 | 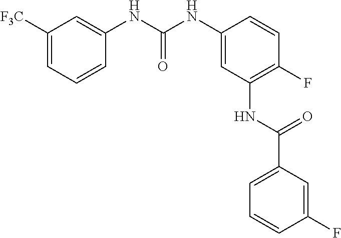 [SMILES: O=C(Nc1cccc(C(F)(F)F)c1)Nc1ccc(F)c(NC(=O)c2cccc(F)c2)c1] |
| 947 | 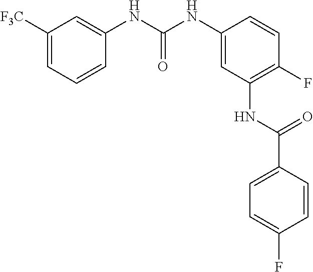 [SMILES: O=C(Nc1cccc(C(F)(F)F)c1)Nc1ccc(F)c(NC(=O)c2ccc(F)cc2)c1] |
| 948 | 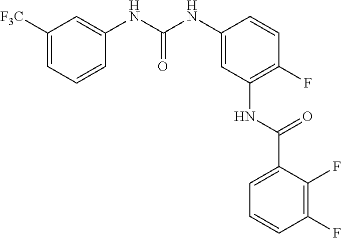 [SMILES: O=C(Nc1cccc(C(F)(F)F)c1)Nc1ccc(F)c(NC(=O)c2cccc(F)c2F)c1] |
| 951 | 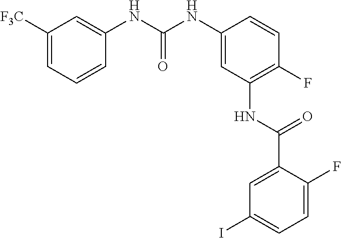 [SMILES: O=C(Nc1cccc(C(F)(F)F)c1)Nc1ccc(F)c(NC(=O)c2cc(I)ccc2F)c1] |
| 954 | 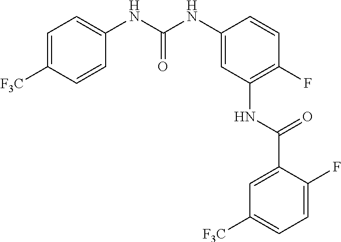 [SMILES: O=C(Nc1ccc(C(F)(F)F)cc1)Nc1ccc(F)c(NC(=O)c2cc(C(F)(F)F)ccc2F)c1] |
| 956 | 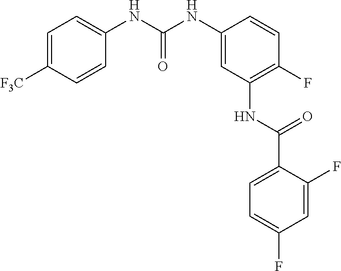 [SMILES: O=C(Nc1ccc(C(F)(F)F)cc1)Nc1ccc(F)c(NC(=O)c2ccc(F)cc2F)c1] |
| 957 | 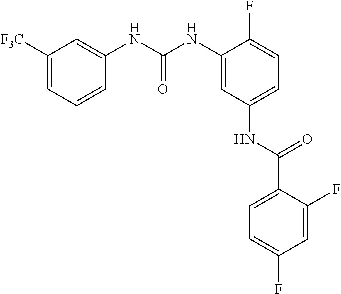 [SMILES: O=C(Nc1cccc(C(F)(F)F)c1)Nc1cc(NC(=O)c2ccc(F)cc2F)ccc1F] |
| 958 | 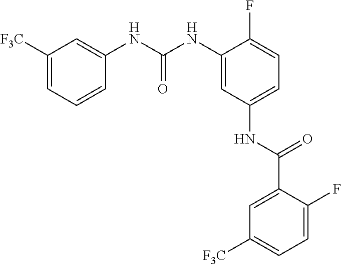 [SMILES: O=C(Nc1cccc(C(F)(F)F)c1)Nc1cc(NC(=O)c2cc(C(F)(F)F)ccc2F)ccc1F] |
| 959 | 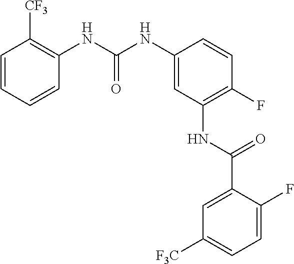 [SMILES: O=C(Nc1ccc(F)c(NC(=O)c2cc(C(F)(F)F)ccc2F)c1)Nc1ccccc1C(F)(F)F] |
| 960 | 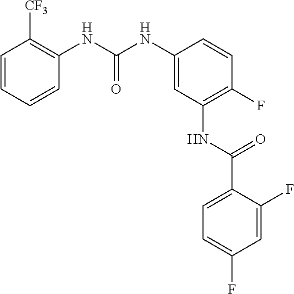 [SMILES: O=C(Nc1ccc(F)c(NC(=O)c2ccc(F)cc2F)c1)Nc1ccccc1C(F)(F)F] |
| 963 | 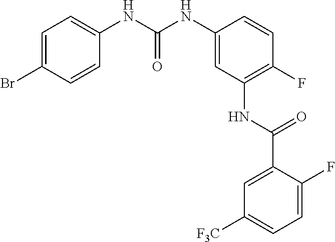 [SMILES: O=C(Nc1ccc(Br)cc1)Nc1ccc(F)c(NC(=O)c2cc(C(F)(F)F)ccc2F)c1] |
| 970 | 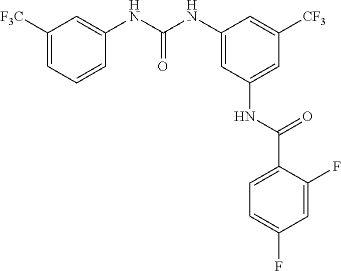 [SMILES: O=C(Nc1cccc(C(F)(F)F)c1)Nc1cc(NC(=O)c2ccc(F)cc2F)cc(C(F)(F)F)c1] |
| 961 | 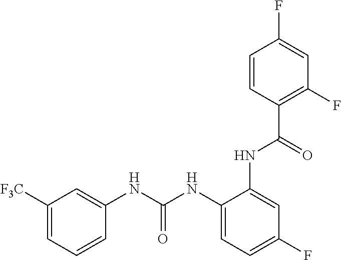 [SMILES: O=C(Nc1cccc(C(F)(F)F)c1)Nc1ccc(F)cc1NC(=O)c1ccc(F)cc1F] |
| 962 | 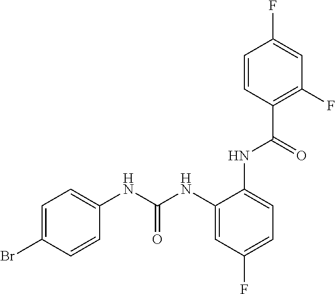 [SMILES: O=C(Nc1ccc(Br)cc1)Nc1cc(F)ccc1NC(=O)c1ccc(F)cc1F] |
| 965 | 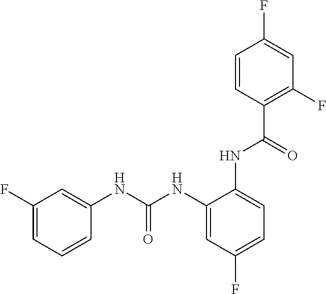 [SMILES: O=C(Nc1cccc(F)c1)Nc1cc(F)ccc1NC(=O)c1ccc(F)cc1F] |
| 968 | 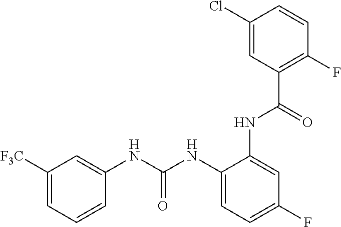 [SMILES: O=C(Nc1cccc(C(F)(F)F)c1)Nc1ccc(F)cc1NC(=O)c1cc(Cl)ccc1F] |
| 969 | 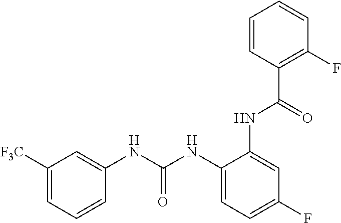 [SMILES: O=C(Nc1cccc(C(F)(F)F)c1)Nc1ccc(F)cc1NC(=O)c1ccccc1F] |
| 974 | 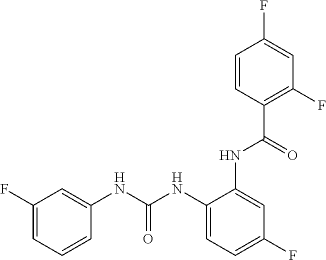 [SMILES: O=C(Nc1cccc(F)c1)Nc1ccc(F)cc1NC(=O)c1ccc(F)cc1F] |
| 975 | 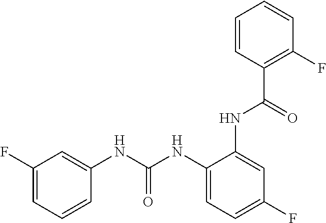 [SMILES: O=C(Nc1cccc(F)c1)Nc1ccc(F)cc1NC(=O)c1ccccc1F] |
| 976 | 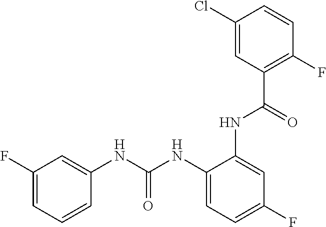 [SMILES: O=C(Nc1cccc(F)c1)Nc1ccc(F)cc1NC(=O)c1cc(Cl)ccc1F] |

5.-6. (canceled)

7. A compound according to claim 2 and having the general formula A2′, which is selected from the group of compounds depicted below

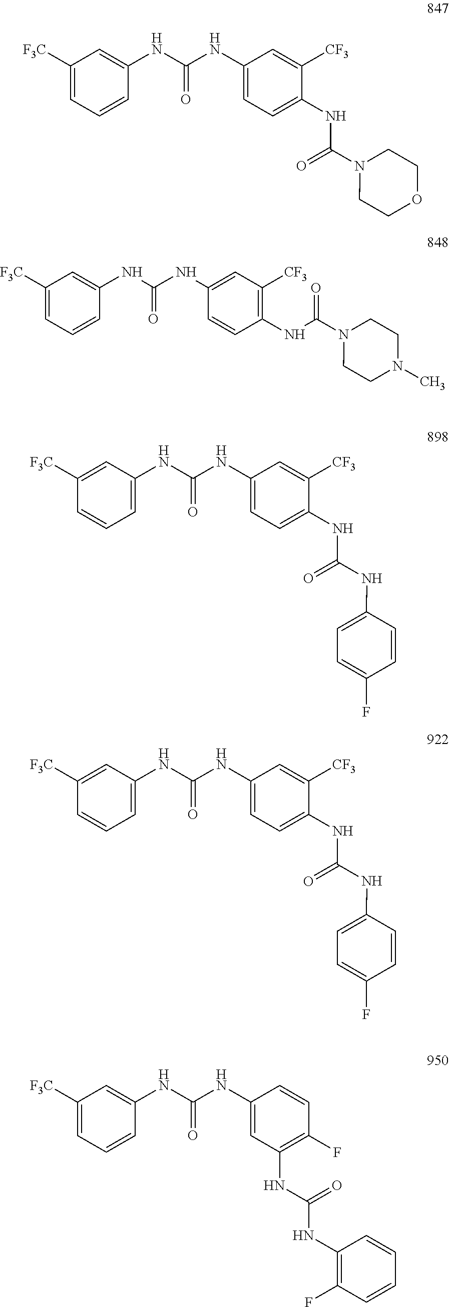

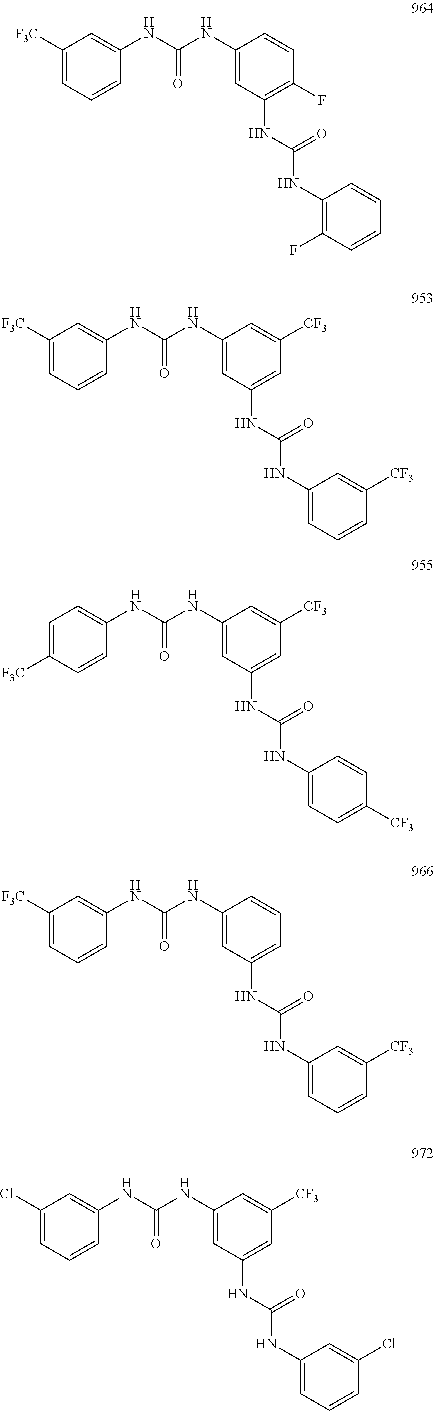

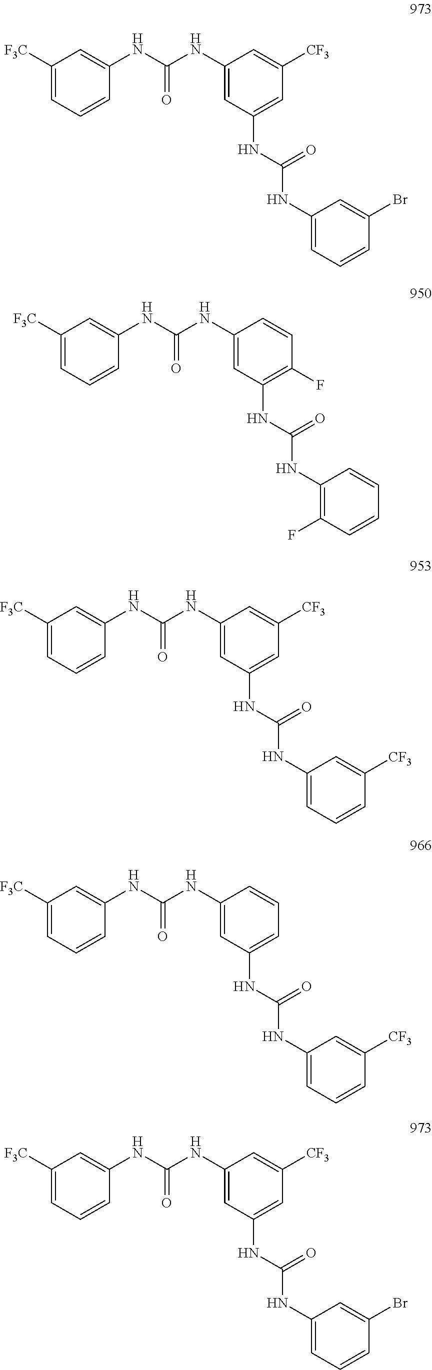

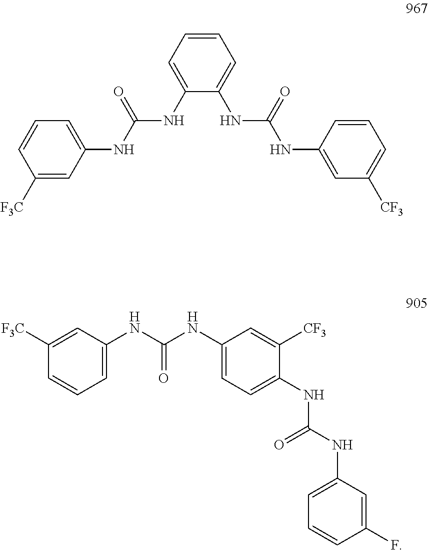

8. (canceled)

9. A compound according to claim 2 and having the general formula A3, which is selected from the group of compounds depicted below

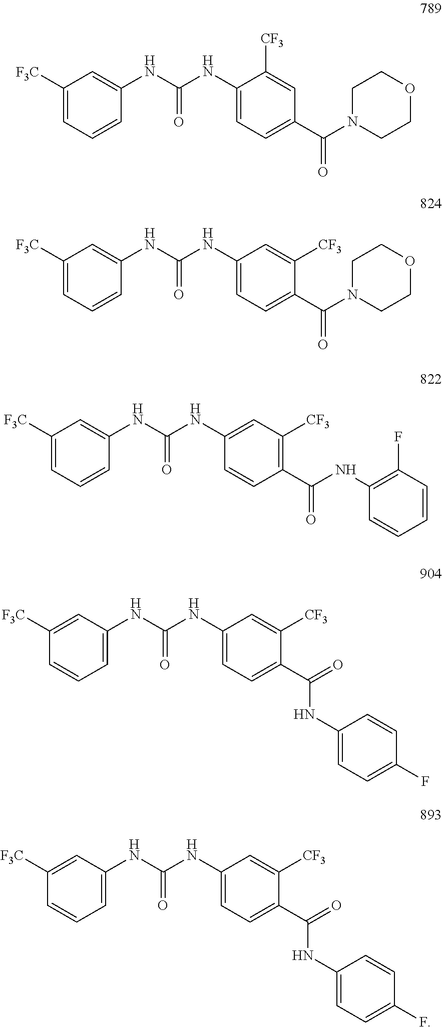

10.-11. (canceled)

12. A compound according to claim 2 and having the general formula A3′ or A3″, which is selected from the group of compounds depicted below

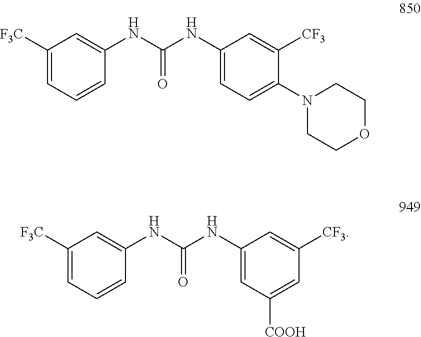

13.-16. (canceled)

17. A compound according to claim 2 and having the general formula A5, which is selected from the group of compounds defined as outlined below

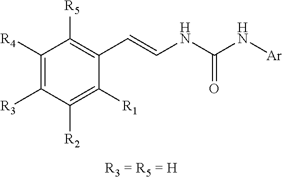

| | Substituents | | | | | | | | |
|---|---|---|---|---|---|---|---|---|---|
| | | | | Ar substituents | | | | | |
| ID | R₁ | R₂ | R₄ | | R₆ | R₇ | R₈ | R₉ | R₁₀ |
| 480 | CF₃ | | CF₃ | B | | | CN | | |
| 481 | CF₃ | | CF₃ | A | | | CN | | |
| 482 | | CF₃ | CF₃ | B | | | CN | | |
| 483 | | CF₃ | CF₃ | A | | | CN | | |
| 487 | CF₃ | | CF₃ | A | | | NO₂ | | |
| 489 | | CF₃ | CF₃ | B | | | NO₂ | | |
| 503 | | CF₃ | CF₃ | B | | | | | |
| 504 | CF₃ | | CF₃ | B | | | | | |
| 510 | | | CF₃ | B | | | CN | | |
| 511 | | | CF₃ | A | | | CN | | |
| 512 | | | | B | | | CN | | |
| 527 | | CF₃ | CF₃ | A | | | | | Br |
| 528 | | CF₃ | CF₃ | D | | | | | |
| 531 | | CF₃ | CF₃ | E | | | | | |
| 533 | | CF₃ | CF₃ | B | Cl | | | | |
| 535 | | CF₃ | CF₃ | B | | F | | | |
| 536 | | CF₃ | CF₃ | B | | | CH₃ | | |
| 537 | | CF₃ | CF₃ | B | | | | | CH₃ |
| 538 | | CF₃ | CF₃ | E | | CF₃ | | | |
| 539 | | CF₃ | CF₃ | B | | Br | | | |
| 540 | | CF₃ | CF₃ | B | | | Br | | |
| 541 | | CF₃ | CF₃ | B | CH₃ | | | | |
| 543 | | CF₃ | CF₃ | E | | Ph | | | |
| 546 | | CF₃ | CF₃ | B | | CH₃ | | | |
| 548 | | CF₃ | CF₃ | C | | | CF₃ | | |
| 549 | | CF₃ | CF₃ | C | | CF₃ | | | |
| 550 | | | CF₃ | B | | F | | | |
| 551 | | | CF₃ | B | Cl | | | | |
| 552 | | | CF₃ | B | | Br | | | |
| 553 | | | CF₃ | B | | | Br | | |
| 554 | | | CF₃ | B | | | CH₃ | | |
| 555 | | | CF₃ | B | CH₃ | | | | |
| 556 | | | CF₃ | B | | CH₃ | | | |
| 557 | | | CF₃ | B | | | | | CH₃ |
| 558 | | | CF₃ | D | | | | | |
| 559 | | | CF₃ | E | | | | | |
| 560 | | | CF₃ | E | | CF₃ | | | |
| 561 | | | CF₃ | E | | Ph | | | |
| 564 | | | CF₃ | C | | | CF₃ | | |
| 583 | CF₃ | | CF₃ | D | | | | | |
| | |
|---|---|
| 542 | 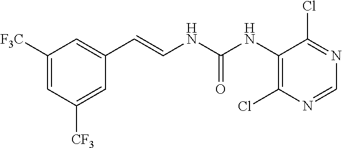 [SMILES: O=C(N/C=C/c1cc(C(F)(F)F)cc(C(F)(F)F)c1)Nc1c(Cl)ncnc1Cl] |
| 544 | 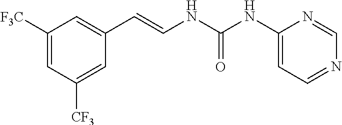 [SMILES: O=C(N/C=C/c1cc(C(F)(F)F)cc(C(F)(F)F)c1)Nc1ccncn1] |
| 545 | 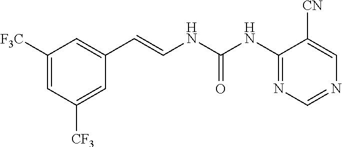 [SMILES: N#Cc1cncnc1NC(=O)N/C=C/c1cc(C(F)(F)F)cc(C(F)(F)F)c1] |
| 562 | 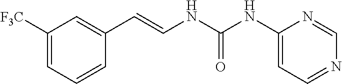 [SMILES: O=C(N/C=C/c1cccc(C(F)(F)F)c1)Nc1ccncn1] |
| 766 | 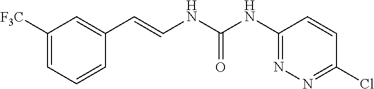 [SMILES: O=C(N/C=C/c1cccc(C(F)(F)F)c1)Nc1ccc(Cl)nn1] |
| 875 | 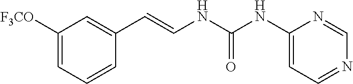 [SMILES: O=C(N/C=C/c1cccc(OC(F)(F)F)c1)Nc1ccncn1] |

Ar =

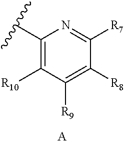

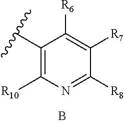

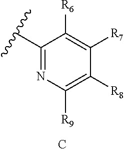

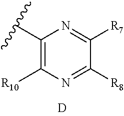

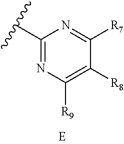

18.-21. (canceled)

22. A compound according to claim 2 and having the general formula A7, which is selected from the group of compounds defined as outlined below

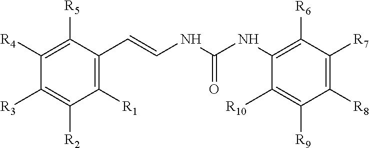

| | Substituents | | | | | | | | | |
|---|---|---|---|---|---|---|---|---|---|---|
| ID | R₁ | R₂ | R₃ | R₄ | R₅ | R₆ | R₇ | R₈ | R₉ | R₁₀ |
| 403 | | | | CF₃ | | | CF₃ | | | |
| 404 | | | | | CF₃ | | CF₃ | NO₂ | | |
| 405 | | | | | CF₃ | | | CN | | |
| 406 | | | | | CF₃ | | CF₃ | | | |
| 407 | | | CF₃ | | | | CF₃ | NO₂ | | |
| 408 | | | CF₃ | | | | CF₃ | | | |
| 409 | | | | CH₃O | | | CF₃ | NO₂ | | |
| 410 | | | | CF₃ | | | CF₃ | CN | | |
| 411 | | | | CH₃O | | | CF₃ | | | |
| 412 | | | | F | | | CF₃ | NO₂ | | |
| 413 | | | | CF₃ | | | | | | |
| 414 | | | | F | | | CF₃ | | | |
| 415 | | | | | | | CF₃ | | | |
| 416 | | | | CF₃ | | | | CN | | |
| 417 | | | | CF₃ | | | CH₃O | | | |
| 421 | | | CF₃ | | | | CF₃ | CN | | |
| 429 | CF₃ | | | | | | CH₃O | | | |
| 430 | | | | CH₃O | | | CH₃O | | | |
| 433 | | | | F | | | CF₃ | CN | | |
| 435 | | | | CF₃ | | | | N(CH₃)₂ | | |
| 436 | CF₃ | | | CF₃ | | | CF₃ | NO₂ | | |
| 437 | | | | CF₃O | | | CF₃ | NO₂ | | |
| 438 | | CF₃ | | CF₃ | | | CF₃ | NO₂ | | |
| 441 | | | | CF₃ | | CH₃O | | NO₂ | | |
| 445 | | | | CF₃ | | CH₃ | | | | |
| 446 | | | | CF₃ | | | | 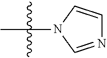 [SMILES: *n1ccnc1] | | |
| 449 | | | | CF₃ | | | | NO₂ | | |
| 456 | | | | NO₂ | | | CF₃ | NO₂ | | |
| 462 | | | CF₃ | | | | | NH₂ | | |
| 463 | | CF₃ | | CF₃ | | | CF₃ | CN | | |
| 464 | CF₃ | | | CF₃ | | | CF₃ | CN | | |
| 468 | CF₃ | | | CF₃ | | | | CN | | |
| 469 | | CF₃ | | CF₃ | | | | CN | | |
| 472 | CF₃ | | | CF₃ | | CH₃O | | NO₂ | | |
| 473 | CF₃ | | | CF₃ | | | | NO₂ | | |
| 474 | CF₃ | | | CF₃ | | CH₃ | | NO₂ | | |
| 488 | CF₃ | | | CF₃ | | | Cl | CN | | |
| 490 | | CF₃ | | CF₃ | | | Cl | CN | | |
| 723 | | CF₃ | | CF₃ | | | | N(CH₃)₂ | | |

23.-26. (canceled)

27. A compound according to claim 2 and having the general formula A9, which is selected from the group of compounds depicted below

| ID | Structure |
|---|---|
| 418 | 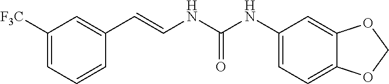 [SMILES: O=C(N/C=C/c1cccc(C(F)(F)F)c1)Nc1ccc2c(c1)OCO2] |
| 427 | 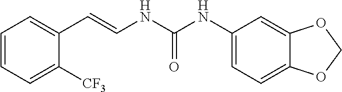 [SMILES: O=C(N/C=C/c1ccccc1C(F)(F)F)Nc1ccc2c(c1)OCO2] |
| 431 | 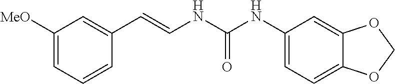 [SMILES: COc1cccc(/C=C/NC(=O)Nc2ccc3c(c2)OCO3)c1] |
| 432 | 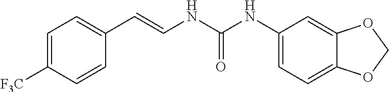 [SMILES: O=C(N/C=C/c1ccc(C(F)(F)F)cc1)Nc1ccc2c(c1)OCO2] |
| 515 | 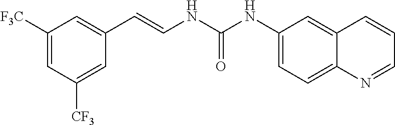 [SMILES: O=C(N/C=C/c1cc(C(F)(F)F)cc(C(F)(F)F)c1)Nc1ccc2ncccc2c1] |
| 516 | 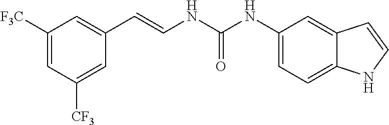 [SMILES: O=C(N/C=C/c1cc(C(F)(F)F)cc(C(F)(F)F)c1)Nc1ccc2[nH]ccc2c1] |
| 517 | 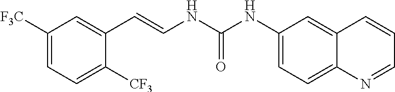 [SMILES: O=C(N/C=C/c1cc(C(F)(F)F)ccc1C(F)(F)F)Nc1ccc2ncccc2c1] |
| 518 | 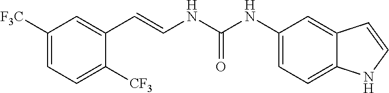 [SMILES: O=C(N/C=C/c1cc(C(F)(F)F)ccc1C(F)(F)F)Nc1ccc2[nH]ccc2c1] |
| 519 | 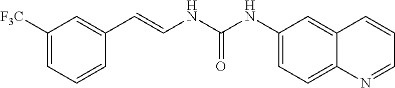 [SMILES: O=C(N/C=C/c1cccc(C(F)(F)F)c1)Nc1ccc2ncccc2c1] |
| 520 | 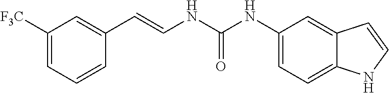 [SMILES: O=C(N/C=C/c1cccc(C(F)(F)F)c1)Nc1ccc2[nH]ccc2c1] |
| 523 | 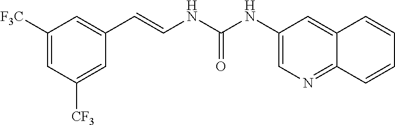 [SMILES: O=C(N/C=C/c1cc(C(F)(F)F)cc(C(F)(F)F)c1)Nc1cnc2ccccc2c1] |
| 524 | 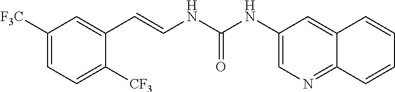 [SMILES: O=C(N/C=C/c1cc(C(F)(F)F)ccc1C(F)(F)F)Nc1cnc2ccccc2c1] |
| 525 | 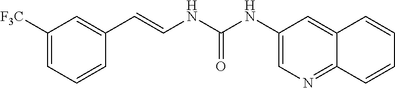 [SMILES: O=C(N/C=C/c1cccc(C(F)(F)F)c1)Nc1cnc2ccccc2c1] |

28.-30. (canceled)

31. A compound according to claim 2 and having the general formula A11, which is selected from the group of compounds depicted below

| ID | Structure |
|---|---|
| 419 | 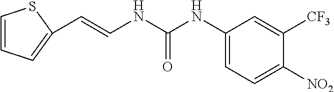 [SMILES: O=C(N/C=C/c1cccs1)Nc1ccc([N+](=O)[O-])c(C(F)(F)F)c1] |
| 420 | 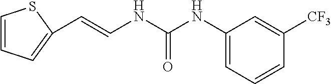 [SMILES: O=C(N/C=C/c1cccs1)Nc1cccc(C(F)(F)F)c1] |
| 424 | 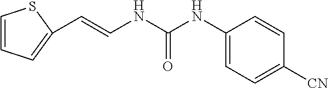 [SMILES: N#Cc1ccc(NC(=O)N/C=C/c2cccs2)cc1] |
| 425 | 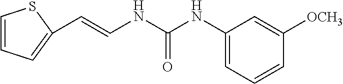 [SMILES: COc1cccc(NC(=O)N/C=C/c2cccs2)c1] |
| 426 | 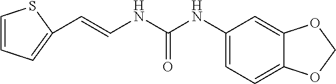 [SMILES: O=C(N/C=C/c1cccs1)Nc1ccc2c(c1)OCO2] |
| 428 | 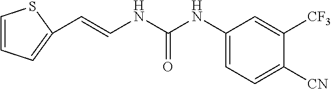 [SMILES: N#Cc1ccc(NC(=O)N/C=C/c2cccs2)cc1C(F)(F)F] |
| 434 | 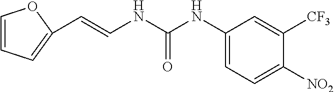 [SMILES: O=C(N/C=C/c1ccco1)Nc1ccc([N+](=O)[O-])c(C(F)(F)F)c1] |
| 443 | 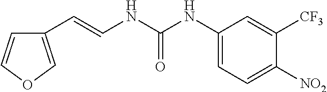 [SMILES: O=C(N/C=C/c1ccoc1)Nc1ccc([N+](=O)[O-])c(C(F)(F)F)c1] |
| 444 | 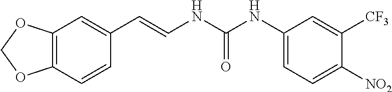 [SMILES: O=C(N/C=C/c1ccc2c(c1)OCO2)Nc1ccc([N+](=O)[O-])c(C(F)(F)F)c1] |
| 447 | 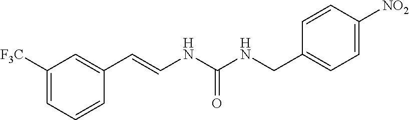 [SMILES: O=C(N/C=C/c1cccc(C(F)(F)F)c1)NCc1ccc([N+](=O)[O-])cc1] |
| 450 | 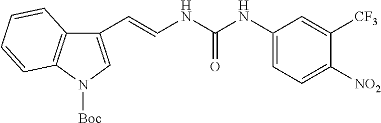 [SMILES: CC(C)(C)OC(=O)n1cc(/C=C/NC(=O)Nc2ccc([N+](=O)[O-])c(C(F)(F)F)c2)c2ccccc21] |
| 453 | 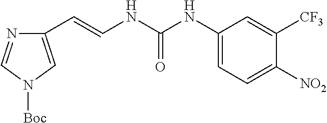 [SMILES: CC(C)(C)OC(=O)n1cnc(/C=C/NC(=O)Nc2ccc([N+](=O)[O-])c(C(F)(F)F)c2)c1] |
| 454 | 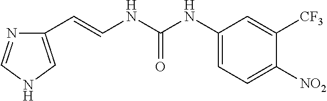 [SMILES: O=C(N/C=C/c1c[nH]cn1)Nc1ccc([N+](=O)[O-])c(C(F)(F)F)c1] |
| 459 | 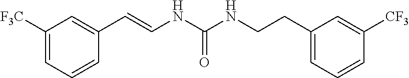 [SMILES: O=C(N/C=C/c1cccc(C(F)(F)F)c1)NCCc1cccc(C(F)(F)F)c1] |
| 460 | 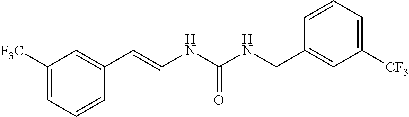 [SMILES: O=C(N/C=C/c1cccc(C(F)(F)F)c1)NCc1cccc(C(F)(F)F)c1] |
| 461 | 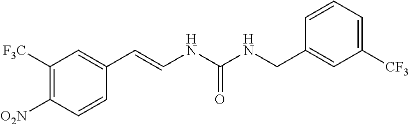 [SMILES: O=C(N/C=C/c1ccc([N+](=O)[O-])c(C(F)(F)F)c1)NCc1cccc(C(F)(F)F)c1] |
| 633 | 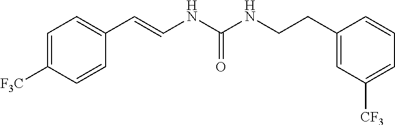 [SMILES: O=C(N/C=C/c1ccc(C(F)(F)F)cc1)NCCc1cccc(C(F)(F)F)c1] |
| 634 | 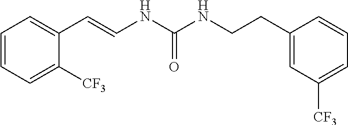 [SMILES: O=C(N/C=C/c1ccccc1C(F)(F)F)NCCc1cccc(C(F)(F)F)c1] |
| 635 | 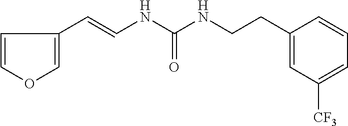 [SMILES: O=C(N/C=C/c1ccoc1)NCCc1cccc(C(F)(F)F)c1] |
| 642 | 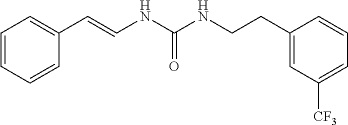 [SMILES: O=C(N/C=C/c1ccccc1)NCCc1cccc(C(F)(F)F)c1] |

32.-33. (canceled)

34. A compound according to claim 2 and having the general formula A12, which is

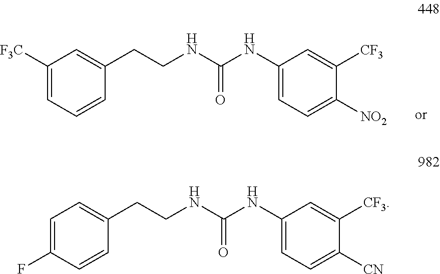

35.-36. (canceled)

37. A compound according to claim 2 and having the general formula A14, which is selected from the group of compounds depicted below

| ID | Structure |
|---|---|
| 534 | 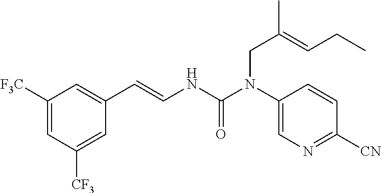 [SMILES: CC/C=C(\C)CN(C(=O)N/C=C/c1cc(C(F)(F)F)cc(C(F)(F)F)c1)c1ccc(C#N)nc1] |
| 547 | 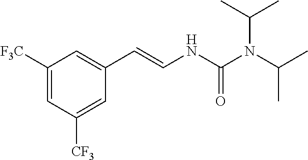 [SMILES: CC(C)N(C(=O)N/C=C/c1cc(C(F)(F)F)cc(C(F)(F)F)c1)C(C)C] |
| 563 | 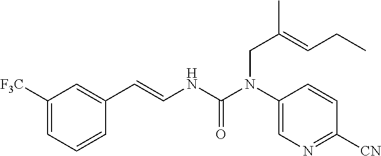 [SMILES: CC/C=C(\C)CN(C(=O)N/C=C/c1cccc(C(F)(F)F)c1)c1ccc(C#N)nc1] |
| 591 | 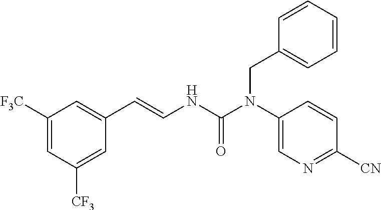 [SMILES: N#Cc1ccc(N(Cc2ccccc2)C(=O)N/C=C/c2cc(C(F)(F)F)cc(C(F)(F)F)c2)cn1] |
| 620 | 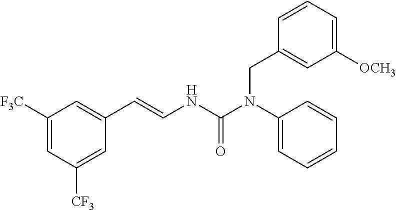 [SMILES: COc1cccc(CN(C(=O)N/C=C/c2cc(C(F)(F)F)cc(C(F)(F)F)c2)c2ccccc2)c1] |
| 621 | 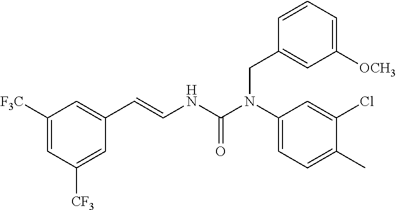 [SMILES: COc1cccc(CN(C(=O)N/C=C/c2cc(C(F)(F)F)cc(C(F)(F)F)c2)c2ccc(C)c(Cl)c2)c1] |
| 622 | 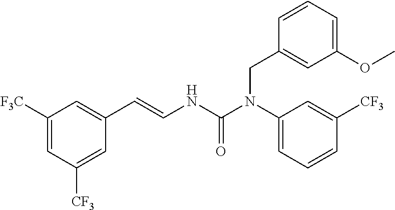 [SMILES: COc1cccc(CN(C(=O)N/C=C/c2cc(C(F)(F)F)cc(C(F)(F)F)c2)c2cccc(C(F)(F)F)c2)c1] |
| 623 | 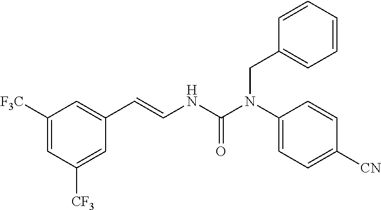 [SMILES: N#Cc1ccc(N(Cc2ccccc2)C(=O)N/C=C/c2cc(C(F)(F)F)cc(C(F)(F)F)c2)cc1] |

38.-40. (canceled)

41. A compound according to claim 2 and having the general formula A16, which is selected from the group of compounds depicted in the table below

| ID. | Structure |
|---|---|
| 804 | 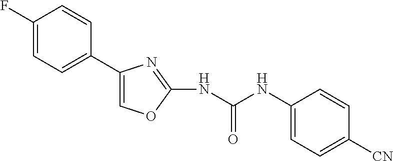 [SMILES: N#Cc1ccc(NC(=O)Nc2nc(-c3ccc(F)cc3)co2)cc1] |
| 790 | 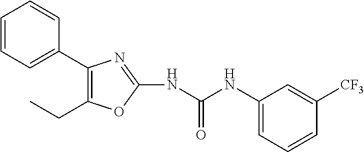 [SMILES: CCc1oc(NC(=O)Nc2cccc(C(F)(F)F)c2)nc1-c1ccccc1] |
| 791 | 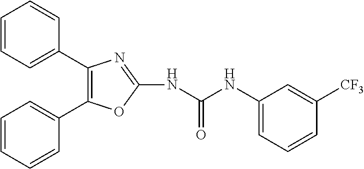 [SMILES: O=C(Nc1cccc(C(F)(F)F)c1)Nc1nc(-c2ccccc2)c(-c2ccccc2)o1] |
| 797 | 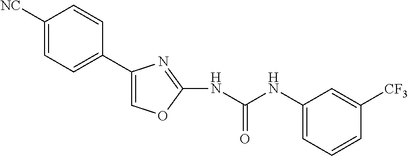 [SMILES: N#Cc1ccc(-c2coc(NC(=O)Nc3cccc(C(F)(F)F)c3)n2)cc1] |
| 798 | 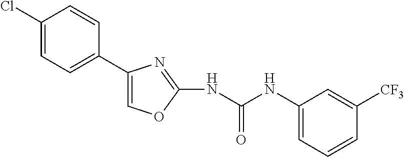 [SMILES: O=C(Nc1cccc(C(F)(F)F)c1)Nc1nc(-c2ccc(Cl)cc2)co1] |
| 799 | 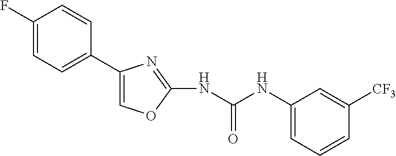 [SMILES: O=C(Nc1cccc(C(F)(F)F)c1)Nc1nc(-c2ccc(F)cc2)co1] |
| 803 | 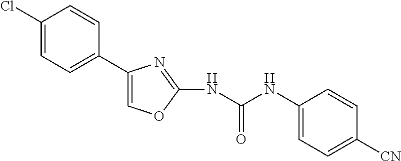 [SMILES: N#Cc1ccc(NC(=O)Nc2nc(-c3ccc(Cl)cc3)co2)cc1] |
| 805 | 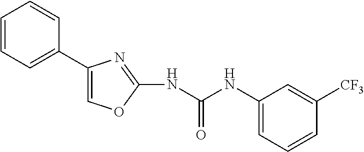 [SMILES: O=C(Nc1cccc(C(F)(F)F)c1)Nc1nc(-c2ccccc2)co1] |
| 802 | 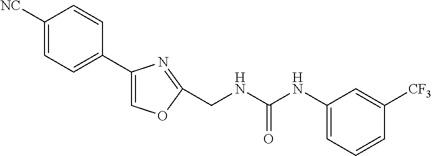 [SMILES: N#Cc1ccc(-c2coc(CNC(=O)Nc3cccc(C(F)(F)F)c3)n2)cc1] |
| 783 | 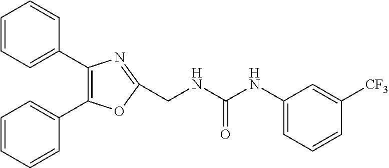 [SMILES: O=C(NCc1nc(-c2ccccc2)c(-c2ccccc2)o1)Nc1cccc(C(F)(F)F)c1] |
| 788 | 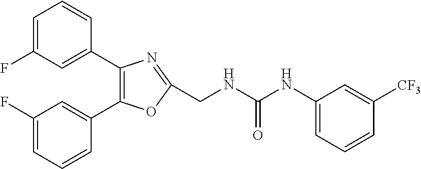 [SMILES: O=C(NCc1nc(-c2cccc(F)c2)c(-c2cccc(F)c2)o1)Nc1cccc(C(F)(F)F)c1] |
| 885 | 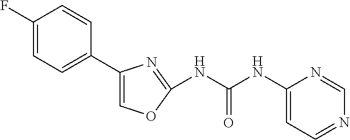 [SMILES: O=C(Nc1ccncn1)Nc1nc(-c2ccc(F)cc2)co1] |

42.-44. (canceled)

45. A compound according to claim 2 and having the general formula A18, which is selected from the group of compounds defined as outlined below
566 Analogues of Formula (I)

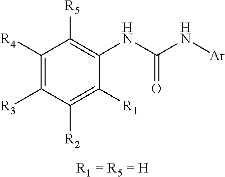

| | Substituents | | | | | | | | |
|---|---|---|---|---|---|---|---|---|---|
| | | | | Ar | | | | | |
| ID | R₂ | R₃ | R₄ | | R₆ | R₇ | R₈ | R₉ | R₁₀ |
| 484 | | | CF₃ | A | | | CN | | |
| 486 | | | CF₃ | B | | | CN | | |
| 491 | | | CF₃ | A | | | NO₂ | | |
| 495 | CF₃ | | CF₃ | A | | | CN | | |
| 496 | CF₃ | | CF₃ | A | | | NO₂ | | |
| 498 | | CF₃ | | B | | | CN | | |
| 499 | | CF₃ | | A | | | CN | | |
| 501 | | CF₃ | | A | | | NO₂ | | |
| 506 | CF₃ | | CF₃ | B | | | | | |
| 507 | | CF₃ | | B | | | | | |
| 565 | CF₃ | | CF₃ | B | Cl | | | | |
| 566 | CF₃ | | CF₃ | B | | | Br | | |
| 567 | | | CF₃ | B | | F | | | |
| 568 | | | CF₃ | B | Cl | | | | |
| 569 | | | CF₃ | B | | | Br | | |
| 570 | | | CF₃ | B | CH₃ | | | | |
| 571 | | | CF₃ | D | | | | | |
| 572 | | | CF₃ | E | | Ph | | | |
| 573 | CF₃ | | CF₃ | B | | F | | | |
| 575 | | | CF₃ | C | | | CF₃ | | |
| 576 | CF₃ | | CF₃ | D | | | | | |
| 579 | CF₃ | | CF₃ | B | | | CH₃ | | |
| 580 | | | CF₃ | E | | CF₃ | | | |
| 584 | CF₃ | | CF₃ | E | | CF₃ | | | |
| 739 | CF₃ | | | B | | | F | | |
| 740 | CF₃ | | | B | | | Cl | | |
| 741 | CF₃ | | | B | | Cl | Cl | | |
| 754 | CF₃ | | CF₃ | B | | | F | | |
| 755 | CF₃ | | CF₃ | B | | | Cl | | |
| 758 | CF₃ | | CF₃ | B | | Cl | Cl | | |
| 763 | CF₃ | | CF₃ | B | CH₃ | | | | Cl |
| 764 | CF₃ | | CF₃ | B | CH₃ | | F | | |
| 773 | CN | | | | | | Br | | |
| | |
|---|---|
| 522 | 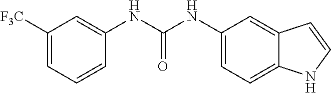 [SMILES: O=C(Nc1cccc(C(F)(F)F)c1)Nc1ccc2[nH]ccc2c1] |
| 530 | 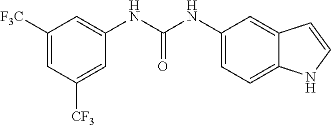 [SMILES: O=C(Nc1cc(C(F)(F)F)cc(C(F)(F)F)c1)Nc1ccc2[nH]ccc2c1] |
| 574 | 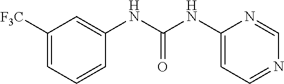 [SMILES: O=C(Nc1cccc(C(F)(F)F)c1)Nc1ccncn1] |
| 578 | 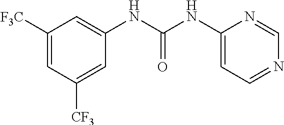 [SMILES: O=C(Nc1cc(C(F)(F)F)cc(C(F)(F)F)c1)Nc1ccncn1] |
| 737 | 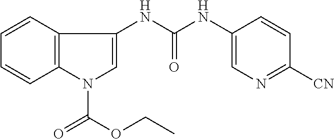 [SMILES: CCOC(=O)n1cc(NC(=O)Nc2ccc(C#N)nc2)c2ccccc21] |
| 738 | 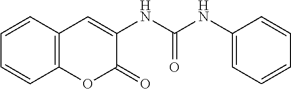 [SMILES: O=C(Nc1ccccc1)Nc1cc2ccccc2oc1=O] |
| 744 | 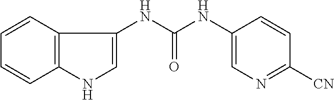 [SMILES: N#Cc1ccc(NC(=O)Nc2c[nH]c3ccccc23)cn1] |
| 753 | 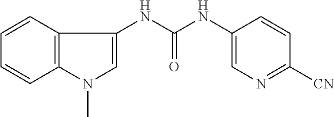 [SMILES: Cn1cc(NC(=O)Nc2ccc(C#N)nc2)c2ccccc21] |

Ar =

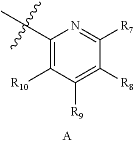

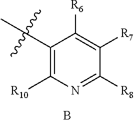

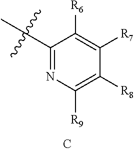

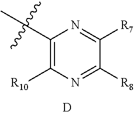

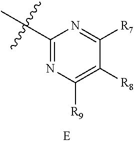

46.-48. (canceled)

49. A compound according to claim 2 and having the general formula A19, which is selected from the group of compounds defined as outlined below
566 Analogues of Formula (II)

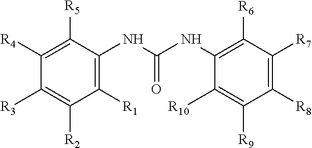

| | Substituents | | | | | | | | | |
|---|---|---|---|---|---|---|---|---|---|---|
| ID | R₁ | R₂ | R₃ | R₄ | R₅ | R₆ | R₇ | R₈ | R₉ | R₁₀ |
| 442 | | | | CF₃ | | | CF₃ | NO₂ | | |
| 465 | | | NO₂ | CF₃ | | | CF₃ | CN | | |
| 467 | | | | CF₃ | | | CF₃ | CN | | |
| 492 | | | | CF₃ | | | Cl | CN | | |
| 494 | | CF₃ | | CF₃ | | | CF₃ | CN | | |
| 500 | | CF₃ | | CF₃ | | | Cl | CN | | |
| 502 | | | CF₃ | | | | Cl | CN | | |
| 509 | | | CF₃ | | | | CF₃ | CN | | |
| 646 | | OCH₃ | | | | | Cl | CN | | |
| 647 | | Cl | | | | | Cl | CN | | |
| 680 | | | | | | | Cl | CN | | |
| 701 | | OCH₃ | | | | | CF₃ | CN | | |
| 702 | | | | | | | CF₃ | CN | | |
| 703 | | CH₃ | | | | | CF₃ | CN | | |
| 704 | | F | | | | | CF₃ | CN | | |
| 705 | | Cl | | | | | CF₃ | CN | | |
| 706 | CF₃ | | | CF₃ | | | CF₃ | CN | | |
| 736 | | CF₃ | | | | | CF₃ | NH₂ | | |
| 745 | | CF₃ | | | | | | NH₂ | | |
| 772 | | CN | | | | | Cl | CN | | |
| 774 | | CN | | | | | CN | CN | | |
| 792 | | CF₃ | | CF₃ | | | F | CN | | |
| 829 | | CF₃ | F | | | | CF₃ | NO₂ | | |
| 887 | | CF₃ | | OCH₃ | | | CF₃ | | | |

50.-52. (canceled)

53. A compound according to claim 2 and having the general formula B2, which is selected from the group of compounds depicted below

| ID | Structure |
|---|---|
| 439 | 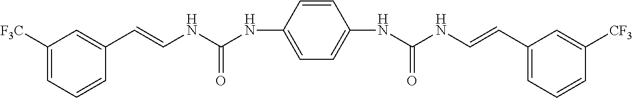 [SMILES: O=C(N/C=C/c1cccc(C(F)(F)F)c1)Nc1ccc(NC(=O)N/C=C/c2cccc(C(F)(F)F)c2)cc1] |
| 440 | 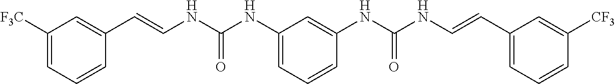 [SMILES: O=C(N/C=C/c1cccc(C(F)(F)F)c1)Nc1cccc(NC(=O)N/C=C/c2cccc(C(F)(F)F)c2)c1] |
| 451 | 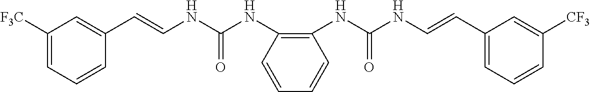 [SMILES: O=C(N/C=C/c1cccc(C(F)(F)F)c1)Nc1ccccc1NC(=O)N/C=C/c1cccc(C(F)(F)F)c1] |
| 452 | 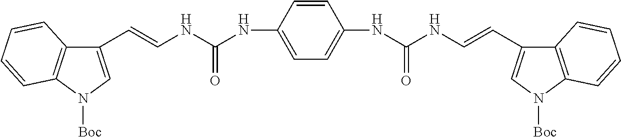 [SMILES: CC(C)(C)OC(=O)n1cc(/C=C/NC(=O)Nc2ccc(NC(=O)N/C=C/c3cn(C(=O)OC(C)(C)C)c4ccccc34)cc2)c2ccccc21] |
| 455 | 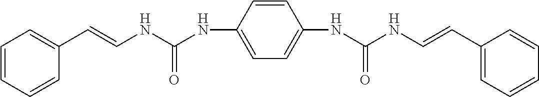 [SMILES: O=C(N/C=C/c1ccccc1)Nc1ccc(NC(=O)N/C=C/c2ccccc2)cc1] |
| 457 | 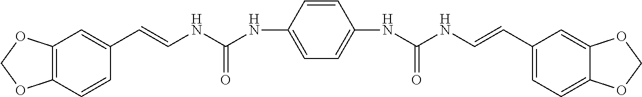 [SMILES: O=C(N/C=C/c1ccc2c(c1)OCO2)Nc1ccc(NC(=O)N/C=C/c2ccc3c(c2)OCO3)cc1] |
| 458 | 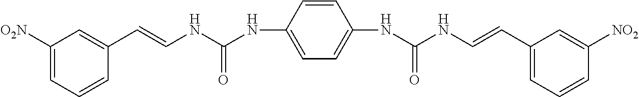 [SMILES: O=C(N/C=C/c1cccc([N+](=O)[O-])c1)Nc1ccc(NC(=O)N/C=C/c2cccc([N+](=O)[O-])c2)cc1] |
| 466 | 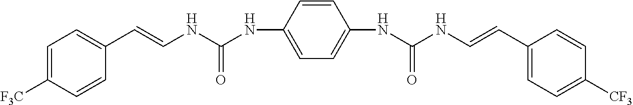 [SMILES: O=C(N/C=C/c1ccc(C(F)(F)F)cc1)Nc1ccc(NC(=O)N/C=C/c2ccc(C(F)(F)F)cc2)cc1] |
| 532 | 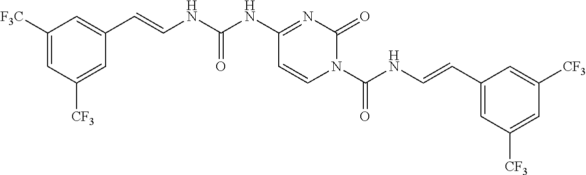 [SMILES: O=C(N/C=C/c1cc(C(F)(F)F)cc(C(F)(F)F)c1)Nc1ccn(C(=O)N/C=C/c2cc(C(F)(F)F)cc(C(F)(F)F)c2)c(=O)n1] |

54.-55. (canceled)

56. A compound according to claim 1, which targets the N-terminal domain of the androgen receptor (AR-NTD); and/or which targets mutants of the androgen receptor; and/or which targets androgen receptor variants; and/or which targets cancer cells lacking any androgen receptor (AR negative cells).

57.-59. (canceled)

60. A pharmaceutical composition comprising a compound as defined in claim 1, and a pharmaceutically acceptable carrier.

61. A method of treating a medical condition that may or may not involve hormones, comprising administering to a subject a therapeutically effective amount of a compound as defined in claim 1; optionally the medical condition is selected from: androgen-dependent diseases or disorders and androgen receptor-mediated diseases or disorders.

62.-80. (canceled)

81. A method according to claim 61, further comprising treating the subject with a second cancer therapy; optionally the subject is a human or a non-human animal, wherein:
the compound is administered intravenously, intra-arterially, subcutaneously, topically or intramuscularly; and/or
the cancer is multi-drug resistant, metastatic and/or recurrent; and/or
the method comprises inhibiting cancer growth, killing cancer cells, reducing tumor burden, reducing tumor size, improving the subject's quality of life and/or prolonging the subject's length of life.

82.-86. (canceled)
